SEQUENCE LISTING (37 sequences)

SEQ ID NO: 1 - protein, 30 aa, Homo sapiens
NPMYNAVSNADLMDFKNLLDHLEEKMPLED

SEQ ID NO: 2 - protein, 37 aa, Homo sapiens
EVVPPQVLSEPNEEAGAALSPLPEVPPWTGEVSPAQR

SEQ ID NO: 3 - protein, 20 aa, Homo sapiens
SSDRSALLKSKLRALLTAPR

SEQ ID NO: 4 - protein, 28 aa, Homo sapiens
SLRRSSCFGGRMDRIGAQSGLGCNSFRY

SEQ ID NO: 5 - protein, 29 aa, Mus musculus
GSPWDPSDRSALLKSKLRALLAGPRSLRR

SEQ ID NO: 6 - protein, 37 aa, Mus musculus
VSNTDLMDFKNLLDHLEEKMPVEDEVMPPQALSEQTE

SEQ ID NO: 7 - protein, 151 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 8 - DNA, 35 bp, Mus musculus
gacggcaagcttactatgggcagcccctgggaccc

SEQ ID NO: 9 - DNA, 33 bp, Mus musculus
acccccctcgagttattatcttcgtaggctccg

SEQ ID NO: 10 - DNA, 33 bp, Mus musculus
aatcctaagcttagtatggtgtccaacacagat

SEQ ID NO: 11 - DNA, 41 bp, Mus musculus
tgcgaactcgagttactcagtctgctcactcagggcctgcg

SEQ ID NO: 12 - DNA, 93 bp, Mus musculus
atgggcagcccctgggacccctccgatagatctgccctcttgaaaagcaaactgagggctctgctcgctggccctcggagcctacgaagataa

SEQ ID NO: 13 - DNA, 117 bp, Mus musculus
atggtgtccaacacagatctgatggatttcaagaacctgctagaccacctggaggagaagatgccggtagaagatgaggtcatgcccccgcaggccctgagtgagcagactgagtaa

SEQ ID NO: 14 - DNA, 845 bp, Homo sapiens
tggcgagggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccgtgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccggtactgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaagtggtttgaaagtgaataaacttcagcaccatggac

SEQ ID NO: 15 - DNA, 2583 bp, Homo sapiens
ggatccatttgtctcgggctgctggctgcctgccatttcctcctctccacccttatttggaggccctgacagctgagccacaaacaaaccaggggagctgggcaccagcaagcgtcaccctctgtttccccgcacggtaccagcgtcgaggagaaagaatcctgaggcacggcggtgagataaccaaggactcttttttactcttctcacacctttgaagtgggagcctcttgagtcaaatcagtaagaatgcggctcttgcagctgagggtctggggggctgttggggctgcccaaggcagagaggggctgtgacaagccctgcggatgataactttaaaagggcatctcctgctggcttctcacttggcagctttatcactgcaagtgacagaatggggagggttctgtctctcctgcgtgcttggagagctggggggctataaaaagaggcggcactgggcagctgggagacagggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccgtgtccaacgcagacctgatggatttcaaggtagggccaggaaagcgggtgcagtctggggccagggggctttctgatgctgtgctcactcctcttgatttcctccaagtcagtgaggtttatccctttccctgtattttccttttctaaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccgggtaagaggaactggggatggaaatgggatgggatggacactactgggagacaccttcagcaggaaagggaccaatgcagaagctcattccctctcaagtttctgccccaacacccagagtgccccatgggtgtcaggacatgccatctattgtccttagctagtctgctgagaaaatgcttaaaaaaaaaagggggggggctgggcacggtcgtcacgcctgtaatcccagcactttgggaggccaggcagcggatcatgaggtcaagagatcaagactatcctggccaacatggtgaaaccccagctctactaaaaatacaaaaattagctgggtgtgtggcgggcacctgtactctcagctacttgggaggctgaggcaggagaatcacttgaacccaggaggcagaggttgcagtgagcagagatcacgccactgcagtccagcctaggtgatagagcgagactgtctcaaaaaaaaaaaaaaaaggccaggcgcggtggctcacgcctgtaatcccagcgctttgggaggccaaggcgggtggatcacgaggtcaggagatggagaccatcctggctaacacggtgaaaccccgtctctactaaaaatacaaaaaattagccaggcgtggtggcaggcgcctgtaagtcctagctactccggaggctgaggcaggagaatggcgtgaacccgggaggcggagcttgcagtgagcagagatggcaccactgcactccagcctgggcgacagagcaagactccgtctcaaaaaaaaaaaaaaaaaaagcaactgccactagcactgggaaattaaaatattcatagagccaagttatctttgcatggctgattagcagttcatattcctccccagaattgcaagatcctgaagggcttaagtgaaatttactctgatgagtaacttgcttatcaattcatgaagctcagagggtcatcaggctggggtgggggccggtgggaagcaggtggtcagtaatcaagttcagaggatgggcacactcatacatgaagctgacttttccaggacagccaggtcaccaagccagatatgtctgtgttctctttgcagtactgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaagtggtttgaaagtgaataaacttcagcaccatggacagaagacaaatgcctgcgttggtgtgctttctttcttcttgggaagagaattc

SEQ ID NO: 16 - protein, 152 aa, Mus musculus
MGSFSITLGFFLVLAFWLPGHIGANPVYSAVSNTDLMDFKNLLDHLEEKMPVEDEVMPPQALSEQTEEAGAALSSLPEVPPWTGEVNPPLRDGSALGRSPWDPSDRSALLKSKLRALLAGPRSLRRSSCFGGRIDRIGAQSGLGCNSFRYRR

SEQ ID NO: 17 - DNA, 878 bp, Mus musculus
caaaagctgagagagagagagaaagaaaccagagtgggcagagacagcaaacatcagatcgtgccccgacccacgccagcatgggctccttctccatcaccctgggcttcttcctcgtcttggccttttggcttccaggccatattggagcaaatcctgtgtacagtgcggtgtccaacacagatctgatggatttcaagaacctgctagaccacctggaggagaagatgccggtagaagatgaggtcatgcccccgcaggccctgagtgagcagactgaggaagcaggggccgcacttagctccctccccgaggtgcctccctggactggggaggtcaacccacctctgagagacggcagtgctctagggcgcagcccctgggacccctccgatagatctgccctcttgaaaagcaaactgagggctctgctcgctggccctcggagcctacgaagatccagctgcttcgggggtaggattgacaggattggagcccagagtggactaggctgcaacagcttccggtaccgaagataacagccaaggaggaaaaggcagtcgattctgcttgagcagatcgcaaaagatcctaagcccttgtggtgtgtcacgcagcttggtcacattgccactgtggcgtggtgaacaccctcctggagctgcggcttcctgccttcatctatcacgatcgatgttaaatgtagatgagtggtctagtggggtcttgcctctcccactctgcatattaaggtagatcctcacccttttcagaaagcagttggaaaaaaaaaaaaagaataaacttcagcaccaaggacagacgccgaggccctgatgtgcttctttggcttctgccctcagttctttgctctcccc

SEQ ID NO: 18 - protein, 1061 aa, Homo sapiens
MPGPRRPAGSRLRLLLLLLLPPLLLLLRGSHAGNLTVAVVLPLANTSYPWSWARVGPAVELALAQVKARPDLLPGWTVRTVLGSSENALGVCSDTAAPLAAVDLKWEHNPAVFLGPGCVYAAAPVGRFTAHWRVPLLTAGAPALGFGVKDEYALTTRAGPSYAKLGDFVAALHRRLGWERQALMLYAYRPGDEEHCFFLVEGLFMRVRDRLNITVDHLEFAEDDLSHYTRLLRTMPRKGRVIYICSSPDAFRTLMLLALEAGLCGEDYVFFHLDIFGQSLQGGQGPAPRRPWERGDGQDVSARQAFQAAKIITYKDPDNPEYLEFLKQLKHLAYEQFNFTMEDVLVNTIPASFHDGLLLYIQAVTETLAHGGTVTDGENITQRMWNRSFQGVTGYLKIDSSGDRETDFSLWDMDPENGAFRVVLNYNGTSQELVAVSGRKLNWPLGYPPPDIPKCGFDNEDPACNQDHLSTLEVLALVGSLSLLGILIVSFFIYRKMQLEKELASELWRVRWEDVEPSSLERHLRSAGSRLTLSGRGSNYGSLLTTEGQFQVFAKTAYYKGNLVAVKRVNRKRIELTRKVLFELKHMRDVQNEHLTRFVGACTDPPNICILTEYCPRGSLQDILENESITLDWMFRYSLTNDIVKGMLFLHNGAICSHGNLKSSNCVVDGRFVLKITDYGLESFRDLDPEQGHTVYAKKLWTAPELLRMASPPVRGSQAGDVYSFGIILQEIALRSGVFHVEGLDLSPKEIIERVTRGEQPPFRPSLALQSHLEELGLLMQRCWAEDPQERPPFQQIRLTLRKFNRENSSNILDNLLSRMEQYANNLEELVEERTQAYLEEKRKAEALLYQILPHSVAEQLKRGETVQAEAFDSVTIYFSDIVGFTALSAESTPMQVVTLLNDLYTCFDAVIDNFDVYKVETIGDAYMVVSGLPVRNGRLHACEVARMALALLDAVRSFRIRHRPQEQLRLRIGIHTGPVCAGVVGLKMPRYCLFGDTVNTASRMESNGEALKIHLSSETKAVLEEFGGFELELRGDVEMKGKGKVRTYWLLGERGSSTRG

SEQ ID NO: 19 - DNA, 4246 bp, Homo sapiens
ggttccctccggatagccggagacttgggccggccggacgccccttctggcacactccctggggcaggcgctcacgcacgctacaaacacacactcctctttcctccctcgcgcgccctctctcatccttcttcacgaagcgctcactcgcaccctttctctctctctctctctctctaacacgcacgcacactcccagttgttcacactcgggtcctctccagcccgacgttctcctggcacccacctgctccgcggcgccctgcgcgcccccctcggtcgcgccccttgcgctctcggcccagaccgtcgcagctacagggggcctcgagccccggggtgagcgtccccgtcccgctcctgctccttcccatagggacgcgcctgatgcctgggaccggccgctgagcccaaggggaccgaggaggccatggtaggagcgctcgcctgctgcggtgcccgctgaggccatgccggggccccggcgccccgctggctcccgcctgcgcctgctcctgctcctgctgctgccgccgctgctgctgctgctccggggcagccacgcgggcaacctgacggtagccgtggtactgccgctggccaatacctcgtacccctggtcgtgggcgcgcgtgggacccgccgtggagctggccctggcccaggtgaaggcgcgccccgacttgctgccgggctggacggtccgcacggtgctgggcagcagcgaaaacgcgctgggcgtctgctccgacaccgcagcgcccctggccgcggtggacctcaagtgggagcacaaccccgctgtgttcctgggccccggctgcgtgtacgccgccgccccagtggggcgcttcaccgcgcactggcgggtcccgctgctgaccgccggcgccccggcgctgggcttcggtgtcaaggacgagtatgcgctgaccacccgcgcggggcccagctacgccaagctgggggacttcgtggcggcgctgcaccgacggctgggctgggagcgccaagcgctcatgctctacgcctaccggccgggtgacgaagagcactgcttcttcctcgtggaggggctgttcatgcgggtccgcgaccgcctcaatattacggtggaccacctggagttcgccgaggacgacctcagccactacaccaggctgctgcggaccatgccgcgcaaaggccgagttatctacatctgcagctcccctgatgccttcagaaccctcatgctcctggccctggaagctggcttgtgtggggaggactacgttttcttccacctggatatctttgggcaaagcctgcaaggtggacagggccctgctccccgcaggccctgggagagaggggatgggcaggatgtcagtgcccgccaggcctttcaggctgccaaaatcattacatataaagacccagataatcccgagtacttggaattcctgaagcagttaaaacacctggcctatgagcagttcaacttcaccatggaggatgtcctggtgaacaccatcccagcatccttccacgacgggctcctgctctatatccaggcagtgacggagactctggcacatgggggaactgttactgatggggagaacatcactcagcggatgtggaaccgaagctttcaaggtgtgacaggatacctgaaaattgatagcagtggcgatcgggaaacagacttctccctctgggatatggatcccgagaatggtgccttcagggttgtactgaactacaatgggacttcccaagagctggtggctgtgtcggggcgcaaactgaactggcccctggggtaccctcctcctgacatccccaaatgtggctttgacaacgaagacccagcatgcaaccaagatcacctttccaccctggaggtgctggctttggtgggcagcctctccttgctcggcattctgattgtctccttcttcatatacaggaagatgcagctggagaaggaactggcctcggagctgtggcgggtgcgctgggaggacgttgagcccagtagccttgagaggcacctgcggagtgcaggcagccggctgaccctgagcgggagaggctccaattacggctccctgctaaccacagagggccagttccaagtctttgccaagacagcatattataagggcaacctcgtggctgtgaaacgtgtgaaccgtaaacgcattgagctgacacgaaaagtcctgtttgaactgaagcatatgcgggatgtgcagaatgaacacctgaccaggtttgtgggagcctgcaccgacccccccaatatctgcatcctcacagagtactgtccccgtgggagcctgcaggacattctggagaatgagagcatcaccctggactggatgttccggtactcactcaccaatgacatcgtcaagggcatgctgtttctacacaatggggctatctgttcccatgggaacctcaagtcatccaactgcgtggtagatgggcgctttgtgctcaagatcaccgactatgggctggagagcttcagggacctggacccagagcaaggacacaccgtttatgccaaaaagctgtggacggcccctgagctcctgcgaatggcttcaccccctgtgcggggctcccaggctggtgacgtatacagctttgggatcatccttcaggagattgccctgaggagtggggtcttccacgtggaaggtttggacctgagccccaaagagatcatcgagcgggtgactcggggtgagcagccccccttccggccctccctggccctgcagagtcacctggaggagttggggctgctcatgcagcggtgctgggctgaggacccacaggagaggccaccattccagcagatccgcctgacgttgcgcaaatttaacagggagaacagcagcaacatcctggacaacctgctgtcccgcatggagcagtacgcgaacaatctggaggaactggtggaggagcggacccaggcatacctggaggagaagcgcaaggctgaggccctgctctaccagatcctgcctcactcagtggctgagcagctgaagcgtggggagacggtgcaggccgaagcctttgacagtgttaccatctacttcagtgacattgtgggtttcacagcgctgtcggcggagagcacgcccatgcaggtggtgaccctgctcaatgacctgtacacttgctttgatgctgtcatagacaactttgatgtgtacaaggtggagacaattggcgatgcctacatggtggtgtcagggctccctgtgcggaacgggcggctacacgcctgcgaggtagcccgcatggccctggcactgctggatgctgtgcgctccttccgaatccgccaccggccccaggagcagctgcgcttgcgcattggcatccacacaggacctgtgtgtgctggagtggtgggactgaagatgccccgttactgtctctttggggatacagtcaacacagcctcaagaatggagtctaatggggaagccctgaagatccacttgtcttctgagaccaaggctgtcctggaggagtttggtggtttcgagctggagcttcgaggggatgtagaaatgaagggcaaaggcaaggttcggacctactggctccttggggagagggggagtagcacccgaggctgacctgcctcctctcctatccctccacacctcccctaccctgtgccagaagcaacagaggtgccaggcctcagcctcacccacagcagccccatcgccaaaggatggaagtaatttgaatagctcaggtgtgcttaccccagtgaagacaccagataggacctctgagaggggactggcatggggggatctcagagcttacaggctgagccaagcccacggccatgcacagggacactcacacaggcacacgcacctgctctccacctggactcaggccgggctgggctgtggattcctgatcccctcccctccccatgctctcctccctcagccttgctaccctgtgacttactgggaggagaaagagtcacctgaaggggaacatgaaaagagactaggtgaagagagggcaggggagcccacatctggggctggcccacaatacctgctcccccgaccccctccacccagcagtagacacagtgcacaggggagaagaggggtggcgcagaagggttgggggcctgtatgccttgcttctaccatgagcagagacaattaaaatctttattccaaaaaaaaaaaaaaaa

SEQ ID NO: 20 - protein, 541 aa, Homo sapiens
MPSLLVLTFSPCVLLGWALLAGGTGGGGVGGGGGGAGIGGGRQEREALPPQKIEVLVLLPQDDSYLFSLTRVRPAIEYALRSVEGNGTGRRLLPPGTRFQVAYEDSDCGNRALFSLVDRVAAARGAKPDLILGPVCEYAAAPVARLASHWDLPMLSAGALAAGFQHKDSEYSHLTRVAPAYAKMGEMMLALFRHHHWSRAALVYSDDKLERNCYFTLEGVHEVFQEEGLHTSIYSFDETKDLDLEDIVRNIQASERVVIMCASSDTIRSIMLVAHRHGMTSGDYAFFNIELFNSSSYGDGSWKRGDKHDFEAKQAYSSLQTVTLLRTVKPEFEKFSMEVKSSVEKQGLNMEDYVNMFVEGFHDAILLYVLALHEVLRAGYSKKDGGKIIQQTWNRTFEGIAGQVSIDANGDRYGDFSVIAMTDVEAGTQEVIGDYFGKEGRFEMRPNVKYPWGPLKLRIDENRIVEHTNSSPCKSSGGLEESAVTGIVVGALLGAGLLMAFYFFRKKYRITIERRTQQEESNLGKHRELREDSIRSHFSVA

SEQ ID NO: 21 - DNA, 53 bp, Artificial Sequence
  an SiRNA for an NPR-A
tattacggtggaccacctgttcaagagacaggtggtccaccgtaatatttttt

SEQ ID NO: 22 - DNA, 53 bp, Artificial Sequence
  an SiRNA for an NPR-A
agaattccagaaacgcagcttcaagagagctgcgtttctggaattcttttttt

SEQ ID NO: 23 - DNA, 72 bp, Artificial Sequence
  an SiRNA for an NPR-A
catatggggcccgggcgctgctgctgctaccctcgaaatggtagcagcagcagcgcccttgaattcccatgg

SEQ ID NO: 24 - DNA, 72 bp, Artificial Sequence
  an SiRNA for an NPR-A
catatggggcccgcggccacgcgagcgacctctcgaaataggtcgctcgcgtggccgcttgaattcccatgg

SEQ ID NO: 25 - DNA, 72 bp, Artificial Sequence
  an SiRNA for an NPR-A
catatggggcccggctcggccggacttgctgctcgaaatcagcaagtccggccgagccttgaattcccatgg

SEQ ID NO: 26 - DNA, 15810 bp, Homo sapiens
ggatcccaaaccagcacacctttccctcttcccccgaggagaccaggtaggaggcgagggaaaaggtggggcgcaagtgggccccggttgcttccacacacaccctccgttcagccgtcctttccatcccggcgagggcgcaccttcagagggtcctgtcctccaaagaggtaggcgtggggcggccgagaccggggaagatggtccacggggaagcgcgcgggctgggcggcggggaggaaggagtctatgatcctggattggctcttctgtcactgagtctgggaggggaagcggctgggagggagggttcggagcttggctcgggtcctccacggttccctccggatagccggagacttgggccggccggacgccccttctggcacactccctggggcaggcgctcacgcacgctacaaacacacactcctctttcctccctcgcgcgccctctctcatccttcttcacgaagcgctcactcgcaccctttctctctctctctctctctctaacacgcacgcacactcccagttgttcacactcgggtcctctccagcccgacgttctcctggcacccacctgctccgcggcgccctgcacgcccccctcggtcgcgccccttgcgctctcggcccagaccgtcgcagctacagggggcctcgagccccggggtgagcgtccccgtcccgctcctgctccttcccatagggacgcgcctgatgcctgggaccggccgctgagcccaaggggaccgaggaggccatggtaggagcgctcgcctgctgcggtgcccgctgaggccatgccggggccccggcgccccgctggctcccgcctgcgcctgctcctgctcctgctgctgccgccgctgctgctgctgctccggggcagccacgcgggcaacctgacggtagccgtggtactgccgctggccaatacctcgtacccctggtcgtgggcgcgcgtgggacccgccgtggagctggccctggcccaggtgaaggcgcgccccgacttgctgccgggctggacggtccgcacggtgctgggcagcagcgaaaacgcgctgggcgtctgctccgacaccgcagcgcccctggccgcggtggacctcaagtgggagcacaaccccgctgtgttcctgggccccggctgcgtgtacgccgccgccccagtggggcgcttcaccgcgcactggcgggtcccgctgctgaccgccggcgccccggcgctgggcttcggtgtcaaggacgagtatgcgctgaccacccgcgcggggcccagctacgccaagctgggggacttcgtggcggcgctgcaccgacggctgggctgggagcgccaagcgctcatgctctacgcctaccggccgggtgacgaagagcactgcttcttcctcgtggaggggctgttcatgcgggtccgcgaccgcctcaatattacggtggaccacctggagttcgccgaggacgacctcagccactacaccaggctgctgcggaccatgccgcgcaaaggccgaggtgagacgctggcacaccccgtcccgccgcttagccgcagggcctcccctctgacctgccggaggcatcgggactttctctctcatctgggggcactcttctttctcctcgccgttcttcattctactttcagctccctggccctttctacagctgagtttctatttccctctcttcttccgccacccccaccacgtctctatcctctcatctccccgacccccactcattccctcccaccctagcacagctcggttccggtccctttttccctcccacattttctctcttccctatagccttctcccttctttcatcctctcctctcatggcgcctcatcccctctcttctccccctccctctccctcctctctccctcctggccccatccttctccaccttcagctccactatccccctctccctacccgttccttcctcccttccgcctcccccttcctcctcccgcccaccgccccgcacccgcccgttccacccttcgactttctcctgctgtggcctaggctgagccgggagttaccacttaactctcactgggtctctcctgcaccctatctctaaacttcctcccttgggtgccccagctttcctactcctgtctctcccgcagtacctaggcttctctctctgactctccgtctttctccagttatctacatctgcagctcccctgatgccttcagaaccctcatgctcctggccctggaagctggcttgtgtggggaggactacgttttcttccacctggatatctttgggcaaagcctgcaaggtggacagggccctgctccccgcaggccctgggagagaggggatgggcaggatgtcagtgcccgccaggcctttcaggtgagtacctaggtttgaagcccaggctgtctcagcttgtggcacatcatttctgggcactgtgtccctcagcatctgaaagaattccagaaaagaggtttttgtctgtttgtttctttatgcactcctggtaactcacagaacagaaaagaggttggtgatgctcactgggaattaggcaatgaagggcaggggactgcccaggggcgcttcgccaccagcaggctaaaaagataagaaaatgggcttgaggcgggaggaggataaagtcccacagcctggacaggacttggagaaggcatcccattggatcccctgctttggaatgggcatcacttcatgcagggcatagggtccagtttgaccttgagctaagcagagacgcagctctgggaggtgggctcccaactgttggggccccacagtactagggaatagtcagctcccaactctctgctctccactgacccctttctcaggctgccaaaatcattacatataaagacccagataatcccgagtacttggaattcctgaagcagttaaaacacctggcctatgagcagttcaacttcaccatggaggatggcctggtaagaaggggtcccgggaccctccagcgtggacctccagcccccactccatgaccctctgccagcctccatccttccctattcccagttctccccttccttccctcccttcccattgttccatgtttctcgtgatgatggaggaggacactggcaagttcagcctctgaaactcaggtcatcatcagtaatatggagacgatacatcctgccctgtctacctagtaggattcaggaagtgatgctaatccaaaggcatcgtttaaatagtaaaatctccctgtgatataggggtgttattttctcccatcctcttccaaaatcccagtgcctcttgttcccttccccacagctcccacctccatgcccttcatatgcccaccccagccgacctctgtttgcccctacaggtgaacaccatcccagcatccttccacgacgggctcctgctctatatccaggcagtgacggagactctggcacatgggggaactgttactgatggggagaacatcactcagcggatgtggaaccgaagctttcaaggtcagggcctggaggtggctggaatgggctgccttgggggatgaatcccaggtgcccagtgtcaagccatgagaagcctattgtcctgcagcagttacctatgcacaccagccttttcctccacagcttttttcaggcccatccctcagaagtcctacaaagtgtccaatctcaatcatccctgctgggcactgagttcttttacctttctttttcttttttcttttttttttgagatggagtctcgctctgtccccaagactggagtgtggtggtgcaatctcggctcacttcaacctccgcctcccaggttcaagcaattctcctgcctcagcctcctgagtagctgggattacaggtgccctccaccaacacttggctaattttttgtattttttttagtagagacagggtttcaccacgttggtcaggctggtcttgaactcctgacgtcaggtgatctgcccgcctcagcctcccaaagtgctgggattacaagcatgagccacagtgcccggccgttttaccatttactatcattctgtatacatgtatgtttggaaggcaaggcaaaaaagattagaggatgaagagatgaagtggggcacccctgaacttctattctctcaaacatagtcatcttcccccatgtcctcaggtgtgacaggatacctgaaaattgatagcagtggcgatcgggaaacagacttctccctctgggatatggatcccgagaatggtgccttcagggtaagtttgtgcacccagaagacagtgccaattccaaatgacatctcaccctcctacttcccccccacagccctgccagggcacctgtttatcctgtagccattccaccatgcctggacacttacaagagccctggataaaacagacccagctccagtctggggaagccaccagaatgatagggactcacaggcatcacacttggggagccccatgcctgaggagggagcacaagcctgccctcggggagctccgaagggaggcaggcaggaccgcctcccagcagagacagggctgtgaaagatgcacattacacagctctgcaagcgagcagggacaggaaggcgctgaggccaatggccacaagggacaggtcatccagagaaggcctcctggaagacgggcacatggactgggcctgcgaatgtaggctaaggtgaacattaccttctcctgttttctaccaagaaaataagtagagaaaaatcaatgcttggttggtacttcaaccaagattataaactccctgagtgtagagatcgggttctaaatggagttttctttataaaccccttgatagttttcaggtgtttccacttgagtactatgtgtgtggtatgaggtcctgtgtccagttgcagtggggacttggtaagcaggtgacaacccagatatatatgtaggctctagaagcagagctggggtaggtgggaggtgagactgctgcactcacagcatgccttccccgcaggccctggcctagccaccactcctgctctcccttaggttgtactgaactacaatgggacttcccaagagctggtggctgtgtcggggcgcaaactgaactggcccctggggtaccctcctcctgacatccccaaatgtggctttgacaacgaagacccagcatgcaaccaaggtgactgccccttgccttccaggcctccatcccagagatgctgcatccttcccctaagcacagtcgagtaggtgctcctgtcccatgctgagggctttctggagaatgactcctgcctttttcttcccttcatccatcatcccagttcactgatggactattagaaagttcttcctcctgctgtctaacccaaatctctcttgctgcaatatggactctctcctgcagatcacctttccaccctggaggtgctggctttggtgggcagcctctccttgctcggcattctgattgtctccttcttcatatacaggtgagctgtgatgtggggggttgagtgaggctgggggacccggagaaccaagagcagaggaggcggtggggacccagagggaagagggcaggggtgaaggggcagcaggggaaaaccaagggagatgaggaagaaaggaggcttaaaagccagaggagaaagaaagagaagggaatggcagggcgaggggaggagacaaggataggaatggccaaggagagtcagaaagatccaagaagcagagaagttgatgggtgacatcataggggcgtggactggttttccttgctactcttgcaggccagataggaagcaactttctgaacctttgcaatcatgcccatgttagctgaggagggtgagccctggtgtgtgccaggtgcccaacctagaatggagaagggagctgaatgagccttgttcctgccgtccagtggaggctaaaatgaagtacaggaggagttaatgatatacaaaagcaaggagggaggggagaaaaatcactgctggttgagcatataatgtgtgccaggcacttccacgtacactatttctttctttctttttttttttttttttttttttttgagacggagtctcgctctgttgccagactggagtgcagtggcatgatctaggctcactgcaacctccgcctcccagtttcaagcaattctcctgcctcagcctcccatgtagctgggactacaggcacatgccaccacgctcagctaatttttgtatttttagtagagacagggtttcaccatgttggccaggatggtctcgatctcttgacctcatgatccacccaccttggcctcccaaagtgctgggattacaggcatgagccactgtgcctggcctcatgttcactatttcttttcattcttataatagttaagaatgaaatagatattgcggcctcattcccaagtaaggacattgaggtgattcccccaaggtccccagtaaggcagaatttcccccagccatcctgattctcagtccagaggatagaattccccctccatctctgagtgcatggtgtggtcccacggctctgaggaggggctgctgagcaccctgccctgggtcagcggctcagccacaggctcagatgcagccttcgtatcccaggaagatgcagctggagaaggaactggcctcggagctgtggcgggtgcgctgggaggacgttgagcccagtagccttgagaggcacctgcggagtgcaggcagccggctgaccctgagcggggtaagaacgctggtgtttgtgttggggggcaataaaggagaggtgggtacaaggggcagtgcctgagggataggtaagggagcaggattctagtcccagctctgctttcacttgctgtgtgaccttgagcgactcatagtccctctccgagactgtctcagatgatgattacagcagcagagcctccctcacagggctcttttaaaggtcagaggagatagtacctgtgaaaacactttaaaaaaaaaaaaagtaaatgaggaggaaattttatgatgtggaacataaagcagggtgggccaggcacagtggctcacatctgcaatcccagcactttgggagaccgaggcaggaggattgcttgtgcctgggagttcaagaccagcctgggcaacagagcaagacatcgtctctacaaagaatacaaagattagcagggcatggtggcgcatacctgtagtcccagctactctggaggctgaggtgaaaggatcatctgagcccaggagtctgaggcggcagtgacctaggatagcaccactgcactccagcctggatgacacaatgatactacatctcaaaaaaaaacccaacaacaaaaaggaagggtgacacaaagataaggcaggataaggcagggaaataaagaccagagcacaagcaatcaggatgcagactgggcccaccggctgaccattcctcctgctctccctcctttcagagaggctccaattacggctccctgctaaccacagagggccagttccaagtctttgccaagacagcatattataaggtgggcctggggaaagatcactgggccttgggactggggcaggagtgtactctgatggaggactggtggggggttctgagggaaggagtaagctggtggggagcagcagatgggggccctgggggtgggctattgggaacaagtgagggtcctgagggcagggatgggctgtcgggagcagctggaattcccaggacatgggaccatgctcttcacagtgacagtctccattccatgcccagggcaacctcgtggctgtgaaacgtgtgaaccgtaaacgcattgagctgacacgaaaagtcctgtttgaactgaagcatgtaatgtggggagtgaggcagtggcatggagaaggggccctcggggacgcaagggagactggccaacagaactagttatggagggacctcagggtaccccaagaaaggggcagggactggagccctggatgaccttcatcttgtggtggagtgggggtatcctaagtaggagaagagaccactgagataacctggaggaatcttgaggggccatatgtgatgtccctgggggagagagggcttaggatgccagagggagtaggagcagattctggggagggtgggctaaaggacatgggtgggaatcaccagggaagatcttagtgatggttgcagaaagtgaataaggagttaagaagagtgagggtccctgaagctagtgagcagcttggtgaggagcgaggtctctgtcaagctcctgatgctggtcccacttgcagatgcgggatgtgcagaatgaacacctgaccaggtttgtgggagcctgcaccgacccccccaatatctgcatcctcacagagtactgtccccgtgggagcctgcaggtgagggggacaaggggtgtcaagaaacctgggttctagccctggctctgcccctgactggccataagaccccaggcatgcctcgccctctttctgacctttctggccccatctgtaaaaatgggagttggggaagggcagtggcactagagtcaatccaaagttttgtcctgttctaccagttcacatcagtaggaccctgcaccctcctccaactcccagggggatctgcaggggattggtcttgactcttattgccccagcaggacattctggagaatgagagcatcaccctggactggatgttccggtactcactcaccaatgacatcgtcaaggtatgcccctaagcacctattggatgtgtagagcaggggccaggcatgcttctcctggccacgggtgtaggtcccactcctggccaatacctctgcccactcacatttccagggcatgctgtttctacacaatggggctatctgttcccatgggaacctcaagtcatccaactgcgtggtagatgggcgctttgtgctcaagatcaccgactatgggctggagagcttcagggacctggacccagagcaaggacacaccgtttatgccagtgagccttgactcttgaacctaacacctgcccccagcaccacccagtagggagactgatgcaaggcctctgatgggcttgggcatgcttgtcctgactccagcctcaattcattcacccatgaaaaagggaaggccagacgaagtggtttctaaggcctcctctagctctaacactctgtgatgcatccagatcagtttcggccacacccttgtttccccctcaccccttagctttgggctccctcactcggtgactaccgacctctgacccacagaaaagctgtggacggcccctgagctcctgcgaatggcttcaccccctgtgcggggctcccaggctggtgacgtatacagctttgggatcatccttcaggagattgccctgaggagtggggtcttccacgtggaaggtttggacctgagccccaaaggtgagaggagcacaccttccttaaacccagccacagtctcaacgaaccccagccccagggagagggtcccctggcagcaccaccacaccttccttctgtaatggggttcagtcaccaccctttgacccattgctgccagtgaccagtcccccgcccccatgccttggtcttggacttcccctgccatctcagctggttgccccagtctctcactaggcccttggccagccccacccctcagctcctctaccccccaatacagagatcatcgagcgggtgactcggggtgagcagccccccttccggccctccctggccctgcagagtcacctggaggagttggggctgctcatgcagcggtgctgggctgaggacccacaggagaggccaccattccagcagatccgcctgacgttgcgcaaatttaacaggtccctggtgtttgtcatggatcccccaggcccttcctccacagccaccatttacctaatgcttctggctctggcttatcccagcagtggcagagggagaccactcacctcctccctgtacatagtcagctccagctcagcacagcctcatgaccctcttcgcaagtacagcatgactcagctgtccccacagtcccctgccattcatgccccttccctccaccatcgacaccccacacccttcctgcccactcgccttgctggcctctagacttctcagcagtgtgtaggatagatgggcctcccgcctcctgccctgtaggctcttggccctccacgggagctcctgccccaccccttgatttcccttccccagcgtgcccaccaggcccagttcctccagacacacccttctgtggacatcactttgtccgcaattgacccttgtcattctccacctcctttacctccttctaactcactgggttcaacaaagatgaacaaaatgtccatatgtctgaagcttcatacttgaccttggggtctcagaaaagaattgaactttcttccttctgttttcccctgctccccggtatcctgctatgccctcaaccctgagcgtctctagagacctcactgcagtctggagggggaagtgcctaggggcgggcgctcacgtaggctgtgctgctcctctcttaccacccccaccgccaccctctgcccccagggagaacagcagcaacatcctggacaacctgctgtcccgcatggagcagtacgcgaacaatctggaggaactggtggaggagcggacccaggcatacctggaggagaagcgcaaggctgaggccctgctctaccagatcctgcctcagtgagtgcctgagtctggggaccccccccaacacaaagcccctgtcccgacccccaactctgatcctgcacctgccctgaccccttagctcagtggctgagcagctgaagcgtggggagacggtgcaggccgaagcctttgacagtgttaccatctacttcagtgacattgtgggtttcacagcgctgtcggcggagagcacacccatgcaggtaggccagggttcagccacaggtgccaggcaagctcagcatctggatcccaccagacctgccttctggttctgctttacccacctgaccccaggtggggtcccctacttcctgtctctcttagcttctcttcccttccaggtggtgaccctgctcaatgacctgtacacttgctttgatgctgtcatagacaactttgatgtgtacaaggtgagggtgggagtggggatgggaagggacagacagacatggacaaggtcagaaaaagatgaggggtaggcagaatgatgtggagtcttaagagaggagatcggggacacgggcagagacagtgacacagggagacccgggaacaggcagagaacccatgtgggatgggggatgagcaaagacagatgagggtacagaatgacagacgctgcacccggtgtgacggtgtggccggccgcacagttgcagccgtcaagtcctgcaccccctcgccactcccacaggtggagacaattggcgatgcctacatggtggtgtcagggctccctgtgcggaacgggcggctacacgcctgcgaggtagcccgcatggccctggcactgctggatgctgtgcgctccttccgaatccgccaccggccccaggagcagctgcgcttgcgcattggcatccacacaggtaaggccactgaaggtgcaggcgggcatccagaggccaaggctttgcaagggaaacttgtcccctggcccagcccctcgccctttcatctctctctctctctctctctctctctctctctctctctgtctctctctctctctctctctctctctctctcacacacacacacacacacacacacagagctgggacctcagatcctgcctcctgcctgtcttggattgtccacctacctcccttaacacccctccctccctcactcgctgatgggctctgctccttcccttgctcctcccaggacctgtgtgtgctggagtggtgggactgaagatgccccgttactgtctctttggggatacagtcaacacagcctcaagaatggagtctaatggggaaggtacagtgccccctcctagagggaatggggagggcagggtggctgagggaaatgccatcctggggcagcctgtgcctgcacagcccgtttcagctcctagccctttcgcctcccaagttccccttctcataatattaagagttcaacctgggctcatcaacttgactgtaaccagagactcaggttcctgctgcccctcttgtcaaacgatgtaaaagtatttccgggccagtgctggagagttcccagcaggaatctgattttaagaccctctgtgggccgggcgtggtgactcacacctgtgatcccagcactttgggaagctgaggcaggcggatcacctgaggtcgggggtttcgagaccagcctgaccaacatgatgaaatcccgtctctactaaaaatacaaaaaactagccaggtgtgatggcaggctcctgtaatcccagctacttgggaggcttgaggcagaagaattgcttgaacccgggaggcagaggttgcgatgagccaagattacaccacgcaccccagcttgggcaataagagttaaactctgtctcaaaaaaaaaaaaaaaaaaaaaaaaaagggccctctgctccacctttgatgtggtaaagatggcttcagagccagcataagtgaggctgtgaatctcagctccacagctggctgtgtgtcagtttgctatacctctctgagccatggttttcctcatctgtaaaaagagggaaaaaatctatctcacaggaattatgtgagaaacccattaaaaatgtctaccacataattgtcatttaacttttccaagccttagcggattatctgtaaaatgatgtctatctcaggattgcaagaagcctagcacaaaccctggtacccagcaggcacctaataaattcttactcctacccgccccttgctcttgcctcctgtttatcttctatccttctgctgtattcgacacaattcaatgcagtaaacatttattgagtgactactgagtgccaggccctgggatagtaacatggcccagatccagagttagctgagaaattcatgtggaccccatctaaaccttatggtgaaagaaaggctgcttgggagccagtcctgggagcccagagggatctagttcggcaaatattccctgggcactatttgggggctgcagagtcagcccttgttgagggtccagtcctcaaggagcacattcccagaaatgttcacattctggcgctggggtgctgtaatcccagcactttgggaggccgaggtgggcagatcacttgaggccaggagtggagactagcctggccaacatggtgacctcctgtctctactaaaaatacaaaaaattagctgggcgtggtggcacgtgcccgtaatcccagctactcaggaggcttgagacatgaaaatcacttgaacccaggaggtggatgttgcagtgagccgagactgcacccctgggcaacagagcgagactctgtctcaaaaaaaaaaaagagagaaagaaagaaaagaaaagaaagaaactgttaaacacaacaaggccactgtgattgatgcaaaccccagaagtagggacatgagttcagacagtggtcaaagagagggtgtggcaatattgggccccactccatcactgacctcctcagccacttgggcagatcaccctgggcctcagttcctcggccacaaaatgagggtatagcatgaaatcatgaaagcaacaatttacatagtgcttcctaggtagcacattccgtttgaatactttatggatgttaaatttaatcctcacaacaaggttttgagatgggtactgacactatcagcattttacagattaggaaaatgaagcagagagaatttattttacatacctaagcaagtatccaagctgaggttcatactgaggcagtgcaggatccaaagtgccagctcctaaccaccatgctgtgtagagccgggtgacactccagagagtgctgtccaacaggatgttccatagtcatgaaaatgttctgtattctgtgctgtccaatacagtagcctctaggcacatatggctacttatcactggaaatgtgacgggtgcaactgaggccctgattttttttttttttttggagacagagtttcgctctgtcgcccagcctggatggagtgcagtggtgcaatctcggctcactgcaacctccgcctcccaggttcaagcgattctcctgcctcagcctcccaagtagctggaattacaggtgagtgccaccacacacagctaatttttgtatttttagtagagacggggtttcgccatattggccaggatggtctcgaactcctggcctcaagtgatcctcctgcctcagcctcccaaagtgctgggattacaggtgtgagccacagcacccagcctgaatttttaactgtatttagtttaaattaatttaagttgaaacaggcacatgtgattagtggctactgtattggattacacagctccagagttctaaatgagaggctaatgtggtcacgcactacattcagggggtggggcccctctgagctagagggcttcctggcccaaaagagggagagagggtacctgtccacctgtccacccccacagtccctggtctcttttgcctctactttcctgctctcctctctcacattgctcaccttcccttctcccctgtcctacccagccctgaagatccacttgtcttctgagaccaaggctgtcctggaggagtttggtggtttcgagctggagcttcgaggggatgtagaaatgaaggtagagcgagaagcctctgccctccccaccttttggggtcctagagggagttacccttctcaagcagccgatgccactcccatccctaaggctctcatctgactggggaaagggcatgtgccactccccagcccatcctcttttttccctccagggcaaaggcaaggttcggacctactggctccttggggagagggggagtagcacccgaggctgacctgcctcctctcctatccctccacacctcccctaccctgtgccagaagcaacagaggtgccaggcctcagcctcacccacagcagccccatcgccaaaggatggaagtaatttgaatagctcaggtgtgctgaccccagtgaagacaccagataggacctctgagaggggactggcatggggggatctcagagcttacaggctgagccaagcccacggccatgcacagggacactcacacaggcacacgcacctgctctccacctggactcaggccgggctgggctgtggattcctgatcccctcccctccccatgctctcctccctcagccttgctaccctgtgacttactgggaggagaaagagtcacctgaaggggaacatgaaaagagactaggtgaagagagggcaggggagcccacatctggggctggcccacaatacctgctcccccgaccccctccacccagcagtagacacagtgcacaggggagaagaggggtggcgcagaagggttgggggcctgtatgccttgcttctaccatgagcagagacaattaaaatctttattccagtgacagtgtctcttcttgagggagagagggttgccagaaaacagtcagttctccactctctacttcaaataagactcacttcttgttctacaagggtctagaaggaaaagtaaaaaaaaaagactctcgattcttaac

SEQ ID NO: 27 - DNA, 22 bp, Artificial Sequence
  an NPRA specific primer
gcaaaggccgagttatctacat

SEQ ID NO: 28 - DNA, 19 bp, Artificial Sequence
  an NPRA specific primer
aacgtagtctcccacacaa

SEQ ID NO: 29 - protein, 59 aa, Mouse
MGSFSITLGFFLVLAFWLPGHIGANPVYSAVSNTDLMDFKNLLDHLEEKMPVEDEVMPP

SEQ ID NO: 30 - protein, 43 aa, Artificial Sequence
  consensus sequence
MSFSTFLLAFLGANPYAVSNDLMDFKNLLDHLEEKMPEDEVPP

SEQ ID NO: 31 - protein, 60 aa, Human
MSSFSTTTVSFLLLLAFQLLGQTRANPMYNAVSNADLMDFKNLLDHLEEKMPLEDEVVPP

SEQ ID NO: 32 - protein, 60 aa, Mouse
  misc_feature (47)..(60): Xaa can be any naturally occurring amino acid
QALSEQTEEAGAALSSLPEVPPWTGEVNPPLRDGSALGRSPWDPSDXXXXXXXXXXXXXX

SEQ ID NO: 33 - protein, 36 aa, Artificial Sequence
  consensus sequence
QLSEEEAGAALSLPEVPPWTGEVPRDGALGRPWDSD

SEQ ID NO: 34 - protein, 60 aa, Human
QVLSEPNEEAGAALSPLPEVPPWTGEVSPAQRDGGALGRGPWDSSDRSALLKSKLRALLT

SEQ ID NO: 35 - protein, 31 aa, Mouse
GPRSLRRSSCFGGRIDRIGAQSGLGCNSFRY

SEQ ID NO: 36 - protein, 29 aa, Artificial Sequence
  consensus sequence
PRSLRRSSCFGGRDRIGAQSGLGCNSFRY

SEQ ID NO: 37 - protein, 31 aa, Human
APRSLRRSSCFGGRMDRIGAQSGLGCNSFRY

(19) United States
(12) Patent Application Publication
(10) Pub. No.: US 2014/0343120 A1
(43) Pub. Date: Nov. 20, 2014

(54) METHODS AND COMPOSITIONS FOR REDUCING ACTIVITY OF THE ATRIAL NATRIURETIC PEPTIDE RECEPTOR AND FOR TREATMENT OF DISEASES

(71) Applicants: SHYAM S. MOHAPATRA, LUTZ, FL (US); WEIDONG XU, EL PASO, TX (US); XIAOYUAN KONG, TAMPA, FL (US); XIAOQIN WANG, XI'AN (CN); SUBHRA MOHAPATRA, LUTZ, FL (US)

(72) Inventors: SHYAM S. MOHAPATRA, LUTZ, FL (US); WEIDONG XU, EL PASO, TX (US); XIAOYUAN KONG, TAMPA, FL (US); XIAOQIN WANG, XI'AN (CN); SUBHRA MOHAPATRA, LUTZ, FL (US)

(73) Assignee: UNIVERSITY OF SOUTH FLORIDA, TAMPA, FL (US)

(21) Appl. No.: 14/102,793

(22) Filed: Dec. 11, 2013

Related U.S. Application Data
(63) Continuation of application No. 11/998,792, filed on Nov. 30, 2007, now abandoned.
Application No. 11/998,792 is a continuation-in-part of application No. 11/059,814, filed on Feb. 17, 2005, now abandoned.
(60) Provisional application No. 60/521,072, filed on Feb. 17, 2004.

(51) Int. Cl. C12N 15/113; A61K 31/713; A61K 47/48

(52) U.S. Cl. CPC C12N 15/1138; A61K 47/4823; A61K 31/713; C12N 2310/11; C12N 2310/14; C12N 2320/30
USPC 514/44 A

(57) ABSTRACT

Methods, compositions and devices are provided by the present invention for reducing activity of a natriuretic peptide receptor and other signals. Therapeutic treatments are provided by use of polynucleotides encoding a natriuretic peptide or by regulating the expression of natriuretic peptide receptor, such as NPRA and NPRC, or combinations of these therapies. Routes used for delivering polynucleotides encoding a natriuretic peptide, or, for example, siRNA that down regulates natriuretic peptide receptor include subcutaneous injection, oral gavage, transdermal and intranasal delivery routes. Compositions can include chitosan, chitosan derivatives, and chitosan derivative and a lipid. Transdermal delivery can use a transdermal cream. Intranasal delivery can use a dropper or an aspirator for delivery of a mist. Oral gavage delivers equivalent to oral delivery. Delivery permits cell and tissue specific targeting of gene therapies resulting in expression of a natriuretic peptide or down regulation of natriuretic peptide receptor. A variety of cancers, asthma and viral diseases can be treated therapeutically using the methods and compositions of the present invention.

### CROSS-REFERENCE TO RELATED APPLICATIONS

This application is a continuation of U.S. patent application Ser. No. 11/998,792, filed Nov. 30, 2007, which is a continuation-in-part of U.S. patent application Ser. No. 11/059,814, filed Feb. 17, 2005, now abandoned, which claims priority to U.S. Provisional Application No. 60/521,072, filed on Feb. 17, 2004, and U.S. patent application Ser. No. 11/059,814 is a continuation-in-part of U.S. patent application Ser. No. 11/799,225 filed Apr. 30, 2007, now U.S. Pat. No. 8,071,560, which claims priority to U.S. Provisional Application Ser. No. 60/796,278, filed Apr. 28, 2006. In addition, U.S. patent application Ser. No. 11/059,814 is a continuation-in-part of U.S. patent application Ser. No. 10/526,584, filed Oct. 11, 2005, now U.S. Pat. No. 7,655,772, which is the National Stage of International Application Number PCT/US2003/028056, filed Sep. 8, 2003, which claims priority to U.S. Provisional Application No. 60/319,529, filed on Sep. 6, 2002, the disclosures of each of which is hereby incorporated by reference in entirety, including any figures, tables, nucleic acid sequences, amino acid sequences, and drawings.

### FIELD OF THE INVENTION

The field relates to methods and compositions for reducing activity of the atrial natriuretic peptide receptor, as well as methods and compositions for treatment of diseases.

### BACKGROUND OF THE INVENTION

The vast majority of cancers of the lung, breast and colon are adenocarcinomas, which arise from pre-existing adenomatous polyps that develop in the normal colonic mucosa. This adenoma-carcinoma sequence is a well-characterized clinical and histopathologic series of events with which discrete molecular genetic alterations have been associated. Lung tumor development and metastasis are complex processes that include transformation, proliferation, resistance to apoptosis, neovascularization, and metastatic spread. A number of gene products have been identified that play critical roles in these processes. It has been suggested that the development of epithelial-derived tumors, the most common class of cancers, involves mutations of tumor suppressors and proto-oncogenes or epigenetic alterations of signaling pathways affecting cell proliferation and/or survival, which in turn may be caused by inflammation induced by infections and reactive oxygen species (ROS) (Ernst, P. Aliment Pharmacol Ther., 1999, 13(1):13-18).

As indicated above, ANF, the 126 amino acid prohormone, gives rise to four peptides: LANP (amino acids 1-30), VD (amino acids 31-67), KP (amino acids 79-98) and ANP (amino acids 99-126, also referred to herein as NP₉₉₋₁₂₆) (Angus R. M. et al, Clin Exp Allergy, 1994, 24:784-788). The ANP sequence particularly the C-terminal portion is highly conserved among species (Seidman et al., Science, 1984, 226: 1206-1209). The natriuretic peptide receptors (NPRs), NPR-A and NPR-B, are expressed in many different tissues of various organs systems, and are coupled to guanylyl cyclase ANP and BNP are thought to signal primarily through NPR-A by increasing cGMP and activating cGMP-dependent protein kinase (PKG). NPR-A is the primary receptor for ANP while NPR-B seems to bind CNP most effectively. PKG activation in turn activates ion transporters and transcription factors, which together affect cell growth and proliferation, apoptosis and inflammation. NPR-C is a clearance receptor for ANP removal, but also appears to signal phospholipase C activation and a decrease in adenylyl cyclase activity through a cGMP-independent pathway (Abbey and Potter, Endocrinology, 2003, 144: 240-246; Silberbach and Roberts, Cell Signal, 2001, 13:221-231). The signaling mechanisms underlying ANP's growth regulatory effects are poorly understood, although a number of reports suggest that ANP acts through mitogen-activated protein kinases (Silberbach and Roberts, Cell Signal, 2001, 13:221-231). Most cells of the mucosal immune system have ANP receptors (NPRs) and there is evidence that natriuretic peptides regulate the immune response and inflammation (Kurihara et al., Biochem Biophys Res Commun 1987, 149:1132-1140). ANP stimulates migration of human neutrophils (Izumi et al., J Clin Invest 2001, 108:203-213), and inhibit nitric oxide and TNF-α production by murine macrophages (Kiemer and Vollmar, J Biol Chem 1998, 273:13444-13451; Kiemer et al., J Immunol 2000, 165:175-81). It has been suggested that the ANP system may be a critical component of the immune response through its actions on both immune and non-immune cells. In patients with lung tumors, the immune response plays a large part in the progression of the disease and, consequently, the NPR system may potentially be involved. The alveolar macrophages in lung cancer patients secrete more pro-inflammatory cytokines, such as IL-6 and IL-1β, after LPS stimulation than in persons with non-malignant disease (Matanic et al., Scand J Immunol 2003, 57: 173-178). Increased IL-6 in lung cancer patients enhances the acute phase response, and is correlated with poor nutritional status and lowered survival (Martin et al., Cytokine 1999, 11; 267-273). The cells of the immune system, such as natural killer (NK) cells, Vα24 NKT, which are necessary for cancer surveillance, may also be reduced in lung tumor patients (Motohashi et al., Int J Cancer 2002, 102:159-165). The most common clinical paraneoplastic syndrome in patients with small-cell lung cancer (SCLC) is hyponatremia, which is believed to be caused by tumor secretion of vasopressin. Tumor biopsies from patients with SCLC and hyponatremia expressed the gene for ANP (Shimizu et al., Cancer 1991, 68: 2284-2288; Bliss et al., J Natl Can Inst, 1990, 82: 305-310). Thus, the reduced sodium levels seen in SCLC patients may be attributed to the secretion of ANP (Bliss et al., J Natl Can Inst, 1990, 82: 305-310). Human SCLC cell lines express functional ANP receptors (Ohsaki et al., Cancer Res 1993, 53: 3165-3171). A majority of SCLC cell lines produce ANP and some produce BNP as well (Oshaki et al., Oncology 1999, 56: 155-159). In contrast, in NSCLC cell lines, which are derived mostly from adenocarcinomas that comprise about two-thirds of all lung cancers, little is known about their growth regulation in response to ANP cascade.

The present inventor has found that the N-terminal natriuretic peptides, such as pNP73-102, are capable of inhibiting NFkB activation (Mohapatra, international application WO 2004/022003, published Mar. 18, 2004, which is incorporated herein by reference in its entirety), and that the ANP cascade plays a critical role in cell proliferation and inflammation. NFkB, a transcription factor and a key player in inflammatory processes, has been implicated in the development of cancer in liver and mammary tissues (Greten F. R. et al. Cell, 2004, 118: 285-296; Pikarsky E. et al. Nature, 2004, 431: 461-466). Activation of the NF-κB pathway enhances tumor development and may act primarily in the late stages of tumorigenesis. Inhibition of NF-κB signaling uniformly suppressed tumor development; however, depending upon the model studied, this salutary effect was attributed to an increase in tumor cell apoptosis, reduced expression of tumor cell growth factors supplied by surrounding stromal cells, or abrogation of a tumor cell dedifferentiation program that is critical for tumor invasion/metastasis.

An atrial peptide with natriuretic and diuretic properties was first reported from rat atrial muscle in 1981. Since then a family of natriuretic hormone peptides (NP) with broad physiologic effects including vasodilation and inhibition of aldosterone secretion has been described. Atrial natriuretic factor (ANF), a 126 amino acid prohormone gives rise to four peptides: long acting natriuretic peptide (LANP, amino acids 1-30), vessel dilator (VD, residues 31-67), kaliuretic peptide (KP, residues 79-98) and atrial natriuretic peptide (ANP, residues 99-126, also referred to here as NP99-126) (Vesely, D L Cardiovasc Res 2001 51:647-58). In addition, renal tubular cells produce urodilatin, a 32 amino acid peptide (residues 95-126 of ANF), which is released to circulation following differential processing of ANF (Forssman et al. Cardiovassc Res, 2001 51:450-62. ANP was reported to possess anti-cancer properties. See Vesely D L. Atrial natriuretic peptides: anticancer agents. J Investig Med 2005; 53:360-5. However, the half life of ANP is very brief, and an effective way of delivering ANP to treat or prevent cancer has not been developed.

There is also a pro-brain natriuretic peptide (BNP) first discovered in porcine brain which is analogous to ANP is found in circulation.

The third type of natriuretic hormone 25 the C-type (CNP) comprises two peptides, 53 and 22 amino acids in length, which are produced by many cell types (Levin, E R et al. N Eng J Med, 1998, 339321-8). Of these peptides, the C-terminal pro-ANF, ANP, has been studied most extensively.

In keeping with the diversity of these NPs, there are three NP receptors (Misono K S Mol Cell Biochem 2002, 230(1-2):49-60; Tremblay, J et al. Mol Cell Biochem, 2002 30 230(1-2):31-47). NPRa and NPRb which are coupled to guanylyl cyclase and the cGMP-independent receptor NPRc. ANP and BNP signal primarily through NPRa, which increases cGMP and activates cGMP-dependent protein kinase (PKG).

PKG activation turns on the ion transport mechanism and activates specific transcription factors, which together affect a range of cellular activities including, cell growth and proliferation apoptosis and inflammation.

NPRC functions as a clearance receptor but also appears to signal phospholipase C activation and a decrease in adenylyl cyclase activity (Silberbach et al. Cell Signal 2001 13:221-31).

Numerous tissues of various organ systems including the lung express these receptors in diverse cells. The NPs are produced in various tissues of the mucosa (lung, gastrointestinal and genitourinary systems), central nervous system and cardiovascular systems and released into the circulation. The signaling mechanisms underlying ANP's growth inhibitory effects are poorly understood, although a number of reports suggest that ANP affects signaling via activation of mitogen-activated protein kinases (Silberbach, M et al. Cell Signal 2001 13:221-31). The potential effects may include inhibition of ERK activation of epidermal growth factor, PKG-induced uncoupling of interaction, or Ras/Raft induction of MKP-, a MAPK phosphatase that inactivates signaling through a number of growth factors such as endothelin, EGF and FGF (Clark, A R J Endocrinol 2003, 178: 512).

ANP has been shown to mediate anti-inflammatory (Kiemer, A K and Vollmar J Biol Chem 1998 273: 134444-51) and cytoprotective (Kiemer, A K et al., J Immunol, 2000) do not express ANP receptors nor do they respond to ANP (Sprenger et al. Immunobiology, 1991 183(1-2):94-101).

The NP system, acting via cells of the innate immune system, modulates the immune response to antigens. Evidence to date suggests that it may augment allergic inflammation by acting on a number of cells in the lung (Kurihara, M et al. Biochem Biophys Res Commun 1987, 149(3):1132-1140). The primary evidence supporting this notion is the finding that ANP acts via its receptor dendritic cells to polarize these cells toward a Th2 phenotype, which is the hallmark of allergic immune response (Morita R et al. J Immunol 2003, 170(12):5869-5875). In asthma, the production of inflammatory mediators secreted from resident epithelial cells and recruited immune cells results in airway hyperreactivity, which characterizes the late-phase airway response. Without intervention, this event leads to non-reversible airway remodeling (including sub-basement-membrane collagen deposition, smooth muscle hyperplasia and hypertrophy, and goblet cell hyperplasia), with subsequent airway narrowing and progression of the asthma, naturally occurring gene-silencing mechanism triggered by double-stranded RNA (dsRNA), designated as small interfering RNA (siRNA), has emerged as a very important tool to suppress or knock down gene expression in many systems. RNA interference is triggered by dsRNA that is cleaved by an RNAse-Ill-like enzyme, Dicer into 21-25 nucleotide fragments with characteristic 5′ and 3′ termini (Provost™, P. D. et al. 20 Embo J 2002, 21:5864). These siRNAs act as guides for a multi-protein complex including a P AZ/PIWI domain containing the protein Argonaute2, that cleaves the target mRNA (Hammond, S. M. et al. Science 2001, 293:1146-1150). These gene-silencing mechanisms are highly specific and potent and can potentially induce inhibition of gene expression throughout an organism. The short interference RNA (siRNA) approach has 25 proven effective in silencing a number of genes of different viruses (Fire, A. Trends Genet. 1999, 15:358-363).

RNA interference (RNAi) is a polynucleotide sequence-specific posttranscriptional gene silencing mechanism effected by double-stranded RNA that results in degradation of a specific messenger RNA (mRNA), thereby reducing the expression of a 30 desired target polypeptide encoded by the mRNA (see WO 99/32619; WO 01175164; U.S. Pat. No. 6,506,559; Fire et al., Nature 391:806-11 (1998); Sharp, Genes Dev. 13:139-41 (1999); Elbashir et al. Nature 411:494-98 (2001); Harborth et al., J Cell 165:175-81; Sprenger, H et al., Immunobiology, 1991, 183:94-101) effects. It has been shown to decrease cytokine and stress stimulated activation of NFKB in various cell types leading to a decrease in pro-inflammatory cytokine production (Kiemer, A K and Vollmar J Biol Chem 1998 273:134444-51; Kiemer, A K et al., J Immunol 2000, 165:175-81; Morita, R et al., J Immunol 2003: 170:5869-75). ANP can reduce tumor necrosis factor-α (TNF-α)-stimulated production of adhesion molecules in endothelium. (Kiemer, A K and 25 Vollmar J Biol Chem 1998 273:134444-51). It has also been shown to attenuate TNF-α induced actin polymerization, through activation of MAPK phosphatase-1 (MKP-1) and inhibition of p38 activity, leading to decreased permeability (Clark, A R J Endocrinol 2003, 178(1):5-12).

ANP stimulates migration of human neutrophils (Izumi, T et al. J Clin Invest 2001, 108(2):203-21345), and inhibits nitric oxide (NO) and TNF-α production by murine macrophages (Vesely, D L et al. Chest 1990 97(6):1295-1298, Vesely, D L Am J Obstet Gynecol 1991, 165(3):567-573). Human peripheral blood monocyte, however, Sci. 14:4557-65 (2001)). RNAi is mediated by double-stranded polynucleotides, such as double-stranded RNA (dsRNA), having sequences that correspond to exonic sequences encoding portions of the polypeptides for which expression is compromised. RNAi reportedly is not effected by double-stranded RNA polynucleotides that share sequence identity with intronic or promoter sequences (Elbashir et al. 2001). RNAi pathways have been best characterized in Drosophila and Caenorhabditis elegans but “small interfering RNA” (siRNA) polynucleotides that interfere with expression of specific polynucleotides in higher eukaryotes such as mammals (including humans) have also been investigated (e.g., Tuschl, 2001 Chembiochem. 2:239-245; Sharp, 2001 Genes Dev. 15:485; Bernstein 10 et. al. 2001 RNA 7:1509; Zamore, 2002 296:1265; Plasterk, 2002 Science 296:1263; Zamore 2001 Nat. Struct. Biol. 8:746; Matzke et al. 2001 Science 293:1080; et al. EMBO Rep. 2:1107).

According to a current non-limiting model, the RNAi pathway is initiated by ATP-dependent, cleavage of long dsRNA into double-stranded fragments of about 1815 (e.g., 20, 21, 22, 23, 24, 25, 26 etc. nucleotide base pairs in length, called small interfering RNAs (siRNAs) (see review by Hutvagner et al., Curro Opin. Gen. Dev. Scadden 2001 12:225-32 (2002); Elbashir et al. 2001; Nyknen et al., Cell 107:309-21 (2001); Zamore et al., Cell 101:25-33 (2000)).

In Drosophila, an enzyme known as “Dicer” cleaves the longer double stranded RNA into siRNAs; Dicer belongs to the RNase III family of dsRNA-specific endonucleases (WO 01168836; Bernstein et al., Nature 409:363 (2001)). Further, according to this non-limiting model, the siRNA duplexes are incorporated into a protein complex, followed by ATP-dependent unwinding of the siRNA, which then generates an active RNA-induced silencing complex (RISC) (WO 01/68836). The complex recognizes and cleaves a target RNA that is complementary to the guide strand of the siRNA, thus interfering with expression of a specific protein (Hutvagner et al., supra).

In C. elegans and Drosophila, RNAi may be mediated by long double-stranded RNA polynucleotides (WO 99/32619; WO 01175164; Fire et al. 1998; Clemens et al. Proc. Natl. Acad. Sci. USA 97:6499-6503 (2000); Kisielow et al., Biochem. J. 363:130 (2002); see also WO 01192513 (RNAi-mediated silencing in yeast)).

In mammalian cells however, transfection with long dsRNA polynucleotides (i.e. greater than 30 base pairs) leads to activation of a non-specific sequence response that globally blocks the initiation of protein synthesis and causes mRNA degradation (Bass 411:428-29 Nature (2001)

Transfection of human and other mammalian cells with double-stranded RNAs of about 18-27 nucleotide base pairs in length interferes in a sequence-specific manner with expression of particular polypeptides encoded by messenger RNAs (mRNA) containing corresponding nucleotide sequences (WO 01175164; Elbashir et al. 2001; Elbashir et al. Genes Dev. 15:188-200 (2001)); Harborth et al., J Cell Sci. 114:4557-65 (2001); Carhew et al., Curro Opin. Cell Biol. 13:244-48 (2001); Mailand et al., Nature Cell Biol. Advance Online Publication (Mar. 18, 2002); Mailand et al. 2002 Nature Cell Biol. 4:317).

siRNA polynucleotides may offer certain advantages over other polynucleotides known in the art for use in sequence-specific alteration or modulation of gene expression to yield altered levels of an encoded polypeptide product. These advantages include lower effective siRNA polynucleotide concentrations, enhanced siRNA polynucleotide stability, and shorter siRNA polynucleotide oligonucleotide lengths relative to such other polynucleotides (e.g. antisense, ribozyme or triplex polynucleotides). By way of a brief background, antisense polynucleotides bind in a sequence-specific manner to target nucleic acids, such as mRNA or DNA, to prevent transcription of DNA or translation of the mRNA (see U.S. Pat. No. 5,168,053; U.S. Pat. No. 5,190,931; U.S. Pat. No. 5,135,917; U.S. Pat. No. 5,087,617; see also Clusel et al. Nucl. Acids 1993 Res. 21:3405-, describing “dumbbell” antisense oligonucleotides). “Ribozyme polynucleotides can be targeted to any RNA transcript and are capable of catalytically cleaving such transcripts, thus impairing translation of mRNA (see U.S. Pat. No. 5,272,262; U.S. Pat. No. 5,144,019; and U.S. Pat. Nos. 5,168,053, 5,180,818 5,116,742 and 5,093,246; U.S. Ser. No. 2002/193579). “Triplex” DNA molecules refers to single DNA strands that bind duplex DNA to form a colinear triplex molecule, thereby preventing transcription (see U.S. Pat. No. 5,176,996, describing methods for making synthetic oligonucleotides that bind to target sites on duplex DNA). Such triple-stranded structures are unstable and form only transiently under physiological conditions. Because single-stranded polynucleotides do not readily diffuse into cells and are therefore susceptible to nuclease digestion, development of single-stranded DNA for antisense or triplex technologies often requires chemically modified nucleotides to improve stability and absorption by cells. siRNAs, by contrast, are readily taken up by intact cells, are effective at interfering with the expression of specific polynucleotides at concentrations that are several orders of magnitude lower than those required for either antisense or ribozyme polynucleotides, and do not require the use of chemically modified nucleotides. Due to its advantages, RNAi has been applied as a target validation tool in research in vitro in vivo and as a potential strategy for target validation and therapeutic product development (Novina, C. D. and Sharp, P. Nature 2004, 430:161-164; Lieberman, L. et al. Trends Mol. Med. 2003, 9(9):397-403). In vivo gene silencing with RNAi has been reported using viral vector delivery, liposomal delivery, and high-pressure, high-volume intravenous (Lv.) injection of synthetic iRNAs (Halder, J. et al. 10 Clin. Cancer Res. 2006, 12(16):4916-4924; Landen, C. N. et al. Cancer Biol. Ther. 2006 5(12):1708-1713; Scherr, M. et al. Oligonucleotides 2003 13:353-363; Song, E. et al., Nature Med., 2003, 9:347-351. In vivo gene silencing has been reported after local direct administration (intravitreal, intranasal, and intrathecal) of siRNAs to sequestered anatomical sites in various models of disease or injury, demonstrating the potential for delivery to organs such as the eye, lungs, and central nervous system (Reich, S. J. et al. Mol. Vis. 2003, 9:210-216; Zhang, X. et al. J Biol. Chem. 2004, 279:10677-10684; Dorn, G. et al. Nucleic Acids Res. 2004, 32, e49; Tolentino, M J. et al. Retina, 2004 24:132-138). Silencing of endogenous genes by systemic administration of siRNAs has also been demonstrated (Zimmerman, T. S. et al., Nature 2006, 441 (7089): 1123-334; 20 Soutschek, et al. Nature 2004, 432: 173-178).

Atrial natriuretic peptide (ANP), comprising the C-terminal amino acid residues 99-126 of the ANP prohormone, has been extensively studied for its functions in relation to blood pressure regulation. (Vesely D L. Atrial natriuretic hormones originating from the N-terminus of the atrial natriuretic factor prohormone. Clin Exp Pharmacol Physiol 1995; 22:108-14; Vesely D L. Atrial natriuretic peptides in pathophysiological diseases. Cardiovasc Res 2001; 51:647-58; Vesely D L. Atrial natriuretic peptide prohormone gene expression: hormones and diseases that upregulate its expression. IUBMB Life 2002; 53:153-9; Vesely D L, Chiou S, Douglass M A, McCormick M T, Rodriguez-Paz G, Schocken D D. Atrial natriuretic peptides negatively and positively modulate circulating endothelin in humans. Metabolism 1996; 45:315-9; Vesely D L, Perez-Lamboy G I, Schocken D D. Vessel dilator, long acting natriuretic peptide, and kaliuretic peptide increase circulating prostaglandin E2. Life Sci 2000; 66:905-13; Vesely D L, Perez-Lamboy G I, Schocken D D. Long-acting natriuretic peptide, vessel dilator, and kaliuretic peptide enhance the urinary excretion rate of beta2-microglobulin. Metabolism 2000; 49:1592-7; Vesely D L, San Miguel G I, Hassan I, Schocken D D. Atrial natriuretic hormone, vessel dilator, long-acting natriuretic hormone, and kaliuretic hormone decrease the circulating concentrations of CRH, corticotropin, and cortisol. J Clin Endocrinol Metab 2001; 86:4244-9; Vesely D L, San Miguel G I, Hassan I, Schocken D D. Atrial natriuretic hormone, vessel dilator, long acting natriuretic hormone, and kaliuretic hormone decrease circulating prolactin concentrations. Horm Metab Res 2002; 34:245-9.)

Its receptor, NPRA, is expressed on cells in many different tissues of various organ systems and signals through guanylyl cyclase. Both ANP and BNP signal through NPRA by increasing cyclic GMP (cGMP) and activating cGMP-dependent protein kinase (PKG). Activated PKG in turn upregulates expression of genes encoding ion transporters and transcription factors, which together affect cell growth, apoptosis, proliferation and inflammation. (Fiscus R R. Involvement of cyclic GMP and protein kinase G in the regulation of apoptosis and survival in neural cells. Neurosignals 2002; 11:175-90; Pedram A, Razandi M, Kehrl J, Levin E R. Natriuretic peptides inhibit G protein activation. Mediation through cross-talk between cyclic GMP-dependent protein kinase and regulators of G protein-signaling proteins. J Biol Chem 2000; 275:7365-72; Silberbach M, Roberts C T Jr. Natriuretic peptide signalling: molecular and cellular pathways to growth regulation. Cell Signal 2001; 13:221-31.

Inflammation is an important feature of lung cancers. Alveolar macrophages from lung cancer patients secrete more proinflammatory cytokines, especially IL-6 and IL-113, after LPS stimulation than do those from persons with nonmalignant disease. (See, Matanic D, Beg-Zec Z, Stojanovic D, Matakoric N, Flego V, Milevoj-Ribic F. Cytokines in patients with lung cancer. Scand J Immunol 2003; 57:173-8. Increased IL-6 in lung cancer patients enhances the acute phase response and is correlated with poor nutritional status and lowered survival (See, Martin J, Quiroga J A, Navas S, Pardo M, Carreno V. Modulation by biologic response modifiers of hepatitis C virus antigen-independent cytokine secretion in blood mononuclear cells. Cytokine 1999; 11:267-73.)

Both ANP and NPRA are expressed by lung cancer cells, and over-secretion of ANP has been linked with hyponatremia. (See, Bliss D P Jr, Battey J F, Linnoila R I, Birrer M J, Gazdar A F, Johnson B E. Expression of the atrial natriuretic factor gene in small cell lung cancer tumors and tumor cell lines. J Natl Cancer Inst 1990; 82:305-10. Ohsaki Y, Gross A J, Le P T, Oie H, Johnson B E. Human small cell lung cancer cells produce brain natriuretic peptide. Oncology 1999; 56:155-9; Ohsaki Y, Yang H K, Le P T, Jensen R T, Johnson B E. Human small cell lung cancer cell lines express functional atrial natriuretic peptide receptors. Cancer Res 1993; 53:3165-71.)

In addition, metastatic melanoma cells produce higher levels of cGMP in response to natriuretic peptides than do other cell types, and ANP may likely contribute to local inflammation in the origin of metastatic melanoma (Izumi T, Saito Y, Kishimoto I, Harada M, et al. Blockade of the natriuretic peptide receptor guanylyl cyclase-A inhibits NF-kappaB activation and alleviates myocardial ischemia/reperfusion injury. J Clin Invest 2001; 108:203-13.)

ANP possesses some topological similarity with melanin-concentrating hormone. Furthermore, the ANP gene, located on chromosome 1p36, is considered a candidate gene for melanomas. (Tunny T J, Jonsson J R, Klemm S A, Ballantine D M, Stowasser M, Gordon R D. Association of restriction fragment length polymorphism at the atrial natriuretic peptide gene locus with aldosterone responsiveness to angiotensin in aldosterone-producing adenoma. Biochem Biophys Res Commun 1994; 204:1312-7.)

Natriuretic peptides including ANP were reported to inhibit proliferation of various cancer cells and tumor growth. (Vesely D L. Atrial natriuretic peptides: anticancer agents. J Investig Med 2005; 53:360-5.)

Previously, an N-terminal ANP prohormone peptide comprising residues 73 to 102 (NP73-102) significantly inhibits activation of several proinflammatory transcription factors, including NFκB, activator protein 1 (AP1) and Erk-1,2, in human bronchial epithelial adenocarcinoma A549 cells (Hellermann G, Kong X, Gunnarsdottir J, et al. Mechanism of bronchoprotective effects of a novel natriuretic hormone peptide. J Allergy Clin Immunol 2004; 113:79-85; Mohapatra S S, Lockey R F, Vesely D L, Gower W R Jr. Natriuretic peptides and genesis of asthma: an emerging paradigm? J Allergy Clin Immunol 2004; 114:520-6.)

Since these transcription factors augment the local inflammatory milieu, it was reasoned that NPRA signaling plays a role in and promotes tumorigenesis. By corollary, blocking NPRA signaling would attenuate tumorigenesis and development of cancers. In this study, we tested tumorigenesis in mice that are deficient in NPRA and those exhibiting attenuated expression of NPRA via treatment with nanoparticles conjugated with siNPRA or pNP73-102.

The present inventors have demonstrated that, in contrast to prior knowledge that ANP decreases inflammatory mechanisms in the macrophages, ANP actually increases lung inflammation and this is caused by ANP-NPRA signaling. The present invention shows this signaling can be blocked by utilizing a small interference RNA (siRNA) approach, in which specific siRNAs targeted to NPRA can significantly decrease the inflammation. This results in amelioration of inflammation in allergic disease which may be caused by allergens and exacerbated by respiratory viral infections, pollutants, and smoke. Alternatively, an approach using a N-terminal ANP peptide comprising residues 73-102 (NP73-102) may also be used therapeutically. Also, this may be beneficial in the amelioration of inflammation and tumorigenesis in cancers.

### BRIEF SUMMARY OF THE INVENTION

The subject invention concerns methods and compositions for reducing activity or expression of a natriuretic peptide receptor. In one embodiment of a method for reducing activity or expression of a natriuretic peptide receptor, a polynucleotide complementary with a portion of a natriuretic peptide receptor gene is selected and administered resulting in a therapeutic effect. The administration of the polynucleotide reduces expression of a natriuretic peptide receptor, such as natriuretic peptide receptor A (NPRA) or natriuretic peptide receptor-C (NPRC). In one example, a plurality of polynucleotides can be administered such that expression of both NPRA and NPRC are regulated to produce a therapeutic effect, synergistically. For example, a method can include complexing one or more polynucleotides with chitosan or a chitosan derivative. The complex can also include a lipid, also.

Examples of polynucleotides contemplated by the present invention include a small interfering RNA (siRNA), an antisense molecule or a ribozyme. In one example, a small interfering RNA is selected from the group consisting of SEQ ID NO: 21, SEQ ID NO: 22, SEQ ID NO: 23, SEQ ID NO: 24, SEQ ID NO: 25 and combinations thereof. A synergistic effect is observed in a therapy combining the administration of a plurality of siRNA polynucleotides including, for example, SEQ ID NO: 23 and SEQ ID NO. 24.

In an alternative example, a polynucleotide may be selected that encodes the expression of a natriuretic peptide (NP), such as atrial natriuretic peptide (ANP), when administered either alone or in combination with an siRNA polynucleotide that is capable of reducing expression of natriuretic peptide receptor.

A polynucleotide of the invention may be administered by a wide variety of routes, such as by inhalation, intramuscular or subcutaneous injection, intravenous, intranasal or transdermal. By complexing the polynucleotide with a chitosan or chitosan derivative, intranasal delivery by drops or mist may be used to deliver the polynucleotide therapeutically in vivo. Therapeutic devices such as a dropper, inhalator, atomizer or nebulizer may be used to deliver complexes intranasally or by inhalation. In another example, transdermal delivery is accomplished by dispersing the complexes in transdermal creams, such as such as imiquimod cream (3M pharmaceuticals, Northridge, Calif.).

An advantage of the methods, compositions, and devices of the present invention is the effectiveness of the treatment for a variety of inflammatory or a cell proliferation disorders treatable by inducing apoptosis, for example. Results show that cancers, such as breast cancer, lung cancer, ovarian cancer, prostrate cancer, and skin cancer, may be treated, resulting in prevention, a reduction in the growth rate or a reduced tumor burden following administration of the polynucleotides according to the methods of the present invention. In addition, inflammatory diseases, such as asthma may be treated resulting in reduced inflammation. Viral diseases, such as respiratory syncytial viral infection, may be treated.

One example of a method for treating an inflammatory disease, a viral disease, or a cell proliferation disorder treatable by inducing apoptosis includes selecting a polynucleotide, the polynucleotide comprising a polynucleotide encoding a natriuretic hormone peptide and an operably linked promoter, or a polynucleotide complementary with a portion of natriuretic peptide receptor gene, or a combination thereof. By administering the polynucleotide according to one of the methods presented, a therapeutic effect is provided. The method may be effective for treating a wide range of mammals and mammalian cells that have similar natriuretic peptide receptor genes, such as mice, rats, apes and humans. In one embodiment, the natriuretic peptide receptor gene portion is a natriuretic peptide receptor A gene. In another embodiment, the portion is a natriuretic peptide receptor-C gene.

As previously noted, various types of polynucleotides complementary with the portion of a natriuretic peptide receptor gene may be selected. In addition, a polynucleotide encoding a natriuretic hormone peptide may include polynucleotides encoding a natriuretic hormone peptide such that one or more peptides from the group consisting of SEQ ID NO. 1, 2, 3, 4, 5 and 6 is expressed. In one example, a polynucleotide encoding SEQ ID NO: 5 is selected such that SEQ ID NO: 5 is expressed.

By complexing a polynucleotide encoding SEQ ID No. 5 with a chitosan or a chitosan derivative, additional routes of administering the polynucleotides are available, such as inhalation using a nebulizer, intramuscular, subcutaneous, intravenous, intranasal using drops or atomizer and transdermal. This method may be therapeutically effective for all of the disorders listed above, as well.

In another embodiment, a polynucleotide targeted to a portion of a natriuretic peptide receptor A gene is complementary with a portion of the natriuretic peptide receptor A gene, and inhibits expression of the natriuretic peptide receptor A gene. In one example, the polynucleotide is selected from the group consisting of SEQ ID NO: 23, SEQ ID NO: 24 and SEQ ID NO: 25. A synergistic effect is seen in combining a plurality of polynucleotides including SEQ ID NO: 23 and SEQ ID NO: 24, for example.

In another example, a method for reducing activity or expression of atrial natriuretic peptide receptor A is useful in treating cell proliferation disorders. For example, small interfering RNA may be selected from the group consisting of SEQ ID NO: 21, SEQ ID NO: 22, SEQ ID NO: 23, SEQ ID NO: 24 and SEQ ID NO: 25 to reduce activity of a NPR-A.

In one embodiment, a pharmaceutical composition for reducing activity of atrial receptor-A comprises a polynucleotide complementary with a portion of a natriuretic peptide receptor A gene. The pharmaceutical composition may include a polynucleotide that is a small interfering RNA selected from the group consisting of SEQ ID NO: 23, SEQ ID NO: 24 and SEQ ID NO: 25. In one embodiment, the small interfering RNA is SEQ ID NO: 23. In one embodiment, the small interfering RNA is SEQ ID NO: 24. In one embodiment, the small interfering RNA is SEQ ID NO: 25. In one example, the chitosan in the pharmaceutical composition is provided in a ratio to the polynucleotide. For example, the ratio of chitosan to polynucleotide may be in a ratio of 5:1 (weight/weight). In another example, the ratio may be 1:1. Anywhere within this range is considered to be an effective range for complexing the polynucleotide with chitosan (or a chitosan derivative).

One advantage of the methods and compositions of the invention is that decreased tumor formation and increased apoptosis occur. Another advantage is that cytokine production is reduced. Yet another advantage is that inflammation is reduced. In another advantage, administration by a route such as transdermal decreases NPRA expression, eosinophilia of the lung and cytokines Still another advantage is that viral infection, such as a respiratory syncytial viral infection, is inhibited. Yet another advantage is that melanoma tumor formation is reduced. Yet another advantage is that tumors from lung carcinoma and ovarian cancer were reduced. Another advantage is that topical administration through intranasal administration, for example, silences NPRA gene expression, causing significant reductions in tumor burden. Yet another advantage is that in situations where the NPRA gene is silenced, a mammal that is treated is resistant to tumor formation. For example, a mammal treated to reduce activation of NPRA gene may be injected with a prostate tumor cell and no tumors grow, while a control shows tumor growth, for example. In another advantage, a breast tumor cell may be injected and the breast tumor either does not grow or grows more slowly than a control.

Yet another advantage, a polynucleotide complementary with a portion of a natriuretic peptide receptor C gene is selected and a polynucleotide complementary with a portion of a natriuretic peptide receptor A gene is selected, such that the combination produces a synergistic effect.

The subject invention also concerns a host cell comprising a nucleic acid encoding a natriuretic peptide, or a nucleic acid complementary with all or a portion of a natriuretic peptide receptor gene, such as an antisense nucleic acid, or an siRNA nucleic acid.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

For a fuller understanding of the nature and objects, reference should be made to the following detailed description, taken in connection with the accompanying drawings, in which:

FIG. 1 shows pNP 73-102 inhibits NPRA expression. Pregnant (12 days) mice were injected i.p. with pVAX (vector), or pNP73-102. After 1 day, mice were sacrificed, thymi removed from the embryo, and homogenized. Cells were centrifuged and erythrocytes were lysed and incubated with anti-NPR-Ab or anti-NPR-C for 1 hour, washed, and incubated with PE-conjugated secondary antibodies. Levels of NPRA and NPRC were determined by flow cytometry.

FIGS. 2A-2D show NPRA deficiency decreases pulmonary inflammation. Groups (n=3) of wild type DBA/2 (wt) (FIG. 2A) and NPR-C deficient (NPRC^{-/-}) (FIG. 2B) mice and wild type C57/BL6 (wt) (FIG. 2C) and NPR-A (NPRA^{-/-}) (FIG. 2D) were sensitized with OVA (20 mg/mouse) and after 2 weeks challenged i.n. with OVA (20 mg/mouse). One day later mice were sacrificed and lung sections were stained with H & E to examine inflammation.

FIGS. 3A-3D demonstrate that A549 cells transfected with pNP₇₃₋₁₀₂ show a significantly higher level of apoptosis compared to control and pANP or pVAX (FIGS. 3A-3C). Cells were transfected with pNP73-102, pANP and pVAX (as control) and cells were stained with PI and annexin and quantified by flow cytometry (FIG. 3D). The proteins were isolated and an equal amount of the cell lysates were western-blotted using an antibody to poly-ADP ribose polymerase (PARP). The results demonstrate that pNP73-102 shows a higher accumulation of apoptotic cells compared to cells transfected with pANP and pVAX controls.

FIG. 4 shows that pNP73-102 decreases tumorigenesis in a colony formation assay by A549. Six centimeter tissue culture plates were covered with 4 ml of 0.5% soft agar. A549 cells were transfected with pANP, pNP₇₃₋₁₀₂ and pVAX plasmid DNA (V) or nothing (C). After 40 h of transfection, cells were suspended in 2 ml of 0.3% soft agar and added to each plate. Cells were plated in duplicate at a density of 2×10⁴ cells/dish and incubated for two weeks. Plates were photographed under a microscope. Cell colonies were counted and plotted. The results of one representative experiment of two are shown.

FIGS. 5A-5E show expression of NP₇₃₋₁₀₂-FLAG in the BAL cells after i.n. administration of chitosan encapsulated plasmid pNP₇₃₋₁₀₂-FLAG construct. BAL was performed in mice (n=3) after 24 hours and BAL cells were stained with either the second antibody control or anti-FLAG antibody (SIGMA) and then with DAPI. A representative staining is shown (FIGS. 5A-5C). FIG. 5D shows lungs removed from mice treated with chitosan nanoparticles carrying pNP₇₃₋₁₀₂ (CPNP73-102) (Rx) or empty plasmid pVAX (control). The lungs of control mice showed several lung nodules in contrast to mice treated with CPNP73-102, which showed very few tumors. Intranasal CPNP73-102 administration abrogated tumor formation in A549 injected nude mice. Nude mice were given 5×10⁶ cells intravenously (tail vein) and weekly injections of nanoparticle carrying either empty plasmid (control) or pNP73-102 (Rx). Three weeks later, mice were sacrificed and lung sections were stained with H & E to examine the lung nodules (FIG. 5D). Control shows nodules and tumor cell mass, whereas the treated group had no tumors. Sections were also stained with antibodies to cyclinB and to phospho-Bad (FIG. 5E). The results show that mice treated with CPNP73-102 had no tumors in the lung and did not show any staining for pro-mitotic Cyclin-B and anti-apoptotic marker phospho-Bad.

FIGS. 6A-6D demonstrate that treatment with chitosan nanoparticles carrying pNP₇₃₋₁₀₂ (CPNP73-102) decreases the tumor burden in a spontaneous tumorigenesis model of immunocompetent BALB/c mice. Two groups of mice (n=4) were administered with the Line-1 tumor cells (100,000 cells/mouse) at the flanks One group was administered with CPNP73-102 the same day, whereas another group was administered with vehicle alone (nanoparticle carrying a plasmid without NP73-102) and the third group was given the saline. Treatment was continued with CPNP73-102 or control at weekly intervals for 5 weeks. The tumors were dissected out from the mice of each group (FIGS. 6A-6C) and the tumor burden was calculated by weighing them on a balance and expressed as tumor mass per g lung weight. Results are shown in FIG. 6D.

FIG. 7 shows that CPNP73-102 induces apoptosis in chemo resistant ovarian cancer cells. C-13 and OV2008 ovarian cancer cells were transfected with pNP73-102. Forty-eight hours later, cells were processed for TUNEL assay to examine apoptosis. The results of one of two representative experiments are shown.

FIG. 8 shows breast cancer MCF-7 cell counts. The cells were transfected with pVAX, pANP, and pANP₇₃₋₁₀₂ and counted at 24 and 48 hours after transfection. 30 ml of Trypan Blue was mixed with 30 ml for measuring the cell viability. The results of one of two representative experiments are shown.

FIGS. 9A and 9B show a diagram depicting that over expression of ANP in the lung augments inflammation and cytokine production in splenocyte. FIG. 9A) Normal BALB/c mice were given Ln. nanoparticles carrying pANP or pV AX and their lungs were examined 3 days after by staining the sections (H&E), showing goblet cell hyperplasia. FIG. 9B) Female BALB1c mice were given i.p. OVA (with alum) and then challenged i.n. OVA. Mice were sacrificed, the spleens aseptically removed and the cells were cultured for 48 hours in the presence of OVA (Sigma) and recombinant IL-2. Cells were removed from culture and stained for surface markers CD4 and CD3 and intracellular cytokines ILIL-10 and IFN-g (BD Pharmingen).

FIG. 10 shows cloning of siNPRA sequences in the p U 6 vector. The siNPRA sequences were designed as shown in Sequence IDs and cloned in pSilencer (U6) vector using standard procedures. The transformants were tested by digestion with Apa I and EcoR I to release the siRNA inserts. Lane 1, 100 bp ladder; lane 2: pSilencer1(U6), Lane 3, siNPRA8, Lane 7-, siNPRA9 are shown for illustration.

FIGS. 11A-11C show the inhibitor y effect of transfected siRNA plasmids on NPRA expression. HEKGCA cells grown in 6-well plates were transfected with psiNPRA (2 ug). Forty eight hours later, total protein was extracted and Western blotted using an antibody to NPRA. Plasmids encoding ANP, Np73-102 and VD were used as controls since they have been shown to down regulate NPRA expression. In the third experiment, HEKGCA cells grown in 6-well plates were transfected with psiNPRA (2 ug), as indicated and forty eight hours later total protein were extracted western blotted using an antibody to NPRA (FIG. 3C). Untransfected cells and cells transfected with U6 vector plasmid without any siNPRA were used as control. Also, filters were stripped and reprobed with antibody to beta-actin.

FIGS. 12A and 12B show inhibitory effect of siRNA in vitro and in vivo. HEKGCA cells grown in 6-well plates were transfected with psiNPRA (2 ug). Forty eight hours later, cells were subjected to flow cytometry to detect NPRA positive cells using an antibody to NPRA. U6 plasmid without any siRNA and plasmid encoding Kp73-102 were used as controls, since the latter has been shown to down regulate NPRA expression. Results are shown in FIG. 12A. Mice (n=4) were intranasally administered with 25 ug siRNA plasmids complexed with 125 ul of chitosan nanoparticles. BAL was done 72 hours later. Cells were stained by NPRA Ab. NPRA expression cells were counted.

FIGS. 13A, 13B-1, and 13B-2 show that SiNPRA treatment appears to reduce cytokine production in BALB/c mice. 4-6 week old BALB/c mice (n=3) were sensitized and challenged with OVA (50 μg). All mice were sensitized intra-peritoneally (i.p.) and then challenged intranasally (i.n.). Mice were given two Si NPRA treatments by lavage and challenged 24 hours later. Thoracic lymph node cells (FIG. 13A) and spleen cells (FIGS. 13B-1 and 13B-2) were removed and cells cultured for 48 hours in the presence of OVA (Sigma Grade V) and recombinant mouse IL-2. Naive mice received no treatment. Cells were treated with GolgiStop (BD Pharmingen) and stained for surface and intracellular cytokines (Antibodies obtained from BD Pharmingen). Percent cytokine secreting cells were quantified by intracellular cytokine staining using flow cytometry.

FIGS. 14A and 14B show that administration of siNPRA decreases inflammation of the lung in BALB/c mice 4-6 week old BALB/c mice (n=3) were sensitized and challenged with OVA (50 μg). All mice were sensitized intra-peritoneally (i. p.) and then challenged intranasally (i.n.). Mice were given two Si NPRA treatments by lavage and challenged 24 hours later. Lungs were obtained 24 hours after challenge, fixed in formalin sectioned and stained with hematoxylin and eosin.

FIGS. 15A-15C show that administration of siNPRA8 by the transdermal route decreases NPRA expression, eosinophilia of the lung and BAL IL-4 cytokine. BALB/c mice (n=5 each group) were sensitized (i.p.) and challenged (i.n.) with 50 μg of OVA. Mice were given siNPRA8 oligonucleotide treatments by transdermal route and challenged 4 hours later. Following 24 hours of challenge two mice were sacrificed to obtain lungs and which were fixed sectioned and immunostained for NPRA expression (FIG. 15A). Mice (n=3) were sacrificed and lavaged and the percentage of eosinophils (FIG. 15B) and IL-4 concentration (FIG. 15C) in the lavage fluid was determined.

FIGS. 16A and 16B show that administration of siNPRA decreases inflammation of the lung in BALB/c mice. BALB/c mice (n=5 each group) were sensitized (i. p.) and challenged (i.d.) with 50 μg of OVA. All mice were sensitized intra-peritoneally (i.p.) and then challenged intranasally (i.n.) Mice were given siNPRA8 oligonucleotide treatments transdermally (si8) and challenged 4 hours later. Lungs were obtained 24 hours after challenge, fixed in formalin, sectioned and stained with hematoxylin and eosin.

FIG. 17 shows that administration of siNPRA inhibits NPRA expression in the respiratory syncytial virus (RSV) infected lung. R T-PCR analysis of NPRA expression in the lung of mice treated with siRNA. psiNPRA9 was encapsulated with chitosan nanoparticles and intranasally delivered to mice. Twenty-four hours later mice were infected with RSV (5×106 pfumouse). Four days later, mice were sacrificed and lung were collected for RNA extraction. NPRA fragment were amplified by RT-PCR and analyzed in 1% agarose gel.

FIGS. 18A and 18B show that administration of siNPRA inhibits the Respiratory syncytial virus infection of A549 cells. A549 cells were grown in 6 well plate, transfected by siNPRA8, siNPRA9 or control U6 plasmid (2.0 ug) and 2 hours after infected by rgRSV (MOI=0.2). Cells were checked for infection 48 hours later, FACS was done and the results are shown in FIG. 18A. A549 cells were grown in 6 well plates infected by rgRSV (MOI=0.2) and 24 hours after infection they were transfected by siNPRA8 siNPRA9 or control U6 plasmid (2. g) and further 24 hours later, flow cytometry was performed to estimate percentage of infected cells. Results are shown in FIG. 18B.

FIG. 19 shows those NPRA deficient mice are resistant to melanoma tumor formation and metastasis in the B16 mouse model. B16 melanoma cells (1.3×10) were injected subcutaneously into twelve-week-old female C57BL/6 mice and NPRA deficient mice. Mice were observed for tumor formation for one month, and then sacrificed on day-22. Tumors were then removed and weighed.

FIGS. 20A-20E show that siNPRA treatment decreases melanoma tumor formation in b16 mouse model. B16 melanoma cells (1.3×10⁵) were injected subcutaneously into twelve-week old female C57BL/6 mice. These mice were then treated with 33 μg of siNPRA-oligos, siNPRA plasmid, or scrambled oligos. All of these were mixed with chitosan at a ratio of 1:2. 5. Mixed chitosan and plasmid or oligos were mixed again with cream before application to the injection area. The control group was given cream only. These treatments were given twice a week. Mice were sacrificed on day—and tumors were removed and weighed.

FIGS. 21A-21C show the effect of NPRA deficiency on melanoma. To test of the anti-melanoma activity of decreased NPRA levels NPRA-mice (n=12) and wild type (n=12) were injected s.c. with B16 melanoma cells. The tumor size (FIG. 21A) over several days post injection and tumor burden (FIG. 21B) at day 18 were measured. FIG. 21C shows that siNPRA treatment decreases melanoma tumor formation in the B16 mouse model. B16 melanoma cells (1.3×10⁵) were injected subcutaneously into twelve-week old female mice. These mice were then treated with 33 μg of siNPRA-oligos, siNPRA plasmid, or scrambled oligos. All of these were mixed with chitosan at a ratio of 1:2. 5. Mixed chitosan and plasmid or oligos were mixed again with cream before application to the injection area. The control group was given cream only and these treatments were given twice a week. Mice and tumors were removed and weighed.

FIGS. 22A and 22B show that siNPRA treatment decreases Lewis lung carcinoma. Groups of wild type and NPRA-mice (n=8 per group) were injected s.c. with 2×10⁶ LLC1 cells. Tumor sizes were measured on day 10, 13, 15 and 17 (FIG. 22A) and tumor weights at day 17 (FIG. 22B) were compared.

FIG. 23 shows that siNPRA treatment decreases ovarian cancer. Groups of wild type and NPRA^{-/-} mice (n=8) were injected s.c. with 2×10⁶ mouse ovarian cancer ID-8 cells and tumor sizes were measured every week after ID8 injection.

FIG. 24 shows that NPRA expression and signaling is involved in lung inflammation. NPRA^{-/-} mice exhibit reduced lung inflammation. Wild type (WT) C57BL/6 and NPRA^{-/-} mice (n=4) were sensitized (i.p.) at day zero and day seven and then challenged twice with OVA. Two days later, mice were sacrificed and lung sections were stained with hematoxylin/eosin.

FIGS. 25A-25B shows that NPRA is over-expressed in various cancer cells compared to normal cells. Whole proteins were extracted from different cell lines and subjected to Western blot using primary antibodies against NPRA. Beta actin is used as a loading control. Cell lines used are as follows. (FIG. 25A) Normal cells: Mouse cell (NIH3T3), Normal human bronchial epithelial cells (NHBE). Cancer cells: LLC-1, Mouse lewis lung carcinoma; A549, human lung adenocarcinoma; B16, mouse melanoma; Skov3, human ovarian cancer, ID8, mouse ovarian cancer cells; DU145, mouse prostate cancer cells and (FIG. 25B) Normal cells, melanocytes; and human melanoma cells: A375, 624, Sk-mel-28, Sk-mel-5; mouse melanoma cells: K1735, CM3205, CM519.

FIGS. 26A-26C show that siNPRA nanoparticles decrease tumor burden. (FIG. 26A) Nanoparticle-transported siRNA, but not naked siRNA is retained in the tumor. BALB/c nude mice injected s.c. with PC3 prostate cancer cells were treated with chitosan-siGLO nanocomplexes or naked siGLO and tumor sections were examined after 48 hrs by fluorescence microscopy. (FIG. 26B) To identify the most effective siRNA, several candidates were screened and three were identified that inhibited NPRA expression. HEK293-GCA cells that overexpress NPRA were transfected with one of these siNPRAs or with scrambled siNPRA, and cell lysates were examined at 48 hours for NPRA expression by western blotting. (FIG. 26C) B16 melanoma cells (1.5×10⁵) were injected subcutaneously into twelve-week old female C57BL/6 mice. These mice were then treated with synthetic siNPRA, vector-driven siNPRA (psiNPRA), or scrambled siNPRA (Scr). All of these were mixed with chitosan at a ratio of 1:2.5. Mixed chitosan and plasmid or oligos were mixed again with a cream before application to the injection area. The control group was given cream only. These treatments were given twice a week. Mice were sacrificed on day twenty second, tumors were removed and weighed. Values shown are mean (n=16)±SD. p<0.01.

FIGS. 27A-27E show that pNP73-102 nanoparticles decrease NPRA expression and lung tumor development. (FIG. 27A) Modulation of NPRA expression by NP73-102 in vivo. Pregnant (12 d) mice were injected with pNP73-102 or pVAX1 (control vector). After 1 day, mice were sacrificed and the expression of NPRA and NPRC was measured by flow cytometry in CD4+-gated cells. (FIG. 27B) Expression of NP73-102-FLAG in BAL cells after i.n. administration of pNP-73-102-FLAG peptide. After 24 hrs, BAL cells were stained with either second antibody as control or anti-FLAG antibody and then with DAPI. (FIG. 27C) Nude mice were given 5×10⁶ A549 cells intravenously and weekly i.n. doses of nanoparticles carrying either empty plasmid (control) or pNP73-102. Three weeks later, mice were sacrificed and lung sections were stained with hematoxylin/eosin and examined for tumor nodules. (FIG. 27D) Lung sections were also stained with antibodies to cyclin B and phosphoBad. (FIG. 27E) BALB/c mice were given pNP73-102 on days 1 and 3, and injected s.c. with 10⁵ Line-1 cells on day 7. From then on, the mice were given pNP73-102 at weekly intervals. Mice were sacrificed on day 40 and their tumor burden was determined based on size and weight. Control group (C) received no treatment and a second control group (V) received nanoparticles containing pVAX. Values shown are mean (n=16)±SD. p<0.01.

FIG. 27F shows that HEK293 cells were cotransfected with pNPRA-Luc and pNP73-102 or pVAX1. Forty-eight hrs later, cells were harvested and lyzed with luciferase reporter lysis buffer. The supernatants were subjected to luciferase assay (*p<0.05, **p<0.01).

FIGS. 28A-28E show a mechanism of tumor suppression by NP73-102 and NPRA deficiency. (FIGS. 28A-28C), NP73-102 induced apoptosis in cancer cells. (FIG. 28A) pNP73-102 does not induce apoptosis of normal cells, only A549 cancer cells. A549 adenocarcinoma or normal IMR90 cells were transfected with pVAX1 or pNP73-102. Cells were stained by TUNEL assay and nuclei were visualized with DAPI. TUNEL-positive cells were counted under a fluorescence microscope and the number was expressed as percent TUNEL-positive cells relative to the total number of cells, less NPRA positive cells were detected after pNP73-102 treatment (p<0.01). (FIG. 28B) Proteins were isolated and equal amounts were western-blotted using an antibody to poly-ADP ribose polymerase (PARP). (FIG. 28C) B16 melanoma cells were transfected with pVAX or pNP73-120, respectively. TUNEL-positive cells were counted under a fluorescence microscope and the number was expressed as percent TUNEL-positive cells relative to the total number of cells. (FIG. 28D, FIG. 28E) NFκB and pRb are involved in tumor suppression in NPRA-deficient mice. (FIG. 28D) NPRA deficiency inactivated NFκB and down regulated VEGF expression. Whole proteins were extracted from lungs of wild type and NPRA^{-/-} mice, and then subjected to Western blot using primary antibodies against NFκB, phospho-NFκB and VEGF. (FIG. 28E) Differential expression of pRb in the lungs of wild type and NPRA−/− mice. Lungs of wild type and NPRA^{-/-} C57BL/6 mice (n=4) were sectioned and examined for pRb expression using phospho-pRb antibody in immunohistological staining Arrows directed to the phospho-pRb-positive cells.

FIG. 28D shows NPRA deficiency inactivated NFκB and down regulated VEGF expression. Whole proteins were extracted from lungs of wild type and NPRA^{-/-} mice, and then subjected to Western blot using primary antibodies against NFκB, phospho-NFκB and VEGF. FIG. 28 (FIG. 28E) shows differential expression of pRb in the lungs of wild type and NPRA−/− mice. Lungs of wild type and NPRA^{-/-} C57BL/6 mice (n=4) were sectioned and examined for pRb expression using phospho-pRb antibody in immunohistological staining

FIGS. 29A-29D show those NPRA knockout mice are resistant to propagate TRAMP-C1 prostate tumor cells.

FIG. 30A depicts an example of where NPRA knockout mice and wild type mice with were injected with breast carcinoma cells.

FIG. 30B shows the difference between NPRA knockout mice and wild type mice in the amount of tumors.

FIG. 30C shows the differences in PARP cleavage by Western blots in human breast cancer cells treated with pNP73-102 and control.

FIG. 30D shows the difference in apoptosis in human breast cancer cells treated with pNP73-102 and psiNRPA8 vs. controls.

FIGS. 31A-31E show NPRA deficiency decreases pulmonary inflammation. Groups (n=3) of wild type DBA/2 (wt) and NPR-C deficient (NPRC^{-/-}) mice (FIG. 31A) and wild type C57/BL6 (wt) and NPR-A (NPRA^{-/-}) (FIG. 31B) were sensitized with OVA (20 μg/mouse) and after 2 weeks challenged i.n. with OVA (20 μg/mouse). One day later mice were sacrificed and lung sections were stained with H & E to examine inflammation. The levels cytokines (IL-4, IL-5 and IL-6) were measured in BAL fluid of WT and NPRA−/− mice (FIG. 31C). (FIGS. 31D-31E) show a diagram depicting that over expression of ANP in the lung augments inflammation and cytokine production in splenocytes. (FIG. 31D) Normal BALB/c mice were given i.n. nanoparticles carrying pANP (b) or pVAX (a) and their lungs were examined 3 days after by staining the sections (H&E), showing goblet cell hyperplasia. (FIG. 31E) Female BALB/c mice were given i.p. OVA (with alum) and then challenged i.n. OVA. Mice were sacrificed, the spleens aseptically removed and the cells were cultured for 48 hours in the presence of OVA (Sigma) and recombinant IL-2. Cells were removed from culture and stained for surface markers CD4 and CD3 and intracellular cytokines IL-4, IL-10 and IFN-γ (BD Pharmingen).

FIGS. 32A-32G illustrate that NPRA^{-/-} mice are resistant to tumorigenesis. (FIG. 32A and FIG. 32B) Groups of wild type and NPRA^{-/-} mice (n=8 per group) were injected s.c. with 2×10⁶ LLC1 cells. Tumor sizes (FIG. 32A) were measured on day 10, 13, 15 and 17 and tumor weights (FIG. 32B) at day 17 were compared (p<0.01). (FIG. 32C and FIG. 32D) Groups of wild type and NPRA^{-/-} mice (n=12) were injected s.c. with 2×10⁶ B16 melanoma cells and tumor sizes (FIG. 32C) were measured on day 10, 13, 15 and 17 and tumor weight (FIG. 32D) were measured and compared at day 18 (p<0.01). Data from one of the two repeated experiments is presented. (FIG. 32E and FIG. 32F) Groups of wild type and NPRA^{-/-} mice (n=12) were injected s.c. with 2×10⁶ MCF7 breast cancer cells and tumor sizes (FIG. 32E) were measured on day 9, 15, 20 and 25 and tumor weight (FIG. 32F) were measured and compared at day 25 (p<0.01). Data from one of the two repeated experiments is presented. (FIG. 32G) Groups of wild type and NPRA^{-/-} mice (n=8) were injected s.c. with 2×10⁶ mouse ovarian cancer ID8 cells and tumor sizes were measured every week after ID8 injection.

FIGS. 33A-33D show that A549 cells transfected with pNP₇₃₋₁₀₂ show a significantly higher level of apoptosis compared to pANP or pVAX control. Cells were transfected with pNP73-102, pANP or pVAX (as control) and cells were stained with PI and annexin and quantified by flow cytometry (FIG. 33A). A significantly higher apoptosis is seen in A549 adenocarcinoma cells compared to normal IMR-90 cells, as shown by TUNEL assay of A549 cells cultured in 8-chamber slide following a 48-hour transfection with either pANP or pNP73-102 (FIG. 33B) and by PARP cleavage as revealed by western blotting (FIG. 33C). (FIG. 33D) shows that pNP73-102 decreases tumorigenesis in a colony formation assay by A549. Six centimeter tissue culture plates were covered with 4 ml of 0.5% soft agar. A549 cells were transfected with pANP, pNP₇₃₋₁₀₂ or pVAX plasmid DNA (V) or nothing. After 40 h of transfection, cells were suspended in 2 ml of 0.3% soft agar and added to each plate. Cells were plated in duplicate at a density of 2×10⁴ cells/dish and incubated for two weeks. Plates were photographed under a microscope. Cell colonies were counted and plotted. The results of one representative experiment of two are shown.

FIGS. 34A-34D show that cells transfected with pNP₇₃₋₁₀₂ undergo a significantly higher level of apoptosis compared to pANP or pVAX control in melanoma, ovarian and breast cancer cells. (FIG. 34A and FIG. 34B) B16 melanoma cells were transfected with pNP73-102, pANP or pVAX (as control) and cells were examined for apoptosis by TUNEL and annexin-PI staining (FIG. 34C) SKOV3 ovarian cancer cells were grown on a 4-well chamber slide. Cells were transfected with 1 ug of pNP73-102 or pVAX1 and examined for apoptosis by TUNEL. Top, green cells indicated apoptosis; bottom, cells were stained by DAPI. Cells were then observed under the fluorescence microscope. (FIG. 34D and FIG. 34E) MCF-7 breast cancer cells transfected with pNP₇₃₋₁₀₂ show a significantly higher level of apoptosis compared to pVAX control. Cells were transfected with pNP73-102, and pVAX (as control) and cells were examined for apoptosis by TUNEL (FIG. 34D).

FIGS. 35A-35D show the anti-inflammatory property of pNP73-102 in experimental model of asthma. (FIG. 35A and FIG. 35B) shows the effectiveness of pNP73-102 nanoparticles in modulating lung inflammation and eosinophilia when given orally. (FIG. 35C) shows the effectiveness of pNP73-102 nanoparticles in modulating lung function when given intranasally. (FIG. 35D) shows the pNP73-102, not pANP, decreases TH2 (IL-4) cytokine response and increases TH1 (IL-12) response in human dendritic cell and naïve T cell co-cultures.

FIGS. 36A-36D show development of siNPRA system for inhibiting NPRA expression. (FIG. 36A) Cloning of siNPRA sequences in the pU6 vector. The siNPRA sequences were designed as shown in Sequence IDs and cloned in pSilencer (U6) vector using standard procedures. The transformants were tested by digestion with Apa I and EcoR I to release the siRNA inserts. Lane 1, 100 bp ladder; lane 2: pSilencer1(U6), Lane 3, siNPRA8, Lane 7-, siNPRA9 are shown for illustration. (FIG. 36B) show the inhibitory effect of transfected siRNA plasmids on NPRA expression. HEKGCA cells grown in 6-well plates were transfected with psiNPRA (2 ug). Forty eight hours later, total protein was extracted and Western blotted using an antibody to NPRA. (FIG. 36C) In another experiment, HEKGCA cells grown in E-well plates were transfected with psiNPRA (2 ug), as indicated and forty eight hours later total protein were extracted western blotted using an antibody to NPRA (FIG. 11C). Untransfected cells and cells transfected with U6 vector plasmid without any siNPRA were used as control. (FIG. 36D) show inhibitory effect of siRNA in vivo. Mice (n=4) were intranasally administered with 25 μg siRNA plasmids complexed with 125 ul of chitosan nanoparticles. Mice were sacrificed 72 hr later and lung sections were stained with NPRA antibody labeled with FITC. NPRA-expressing cells were observed by fluorescence microscopy. NPRA positive cells were quantified and plotted.

FIGS. 37A-37D show that topical delivery of siRNA chitosan nanoparticles in vitro and in vivo. (FIG. 37A) HEK293 cells were transfected with 200 pmol of siGLO which was complexed with 5 μg of chitosan nanoparticles. Fluorescent cells which contained siGLO were observed by fluorescence microscopy. HEK293 cells transfected with pEGFP-N2 chitosan nanoparticles were included as positive control. (FIG. 37B) Nanoparticle-transported siRNA, but not naked siRNA is retained in the tumor. BALB/c nude mice injected s.c. with PC3 prostate cancer cells were treated with chitosan-siGLO nanocomplexes or naked siGLO and tumor sections were examined after 48 hrs by fluorescence microscopy. (FIG. 37C) The green fluorescence from the frozen lung sections of mice treated by transdermal siGLO nanoparticles or intranasal pEGFP-N2 nanoparticles was monitored by fluorescence microscopy. Untreated lung section (naïve) is shown for comparison. (FIG. 37D) siGLO nanoparticle cream containing 2 nmol of siGLO was spread on the back of Balb/c nude mice. The same dose of siGLO nanoparticles was administered 24 h later. The topically-delivered siGLO were detected 48 h after the initial treatment by in vivo imaging using Xenogen IVIS system. Mice receiving intranasal pEGFP-N2 chitosan nanoparticles were included as positive control. Mice with no treatment (naïve) is shown for comparison.

FIGS. 38A-38E show that administration of siNPRA8 by the topical (transdermal) route decreases NPRA expression, eosinophilia of the lung and BAL IL-4 cytokine BALB/c mice (n=5 each group) were sensitized (i.p.) and challenged (i.n.) with 50 μg of OVA. Mice were given siNPRA8 or scrambled oligonucleotide treatments by transdermal route and challenged 4 hours later. Following 24 hours of challenge two mice were sacrificed to obtain lungs and which were fixed sectioned and immunostained for NPRA expression (FIG. 38A). Lung sections of naïve mouse is shown for comparison. (FIG. 38B) Transdermally-delivered siNPRA reduced airway hyperreactivity. AHR was recorded on day 22 in a whole-body plethysmograph which measures the enhanced pause (PENH). The Penh values were averaged and expressed for each MCh concentration as a percentage of the PBS baseline reading. (FIG. 38C) Transdermally-delivered siNPRA reduced inflammation of the lung. Lungs were obtained 24 hours after challenge, fixed in formalin, sectioned and stained with hematoxylin and eosin. (FIG. 38D) Reduction of eosinophils by siNPRA-imiquimod treatment. Mice (n=4) were sacrificed and lavaged and the percentage of eosinophils. BAL cells were air dried and stained with a modified Wright's stain. Total cell numbers were approximately the same in each group and the number of eosinophils is given as percentage of the total (**p<0.01). (FIG. 38E) IL-4 in BAL fluid was measured by IL-4 ELISA. Significant reduction of IL-4 was achieved by siNPRA-imiquimod treatment when compared with OVA controls (**p<0.01).

FIG. 39 Lungs of all animals from the four groups were removed and homogenized. The levels of IL-2, IL-5, IFN-γ and TNFα in lung homogenate were measured using a mouse Th1/Th2 Cytokine CBA kit following the manufacturer's instruction (BD Bioscience, CA). IL-5 was also significantly downregulated by siNPRA treatment (*p<0.05).

FIGS. 40A-40D show that SiNPRA treatment reduces lung inflammation and alters cytokine production profile in BALB/c mice. BALB/c mice (4-6 week old, n=6) were sensitized and challenged with OVA (50 μg). All mice were sensitized intra-peritoneally (i.p.) and then challenged intranasally (i.n.). Mice were given two Si NPRA treatments by gavage and challenged 24 hours later. Controls were given scrambled siNPRA (Scr). (FIG. 40A) To determine whether siNPRA can prevent AHR, groups of mice were challenged with 6.25% and 25% methacholine on day 22 and AHR was measured. (FIG. 40B) Lungs were obtained 24 hours after challenge, fixed in formalin, sectioned and stained with hematoxylin and eosin. (FIG. 40C) A lavage was performed and the percentage of eosinophils was determined. (FIG. 40D) spleen cells were removed and cells cultured for 48 hours in the presence of OVA (Sigma Grade V) and recombinant mouse IL-2. Naïve mice received no treatment. Cells were treated with GolgiStop (BD Pharmingen) and stained for surface and intracellular cytokines (Antibodies obtained from BD Pharmingen). Percent cytokine secreting cells were quantified by intracellular cytokine staining using flow cytometry.

FIGS. 41A-41C show that administration of siNPRA inhibits NPRA expression in the respiratory syncytial virus (RSV) infected lung. (FIG. 41A) RT-PCR analysis of NPRA expression in the lung of mice treated with siRNA. psiNPRA9 was encapsulated with chitosan nanoparticles and intranasally delivered to mice. Twenty-four hours later mice were infected with RSV (5×10⁶ pfu/mouse). Four days later, mice were sacrificed and lung were collected for RNA extraction. NPRA fragment were amplified by RT-PCR and analyzed in 1% agarose gel. FIG. 41B and FIG. 41C show that administration of siNPRA inhibits the Respiratory syncytial virus infection of A549 cells. A549 cells were grown in 6 well plate, transfected by siNPRA8, siNPRA9 or control U6 plasmid (2.0 ug) and 2 hours after infected by rgRSV (MOI=0.2). Cells were checked for infection 48 hours later, FACS was done and the results are shown in FIG. 41B. A549 cells were grown in 6 well plates infected by rgRSV (MOI=0.2) and 24 hours after infection they were transfected by siNPRA8 siNPRA9 or control U6 plasmid (2. g) and further 24 hours later, flow cytometry was performed to estimate percentage of infected cells. Results are shown in FIG. 41C.

### BRIEF DESCRIPTION OF THE SEQUENCES

SEQ ID NO:1 is the amino acid sequence of human “long acting natriuretic peptide” or NP₁₋₃₀: ¹NPMYN AVSNADLMDF KNLLDHLEEK MPLED³⁰ (SEQ ID NO:1).

SEQ ID NO:2 is the amino acid sequence of human “vessel dilator” or NP₃₁₋₆₇: ³¹EVVPP QVLSEPNEEA GAALSPLPEV PPWTGEVSPA QR⁶⁷ (SEQ ID NO:2).

SEQ ID NO:3 is the amino acid sequence of human “kaliuretic peptide” or NP₇₉₋₉₈: ⁷⁹SSDRSAL LKSKLRALLT APR⁹⁸ (SEQ ID NO:3).

SEQ ID NO:4 is the amino acid sequence of human “atrial natriuretic peptide” (ANP) or NP_{99-126.} ⁹⁹SLRRSSC FGGRMDRIGA QSGLGCNSFR Y¹²⁶ (SEQ ID NO:4).

SEQ ID NO:5 is the amino acid sequence of cloned mouse pNP₇₃₋₁₀₂: ⁷³ GSPWDPSDRS ALLKSKLRAL LAGPRSLRR¹⁰² (SEQ ID NO:5).

SEQ ID NO:6 is the amino acid sequence of cloned mouse NP fragment: VSNTDLMDFK NLLDHLEEKM PVEDEVMPPQ ALSEQTE (SEQ ID NO:6).

SEQ ID NO:7 is the amino acid sequence for the human preproANP (NCBI ACCESSION # NM_{—}006172) wherein the underlined amino acids represent the signal sequence which is cleaved off to form the mature peptide:

(SEQ ID NO: 7)
¹MSSFSTTTVS FLLLLAFQLL GQTRANPMYN AVSNADLMDF
KNLLDHLEEK MPLEDEVVPP QVLSEPNEEA GAALSPLPEV
PPWTGEVSPA QRDGGALGRG PWDSSDRSAL LKSKLRALLT
APRSLRRSSC FGGRMDRIGA QSGLGCNSFR Y¹⁵¹.

SEQ ID NO:8 is a forward primer for the cDNA sequence encoding mouse prepro ANF protein: 5′-gac ggc aag ctt act atg ggc agc ccc tgg gac cc-3′ (SEQ ID NO:8).

SEQ ID NO:9 is a reverse primer for the cDNA sequence encoding mouse pre-proANF protein: 5′-acc ccc ctc gag tta tta tct tcg tag get ccg-3′ (SEQ ID NO:9).

SEQ ID NO:10 is a forward primer for the cDNA sequence encoding mouse NP fragment: 5′-aat cct aag ctt agt atg gtg tcc aac aca gat-3′ (SEQ ID NO:10).

SEQ ID NO:11 is a reverse primer for the cDNA sequence encoding mouse NP fragment: 5′-tgc gaa ctc gag tta ctc agt ctg ctc act cag ggc ctg cg-3′ (SEQ ID NO:11).

SEQ ID NO:12 is the nucleotide sequence encoding cloned mouse pNP₇₃₋₁₀₂: atg ggc agc ccc tgg gac ccc tcc gat aga tct gcc ctc ttg aaa agc aaa ctg agg gct ctg ctc gct ggc cct cgg agc cta cga aga taa (SEQ ID NO:12).

SEQ ID NO:13 is the nucleotide sequence encoding cloned mouse pNP fragment: atg gtg tcc aac aca gat ctg atg gat ttc aag aac ctg cta gac cac ctg gag gag aag atg ccg gta gaa gat gag gtc atg ccc ccg cag gcc ctg agt gag cag act gag taa (SEQ ID NO:13).

SEQ ID NO:14 is the mRNA nucleotide sequence encoding human ANP (NCBI Accession # NM_{—}006172:

| | (SEQ ID NO: 14) |
|---|---|
| 1 | tggcgaggga cagacgtagg ccaagagagg ggaaccagag |
| | aggaaccaga ggggagagac |
| 61 | agagcagcaa gcagtggatt gctccttgac gacgccagca |
| | tgagctcctt ctccaccacc |
| 121 | accgtgagct tcctcctttt actggcattc cagctcctag |
| | gtcagaccag agctaatccc |
| 181 | atgtacaatg ccgtgtccaa cgcagacctg atggatttca |
| | agaatttgct ggaccatttg |
| 241 | gaagaaaaga tgcctttaga agatgaggtc gtgcccccac |
| | aagtgctcag tgagccgaat |
| 301 | gaagaagcgg gggctgctct cagccccctc cctgaggtgc |
| | ctccctggac cggggaagtc |
| 361 | agcccagccc agagagatgg aggtgccctc gggcggggcc |
| | cctgggactc ctctgatcga |
| 421 | tctgccctcc taaaaagcaa gctgagggcg ctgctcactg |
| | cccctcggag cctgcggaga |
| 481 | tccagctgct tcgggggcag gatggacagg attggagccc |
| | agagcggact gggctgtaac |
| 541 | agcttccggt actgaagata acagccaggg aggacaagca |
| | gggctgggcc tagggacaga |
| 601 | ctgcaagagg ctcctgtccc ctggggtctc tgctgcattt |
| | gtgtcatctt gttgccatgg |
| 661 | agttgtgatc atcccatcta agctgcagct tcctgtcaac |
| | acttctcaca tcttatgcta |
| 721 | actgtagata aagtggtttg atggtgactt cctcgcctct |
| | cccaccccat gcattaaatt |
| 781 | ttaaggtaga acctcacctg ttactgaaag tggtttgaaa |
| | gtgaataaac ttcagcacca |
| 841 | tggac. |

SEQ ID NO:15 is the human gene for atrial natriuretic factor propeptide

(coding sequence includes—join (570 . . . 692, 815 . . . 1141, 2235 . . . 2240); sig. peptide=570 . . . 644; mat. peptide=join (645 . . . 692, 815 . . . 1141, 2235 . . . 2237), (NCBI ACCESSION NO: X01471; Greenberg, B. D. et al., Nature, 1984, 312(5995):656-658):

| | (SEQ ID NO: 15) |
|---|---|
| 1 | ggatccattt gtctcgggct gctggctgcc tgccatttcc |
| | tcctctccac ccttatttgg |
| 61 | aggccctgac agctgagcca caaacaaacc aggggagctg |
| | ggcaccagca agcgtcaccc |
| 121 | tctgtttccc cgcacggtac cagcgtcgag gagaaagaat |
| | cctgaggcac ggcggtgaga |
| 181 | taaccaagga ctctttttta ctcttctcac acctttgaag |
| | tgggagcctc ttgagtcaaa |
| 241 | tcagtaagaa tgcggctctt gcagctgagg gtctgggggg |
| | ctgttggggc tgcccaaggc |
| 301 | agagaggggc tgtgacaagc cctgcggatg ataactttaa |
| | aagggcatct cctgctggct |
| 361 | tctcacttgg cagctttatc actgcaagtg acagaatggg |
| | gagggttctg tctctcctgc |
| 421 | gtgcttggag agctgggggg ctataaaaag aggcggcact |
| | gggcagctgg gagacaggga |
| 481 | cagacgtagg ccaagagagg ggaaccagag aggaaccaga |
| | ggggagagac agagcagcaa |
| 541 | gcagtggatt gctccttgac gacgccagca tgagctcctt |
| | ctccaccacc accgtgagct |
| 601 | tcctcctttt actggcattc cagctcctag gtcagaccag |
| | agctaatccc atgtacaatg |
| 661 | ccgtgtccaa cgcagacctg atggatttca aggtagggcc |
| | aggaaagcgg gtgcagtctg |
| 721 | gggccagggg gctttctgat gctgtgctca ctcctcttga |
| | tttcctccaa gtcagtgagg |
| 781 | tttatccctt tccctgtatt ttccttttct aaagaatttg |
| | ctggaccatt tggaagaaaa |
| 841 | gatgccttta gaagatgagg tcgtgccccc acaagtgctc |
| | agtgagccga atgaagaagc |
| 901 | gggggctgct ctcagccccc tccctgaggt gcctccctgg |
| | accggggaag tcagcccagc |
| 961 | ccagagagat ggaggtgccc tcgggcgggg cccctgggac |
| | tcctctgatc gatctgccct |
| 1021 | cctaaaaagc aagctgaggg cgctgctcac tgcccctcgg |
| | agcctgcgga gatccagctg |
| 1081 | cttcgggggc aggatggaca ggattggagc ccagagcgga |
| | ctgggctgta acagcttccg |
| 1141 | ggtaagagga actggggatg gaaatgggat gggatggaca |
| | ctactgggag acaccttcag |
| 1201 | caggaaaggg accaatgcag aagctcattc cctctcaagt |
| | ttctgcccca acacccagag |
| 1261 | tgccccatgg gtgtcaggac atgccatcta ttgtccttag |
| | ctagtctgct gagaaaatgc |
| 1321 | ttaaaaaaaa aagggggggg gctgggcacg gtcgtcacgc |
| | ctgtaatccc agcactttgg |
| 1381 | gaggccaggc agcggatcat gaggtcaaga gatcaagact |
| | atcctggcca acatggtgaa |
| 1441 | accccagctc tactaaaaat acaaaaatta gctgggtgtg |
| | tggcgggcac ctgtactctc |
| 1501 | agctacttgg gaggctgagg caggagaatc acttgaaccc |
| | aggaggcaga ggttgcagtg |
| 1561 | agcagagatc acgccactgc agtccagcct aggtgataga |
| | gcgagactgt ctcaaaaaaa |
| 1621 | aaaaaaaaag gccaggcgcg gtggctcacg cctgtaatcc |
| | cagcgctttg ggaggccaag |
| 1681 | gcgggtggat cacgaggtca ggagatggag accatcctgg |
| | ctaacacggt gaaaccccgt |
| 1741 | ctctactaaa aatacaaaaa attagccagg cgtggtggca |
| | ggcgcctgta agtcctagct |
| 1801 | actccggagg ctgaggcagg agaatggcgt gaacccggga |
| | ggcggagctt gcagtgagca |
| 1861 | gagatggcac cactgcactc cagcctgggc gacagagcaa |
| | gactccgtct caaaaaaaaa |
| 1921 | aaaaaaaaaa gcaactgcca ctagcactgg gaaattaaaa |
| | tattcataga gccaagttat |
| 1981 | ctttgcatgg ctgattagca gttcatattc ctccccagaa |
| | ttgcaagatc ctgaagggct |
| 2041 | taagtgaaat ttactctgat gagtaacttg cttatcaatt |
| | catgaagctc agagggtcat |
| 2101 | caggctgggg tgggggccgg tgggaagcag gtggtcagta |
| | atcaagttca gaggatgggc |
| 2161 | acactcatac atgaagctga cttttccagg acagccaggt |
| | caccaagcca gatatgtctg |
| 2221 | tgttctcttt gcagtactga agataacagc cagggaggac |
| | aagcagggct gggcctaggg |
| 2281 | acagactgca agaggctcct gtcccctggg gtctctgctg |
| | catttgtgtc atcttgttgc |
| 2341 | catggagttg tgatcatccc atctaagctg cagcttcctg |
| | tcaacacttc tcacatctta |
| 2401 | tgctaactgt agataaagtg gtttgatggt gacttcctcg |
| | cctctcccac cccatgcatt |
| 2461 | aaattttaag gtagaacctc acctgttact gaaagtggtt |
| | tgaaagtgaa taaacttcag |
| 2521 | caccatggac agaagacaaa tgcctgcgtt ggtgtgcttt |
| | ctttcttctt gggaagagaa |
| 2581 | ttc. |

SEQ ID NO:16 is the amino acid sequence for the mouse preproANP peptide:

(SEQ ID NO: 16)
MGSFSITLGF FLVLAFWLPG HIGANPVYSA VSNTDLMDFK
NLLDHLEEKM PVEDEVMPPQ ALSEQTEEAG AALSSLPEVP
PWTGEVNPPL RDGSALGRSP WDPSDRSALL KSKLRALLAG
PRSLRRSSCF GGRIDRIGAQ SGLGCNSFRY RR.

SEQ ID NO: 17 is the genetic sequence for the mouse preproANP peptide wherein the coding sequence starts at nucleic acid molecule position 81 and ends at nucleic acid molecule position 539:

(SEQ ID NO: 17)

| | |
|---|---|
| 1 | caaaagctga gagagagaga gaaagaaacc agagtgggca |
| | gagacagcaa acatcagatc |
| 61 | gtgccccgac ccacgccagc atgggctcct tctccatcac |
| | cctgggcttc ttcctcgtct |
| 121 | tggccttttg gcttccaggc catattggag caaatcctgt |
| | gtacagtgcg gtgtccaaca |
| 181 | cagatctgat ggatttcaag aacctgctag accacctgga |
| | ggagaagatg ccggtagaag |
| 241 | atgaggtcat gcccccgcag gccctgagtg agcagactga |
| | ggaagcaggg gccgcactta |
| 301 | gctccctccc cgaggtgcct ccctggactg gggaggtcaa |
| | cccacctctg agagacggca |
| 361 | gtgctctagg gcgcagcccc tgggacccct ccgatagatc |
| | tgccctcttg aaaagcaaac |
| 421 | tgagggctct gctcgctggc cctcggagcc tacgaagatc |
| | cagctgcttc gggggtagga |
| 481 | ttgacaggat tggagcccag agtggactag gctgcaacag |
| | cttccggtac cgaagataac |
| 541 | agccaaggag gaaaaggcag tcgattctgc ttgagcagat |
| | cgcaaaagat cctaagccct |
| 601 | tgtggtgtgt cacgcagctt ggtcacattg ccactgtggc |
| | gtggtgaaca ccctcctgga |
| 661 | gctgcggctt cctgccttca tctatcacga tcgatgttaa |
| | atgtagatga gtggtctagt |
| 721 | ggggtcttgc ctctcccact ctgcatatta aggtagatcc |
| | tcaccctttt cagaaagcag |
| 781 | ttggaaaaaa aaaaaaagaa taaacttcag caccaaggac |
| | agacgccgag gccctgatgt |
| 841 | gcttctttgg cttctgccct cagttctttg ctctcccc. |

SEQ ID NO:18 is amino acid sequence of human natriuretic peptide receptor-A (NPR-A):

(SEQ ID NO: 18)
MPGPRRPAGSRLRLLLLLLLPPLLLLLRGSHAGNLTVAVVLPLANTSYP
WSWARVGPAVELALAQVKARPDLLPGWTVRTVLGSSENALGVCSDTAAP
LAAVDLKWEHNPAVFLGPGCVYAAAPVGRFTAHWRVPLLTAGAPALGFG
VKDEYALTTRAGPSYAKLGDFVAALHRRLGWERQALMLYAYRPGDEEHC
FFLVEGLFMRVRDRLNITVDHLEFAEDDLSHYTRLLRTMPRKGRVIYIC
SSPDAFRTLMLLALEAGLCGEDYVFFHLDIFGQSLQGGQGPAPRRPWER
GDGQDVSARQAFQAAKIITYKDPDNPEYLEFLKQLKHLAYEQFNFTMED
VLVNTIPASFHDGLLLYIQAVTETLAHGGTVTDGENITQRMWNRSFQGV
TGYLKIDSSGDRETDFSLWDMDPENGAFRVVLNYNGTSQELVAVSGRKL
NWPLGYPPPDIPKCGFDNEDPACNQDHLSTLEVLALVGSLSLLGILIVS
FFIYRKMQLEKELASELWRVRWEDVEPSSLERHLRSAGSRLTLSGRGSN
YGSLLTTEGQFQVFAKTAYYKGNLVAVKRVNRKRIELTRKVLFELKHMR
DVQNEHLTRFVGACTDPPNICILTEYCPRGSLQDILENESITLDWMFRY
SLTNDIVKGMLFLHNGAICSHGNLKSSNCVVDGRFVLKITDYGLESFRD
LDPEQGHTVYAKKLWTAPELLRMASPPVRGSQAGDVYSFGIILQEIALR
SGVFHVEGLDLSPKEIIERVTRGEQPPFRPSLALQSHLEELGLLMQRCW
AEDPQERPPFQQIRLTLRKFNRENSSNILDNLLSRMEQYANNLEELVEE
RTQAYLEEKRKAEALLYQILPHSVAEQLKRGETVQAEAFDSVTIYFSDI
VGFTALSAESTPMQVVTLLNDLYTCFDAVIDNFDVYKVETIGDAYMVVS
GLPVRNGRLHACEVARMALALLDAVRSFRIRHRPQEQLRLRIGIHTGPV
CAGVVGLKMPRYCLFGDTVNTASRMESNGEALKIHLSSETKAVLEEFGG
FELELRGDVEMKGKGKVRTYWLLGERGSSTRG.

(NCBI ACCESSION NO. NM_{—}000906; Airhart N. et al., J. Biol. Chem., 2003, 278(40):38693-38698; Pitzalis M. V. et al., J. Hypertens., 2003, 21(8):1491-1496; Mokentin J. D. J. Clin. Invest., 2003, 111(9):1275-1277; De L. et al., J. Biol. Chem., 2003, 278(13):11159-11166; Knowles J. W. et al., Hum. Genet., 2003, 12(1):62-70; Pandy K. N. et al., J. Biol. Chem., 2002, 277(7):4618-4627).

SEQ ID NO:19 is the nucleotide coding sequence for human natriuretic peptide receptor-A (NPR-A):

| | |
|---|---|
| | (SEQ ID NO: 19) |
| | ggttccctcc ggatagccgg agacttgggc cggccggacg |
| | ccccttctgg cacactccct |
| 61 | ggggcaggcg ctcacgcacg ctacaaacac acactcctct |
| | ttcctccctc gcgcgccctc |
| 121 | tctcatcctt cttcacgaag cgctcactcg caccctttct |
| | ctctctctct ctctctctaa |
| 181 | cacgcacgca cactcccagt tgttcacact cgggtcctct |
| | ccagcccgac gttctcctgg |
| 241 | cacccacctg ctccgcggcg ccctgcgcgc ccccctcggt |
| | cgcgcccctt gcgctctcgg |
| 301 | cccagaccgt cgcagctaca gggggcctcg agccccgggg |
| | tgagcgtccc cgtcccgctc |
| 361 | ctgctccttc ccatagggac gcgcctgatg cctgggaccg |
| | gccgctgagc ccaaggggac |
| 421 | cgaggaggcc atggtaggag cgctcgcctg ctgcggtgcc |
| | cgctgaggcc atgccggggc |
| 481 | cccggcgccc cgctggctcc cgcctgcgcc tgctcctgct |
| | cctgctgctg ccgccgctgc |
| 541 | tgctgctgct ccggggcagc cacgcgggca acctgacggt |
| | agccgtggta ctgccgctgg |
| 601 | ccaatacctc gtacccctgg tcgtgggcgc gcgtgggacc |
| | cgccgtggag ctggccctgg |
| 661 | cccaggtgaa ggcgcgcccc gacttgctgc cgggctggac |
| | ggtccgcacg gtgctgggca |
| 721 | gcagcgaaaa cgcgctgggc gtctgctccg acaccgcagc |
| | gcccctggcc gcggtggacc |
| 781 | tcaagtggga gcacaacccc gctgtgttcc tgggccccgg |
| | ctgcgtgtac gccgccgccc |
| 841 | cagtggggcg cttcaccgcg cactggcggg tcccgctgct |
| | gaccgccggc gccccggcgc |
| 901 | tgggcttcgg tgtcaaggac gagtatgcgc tgaccacccg |
| | cgcggggccc agctacgcca |
| 961 | agctggggga cttcgtggcg gcgctgcacc gacggctggg |
| | ctgggagcgc caagcgctca |
| 1021 | tgctctacgc ctaccggccg ggtgacgaag agcactgctt |
| | cttcctcgtg gaggggctgt |
| 1081 | tcatgcgggt ccgcgaccgc ctcaatatta cggtggacca |
| | cctggagttc gccgaggacg |
| 1141 | acctcagcca ctacaccagg ctgctgcgga ccatgccgcg |
| | caaaggccga gttatctaca |
| 1201 | tctgcagctc ccctgatgcc ttcagaaccc tcatgctcct |
| | ggccctggaa gctggcttgt |
| 1261 | gtggggagga ctacgttttc ttccacctgg atatctttgg |
| | gcaaagcctg caaggtggac |
| 1321 | agggccctgc tccccgcagg ccctgggaga gaggggatgg |
| | gcaggatgtc agtgcccgcc |
| 1381 | aggcctttca ggctgccaaa atcattacat ataaagaccc |
| | agataatccc gagtacttgg |
| 1441 | aattcctgaa gcagttaaaa cacctggcct atgagcagtt |
| | caacttcacc atggaggatg |
| 1501 | tcctggtgaa caccatccca gcatccttcc acgacgggct |
| | cctgctctat atccaggcag |
| 1561 | tgacggagac tctggcacat gggggaactg ttactgatgg |
| | ggagaacatc actcagcgga |
| 1621 | tgtggaaccg aagctttcaa ggtgtgacag gatacctgaa |
| | aattgatagc agtggcgatc |
| 1681 | gggaaacaga cttctccctc tgggatatgg atcccgagaa |
| | tggtgccttc agggttgtac |
| 1741 | tgaactacaa tgggacttcc caagagctgg tggctgtgtc |
| | ggggcgcaaa ctgaactggc |
| 1801 | ccctggggta ccctcctcct gacatcccca aatgtggctt |
| | tgacaacgaa gacccagcat |
| 1861 | gcaaccaaga tcacctttcc accctggagg tgctggcttt |
| | ggtgggcagc ctctccttgc |
| 1921 | tcggcattct gattgtctcc ttcttcatat acaggaagat |
| | gcagctggag aaggaactgg |
| 1981 | cctcggagct gtggcgggtg cgctgggagg acgttgagcc |
| | cagtagcctt gagaggcacc |
| 2041 | tgcggagtgc aggcagccgg ctgaccctga gcgggagagg |
| | ctccaattac ggctccctgc |
| 2101 | taaccacaga gggccagttc caagtctttg ccaagacagc |
| | atattataag ggcaacctcg |
| 2161 | tggctgtgaa acgtgtgaac cgtaaacgca ttgagctgac |
| | acgaaaagtc ctgtttgaac |
| 2221 | tgaagcatat gcgggatgtg cagaatgaac acctgaccag |
| | gtttgtggga gcctgcaccg |
| 2281 | acccccccaa tatctgcatc ctcacagagt actgtccccg |
| | tgggagcctg caggacattc |
| 2341 | tggagaatga gagcatcacc ctggactgga tgttccggta |
| | ctcactcacc aatgacatcg |
| 2401 | tcaagggcat gctgtttcta cacaatgggg ctatctgttc |
| | ccatgggaac ctcaagtcat |
| 2461 | ccaactgcgt ggtagatggg cgctttgtgc tcaagatcac |
| | cgactatggg ctggagagct |
| 2521 | tcagggacct ggacccagag caaggacaca ccgtttatgc |
| | caaaaagctg tggacggccc |
| 2581 | ctgagctcct gcgaatggct tcaccccctg tgcggggctc |
| | ccaggctggt gacgtataca |
| 2641 | gctttgggat catccttcag gagattgccc tgaggagtgg |
| | ggtcttccac gtggaaggtt |
| 2701 | tggacctgag ccccaaagag atcatcgagc gggtgactcg |
| | gggtgagcag ccccccttcc |
| 2761 | ggccctccct ggccctgcag agtcacctgg aggagttggg |
| | gctgctcatg cagcggtgct |
| 2821 | gggctgagga cccacaggag aggccaccat tccagcagat |
| | ccgcctgacg ttgcgcaaat |
| 2881 | ttaacaggga gaacagcagc aacatcctgg acaacctgct |
| | gtcccgcatg gagcagtacg |
| 2941 | cgaacaatct ggaggaactg gtggaggagc ggacccaggc |
| | atacctggag gagaagcgca |
| 3001 | aggctgaggc cctgctctac cagatcctgc ctcactcagt |
| | ggctgagcag ctgaagcgtg |
| 3061 | gggagacggt gcaggccgaa gcctttgaca gtgttaccat |
| | ctacttcagt gacattgtgg |
| 3121 | gtttcacagc gctgtcggcg gagagcacgc ccatgcaggt |
| | ggtgaccctg ctcaatgacc |
| 3181 | tgtacacttg ctttgatgct gtcatagaca actttgatgt |
| | gtacaaggtg gagacaattg |
| 3241 | gcgatgccta catggtggtg tcagggctcc ctgtgcggaa |
| | cgggcggcta cacgcctgcg |
| 3301 | aggtagcccg catggccctg gcactgctgg atgctgtgcg |
| | ctccttccga atccgccacc |
| 3361 | ggccccagga gcagctgcgc ttgcgcattg gcatccacac |
| | aggacctgtg tgtgctggag |
| 3421 | tggtgggact gaagatgccc cgttactgtc tctttgggga |
| | tacagtcaac acagcctcaa |
| 3481 | gaatggagtc taatggggaa gccctgaaga tccacttgtc |
| | ttctgagacc aaggctgtcc |
| 3541 | tggaggagtt tggtggtttc gagctggagc ttcgagggga |
| | tgtagaaatg aagggcaaag |
| 3601 | gcaaggttcg gacctactgg ctccttgggg agagggggag |
| | tagcacccga ggctgacctg |
| 3661 | cctcctctcc tatccctcca cacctcccct accctgtgcc |
| | agaagcaaca gaggtgccag |
| 3721 | gcctcagcct cacccacagc agccccatcg ccaaaggatg |
| | gaagtaattt gaatagctca |
| 3781 | ggtgtgctta ccccagtgaa gacaccagat aggacctctg |
| | agaggggact ggcatggggg |
| 3841 | gatctcagag cttacaggct gagccaagcc cacggccatg |
| | cacagggaca ctcacacagg |
| 3901 | cacacgcacc tgctctccac ctggactcag gccgggctgg |
| | gctgtggatt cctgatcccc |
| 3961 | tcccctcccc atgctctcct ccctcagcct tgctaccctg |
| | tgacttactg ggaggagaaa |
| 4021 | gagtcacctg aaggggaaca tgaaaagaga ctaggtgaag |
| | agagggcagg ggagcccaca |
| 4081 | tctggggctg gcccacaata cctgctcccc cgaccccctc |
| | cacccagcag tagacacagt |
| 4141 | gcacagggga gaagaggggt ggcgcagaag ggttgggggc |
| | ctgtatgcct tgcttctacc |
| 4201 | atgagcagag acaattaaaa tctttattcc aaaaaaaaaa |
| | aaaaaa |

(NCBI ACCESSION NO. NM 000906; Airhart N. et al., J. Biol. Chem., 2003, 278(40):38693-38698; Pitzalis M. V. et al., J. Hypertens., 2003, 21(8):1491-1496; Mokentin J. D. J. Clin. Invest., 2003, 111(9):1275-1277; De L. et al., J. Biol. Chem., 2003, 278(13):11159-11166; Knowles J. W. et al., Hum. Genet., 2003, 12(1):62-70; Pandy K. N. et al., J. Biol. Chem., 2002, 277(7):4618-4627).

SEQ ID NO:20 is amino acid sequence of the human atrial natriuretic peptide clearance receptor precursor (ANP-C; also referred to as NPR-C, NPRC, and atrial natriuretic peptide C-type receptor):

(SEQ ID NO: 20)
MPSLLVLTFS PCVLLGWALL AGGTGGGGVG GGGGGAGIGG
GRQEREALPP QKIEVLVLLP QDDSYLFSLT RVRPAIEYAL
RSVEGNGTGR RLLPPGTRFQ VAYEDSDCGN RALFSLVDRV
AAARGAKPDL ILGPVCEYAA APVARLASHW DLPMLSAGAL
AAGFQHKDSE YSHLTRVAPA YAKMGEMMLA LFRHHHWSRA
ALVYSDDKLE RNCYFTLEGV HEVFQEEGLH TSIYSFDETK
DLDLEDIVRN IQASERVVIM CASSDTIRSI MLVAHRHGMT
SGDYAFFNIE LFNSSSYGDG SWKRGDKHDF EAKQAYSSLQ
TVTLLRTVKP EFEKFSMEVK SSVEKQGLNM EDYVNMFVEG
FHDAILLYVL ALHEVLRAGY SKKDGGKIIQ QTWNRTFEGI
AGQVSIDANG DRYGDFSVIA MTDVEAGTQE VIGDYFGKEG
RFEMRPNVKY PWGPLKLRID ENRIVEHTNS SPCKSSGGLE
ESAVTGIVVG ALLGAGLLMA FYFFRKKYRI TIERRTQQEE
SNLGKHRELR EDSIRSHFSV A

(NCBI ACCESSION NO. P17342; Lowe D. G. et al., Nucleic Acids Res., 1990, 18(11):3412; Porter J. G. et al., Biochem. Biophys. Res. Commun., 1990, 171(2):796-803; Stults J. T. et al., Biochemistry, 1994, 33(37):11372-11381).

SEQ ID NO: 21 is an siRNA specific for
NPR-A (human).
tat tac ggt gga cca cct gtt caa gag aca ggt ggt
cca ccg taa tat ttttt
SEQ ID NO: 22 is an siRNA specific for
NPR-A (human).
aga att cca gaa acg cag ctt caa gag agc tgc gtt
tct gga att ctt ttttt
SEQ ID NO: 23 is the nucleotide sequence of an
siRNA for NPRA (siNPRA8): (targeting
position 33):
5′-CAT ATG ggg ccc GGG CGC TGC TGC TGC TAC
Cct cga aat GGT AGC AGC AGC AGC GCC CTT gaa
ttc CCA TGG-3′
SEQ ID NO: 24 is the nucleotide sequence
of an siRNA for mouse NPRA (siNPRA9):
5′-CAT ATG ggg ccc GCG GCC ACG CGA GCG ACC Tct
cga aat AGG TCG CTC GCG TGG CCG CTTgaa ttc CCA
TGG-3′.
SEQ ID NO: 25 is the nucleotide sequence of an
siRNA for NPRA (siNPRA10): (targeting position 33)
siNPRA187top (si10):
5′-CAT ATG ggg ccc GGC TCG GCC GGA CTT GCT Gct cga
aat CAG CAA GTC CGG CCG AGC CTT gaa ttc CCA TGG-3′.

SEQ ID NO:26 is the nucleotide sequence encoding human NPRA (NCBI Accession #AF190631:

| | |
|---|---|
| 1 | ggatcccaaa ccagcacacc tttccctctt cccccgagga |
| | gaccaggtag gaggcgaggg |
| 61 | aaaaggtggg gcgcaagtgg gccccggttg cttccacaca |
| | caccctccgt tcagccgtcc |
| 121 | tttccatccc ggcgagggcg caccttcaga gggtcctgtc |
| | ctccaaagag gtaggcgtgg |
| 181 | ggcggccgag accggggaag atggtccacg gggaagcgcg |
| | cgggctgggc ggcggggagg |
| 241 | aaggagtcta tgatcctgga ttggctcttc tgtcactgag |
| | tctgggaggg gaagcggctg |
| 301 | ggagggaggg ttcggagctt ggctcgggtc ctccacggtt |
| | ccctccggat agccggagac |
| 361 | ttgggccggc cggacgcccc ttctggcaca ctccctgggg |
| | caggcgctca cgcacgctac |
| 421 | aaacacacac tcctctttcc tccctcgcgc gccctctctc |
| | atccttcttc acgaagcgct |
| 481 | cactcgcacc ctttctctct ctctctctct ctctaacacg |
| | cacgcacact cccagttgtt |
| 541 | cacactcggg tcctctccag cccgacgttc tcctggcacc |
| | cacctgctcc gcggcgccct |
| 601 | gcacgccccc ctcggtcgcg ccccttgcgc tctcggccca |
| | gaccgtcgca gctacagggg |
| 661 | gcctcgagcc ccggggtgag cgtccccgtc ccgctcctgc |
| | tccttcccat agggacgcgc |
| 721 | ctgatgcctg ggaccggccg ctgagcccaa ggggaccgag |
| | gaggccatgg taggagcgct |
| 781 | cgcctgctgc ggtgcccgct gaggccatgc cggggccccg |
| | gcgccccgct ggctcccgcc |
| 841 | tgcgcctgct cctgctcctg ctgctgccgc cgctgctgct |
| | gctgctccgg ggcagccacg |
| 901 | cgggcaacct gacggtagcc gtggtactgc cgctggccaa |
| | tacctcgtac ccctggtcgt |
| 961 | gggcgcgcgt gggacccgcc gtggagctgg ccctggccca |
| | ggtgaaggcg cgccccgact |
| 1021 | tgctgccggg ctggacggtc cgcacggtgc tgggcagcag |
| | cgaaaacgcg ctgggcgtct |
| 1081 | gctccgacac cgcagcgccc ctggccgcgg tggacctcaa |
| | gtgggagcac aaccccgctg |
| 1141 | tgttcctggg ccccggctgc gtgtacgccg ccgccccagt |
| | ggggcgcttc accgcgcact |
| 1201 | ggcgggtccc gctgctgacc gccggcgccc cggcgctggg |
| | cttcggtgtc aaggacgagt |
| 1261 | atgcgctgac cacccgcgcg gggcccagct acgccaagct |
| | gggggacttc gtggcggcgc |
| 1321 | tgcaccgacg gctgggctgg gagcgccaag cgctcatgct |
| | ctacgcctac cggccgggtg |
| 1381 | acgaagagca ctgcttcttc ctcgtggagg ggctgttcat |
| | gcgggtccgc gaccgcctca |
| 1441 | atattacggt ggaccacctg gagttcgccg aggacgacct |
| | cagccactac accaggctgc |
| 1501 | tgcggaccat gccgcgcaaa ggccgaggtg agacgctggc |
| | acaccccgtc ccgccgctta |
| 1561 | gccgcagggc ctcccctctg acctgccgga ggcatcggga |
| | ctttctctct catctggggg |
| 1621 | cactcttctt tctcctcgcc gttcttcatt ctactttcag |
| | ctccctggcc ctttctacag |
| 1681 | ctgagtttct atttccctct cttcttccgc cacccccacc |
| | acgtctctat cctctcatct |
| 1741 | ccccgacccc cactcattcc ctcccaccct agcacagctc |
| | ggttccggtc cctttttccc |
| 1801 | tcccacattt tctctcttcc ctatagcctt ctcccttctt |
| | tcatcctctc ctctcatggc |
| 1861 | gcctcatccc ctctcttctc cccctccctc tccctcctct |
| | ctccctcctg gccccatcct |
| 1921 | tctccacctt cagctccact atccccctct ccctacccgt |
| | tccttcctcc cttccgcctc |
| 1981 | ccccttcctc ctcccgccca ccgccccgca cccgcccgtt |
| | ccacccttcg actttctcct |
| 2041 | gctgtggcct aggctgagcc gggagttacc acttaactct |
| | cactgggtct ctcctgcacc |
| 2101 | ctatctctaa acttcctccc ttgggtgccc cagctttcct |
| | actcctgtct ctcccgcagt |
| 2161 | acctaggctt ctctctctga ctctccgtct ttctccagtt |
| | atctacatct gcagctcccc |
| 2221 | tgatgccttc agaaccctca tgctcctggc cctggaagct |
| | ggcttgtgtg gggaggacta |
| 2281 | cgttttcttc cacctggata tctttgggca aagcctgcaa |
| | ggtggacagg gccctgctcc |
| 2341 | ccgcaggccc tgggagagag gggatgggca ggatgtcagt |
| | gcccgccagg cctttcaggt |
| 2401 | gagtacctag gtttgaagcc caggctgtct cagcttgtgg |
| | cacatcattt ctgggcactg |
| 2461 | tgtccctcag catctgaaag aattccagaa aagaggtttt |
| | tgtctgtttg tttctttatg |
| 2521 | cactcctggt aactcacaga acagaaaaga ggttggtgat |
| | gctcactggg aattaggcaa |
| 2581 | tgaagggcag gggactgccc aggggcgctt cgccaccagc |
| | aggctaaaaa gataagaaaa |
| 2641 | tgggcttgag gcgggaggag gataaagtcc cacagcctgg |
| | acaggacttg gagaaggcat |
| 2701 | cccattggat cccctgcttt ggaatgggca tcacttcatg |
| | cagggcatag ggtccagttt |
| 2761 | gaccttgagc taagcagaga cgcagctctg ggaggtgggc |
| | tcccaactgt tggggcccca |
| 2821 | cagtactagg gaatagtcag ctcccaactc tctgctctcc |
| | actgacccct ttctcaggct |
| 2881 | gccaaaatca ttacatataa agacccagat aatcccgagt |
| | acttggaatt cctgaagcag |
| 2941 | ttaaaacacc tggcctatga gcagttcaac ttcaccatgg |
| | aggatggcct ggtaagaagg |
| 3001 | ggtcccggga ccctccagcg tggacctcca gcccccactc |
| | catgaccctc tgccagcctc |
| 3061 | catccttccc tattcccagt tctccccttc cttccctccc |
| | ttcccattgt tccatgtttc |
| 3121 | tcgtgatgat ggaggaggac actggcaagt tcagcctctg |
| | aaactcaggt catcatcagt |
| 3181 | aatatggaga cgatacatcc tgccctgtct acctagtagg |
| | attcaggaag tgatgctaat |
| 3241 | ccaaaggcat cgtttaaata gtaaaatctc cctgtgatat |
| | aggggtgtta ttttctccca |
| 3301 | tcctcttcca aaatcccagt gcctcttgtt cccttcccca |
| | cagctcccac ctccatgccc |
| 3361 | ttcatatgcc caccccagcc gacctctgtt tgcccctaca |
| | ggtgaacacc atcccagcat |
| 3421 | ccttccacga cgggctcctg ctctatatcc aggcagtgac |
| | ggagactctg gcacatgggg |
| 3481 | gaactgttac tgatggggag aacatcactc agcggatgtg |
| | gaaccgaagc tttcaaggtc |
| 3541 | agggcctgga ggtggctgga atgggctgcc ttgggggatg |
| | aatcccaggt gcccagtgtc |
| 3601 | aagccatgag aagcctattg tcctgcagca gttacctatg |
| | cacaccagcc ttttcctcca |
| 3661 | cagctttttt caggcccatc cctcagaagt cctacaaagt |
| | gtccaatctc aatcatccct |
| 3721 | gctgggcact gagttctttt acctttcttt ttcttttttc |
| | tttttttttt gagatggagt |
| 3781 | ctcgctctgt ccccaagact ggagtgtggt ggtgcaatct |
| | cggctcactt caacctccgc |
| 3841 | ctcccaggtt caagcaattc tcctgcctca gcctcctgag |
| | tagctgggat tacaggtgcc |
| 3901 | ctccaccaac acttggctaa ttttttgtat tttttttagt |
| | agagacaggg tttcaccacg |
| 3961 | ttggtcaggc tggtcttgaa ctcctgacgt caggtgatct |
| | gcccgcctca gcctcccaaa |
| 4021 | gtgctgggat tacaagcatg agccacagtg cccggccgtt |
| | ttaccattta ctatcattct |
| 4081 | gtatacatgt atgtttggaa ggcaaggcaa aaaagattag |
| | aggatgaaga gatgaagtgg |
| 4141 | ggcacccctg aacttctatt ctctcaaaca tagtcatctt |
| | cccccatgtc ctcaggtgtg |
| 4201 | acaggatacc tgaaaattga tagcagtggc gatcgggaaa |
| | cagacttctc cctctgggat |
| 4261 | atggatcccg agaatggtgc cttcagggta agtttgtgca |
| | cccagaagac agtgccaatt |
| 4321 | ccaaatgaca tctcaccctc ctacttcccc cccacagccc |
| | tgccagggca cctgtttatc |
| 4381 | ctgtagccat tccaccatgc ctggacactt acaagagccc |
| | tggataaaac agacccagct |
| 4441 | ccagtctggg gaagccacca gaatgatagg gactcacagg |
| | catcacactt ggggagcccc |
| 4501 | atgcctgagg agggagcaca agcctgccct cggggagctc |
| | cgaagggagg caggcaggac |
| 4561 | cgcctcccag cagagacagg gctgtgaaag atgcacatta |
| | cacagctctg caagcgagca |
| 4621 | gggacaggaa ggcgctgagg ccaatggcca caagggacag |
| | gtcatccaga gaaggcctcc |
| 4681 | tggaagacgg gcacatggac tgggcctgcg aatgtaggct |
| | aaggtgaaca ttaccttctc |
| 4741 | ctgttttcta ccaagaaaat aagtagagaa aaatcaatgc |
| | ttggttggta cttcaaccaa |
| 4801 | gattataaac tccctgagtg tagagatcgg gttctaaatg |
| | gagttttctt tataaacccc |
| 4861 | ttgatagttt tcaggtgttt ccacttgagt actatgtgtg |
| | tggtatgagg tcctgtgtcc |
| 4921 | agttgcagtg gggacttggt aagcaggtga caacccagat |
| | atatatgtag gctctagaag |
| 4981 | cagagctggg gtaggtggga ggtgagactg ctgcactcac |
| | agcatgcctt ccccgcaggc |
| 5041 | cctggcctag ccaccactcc tgctctccct taggttgtac |
| | tgaactacaa tgggacttcc |
| 5101 | caagagctgg tggctgtgtc ggggcgcaaa ctgaactggc |
| | ccctggggta ccctcctcct |
| 5161 | gacatcccca aatgtggctt tgacaacgaa gacccagcat |
| | gcaaccaagg tgactgcccc |
| 5221 | ttgccttcca ggcctccatc ccagagatgc tgcatccttc |
| | ccctaagcac agtcgagtag |
| 5281 | gtgctcctgt cccatgctga gggctttctg gagaatgact |
| | cctgcctttt tcttcccttc |
| 5341 | atccatcatc ccagttcact gatggactat tagaaagttc |
| | ttcctcctgc tgtctaaccc |
| 5401 | aaatctctct tgctgcaata tggactctct cctgcagatc |
| | acctttccac cctggaggtg |
| 5461 | ctggctttgg tgggcagcct ctccttgctc ggcattctga |
| | ttgtctcctt cttcatatac |
| 5521 | aggtgagctg tgatgtgggg ggttgagtga ggctggggga |
| | cccggagaac caagagcaga |
| 5581 | ggaggcggtg gggacccaga gggaagaggg caggggtgaa |
| | ggggcagcag gggaaaacca |
| 5641 | agggagatga ggaagaaagg aggcttaaaa gccagaggag |
| | aaagaaagag aagggaatgg |
| 5701 | cagggcgagg ggaggagaca aggataggaa tggccaagga |
| | gagtcagaaa gatccaagaa |
| 5761 | gcagagaagt tgatgggtga catcataggg gcgtggactg |
| | gttttccttg ctactcttgc |
| 5821 | aggccagata ggaagcaact ttctgaacct ttgcaatcat |
| | gcccatgtta gctgaggagg |
| 5881 | gtgagccctg gtgtgtgcca ggtgcccaac ctagaatgga |
| | gaagggagct gaatgagcct |
| 5941 | tgttcctgcc gtccagtgga ggctaaaatg aagtacagga |
| | ggagttaatg atatacaaaa |
| 6001 | gcaaggaggg aggggagaaa aatcactgct ggttgagcat |
| | ataatgtgtg ccaggcactt |
| 6061 | ccacgtacac tatttctttc tttctttttt tttttttttt |
| | tttttttttg agacggagtc |
| 6121 | tcgctctgtt gccagactgg agtgcagtgg catgatctag |
| | gctcactgca acctccgcct |
| 6181 | cccagtttca agcaattctc ctgcctcagc ctcccatgta |
| | gctgggacta caggcacatg |
| 6241 | ccaccacgct cagctaattt ttgtattttt agtagagaca |
| | gggtttcacc atgttggcca |
| 6301 | ggatggtctc gatctcttga cctcatgatc cacccacctt |
| | ggcctcccaa agtgctggga |
| 6361 | ttacaggcat gagccactgt gcctggcctc atgttcacta |
| | tttcttttca ttcttataat |
| 6421 | agttaagaat gaaatagata ttgcggcctc attcccaagt |
| | aaggacattg aggtgattcc |
| 6481 | cccaaggtcc ccagtaaggc agaatttccc ccagccatcc |
| | tgattctcag tccagaggat |
| 6541 | agaattcccc ctccatctct gagtgcatgg tgtggtccca |
| | cggctctgag gaggggctgc |
| 6601 | tgagcaccct gccctgggtc agcggctcag ccacaggctc |
| | agatgcagcc ttcgtatccc |
| 6661 | aggaagatgc agctggagaa ggaactggcc tcggagctgt |
| | ggcgggtgcg ctgggaggac |
| 6721 | gttgagccca gtagccttga gaggcacctg cggagtgcag |
| | gcagccggct gaccctgagc |
| 6781 | ggggtaagaa cgctggtgtt tgtgttgggg ggcaataaag |
| | gagaggtggg tacaaggggc |
| 6841 | agtgcctgag ggataggtaa gggagcagga ttctagtccc |
| | agctctgctt tcacttgctg |
| 6901 | tgtgaccttg agcgactcat agtccctctc cgagactgtc |
| | tcagatgatg attacagcag |
| 6961 | cagagcctcc ctcacagggc tcttttaaag gtcagaggag |
| | atagtacctg tgaaaacact |
| 7021 | ttaaaaaaaa aaaaagtaaa tgaggaggaa attttatgat |
| | gtggaacata aagcagggtg |
| 7081 | ggccaggcac agtggctcac atctgcaatc ccagcacttt |
| | gggagaccga ggcaggagga |
| 7141 | ttgcttgtgc ctgggagttc aagaccagcc tgggcaacag |
| | agcaagacat cgtctctaca |
| 7201 | aagaatacaa agattagcag ggcatggtgg cgcatacctg |
| | tagtcccagc tactctggag |
| 7261 | gctgaggtga aaggatcatc tgagcccagg agtctgaggc |
| | ggcagtgacc taggatagca |
| 7321 | ccactgcact ccagcctgga tgacacaatg atactacatc |
| | tcaaaaaaaa acccaacaac |
| 7381 | aaaaaggaag ggtgacacaa agataaggca ggataaggca |
| | gggaaataaa gaccagagca |
| 7441 | caagcaatca ggatgcagac tgggcccacc ggctgaccat |
| | tcctcctgct ctccctcctt |
| 7501 | tcagagaggc tccaattacg gctccctgct aaccacagag |
| | ggccagttcc aagtctttgc |
| 7561 | caagacagca tattataagg tgggcctggg gaaagatcac |
| | tgggccttgg gactggggca |
| 7621 | ggagtgtact ctgatggagg actggtgggg ggttctgagg |
| | gaaggagtaa gctggtgggg |
| 7681 | agcagcagat gggggccctg ggggtgggct attgggaaca |
| | agtgagggtc ctgagggcag |
| 7741 | ggatgggctg tcgggagcag ctggaattcc caggacatgg |
| | gaccatgctc ttcacagtga |
| 7801 | cagtctccat tccatgccca gggcaacctc gtggctgtga |
| | aacgtgtgaa ccgtaaacgc |
| 7861 | attgagctga cacgaaaagt cctgtttgaa ctgaagcatg |
| | taatgtgggg agtgaggcag |
| 7921 | tggcatggag aaggggccct cggggacgca agggagactg |
| | gccaacagaa ctagttatgg |
| 7981 | agggacctca gggtacccca agaaaggggc agggactgga |
| | gccctggatg accttcatct |
| 8041 | tgtggtggag tgggggtatc ctaagtagga gaagagacca |
| | ctgagataac ctggaggaat |
| 8101 | cttgaggggc catatgtgat gtccctgggg gagagagggc |
| | ttaggatgcc agagggagta |
| 8161 | ggagcagatt ctggggaggg tgggctaaag gacatgggtg |
| | ggaatcacca gggaagatct |
| 8221 | tagtgatggt tgcagaaagt gaataaggag ttaagaagag |
| | tgagggtccc tgaagctagt |
| 8281 | gagcagcttg gtgaggagcg aggtctctgt caagctcctg |
| | atgctggtcc cacttgcaga |
| 8341 | tgcgggatgt gcagaatgaa cacctgacca ggtttgtggg |
| | agcctgcacc gaccccccca |
| 8401 | atatctgcat cctcacagag tactgtcccc gtgggagcct |
| | gcaggtgagg gggacaaggg |
| 8461 | gtgtcaagaa acctgggttc tagccctggc tctgcccctg |
| | actggccata agaccccagg |
| 8521 | catgcctcgc cctctttctg acctttctgg ccccatctgt |
| | aaaaatggga gttggggaag |
| 8581 | ggcagtggca ctagagtcaa tccaaagttt tgtcctgttc |
| | taccagttca catcagtagg |
| 8641 | accctgcacc ctcctccaac tcccaggggg atctgcaggg |
| | gattggtctt gactcttatt |
| 8701 | gccccagcag gacattctgg agaatgagag catcaccctg |
| | gactggatgt tccggtactc |
| 8761 | actcaccaat gacatcgtca aggtatgccc ctaagcacct |
| | attggatgtg tagagcaggg |
| 8821 | gccaggcatg cttctcctgg ccacgggtgt aggtcccact |
| | cctggccaat acctctgccc |
| 8881 | actcacattt ccagggcatg ctgtttctac acaatggggc |
| | tatctgttcc catgggaacc |
| 8941 | tcaagtcatc caactgcgtg gtagatgggc gctttgtgct |
| | caagatcacc gactatgggc |
| 9001 | tggagagctt cagggacctg gacccagagc aaggacacac |
| | cgtttatgcc agtgagcctt |
| 9061 | gactcttgaa cctaacacct gcccccagca ccacccagta |
| | gggagactga tgcaaggcct |
| 9121 | ctgatgggct tgggcatgct tgtcctgact ccagcctcaa |
| | ttcattcacc catgaaaaag |
| 9181 | ggaaggccag acgaagtggt ttctaaggcc tcctctagct |
| | ctaacactct gtgatgcatc |
| 9241 | cagatcagtt tcggccacac ccttgtttcc ccctcacccc |
| | ttagctttgg gctccctcac |
| 9301 | tcggtgacta ccgacctctg acccacagaa aagctgtgga |
| | cggcccctga gctcctgcga |
| 9361 | atggcttcac cccctgtgcg gggctcccag gctggtgacg |
| | tatacagctt tgggatcatc |
| 9421 | cttcaggaga ttgccctgag gagtggggtc ttccacgtgg |
| | aaggtttgga cctgagcccc |
| 9481 | aaaggtgaga ggagcacacc ttccttaaac ccagccacag |
| | tctcaacgaa ccccagcccc |
| 9541 | agggagaggg tcccctggca gcaccaccac accttccttc |
| | tgtaatgggg ttcagtcacc |
| 9601 | accctttgac ccattgctgc cagtgaccag tcccccgccc |
| | ccatgccttg gtcttggact |
| 9661 | tcccctgcca tctcagctgg ttgccccagt ctctcactag |
| | gcccttggcc agccccaccc |
| 9721 | ctcagctcct ctacccccca atacagagat catcgagcgg |
| | gtgactcggg gtgagcagcc |
| 9781 | ccccttccgg ccctccctgg ccctgcagag tcacctggag |
| | gagttggggc tgctcatgca |
| 9841 | gcggtgctgg gctgaggacc cacaggagag gccaccattc |
| | cagcagatcc gcctgacgtt |
| 9901 | gcgcaaattt aacaggtccc tggtgtttgt catggatccc |
| | ccaggccctt cctccacagc |
| 9961 | caccatttac ctaatgcttc tggctctggc ttatcccagc |
| | agtggcagag ggagaccact |
| 10021 | cacctcctcc ctgtacatag tcagctccag ctcagcacag |
| | cctcatgacc ctcttcgcaa |
| 10081 | gtacagcatg actcagctgt ccccacagtc ccctgccatt |
| | catgcccctt ccctccacca |
| 10141 | tcgacacccc acacccttcc tgcccactcg ccttgctggc |
| | ctctagactt ctcagcagtg |
| 10201 | tgtaggatag atgggcctcc cgcctcctgc cctgtaggct |
| | cttggccctc cacgggagct |
| 10261 | cctgccccac cccttgattt cccttcccca gcgtgcccac |
| | caggcccagt tcctccagac |
| 10321 | acacccttct gtggacatca ctttgtccgc aattgaccct |
| | tgtcattctc cacctccttt |
| 10381 | acctccttct aactcactgg gttcaacaaa gatgaacaaa |
| | atgtccatat gtctgaagct |
| 10441 | tcatacttga ccttggggtc tcagaaaaga attgaacttt |
| | cttccttctg ttttcccctg |
| 10501 | ctccccggta tcctgctatg ccctcaaccc tgagcgtctc |
| | tagagacctc actgcagtct |
| 10561 | ggagggggaa gtgcctaggg gcgggcgctc acgtaggctg |
| | tgctgctcct ctcttaccac |
| 10621 | ccccaccgcc accctctgcc cccagggaga acagcagcaa |
| | catcctggac aacctgctgt |
| 10681 | cccgcatgga gcagtacgcg aacaatctgg aggaactggt |
| | ggaggagcgg acccaggcat |
| 10741 | acctggagga gaagcgcaag gctgaggccc tgctctacca |
| | gatcctgcct cagtgagtgc |
| 10801 | ctgagtctgg ggaccccccc caacacaaag cccctgtccc |
| | gacccccaac tctgatcctg |
| 10861 | cacctgccct gaccccttag ctcagtggct gagcagctga |
| | agcgtgggga gacggtgcag |
| 10921 | gccgaagcct ttgacagtgt taccatctac ttcagtgaca |
| | ttgtgggttt cacagcgctg |
| 10981 | tcggcggaga gcacacccat gcaggtaggc cagggttcag |
| | ccacaggtgc caggcaagct |
| 11041 | cagcatctgg atcccaccag acctgccttc tggttctgct |
| | ttacccacct gaccccaggt |
| 11101 | ggggtcccct acttcctgtc tctcttagct tctcttccct |
| | tccaggtggt gaccctgctc |
| 11161 | aatgacctgt acacttgctt tgatgctgtc atagacaact |
| | ttgatgtgta caaggtgagg |
| 11221 | gtgggagtgg ggatgggaag ggacagacag acatggacaa |
| | ggtcagaaaa agatgagggg |
| 11281 | taggcagaat gatgtggagt cttaagagag gagatcgggg |
| | acacgggcag agacagtgac |
| 11341 | acagggagac ccgggaacag gcagagaacc catgtgggat |
| | gggggatgag caaagacaga |
| 11401 | tgagggtaca gaatgacaga cgctgcaccc ggtgtgacgg |
| | tgtggccggc cgcacagttg |
| 11461 | cagccgtcaa gtcctgcacc ccctcgccac tcccacaggt |
| | ggagacaatt ggcgatgcct |
| 11521 | acatggtggt gtcagggctc cctgtgcgga acgggcggct |
| | acacgcctgc gaggtagccc |
| 11581 | gcatggccct ggcactgctg gatgctgtgc gctccttccg |
| | aatccgccac cggccccagg |
| 11641 | agcagctgcg cttgcgcatt ggcatccaca caggtaaggc |
| | cactgaaggt gcaggcgggc |
| 11701 | atccagaggc caaggctttg caagggaaac ttgtcccctg |
| | gcccagcccc tcgccctttc |
| 11761 | atctctctct ctctctctct ctctctctct ctctctctct |
| | gtctctctct ctctctctct |
| 11821 | ctctctctct ctcacacaca cacacacaca cacacacaga |
| | gctgggacct cagatcctgc |
| 11881 | ctcctgcctg tcttggattg tccacctacc tcccttaaca |
| | cccctccctc cctcactcgc |
| 11941 | tgatgggctc tgctccttcc cttgctcctc ccaggacctg |
| | tgtgtgctgg agtggtggga |
| 12001 | ctgaagatgc cccgttactg tctctttggg gatacagtca |
| | acacagcctc aagaatggag |
| 12061 | tctaatgggg aaggtacagt gccccctcct agagggaatg |
| | gggagggcag ggtggctgag |
| 12121 | ggaaatgcca tcctggggca gcctgtgcct gcacagcccg |
| | tttcagctcc tagccctttc |
| 12181 | gcctcccaag ttccccttct cataatatta agagttcaac |
| | ctgggctcat caacttgact |
| 12241 | gtaaccagag actcaggttc ctgctgcccc tcttgtcaaa |
| | cgatgtaaaa gtatttccgg |
| 12301 | gccagtgctg gagagttccc agcaggaatc tgattttaag |
| | accctctgtg ggccgggcgt |
| 12361 | ggtgactcac acctgtgatc ccagcacttt gggaagctga |
| | ggcaggcgga tcacctgagg |
| 12421 | tcgggggttt cgagaccagc ctgaccaaca tgatgaaatc |
| | ccgtctctac taaaaataca |
| 12481 | aaaaactagc caggtgtgat ggcaggctcc tgtaatccca |
| | gctacttggg aggcttgagg |
| 12541 | cagaagaatt gcttgaaccc gggaggcaga ggttgcgatg |
| | agccaagatt acaccacgca |
| 12601 | ccccagcttg ggcaataaga gttaaactct gtctcaaaaa |
| | aaaaaaaaaa aaaaaaaaaa |
| 12661 | agggccctct gctccacctt tgatgtggta aagatggctt |
| | cagagccagc ataagtgagg |
| 12721 | ctgtgaatct cagctccaca gctggctgtg tgtcagtttg |
| | ctatacctct ctgagccatg |
| 12781 | gttttcctca tctgtaaaaa gagggaaaaa atctatctca |
| | caggaattat gtgagaaacc |
| 12841 | cattaaaaat gtctaccaca taattgtcat ttaacttttc |
| | caagccttag cggattatct |
| 12901 | gtaaaatgat gtctatctca ggattgcaag aagcctagca |
| | caaaccctgg tacccagcag |
| 12961 | gcacctaata aattcttact cctacccgcc ccttgctctt |
| | gcctcctgtt tatcttctat |
| 13021 | ccttctgctg tattcgacac aattcaatgc agtaaacatt |
| | tattgagtga ctactgagtg |
| 13081 | ccaggccctg ggatagtaac atggcccaga tccagagtta |
| | gctgagaaat tcatgtggac |
| 13141 | cccatctaaa ccttatggtg aaagaaaggc tgcttgggag |
| | ccagtcctgg gagcccagag |
| 13201 | ggatctagtt cggcaaatat tccctgggca ctatttgggg |
| | gctgcagagt cagcccttgt |
| 13261 | tgagggtcca gtcctcaagg agcacattcc cagaaatgtt |
| | cacattctgg cgctggggtg |
| 13321 | ctgtaatccc agcactttgg gaggccgagg tgggcagatc |
| | acttgaggcc aggagtggag |
| 13381 | actagcctgg ccaacatggt gacctcctgt ctctactaaa |
| | aatacaaaaa attagctggg |
| 13441 | cgtggtggca cgtgcccgta atcccagcta ctcaggaggc |
| | ttgagacatg aaaatcactt |
| 13501 | gaacccagga ggtggatgtt gcagtgagcc gagactgcac |
| | ccctgggcaa cagagcgaga |
| 13561 | ctctgtctca aaaaaaaaaa agagagaaag aaagaaaaga |
| | aaagaaagaa actgttaaac |
| 13621 | acaacaaggc cactgtgatt gatgcaaacc ccagaagtag |
| | ggacatgagt tcagacagtg |
| 13681 | gtcaaagaga gggtgtggca atattgggcc ccactccatc |
| | actgacctcc tcagccactt |
| 13741 | gggcagatca ccctgggcct cagttcctcg gccacaaaat |
| | gagggtatag catgaaatca |
| 13801 | tgaaagcaac aatttacata gtgcttccta ggtagcacat |
| | tccgtttgaa tactttatgg |
| 13861 | atgttaaatt taatcctcac aacaaggttt tgagatgggt |
| | actgacacta tcagcatttt |
| 13921 | acagattagg aaaatgaagc agagagaatt tattttacat |
| | acctaagcaa gtatccaagc |
| 13981 | tgaggttcat actgaggcag tgcaggatcc aaagtgccag |
| | ctcctaacca ccatgctgtg |
| 14041 | tagagccggg tgacactcca gagagtgctg tccaacagga |
| | tgttccatag tcatgaaaat |
| 14101 | gttctgtatt ctgtgctgtc caatacagta gcctctaggc |
| | acatatggct acttatcact |
| 14161 | ggaaatgtga cgggtgcaac tgaggccctg attttttttt |
| | tttttttgga gacagagttt |
| 14221 | cgctctgtcg cccagcctgg atggagtgca gtggtgcaat |
| | ctcggctcac tgcaacctcc |
| 14281 | gcctcccagg ttcaagcgat tctcctgcct cagcctccca |
| | agtagctgga attacaggtg |
| 14341 | agtgccacca cacacagcta atttttgtat ttttagtaga |
| | gacggggttt cgccatattg |
| 14401 | gccaggatgg tctcgaactc ctggcctcaa gtgatcctcc |
| | tgcctcagcc tcccaaagtg |
| 14461 | ctgggattac aggtgtgagc cacagcaccc agcctgaatt |
| | tttaactgta tttagtttaa |
| 14521 | attaatttaa gttgaaacag gcacatgtga ttagtggcta |
| | ctgtattgga ttacacagct |
| 14581 | ccagagttct aaatgagagg ctaatgtggt cacgcactac |
| | attcaggggg tggggcccct |
| 14641 | ctgagctaga gggcttcctg gcccaaaaga gggagagagg |
| | gtacctgtcc acctgtccac |
| 14701 | ccccacagtc cctggtctct tttgcctcta ctttcctgct |
| | ctcctctctc acattgctca |
| 14761 | ccttcccttc tcccctgtcc tacccagccc tgaagatcca |
| | cttgtcttct gagaccaagg |
| 14821 | ctgtcctgga ggagtttggt ggtttcgagc tggagcttcg |
| | aggggatgta gaaatgaagg |
| 14881 | tagagcgaga agcctctgcc ctccccacct tttggggtcc |
| | tagagggagt tacccttctc |
| 14941 | aagcagccga tgccactccc atccctaagg ctctcatctg |
| | actggggaaa gggcatgtgc |
| 15001 | cactccccag cccatcctct tttttccctc cagggcaaag |
| | gcaaggttcg gacctactgg |
| 15061 | ctccttgggg agagggggag tagcacccga ggctgacctg |
| | cctcctctcc tatccctcca |
| 15121 | cacctcccct accctgtgcc agaagcaaca gaggtgccag |
| | gcctcagcct cacccacagc |
| 15181 | agccccatcg ccaaaggatg gaagtaattt gaatagctca |
| | ggtgtgctga ccccagtgaa |
| 15241 | gacaccagat aggacctctg agaggggact ggcatggggg |
| | gatctcagag cttacaggct |
| 15301 | gagccaagcc cacggccatg cacagggaca ctcacacagg |
| | cacacgcacc tgctctccac |
| 15361 | ctggactcag gccgggctgg gctgtggatt cctgatcccc |
| | tcccctcccc atgctctcct |
| 15421 | ccctcagcct tgctaccctg tgacttactg ggaggagaaa |
| | gagtcacctg aaggggaaca |
| 15481 | tgaaaagaga ctaggtgaag agagggcagg ggagcccaca |
| | tctggggctg gcccacaata |
| 15541 | cctgctcccc cgaccccctc cacccagcag tagacacagt |
| | gcacagggga gaagaggggt |
| 15601 | ggcgcagaag ggttgggggc ctgtatgcct tgcttctacc |
| | atgagcagag acaattaaaa |
| 15661 | tctttattcc agtgacagtg tctcttcttg agggagagag |
| | ggttgccaga aaacagtcag |
| 15721 | ttctccactc tctacttcaa ataagactca cttcttgttc |
| | tacaagggtc tagaaggaaa |
| 15781 | agtaaaaaaa aaagactctc gattcttaac |

SEQ ID NO: 27 is a NPRA specific primer F:
5′ GCA AAG GCC GAG TTA TCT ACA Te-
SEQ ID NO: 28 is a NPRA specific primer R:
5′ AAC GTA GTC eTC CeC ACA CAA -3

### DETAILED DISCLOSURE OF THE INVENTION

The examples described and the drawings rendered are illustrative and are not to be read as limiting the scope of the invention as it is defined by the appended claims.

The term “chitosan”, as used herein, will be understood by those skilled in the art to include chitosan, derivatives of chitosan, or poly-N-acyl-D-glucosamine (including all polyglucosamine and oligomers of glucosamine materials of different molecular weights), in which the greater proportion of the N-acetyl groups have been removed through hydrolysis. Generally, chitosans are a family of cationic, binary hetero-polysaccharides composed of (1→4)-linked 2-acetamido-2-deoxy-β-D-glucose (GlcNAc, A-unit) and 2-amino-2-deoxy-β-D-glucose, (GlcN; D-unit) (Varum K. M. et al., Carbohydr. Res., 1991, 217:19-27; Sannan T. et al., Macromol. Chem., 1776, 177:3589-3600). Preferably, the chitosan has a positive charge. Chitosan derivatives or salts (e.g., nitrate, phosphate, sulphate, hydrochloride, glutamate, lactate or acetate salts) of chitosan may be used and are included within the meaning of the term “chitosan”. As used herein, the term “chitosan derivatives” are defined to include ester, ether or other derivatives formed by bonding of acyl and/or alkyl groups with OH groups, but not the NH₂ groups, of chitosan. Examples are O-alkyl ethers of chitosan and O-acyl esters of chitosan. Modified chitosans, particularly those conjugated to polyethylene glycol, are included in this definition. Low and medium viscosity chitosans (for example CL113, G210 and CL110) may be obtained from various sources, including PRONOVA Biopolymer, Ltd. (UK); SEIGAGAKU America Inc. (Maryland, USA); MERON (India) Pvt, Ltd. (India); VANSON Ltd. (Virginia, USA); and AMS Biotechnology Ltd. (UK). Suitable derivatives include those which are disclosed in Roberts, Chitin Chemistry, MacMillan Press Ltd., London (1992). Optimization of structural variables such as the charge density and molecular weight of the chitosan for efficiency of polynucleotide delivery and expression is contemplated and encompassed by the present invention.

The chitosan (or chitosan derivative or salt) used preferably has a molecular weight of 4,000 Dalton or more, preferably in the range 25,000 to 2,000,000 Dalton, and most preferably about 50,000 to 300,000 Dalton. Chitosans of different low molecular weights can be prepared by enzymatic degradation of chitosan using chitosanase or by the addition of nitrous acid. Both procedures are well known to those skilled in the art and are described in various publications (Li et al., Plant Physiol. Biochem., 1995, 33: 599-603; Allan and Peyron, Carbohydrate Research, 1995, 277:257-272; Damard and Cartier, Int. J. Biol. Macromol., 1989, 11: 297-302). Preferably, the chitosan is water-soluble and may be produced from chitin by deacetylation to a degree of greater than 40%, preferably between 50% and 98%, and more preferably between 70% and 90%.

The disclosure relates to methods for reducing natriuretic peptide receptor-A (also known in the art as NPRA, NPR-A, and guanylate cyclase A) activity in vitro or in vivo. In one aspect, the method, in one example, may be used for treating inflammatory and cell proliferation disorders, such as cancer. In another aspect, the present invention concerns methods for identifying agents useful for treating inflammatory and cell proliferation disorders by determining whether the candidate agent reduces activity of the natriuretic peptide receptor-A (also known in the art as NPRA, NPR-A, and guanylate cyclase A) in vitro and/or in vivo (also referred to herein as the diagnostic method or assay).

As used herein, an “inflammatory disorder” includes those conditions characterized by an aberrant increase in one or more of the following: IL-6, IL-1 beta, TNF-alpha, IL-8, eosinophil production, neutrophil production, release of histamines, proliferants, hyperplasia, and cell adhesion molecule expression. As used herein, a “cell proliferation disorder” is characterized by one or more of the following: uncontrolled proliferation, a high mitogenic index, over-expression of cyclin D1, cyclin B1, expression of an oncogene such as c-jun and/or c-fos, aberrant activation of NFkB and/or ERK (extracellular receptor kinase), and matrix metalloproteinase expression (such as MMP-2 and/or MMP-9).

In one embodiment, the inflammatory disorder and cell proliferation disorder is not one that is amenable to effective treatment by administration of a vasodilator. In one embodiment, the inflammatory disorder and cell proliferation disorder is not a cardiovascular disorder (such as hypertension or stroke). In another embodiment, the inflammatory disorder and cell proliferation disorder is not a disorder of the central nervous system (such as Alzheimer's disease or other dementia). In another embodiment, the inflammatory disorder and cell proliferation disorder is not kidney failure or other kidney disorder.

The agent used to reduce NPR-A activity in vitro or in vivo can be virtually any substance and can encompass numerous chemical classes, including organic compounds or inorganic compounds. Preferably, an effective amount of the agent is administered to the cells with a pharmaceutically acceptable carrier. The agent may be a substance such as genetic material, protein, lipid, carbohydrate, small molecules, a combination of any of two or more of foregoing, or other compositions. The agent may be naturally occurring or synthetic, and may be a single substance or a mixture. The agent can be obtained from a wide variety of sources including libraries of compounds. The agent can be or include, for example, a polypeptide, peptidomimetic, amino acid(s), amino acid analog(s), function-blocking antibody, polynucleotide(s), polynucleotide analog(s), nucleotide(s), nucleotide analog(s), or other small molecule(s). A polynucleotide may encode a polypeptide that potentially reduces NPR-A activity within the cell, or the polynucleotide may be a short interfering RNA (siRNA), a hairpin RNA (shRNA), antisense oligonucleotide, ribozyme, or other polynucleotide that targets an endogenous or exogenous gene for silencing of gene expression and potentially NPR-A activity within the cell.

In one embodiment, the agent used to reduce NPR-A activity is an interfering RNA specific for NPR-A mRNA, preferably human NPR-A mRNA. Interfering RNA is capable of hybridizing with the mRNA of a target gene and reduce and/or eliminate translation through the mechanism of RNA interference. Examples of such interfering RNA include SEQ ID NO:21 and SEQ ID NO:22, which may reduce NPR-A activity using an siRNA Target Finder program (AMBION) and in accordance with published guidelines (Tuschl T., Nature Biotechnol., 2002, 20:446448). As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

As used herein, the term “small interfering RNA” (“siRNA”) (also referred to in the art as “short interfering RNAs”) refers to an RNA (or RNA analog) that is capable of directing or mediating RNA interference. In one embodiment, the siRNA is between about 10-50 nucleotides (or nucleotide analogs). Optionally, a polynucleotide (e.g., DNA) encoding the siRNA may be administered to cells in vitro or in vivo, such as in a vector, wherein the DNA is transcribed.

As used herein, a siRNA having a “sequence sufficiently complementary to a target mRNA sequence to direct target-specific RNA interference (RNAi)” means that the siRNA has a sequence sufficient to trigger the destruction of the target mRNA by the RNAi machinery or process. “mRNA” or “messenger RNA” or “transcript” is single-stranded RNA that specifies the amino acid sequence of one or more polypeptides. This information is translated during protein synthesis when ribosomes bind to the mRNA.

The scientific literature is replete with reports of endogenous and exogenous gene expression silencing using siRNA, highlighting their therapeutic potential (Gupta, S. et al. PNAS, 2004, 101:1927-1932; Takaku, H. Antivir Chem. Chemother, 2004, 15:57-65; Pardridge, W. M. Expert Opin. Biol. Ther., 2004, 4:1103-1113; Zheng, B. J. Antivir. Ther., 2004, 9:365-374; Shen, W. G. Chin. Med. J. (Engl), 2004, 117:1084-1091; Fuchs, U. et al. Curr. Mol. Med., 2004, 4:507-517; Wadhwa, R. et al. Mutat. Res., 2004, 567:71-84; Ichim, T. E. et al. Am. J. Transplant, 2004, 4:1227-1236; Jana, S. et al. Appl. Microbiol. Biotechnol., 2004, 65:649-657; Ryther, R. C. et al. Gene Ther., 2005, 12:5-11; Chae, S-S. et al., J. Clin. Invest., 2004, 114:1082-1089; Fougerolles, A. et al., Methods Enzymol., 2005, 392:278-296), each of which is incorporated herein by reference in its entirety. Therapeutic silencing of endogenous genes by systemic administration of siRNAs has been described in the literature (Kim B. et al., American Journal of Pathology, 2004, 165:2177-2185; Soutschek J. et al., Nature, 2004, 432:173-178; Pardridge W. M., Expert Opin. Biol. Ther., 2004, July, 4(7): 1103-1113), each of which is incorporated herein by reference in its entirety.

In another embodiment, the decrease in NPR-A activity (e.g., a reduction in NPR-A expression) may be achieved by administering an analogue of ANP (e.g., ANP4-23) or non-peptide antagonists (e.g., HS-142-1; Rutherford et al., Br. J. Pharmacol., 1994, 113: 931-939; El-Ayoubi et al., Br. J. Pharmacol., 2005, Feb. 7, Epub ahead of print; Delport C. et al., Eur. J. Pharmacol., 1992, 224(2-3):183-188; Ohyama Y. et al., Biochem. Biophys. Res. Commun., 1992, 189(1):336-342). In another embodiment, the agent is an anti-human NPR-A function-blocking antibody (preferably, humanized), or soluble NPR-A or NPR-C (as a receptor decoy). Other examples of agents include NPR-A antagonists that specifically inhibit cGMP-dependent protein kinase (PKG) such as A71915 and KT5823 (Pandey K. N. et al., Biochemical and Biophysical Research Communications, 2000, 271:374-379).

The methods may include further steps. In some embodiments, a subject with the relevant inflammatory disorder and/or cell proliferation disorder is identified or a patient at risk for the disorder is identified. A patient may be someone who has not been diagnosed with the disease or condition (diagnosis, prognosis, and/or staging) or someone diagnosed with disease or condition (diagnosis, prognosis, monitoring, and/or staging), including someone treated for the disease or condition (prognosis, staging, and/or monitoring). Alternatively, the person may not have been diagnosed with the disease or condition but suspected of having the disease or condition based either on patient history or family history, or the exhibition or observation of characteristic symptoms.

In one example, the therapeutic method involves administering a natriuretic hormone peptide (NP), or a fragment, homolog or variant thereof, or a nucleic acid sequence encoding an NP, or a fragment, homolog, or variant thereof, to a patient. The present inventor has demonstrated that a prolonged, substantial reduction of tumor burden in lungs can be achieved by intranasal delivery of pDNA-encoding a peptide comprising amino acid residues 73 to 102 (NP73-102). Without being bound by theory, the NP decreased viability due to the induction of apoptosis in a lung adenocarcinoma cell line A549 cell, and can reduce tumorigenesis and metastasis in a number of cancers.

In specific embodiments, the peptides used in the subject invention comprise at least one amino acid sequence selected from the group consisting of NP₁₋₃₀, NP₃₁₋₆₇, NP₇₉₋₉₈, and NP₇₃₋₁₀₂, (SEQ ID NO:1, SEQ ID NO:2, SEQ ID NO:3, and SEQ ID NO:5, respectively), SEQ ID NO:6, or a biologically active fragment or homolog thereof. In some embodiments, a combination of NP or NP-encoding nucleic acid sequences is utilized. In one embodiment, the peptide utilized does not consist of the amino acid sequence of NP₉₉₋₁₂₆ (SEQ ID NO: 4).

In another aspect, the therapeutic method involves administering an agent that reduces activity of the natriuretic peptide receptor-A (also known in the art as NPRA, NPR-A, and guanylate cyclase A) to a patient, wherein the agent is administered in an amount effective to reduce receptor (NPR-A) activity. NPR-A activity can be determined, for example, by one or more of the following biological parameters: production/accumulation of cGMP, expression of the NPR-A (transcription or translation), and/or cellular internalization of the NPR-A.

According to one example of the gene therapy method, the NP-encoding nucleic acid sequence is administered locally at the target site (e.g., at the site of cancer or pre-cancer), or systemically to the patient. In order to treat cancer of the lung, for example, the NP-encoding nucleic acid sequence is preferably administered to the airways of the patient, e.g., nose, sinus, throat and lung, for example, as nose drops, by nebulization, vaporization, or other methods known in the art. More preferably, the nucleic acid sequence encoding NP is administered to the patient orally or intranasally, or otherwise intratracheally. For example, the nucleic acid sequence can be inhaled by the patient through the oral or intranasal routes, or injected directly into tracheal or bronchial tissue.

In specific embodiments, the nucleic acid sequences used in the subject invention encode at least one amino acid sequence selected from the group consisting of NP₁₋₃₀, NP₃₁₋₆₇, NP₇₉₋₉₈, NP₉₉₋₁₂₆, and NP₇₃₋₁₀₂, (SEQ ID NO:1, SEQ ID NO:2, SEQ ID NO:3, SEQ ID NO:4, and SEQ ID NO:5, respectively), SEQ ID NO:6, or a biologically active fragment or homolog of any of the foregoing.

Preferably, the nucleic acid sequence encoding the NP is administered with a nucleic acid sequence that is operatively linked with the NP-encoding nucleic acid sequence and operates as a regulatory sequence. For example, the regulatory sequence can be a promoter sequence that controls transcription and drives expression of the NP-encoding nucleic acid sequence at the desired site, such as at, or adjacent to, the patient's respiratory epithelial cells. The promoter can be a constitutive or inducible promoter to allow selective transcription. The promoter can be a vertebrate or viral promoter. Optionally, enhancers may be used to obtain desired transcription levels. An enhancer is generally any non-translated nucleic acid sequence that works contiguously with the coding sequence (in cis) to change the basal transcription level dictated by the promoter.

The NP-encoding nucleic acid sequences used in the methods, expression vectors, and pharmaceutical compositions of the present invention are preferably isolated. According to the present invention, an isolated nucleic acid molecule or nucleic acid sequence, is a nucleic acid molecule or sequence that has been removed from its natural milieu. As such, “isolated” does not necessarily reflect the extent to which the nucleic acid molecule has been purified. An isolated nucleic acid molecule or sequence useful in the present composition can include DNA, RNA, or any derivatives of either DNA or RNA. An isolated nucleic acid molecule or sequence can be double stranded (i.e., containing both a coding strand and a complementary strand) or single stranded.

A nucleic acid molecule can be isolated from a natural source, or it can be produced using recombinant DNA technology (e.g., polymerase chain reaction (PCR) amplification, cloning) or chemical synthesis. Nucleic acid molecules can be generated or modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules, and combinations thereof.

Although the phrase “nucleic acid molecule” primarily refers to the physical nucleic acid molecule and the phrase “nucleic acid sequence” primarily refers to the sequence of nucleotides on the nucleic acid molecule, the two phrases are used interchangeably herein. As used herein, a “coding” nucleic acid sequence refers to a nucleic acid sequence that encodes at least a portion of a peptide or protein (e.g., a portion of an open reading frame), and can more particularly refer to a nucleic acid sequence encoding a peptide or protein which, when operatively linked to a transcription control sequence (e.g., a promoter sequence), can express the peptide or protein.

The nucleotide sequences encoding NP include “homologous” or “modified” nucleotide sequences. Modified nucleic acid sequences will be understood to mean any nucleotide sequence obtained by mutagenesis according to techniques well known to persons skilled in the art, and exhibiting modifications in relation to the normal sequences. For example, mutations in the regulatory and/or promoter sequences for the expression of a polypeptide that result in a modification of the level of expression of a polypeptide according to one example provide for a “modified nucleotide sequence”. Likewise, substitutions, deletions, or additions of nucleic acids to the polynucleotides in one example, provide for “homologous” or “modified” nucleotide sequences. In various embodiments, “homologous” or “modified” nucleic acid sequences have substantially the same biological or serological activity as the native (naturally occurring) natriuretic peptide. A “homologous” or “modified” nucleotide sequence will also be understood to mean a splice variant of the polynucleotides of the instant invention or any nucleotide sequence encoding a “modified polypeptide” as defined below.

A homologous nucleotide sequence, for the purposes of the present invention, encompasses a nucleotide sequence having a percentage identity with the bases of the nucleotide sequences of between at least (or at least about) 20.00% to 99.99% (inclusive). The aforementioned range of percent identity is to be taken as including, and providing written description and support for, any fractional percentage, in intervals of 0.01%, between 20.00% and 99.99%. These percentages are purely statistical and differences between two nucleic acid sequences can be distributed randomly and over the entire sequence length.

In various embodiments, homologous sequences exhibiting a percentage identity with the bases of the nucleotide sequences of the present invention can have 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity with the polynucleotide sequences of the instant invention. Homologous nucleotide and amino acid sequences include mammalian homologs of the human NP sequences.

The NP homologs include peptides containing, as a primary amino acid sequence, all or part of an exemplified NP polypeptide sequence. The NP homologs thus include NP polypeptides having conservative substitutions, i.e., altered sequences in which functionally equivalent amino acid residues are substituted for residues within the sequence resulting in a peptide which is biologically active. For example, one or more amino acid residues within the sequence can be substituted by another amino acid of a similar polarity which acts as a functional equivalent, resulting in a silent alteration. In one aspect of the present invention, conservative substitutions for an amino acid within the sequence may be selected from other members of the class to which the amino acid belongs (see Table 1). Conservative substitutions also include substitutions by amino acids having chemically modified side chains which do not eliminate the biological activity of the resulting NP homolog.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

Both protein and nucleic acid sequence homologies may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman Proc. Natl. Acad. Sci. USA, 1988, 85(8):2444-2448; Altschul et al. J. Mol. Biol., 1990, 215(3):403-410; Thompson et al. Nucleic Acids Res., 1994, 22(2):4673-4680; Higgins et al. Methods Enzymol., 1996, 266:383-402; Altschul et al. J. Mol. Biol., 1990, 215(3):403-410; Altschul et al. Nature Genetics, 1993, 3:266-272).

Identity and similarity of related nucleic acid molecules and polypeptides can be readily calculated by known methods. Such methods include, but are not limited to, those described in Computational Molecular Biology, Lesk, A. M., ed., Oxford University Press, New York, 1988; York (1988); Biocomputing: Informatics and Genome Projects, Smith, D. W., ed., Academic Press, New York, 1993; York (1993); Computer Analysis of Sequence Data, Part 1, Griffin, A. M., and Griffin, H. G., eds., Humana Press, New Jersey, 1994; Jersey (1994); Sequence Analysis in Molecular Biology, von Heinje, G., Academic Press, 1987; Press (1987); Sequence Analysis Primer, Gribskov, M. and Devereux, J., eds., M. Stockton Press, New York, 1991; York (1991); and Carillo et al., SIAM J. Applied Math., 48:1073 (1988).

The methods, pharmaceutical compositions, and vectors may utilize biologically active fragments of nucleic acid sequences encoding the 126-amino acid atrial natriuretic factor (ANF) prohormone, such as nucleic acid sequences encoding NP₁₋₃₀, NP₃₁₋₆₇, NP₇₉₋₉₈, NP₉₉₋₁₂₆, and NP₇₃₋₁₀₂, (SEQ ID NO:1, SEQ ID NO:2, SEQ ID NO:3, SEQ ID NO:4, and SEQ ID NO:5, respectively), SEQ ID NO:6, and including biologically active fragments of the nucleic acid sequences encoding SEQ ID NO:1, SEQ ID NO:2, SEQ ID NO:3, SEQ ID NO:4, SEQ ID NO:5, and SEQ ID NO:6.

Representative fragments of the nucleotide sequences according to one example will be understood to mean any polynucleotide fragment having at least 8 or 9 consecutive nucleotides, preferably at least 12 consecutive nucleotides, and still more preferably at least 15 or at least 20 consecutive nucleotides of the sequence from which it is derived, with retention of biological activity as described herein. The upper limit for such fragments is one nucleotide less than the total number of nucleotides found in the full-length sequence (or, in certain embodiments, of the full length open reading frame (ORF) identified herein).

In other embodiments, fragments of nucleic acid sequences can comprise consecutive nucleotides of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, and up to one nucleotide less than the polynucleotide encoding full length ANF prohormone. In some embodiments, fragments comprise biologically active fragments of SEQ ID NO:1, SEQ ID NO:2, SEQ ID NO:3, SEQ ID NO:4, SEQ ID NO:5, or SEQ ID NO:6.

It is also well known in the art that restriction enzymes can be used to obtain biologically active fragments of the nucleic acid sequences, such as those encoding SEQ ID NO:1, SEQ ID NO:2, SEQ ID NO:3, SEQ ID NO:4, SEQ ID NO:5, and SEQ ID NO:6. For example, Bal31 exonuclease can be conveniently used for time-controlled limited digestion of DNA (commonly referred to as “erase-a-base” procedures). See, for example, Maniatis et al. [1982] Molecular Cloning: A Laboratory Manual, Cold Spring Harbor Laboratory, New York; Wei et al., J. Biol. Chem., 1983, 258:13006-13512.

The methods and pharmaceutical compositions may utilize amino acid sequences that are biologically active fragments of the 126-amino acid atrial natriuretic factor (ANF) prohormone, such as NP₁₋₃₀, NP₃₁₋₆₇, NP₇₉₋₉₈, NP₉₉₋₁₂₆, and NP₇₃₋₁₀₂ (SEQ ID NO:1, SEQ ID NO:2, SEQ ID NO:3, SEQ ID NO:4, and SEQ ID NO:5, respectively), SEQ ID NO:6, and including biologically active fragments of SEQ ID NO:1, SEQ ID NO:2, SEQ ID NO:3, SEQ ID NO:4, SEQ ID NO:5, and SEQ ID NO:6.

Representative fragments of the polypeptides according to one example, will be understood to mean any polypeptide fragment having at least 8 or 9 consecutive amino acids, preferably at least 12 amino acids, and still more preferably at least 15 or at least 20 consecutive amino acids of the polypeptide sequence from which it is derived, with retention of biological activity as described herein. The upper limit for such fragments is one amino acid less than the total number of amino acids found in the full-length sequence.

In other embodiments, fragments of the polypeptides can comprise consecutive amino acids of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, and up to one amino acid less than the full-length ANF prohormone. Fragments of polypeptides can be any portion of the full-length ANF prohormone amino acid sequence (including human or non-human mammalian homologs of the ANF prohormone) that exhibit biological activity as described herein, e.g., a C-terminally or N-terminally truncated version of the ANF prohormone, or an intervening portion of the ANF prohormone. In some embodiments, fragments comprise biologically active fragments of SEQ ID NO:1, SEQ ID NO:2, SEQ ID NO:3, SEQ ID NO:4, SEQ ID NO:5, and SEQ ID NO:6.

Other biologically equivalent forms of ANF fragments or homologs thereof may also be used, as can be appreciated by the sequence comparison below. Sequence similarities between mouse and human forms of ANP are shown where areas of conservation are clearly seen.

NCBI BLAST Comparison of Mouse (Query) to Human (Sbjct) ANP a.a. Sequences.

Query:
(SEQ ID NO: 29)

| | |
|---|---|
| 1 | MGSFSITLGFFLVLAFWLPGHIGANPVYSAVSNTDLMDFKNLLDH |
| | LEEKMPVEDEVMPP |

(SEQ ID NO: 30)

M SFS T + F L+LAF L G ANP+Y+AVSN DLMDFKNLLDHL
EEKMP+EDEV+PP

Sbjct:
(SEQ ID NO: 31)

| | |
|---|---|
| 1 | MSSFSTTTVSFLLLLAFQLLGQTRANPMYNAVSNADLMDFKNLLD |
| | HLEEKMPLEDEVVPP |

Query:
(SEQ ID NO: 32)

| | |
|---|---|
| 60 | QALSEQTEEAGAALSSLPEVPPWTGEVNPPLRDGSALGRSPWDPS |
| | DXXXXXXXXXXXXXX |

(SEQ ID NO: 33)

Q LSE EEAGAALS LPEVPPWTGEV+P RDG ALGR PWD SD

Sbjct:
(SEQ ID NO: 34)

| | |
|---|---|
| 61 | QVLSEPNEEAGAALSPLPEVPPWTGEVSPAQRDGGALGRGPWDSS |
| | DRSALLKSKLRALLT |

Query:
(SEQ ID NO: 35)

| | |
|---|---|
| 120 | GPRSLRRSSCFGGRIDRIGAQSGLGCNSFRY |

(SEQ ID NO: 36)

PRSLRRSSCFGGR+DRIGAQSGLGCNSFRY

Sbjct:
(SEQ ID NO: 37)

| | |
|---|---|
| 121 | APRSLRRSSCFGGRMDRIGAQSGLGCNSFRY |

The NP may be peptide derivatives, such as those disclosed in U.S. Patent Publication No. 2004/0266673 (Bakis et al.), which is incorporated herein by reference in its entirety. These NP derivates include an NP and a reactive entity coupled to the NP peptide. The reactive entity is able to covalently bond with a functionality on a blood component. Such NP derivatives are reported to have an extended half-life in vivo. The NP utilized in the subject invention can be a modified NP, such as those described in U.S. Patent Publication No. 2004/0002458 (Seilhamer et al.) and U.S. Patent Publication No. 2003/0204063 (Gravel et al.), which are incorporated herein by reference in their entirety.

The NP utilized may be a fusion polypeptide comprising an NP, or fragment or homolog thereof, and one or more additional polypeptides, such as another NP or a carrier protein, including those described in U.S. Patent Publication No. 2004/0138134 (Golembo et al.), which is incorporated herein by reference in its entirety. The NP utilized may be a chimeric polypeptide, such as those described in U.S. Patent Publication No. 2003/0069186 (Burnett et al.), which is incorporated herein by reference in its entirety. The fusion polypeptide or chimeric polypeptide may be administered to cells in vitro or in vivo directly (i.e., as a polypeptide), or the fusion polypeptide may be administered as a polynucleotide encoding the fusion polypeptide with an operably linked promoter sequence. See, for example, Wang W. et al., “Albubnp, a Recombinant B-type Natriuretic Peptide and Human Serum Albumin Fusion Hormone, as a Long-Term Therapy of Congestive Heart Failure”, Pharmaceutical Research, Springer Science and Business Media B. V., Formerly Kluwer Academic Publishers B.V., ISSN:0724-8741, volume 21, Number 11, November, 2004, pages 2105-2111.

The NP includes all hydrates and salts of natriuretic peptides that can be prepared by those of skill in the art. Under conditions where the compounds in one example are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, sulfate, nitrate, bicarbonate, and carbonate salts.

Pharmaceutically acceptable salts of NP may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The NP may be prepared by well-known synthetic procedures. For example, the polypeptides can be prepared by the well-known Merrifield solid support method. See Merrifield, J. Amer. Chem. Soc., 1963, 85:2149-2154 and Merrifield (1965) Science 150:178-185. This procedure, using many of the same chemical reactions and blocking groups of classical peptide synthesis, provides a growing peptide chain anchored by its carboxyl terminus to a solid support, usually cross-linked polystyrene or styrenedivinylbenzene copolymer. This method conveniently simplifies the number of procedural manipulations since removal of the excess reagents at each step is effected simply by washing of the polymer.

Alternatively, these peptides can be prepared by use of well-known molecular biology procedures. Polynucleotides, such as DNA sequences, encoding the NP according to one example can be readily synthesized. Such polynucleotides are a further aspect of the present invention. These polynucleotides can be used to genetically engineer eukaryotic or prokaryotic cells, for example, bacteria cells, insect cells, algae cells, plant cells, mammalian cells, yeast cells or fungi cells for synthesis of the peptides.

The biological activity attributable to the homologs and fragments of NP and NP-encoding nucleic acid sequences means the capability to prevent or alleviate symptoms associated with inflammatory and/or cell proliferation disorders such as cancer. This biological activity can be mediated by one or more of the following mechanisms: increased production of intracellular Ca⁺⁺ concentration (e.g., in epithelial cells), increased production of nitric oxide (NO), and decreased activation of transcription factors such as NFkB, ERK1, 2 and/or AP1.

The methods also include the administration of cells that have been genetically modified to produce NP, or biologically active fragments, variants, or homologs thereof. Such genetically modified cells can be administered alone or in combinations with different types of cells. Thus, genetically modified cells can be co-administered with other cells, which can include genetically modified cells or non-genetically modified cells. Genetically modified cells may serve to support the survival and function of the co-administered cells, for example.

The term “genetic modification” as used herein refers to the stable or transient alteration of the genotype of a cell of the subject invention by intentional introduction of exogenous nucleic acids by any means known in the art (including for example, direct transmission of a polynucleotide sequence from a cell or virus particle, transmission of infective virus particles, and transmission by any known polynucleotide-bearing substance) resulting in a permanent or temporary alteration of genotype. The nucleic acids may be synthetic, or naturally derived, and may contain genes, portions of genes, or other useful polynucleotides in addition to those encoding NP. A translation initiation codon can be inserted as necessary, making methionine the first amino acid in the sequence. The term “genetic modification” is not intended to include naturally occurring alterations such as that which occurs through natural viral activity, natural genetic recombination, or the like. The genetic modification may confer the ability to produce NP, wherein the cell did not previously have the capability, or the modification may increase the amount of NP endogenously produced by the cell, e.g., through increased expression.

Exogenous nucleic acids and/or vectors encoding NP can be introduced into a cell by viral vectors (retrovirus, modified herpes virus, herpes virus, adenovirus, adeno-associated virus, lentivirus, and the like) or direct DNA transfection (lipofection, chitosan-nanoparticle mediated transfection, calcium phosphate transfection, DEAE-dextran, electroporation, and the like), microinjection, cationic lipid-mediated transfection, transduction, scrape loading, ballistic introduction and infection (see, for example, Sambrook et al. [1989] Molecular Cloning: A Laboratory Manual, 2^{nd} Ed., Cold Spring Harbor Laboratory Press, Cold Spring Harbor, N.Y.).

Preferably, the exogenous polynucleotide encoding the NP is operably linked to a promoter sequence that permits expression of the polynucleotide in a desired tissue within the patient. The promoters can be inducible, tissue-specific, or event-specific, as necessary.

The genetically modified cell may be chosen from eukaryotic or prokaryotic systems, for example, bacterial cells (Gram negative or Gram positive), yeast cells, animal cells, plant cells, and/or insect cells using baculovirus vectors, for example. In some embodiments, the genetically modified cell for expression of the nucleic acid sequences encoding NP, are human or non-human mammal cells.

According to the methods in one example, NP or polynucleotides encoding NP may be administered to a patient in order to alleviate (e.g., reduce or eliminate) a variety of symptoms associated with cancers, in various stages of pathological development. Treatment with NP or nucleic acid sequences encoding NP is intended to include prophylactic intervention to prevent or reduce cancer cell growth (e.g., tumor growth) and onset of the symptoms associated with cancer cell growth (e.g., tumor growth), such as pain. The nucleic acid sequences and pharmaceutical compositions may be co-administered (concurrently or consecutively) to a patient with other therapeutic agents useful for treating cancers of the lung, ovarian, breast, as well as melanomas.

Suitable expression vectors for NP include any that are known in the art or yet to be identified that will cause expression of NP-encoding nucleic acid sequences in mammalian cells. Suitable promoters and other regulatory sequences can be selected as is desirable for a particular application. The promoters can be inducible, tissue-specific, or event-specific, as necessary. For example, the cytomegalovirus (CMV) promoter (Boshart et al., Cell, 1985, 41:521-530) and SV40 promoter (Subramani et al., Mol. Cell. Biol., 1981, 1:854-864) have been found to be suitable, but others can be used as well. Optionally, the NP-encoding nucleic acid sequences used in the subject invention include a sequence encoding a signal peptide upstream of the NP-encoding sequence, thereby permitting secretion of the NP from a host cell. Also, various promoters may be used to limit the expression of the peptide in specific cells or tissues, such as lung cells.

A tissue-specific and/or event-specific promoter or transcription element that responds to the target microenvironment and physiology can also be utilized for increased transgene expression at the desired site. There has been an immense amount of research activity directed at strategies for enhancing the transcriptional activity of weak tissue-specific promoters or otherwise increasing transgene expression with viral vectors. It is possible for such strategies to provide enhancement of gene expression equal to one or two orders of magnitude, for example (see Nettelbeck et al., Gene Ther., 1998, 5(12):1656-1664 and Qin et al., Hum. Gene Ther., 1997, 8(17):2019-2019, the abstracts of which are submitted herewith for the Examiner's convenience). Examples of cardiac-specific promoters are the ventricular form of MLC-2v promoter (see, Zhu et al., Mol. Cell. Biol., 1993, 13:4432-4444, Navankasattusas et al., Mol. Cell. Biol., 1992, 12:1469-1479, 1992) and myosin light chain-2 promoter (Franz et al., Circ. Res., 1993, 73:629-638). The E-cadherin promoter directs expression specific to epithelial cells (Behrens et al., PNAS, 1991, 88:11495-11499), while the estrogen receptor (ER) 3 gene promoter directs expression specifically to the breast epithelium (Hopp et al., J. Mammary Gland Biol. Neoplasia, 1998, 3:73-83). The human C-reactive protein (CRP) gene promoter (Ruther et al., Oncogene 8:87-93, 1993) is a liver-specific promoter. An example of a muscle-specific gene promoter is human enolase (ENO3) (Peshavaria et al., Biochem. J., 1993, 292(Pt 3):701-704). A number of brain-specific promoters are available such as the thy-1 antigen and gamma-enolase promoters (Vibert et al., Eur. J. Biochem. 181:33-39, 1989). The prostate-specific antigen promoter provides prostate tissue specificity (Pang et al., Gene Ther., 1995, 6(11):1417-1426; Lee et al., Anticancer Res., 1996, 16(4A): 1805-1811). The surfactant protein B promoter provides lung specificity (Strayer et al., Am. J. Respir. Cell Mol. Biol., 1998, 18(1):23-33). Any of the aforementioned promoters may be selected for targeted or regulated expression of the NP-encoding polynucleotide.

Various viral or non-viral vectors may be used to deliver polynucleotides encoding NP to cells in vitro or in vivo, resulting in expression and production of NP. Tissue-specific promoters or event-specific promoters may be utilized with polynucleotides encoding NP to further optimize and localize expression at target sites, such as within diseased tissues (e.g., cancer cells or tissues containing cancer cells). Robson et al. review various methodologies and vectors available for delivering and expressing a polynucleotide in vivo for the purpose of treating cancer (Robson, T. Hirst, D. G., J. Biomed. and Biotechnol., 2003, 2003(2): 110-137). Among the various targeting techniques available, transcriptional targeting using tissue-specific and event-specific transcriptional control elements is discussed. For example, Table 1 at page 112 of the Robson et al. publication lists several tissue-specific promoters useful in cancer therapy. Tables 2-4 of the Robson et al. publication list tumor-specific promoters, tumor environment-specific promoters, and exogenously controlled inducible promoters, many of which were available at the time the patent application was filed. The successful delivery and expression of the p53 tumor suppressor gene in vivo has been documented (Horowitz, J. Curr. Opin. Mol. Ther., 1999, 1(4):500-509; Von Gruenigen, V. E. et al. Int. J. Gynecol. Cancer, 1999, 9(5):365-372; Fujiwara, T. et al., Mol. Urol., 2000, 4(2):51-54, respectively).

Many techniques for delivery of drugs and proteins are available in the art to reduce the effects of enzymatic degradation, to facilitate cell uptake, and to reduce any potential toxicity to normal (undiseased) cells, etc. Such methods and reagents can be utilized for administration of NP to cells in vitro or in vivo. For example, peptides known as “cell penetrating peptides” (CPP) or “protein transduction domains” (PTD) have an ability to cross the cell membrane and enter the cell. PTDs can be linked to a cargo moiety such as a drug, peptide, or full-length protein, and can transport the moiety across the cell membrane. One well characterized PTD is the human immunodeficient virus (HIV)-1 Tat peptide (see, for example, Frankel et al., U.S. Pat. Nos. 5,804,604; 5,747,641; 6,674,980; 5,670,617; and 5,652,122; Fawell, S. et al., Proc. Natl. Acad. Sci. U.S.A., 1994, 91:664-668). Peptides such as the homeodomain of Drosophila Antennapedia (ANTp) and arginine-rich peptides display similar properties (Derossi, D. et al., J. Biol. Chem., 1994, 269:10444-10450; Derossi, D. et al., Trends Cell Biol., 1998, 8:84-87; Rojas, M. et al., Nat. Biotechnol., 1998, 16:370-375; Futaki, S. et al., J. Biol. Chem., 2001, 276:5836-5840). VP22, a tegument protein from Herpes simplex virus type 1 (HSV-1), also has the ability to transport proteins across a cell membrane (Elliot et al., Cell, 1997, 88:223-233; Schwarze S. R. et al., Trends Pharmacol. Sci., 2000, 21:45-48). A common feature of these carriers is that they are highly basic and hydrophilic (Schwarze S. R. et al., Trends Cell Biol., 2000, 10:290-295). Coupling of these carriers to marker proteins such as beta-galactosidase has been shown to confer efficient internalization of the marker protein into cells. More recently, chimeric, in-frame fusion proteins containing these carriers have been used to deliver proteins to a wide spectrum of cell types both in vitro and in vivo. For example, VP22-p53 chimeric protein retained its ability to spread between cells and its pro-apoptotic activity, and had a widespread cytotoxic effect in p53 negative human osteosarcoma cells in vitro (Phelan, A. et al., Nature Biotechnol., 1998, 16:440-443). Intraperitoneal injection of the beta-galactosidase protein fused to the HIV-1 Tat peptide resulted in delivery of the biologically active fusion protein to all tissues in mice, including the brain (Schwarze S. R. et al., Science, 1999, 285:1569-1572).

Liposomes of various compositions can also be used for site-specific delivery of proteins and drugs (Witschi, C. et al., Pharm. Res., 1999, 16:382-390; Yeh, M. K. et al., Pharm. Res., 1996, 1693-1698). The interaction between the liposomes and the protein cargo usually relies on hydrophobic interactions or charge attractions, particularly in the case of cationic lipid delivery systems (Zelphati, O. et al., J. Biol. Chem., 2001, 276:35103-35110). Tat peptide-bearing liposomes have also been constructed and used to deliver cargo directly into the cytoplasm, bypassing the endocytotic pathway (Torchilin V. P. et al., Biochim. Biophys. Acta—Biomembranes, 2001, 1511:397-411; Torchilin V. P. et al., Proc. Natl. Acad. Sci. USA, 2001, 98:8786-8791). When encapsulated in sugar-grafted liposomes, pentamidine isethionate and a derivative have been found to be more potent in comparison to normal liposome-encapsulated drug or to the free drug (Banerjee, G. et al., J. Antimicrob. Chemother., 1996, 38(1):145-150). A thermo-sensitive liposomal taxol formulation (heat-mediated targeted drug delivery) has been administered in vivo to tumor-bearing mice in combination with local hyperthermia, and a significant reduction in tumor volume and an increase in survival time was observed compared to the equivalent dose of free taxol with or without hyperthermia (Sharma, D. et al., Melanoma Res., 1998, 8(3):240-244). Topical application of liposome preparations for delivery of insulin, IFN-alpha, IFN-gamma, and prostaglandin E1 have met with some success (Cevc G. et al., Biochim. Biophys, Acta, 1998, 1368:201-215; Foldvari M. et al., J. Liposome Res., 1997, 7:115-126; Short S. M. et al., Pharm. Res., 1996, 13:1020-1027; Foldvari M. et al., Urology, 1998, 52(5):838-843; U.S. Pat. No. 5,853,755).

Antibodies represent another targeting device that may make liposome uptake tissue-specific or cell-specific (Mastrobattista, E. et al., Biochim. Biophys. Acta, 1999, 1419(2):353-363; Mastrobattista, E. et al., Adv. Drug Deliv. Rev., 1999, 40(1-2):103-127). The liposome approach offers several advantages, including the ability to slowly release encapsulated drugs and proteins, the capability of evading the immune system and proteolytic enzymes, and the ability to target tumors and cause preferentially accumulation in tumor tissues and their metastases by extravasation through their leaky neovasculature. Other carriers have also been used to deliver anti-cancer drugs to neoplastic cells, such as polyvinylpyrrolidone nanoparticles and maleylated bovine serum albumin (Sharma, D. et al., Oncol. Res., 1996, 8(7-8):281-286; Mukhopadhyay, A. et al., FEBS Lett., 1995, 376(1-2):95-98). Thus, using targeting and encapsulation technologies, which are very versatile and amenable to rational design and modification, delivery of NP to desired cells can be facilitated. Furthermore, because many liposome compositions are also viable delivery vehicles for genetic material, many of the advantages of liposomes are equally applicable to polynucleotides encoding NP.

As indicated above, the pharmaceutical composition may include a liposome component. According to one example, a liposome comprises a lipid composition that is capable of fusing with the plasma membrane of a cell, thereby allowing the liposome to deliver a nucleic acid molecule and/or a protein composition into a cell. Some preferred liposomes include those liposomes commonly used in gene delivery methods known to those of skill in the art. Some preferred liposome delivery vehicles comprise multilamellar vesicle (MLV) lipids and extruded lipids, although not limited to such liposomes. Methods for preparation of MLVs are well known in the art. “Extruded lipids” are also contemplated. Extruded lipids are lipids that are prepared similarly to MLV lipids, but which are subsequently extruded through filters of decreasing size, as described in Templeton et al., Nature Biotech., 1997, 15:647-652, which is incorporated herein by reference in its entirety. Small unilamellar vesicle (SUV) lipids can also be used in the compositions and methods of the present invention. Other preferred liposome delivery vehicles comprise liposomes having a polycationic lipid composition (i.e., cationic liposomes). For example, cationic liposome compositions include, but are not limited to, any cationic liposome complexed with cholesterol, and without limitation, include DOTMA and cholesterol, DOTAP and cholesterol, DOTIM and cholesterol, and DDAB and cholesterol. Liposomes utilized in the present invention can be any size, including from about 10 to 1000 nanometers (nm), or any size in between.

A liposome delivery vehicle can be modified to target a particular site in a mammal, thereby targeting and making use of an NP-encoding nucleic acid molecule of the present invention at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. In one embodiment, other targeting mechanisms, such as targeting by addition of exogenous targeting molecules to a liposome (i.e., antibodies) may not be a necessary component of the liposome of the present invention, since effective immune activation at immunologically active organs can already be provided by the composition when the route of delivery is intravenous or intraperitoneal, without the aid of additional targeting mechanisms. However, in some embodiments, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., Biochemistry, 1986, 25: 5500-6; Ho et al., J Biol Chem, 1987a, 262: 13979-84; Ho et al., J Biol Chem, 1987b, 262: 13973-8; and U.S. Pat. No. 4,957,735 to Huang et al., each of which is incorporated herein by reference in its entirety). In one embodiment, if avoidance of the efficient uptake of injected liposomes by reticuloendothelial system cells due to opsonization of liposomes by plasma proteins or other factors is desired, hydrophilic lipids, such as gangliosides (Allen et al., FEBS Lett, 1987, 223: 42-6) or polyethylene glycol (PEG)-derived lipids (Klibanov et al., FEBS Lett, 1990, 268: 235-7), can be incorporated into the bilayer of a conventional liposome to form the so-called sterically-stabilized or “stealth” liposomes (Woodle et al., Biochim Biophys Acta, 1992, 1113: 171-99). Variations of such liposomes are described, for example, in U.S. Pat. No. 5,705,187 to Unger et al., U.S. Pat. No. 5,820,873 to Choi et al., U.S. Pat. No. 5,817,856 to Tirosh et al.; U.S. Pat. No. 5,686,101 to Tagawa et al.; U.S. Pat. No. 5,043,164 to Huang et al., and U.S. Pat. No. 5,013,556 to Woodle et al., all of which are incorporated herein by reference in their entireties).

The NP-encoding nucleic acid sequences may conjugate with chitosan. For example, DNA chitosan nanospheres can be generated, as described by Roy, K. et al. (1999, Nat Med 5:387). Chitosan allows increased bioavailability of the NP-encoding nucleic acid sequences because of protection from degradation by serum nucleases in the matrix and thus has great potential as a mucosal gene delivery system. Chitosan also has many beneficial effects, including anticoagulant activity, wound-healing properties, and immunostimulatory activity, and is capable of modulating immunity of the mucosa and bronchus-associated lymphoid tissue.

Mammalian species which benefit from the disclosed methods of treatment include, and are not limited to, apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. The terms “patient” and “subject” are used interchangeably herein are intended to include such human and non-human mammalian species. According to the method of the present invention, human or non-human mammalian NP (or nucleic acid sequences encoding human or non-human mammalian NP) can be administered to the patient. The NP may be naturally occurring within the patient's species or a different mammalian species. The expression vectors used in the subject invention can comprise nucleic acid sequences encoding any human or non-human mammalian NP. In instances where genetically modified cells are administered to a patient, the cells may be autogenic, allogeneic, or xenogeneic, for example.

In another example, pharmaceutical compositions containing a therapeutically effective amount of agent that reduces NPR-A activity, such as an NP, or polynucleotides encoding NP, and a pharmaceutically acceptable carrier. Preferably, if the agent is a polynucleotide, such as an NP-encoding nucleic acid sequence, the polynucleotide is contained within an expression vector, such as plasmid DNA or a virus. Pharmaceutical compositions including a therapeutically effective amount of an agent that reduces NPR-A activity such as NP, or nucleic acid sequences encoding NP, and a pharmaceutically acceptable carrier, can be administered to a patient by any effective route, including local or systemic delivery. Administration can be continuous or at distinct intervals as can be determined by a person skilled in the art.

The agent that reduces NPR-A activity, such as NP or polynucleotides encoding NP (and pharmaceutical compositions containing them), can be administered to a patient by any route that results in prevention (or reduction of onset) or alleviation of symptoms associated with cancer, such as pain. For example, the agent (e.g., NP or NP-encoding nucleic acid molecule) can be administered parenterally, intravenously (I.V.), intramuscularly (I.M.), subcutaneously (S.C.), intradermally (I.D.), topically, transdermally, orally, intranasally, etc.

If desired, the pharmaceutical composition may be adapted for administration to the airways of the patient, e.g., nose, sinus, throat and lung, for example, as nose drops, as nasal drops, by nebulization as an inhalant, vaporization, or other methods known in the art. Examples of intranasal administration can be by means of a spray, drops, powder or gel and also described in U.S. Pat. No. 6,489,306, which is incorporated herein by reference in its entirety. One embodiment is administering the composition as a nasal spray. Alternate embodiments include administration through any oral or mucosal routes, sublingual administration and even eye drops. However, other means of drug administrations are well within the scope of the composition.

The pharmaceutical compositions may be formulated according to known methods for preparing pharmaceutically useful compositions. Furthermore, as used herein, the phrase “pharmaceutically acceptable carrier” includes any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients used in the compositions. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E. W., 1995, Easton Pa., Mack Publishing Company, 19^{th} ed.), which is incorporated herein by reference in its entirety, describes formulations that can be used in connection with the compositions.

Pharmaceutical compositions useful for parenteral injection may include pharmaceutically acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyol (such as glycerol, propylene glycol, polyethylene, lycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants. Formulations suitable for parenteral administration include, for example, aqueous injectable solutions that may contain antioxidants, buffers, and solutes which render the formulation isotonic with the blood of the intended recipient; and aqueous and nonaqueous sterile suspensions, which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that, in addition to the ingredients particularly mentioned above, the formulations of the subject invention can include other agents conventional in the art having regard to the type of formulation in question.

The pharmaceutical compositions used in the methods may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption, such as aluminum monostearate and gelatin.

In some cases, in order to prolong the effect of the active agent (e.g. NP), it is desirable to slow the absorption from subcutaneous or intramuscular injection. This may be accomplished by the use of a liquid suspension of crystalline or amorphous material with poor water solubility. The rate of absorption of the NP or NP-encoding polynucleotide then depends upon its rate of dissolution which, in turn, may depend upon crystal size and crystalline form. Alternatively, delayed absorption of a parenterally administered NP or NP-encoding polynucleotide is accomplished by dissolving or suspending the NP in an oil vehicle.

Injestable depot forms are made by forming microencapsule matrices of the agent (e.g., NP or NP-encoding polynucleotide) in biodegradable polymers such as polylactide-polyglycolide. Depending upon the ratio of active agent (e.g., NP or NP-encoding polynucleotide) to polymer and the nature of the particular polymer employed, the rate of release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active agents (NP or NP-encoding polynucleotide) are mixed with it least one pharmaceutically acceptable excipient or carrier such as sodium nitrate or dicalcium phosphate and/or a) fillers or extenders such as starches, lactose, sucrose, glucose, mannitol, and silicic acid; b) binders such as, for example, carboxymethylcellulose, alginates, gelatin, polyvinylpyrrolidone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. Optionally, the solid dosage forms contain opacifying agents, and can be of a composition that releases the NP or NP-encoding polynucleotide only, or preferentially, in a certain part of the intestinal tract, optionally, in a delayed manner. Examples of embedding compositions that can be used include polymeric substances and waxes.

The active agents (NP or NP-encoding polynucleotide) can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the NP or NP-encoding polynucleotide, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Topical administration includes administration to the skin or mucosa, including surfaces of the lung and eye. Compositions for topical administration, including those for inhalation, may be prepared as a dry powder, which may be pressurized or non-pressurized. In non-pressurized powder compositions, the active ingredients in finely divided form may be used in admixture with a larger-sized pharmaceutically acceptable inert carrier comprising particles having a size, for example, of up to 100 μm in diameter. Suitable inert carriers include sugars such as lactose. Desirably, at least 95% by weight of the particles of the active ingredient have an effective particle size in the range of 0.01 to 10 μm.

Alternatively, the pharmaceutical composition may be pressurized and contain a compressed gas, such as nitrogen or a liquefied gas propellant. The liquefied propellant medium or the entire composition is preferably such that the active ingredients do not dissolve therein to any substantial extent. The pressurized composition may also contain a surface active agent. The surface active agent may be a liquid or solid non-ionic surface active agent or may be a solid anionic surface active agent. It is preferred to use the solid anionic surface active agent in the form of a sodium salt.

The compositions and methods may further incorporate permeation enhancers, such as those described in U.S. Patent Publication No. 2003/0147943 (Luo et al.), penetrating peptides capable of facilitating penetration of an NP, or an NP-encoding polynucleotide, across a biological barrier, such as those described in U.S. Patent Publication No. 2004/0146549 (Ben-Sasson et al.), enhancer compounds that enhance the absorption of a polypeptide in the respiratory tract, such as those described in U.S. Patent Publication No. 2004/0171550 (Backstrom et al.), and suspension vehicles, such as those described in U.S. Patent Publication No. 2004/0224903 (Berry et al.), each of which are incorporated herein by reference in their entirety.

The agent that reduces NPR-A activity (such as NP or NP-encoding polynucleotide) is administered and dosed in accordance with good medical practice, taking into account the clinical condition of the individual patient, the site and method of administration, scheduling of administration, patient age, sex, body weight, and other factors known to medical practitioners. The pharmaceutically “effective amount” for purposes herein is thus determined by such considerations as are known in the art. For example, an effective amount of NP-encoding polynucleotide is that amount necessary to provide an effective amount of NP, when expressed in vivo or in vitro. The amount of the agent (e.g., NP or NP-encoding nucleic acid molecule) must be effective to achieve some improvement including, but not limited to, improved survival rate, more rapid recovery, total prevention of symptoms associated with an inflammatory or cell proliferation disorder, such as cancer, or improvement or elimination of symptoms associated with an inflammatory or cell proliferation disorder, such as cancer, and other indicators as are selected as appropriate measures by those skilled in the art. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for local or systemic administration based on the size of a mammal and the route of administration.

In one example, a mammal (such as a human) that is predisposed to or suffering from a physical disorder may be treated by administering to the mammal an effective amount of an agent that reduces NPR-A activity (such as NP or NP-encoding polynucleotide), in combination with a pharmaceutically acceptable carrier or excipient therefore (as described below). Physical disorders treatable with the compositions and methods of the present invention include any physical disorder that may be delayed, prevented cured or otherwise treated by administration of an agent that reduces NPR-A activity (such as NP or NP-encoding polynucleotide) in a mammal suffering from or predisposed to the physical disorder. Such physical disorders include, but are not limited to, a variety of carcinomas and other cancers, such as skin cancers (including melanomas and Kaposi's Sarcoma), oral cavity cancers, lung cancers, breast cancers, prostatic cancers, bladder cancers, liver cancers, pancreatic cancers, cervical cancers, ovarian cancers, head and neck cancers, colon cancers, germ cell cancers (including teratocarcinomas) and leukemias. Other physical disorders treatable by the methods of the present invention include inflammatory disorders such as rheumatoid arthritis, multiple sclerosis, systemic lupus erythematosis, pelvic inflammatory disease, and Crohn's disease. The methods may also be used to treat a mammal suffering from or predisposed to a fibrotic disorder, including pulmonary fibrosis, cystic fibrosis, endomyocardial fibrosis, hepatic fibrosis (particularly hepatic cirrhosis), myelofibrosis, scleroderma, and systemic sclerosis. Other physical disorders treatable by the methods in one example, include osteoporosis, atherosclerosis, and ocular disorders such as corneal ulceration and diabetic retinopathy. The methods of the present invention may also be used in the prevention of disease progression, such as in chemoprevention of the progression of a premalignant lesion to a malignant lesion, and to treat a mammal suffering from, or predisposed to, other physical disorders that respond to treatment with compositions that differentially modulate gene expression.

Cell proliferation disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Cancers of any organ can be treated, including cancers of, but are not limited to, e.g., colon, pancreas, breast, prostate, bone, liver, kidney, lung, testes, skin, pancreas, stomach, colorectal cancer, renal cell carcinoma, hepatocellular carcinoma, melanoma, etc.

Examples of breast cancer include, but are not limited to, invasive ductal carcinoma, invasive lobular carcinoma, ductal carcinoma in situ, and lobular carcinoma in situ. Examples of cancers of the respiratory tract include, but are not limited to, small-cell and non-small-cell lung carcinoma, as well as bronchial adenoma and pleuropulmonary blastoma. Examples of brain cancers include, but are not limited to, brain stem and hypothalamic glioma, cerebellar and cerebral astrocytoma, medulloblastoma, ependymoma, as well as neuroectodermal and pineal tumor. Tumors of the male reproductive organs include, but are not limited to, prostate and testicular cancer. Tumors of the female reproductive organs include, but are not limited to, endometrial, cervical, ovarian, vaginal, and vulvar cancer, as well as sarcoma of the uterus. Tumors of the digestive tract include, but are not limited to, anal, colon, colorectal, esophageal, gallbladder, gastric, pancreatic, rectal, small-intestine, and salivary gland cancers. Tumors of the urinary tract include, but are not limited to, bladder, penile, kidney, renal pelvis, ureter, and urethral cancers. Eye cancers include, but are not limited to, intraocular melanoma and retinoblastoma. Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to, squamous cell carcinoma, Kaposi's sarcoma, malignant melanoma, Merkel cell skin cancer, and non-melanoma skin cancer. Head-and-neck cancers include, but are not limited to, laryngeal, hypopharyngeal, nasopharyngeal, and/or oropharyngeal cancers, and lip and oral cavity cancer. Lymphomas include, but are not limited to, AIDS-related lymphoma, non-Hodgkin's lymphoma, cutaneous T-cell lymphoma, Hodgkin's disease, and lymphoma of the central nervous system. Sarcomas include, but are not limited to, sarcoma of the soft tissue, osteosarcoma, malignant fibrous histiocytoma, lymphosarcoma, and rhabdomyosarcoma. Leukemias include, but are not limited to, acute myeloid leukemia, acute lymphoblastic leukemia, chronic lymphocytic leukemia, chronic myelogenous leukemia, and hairy cell leukemia. In addition to reducing the proliferation of tumor cells, agents that reduce NPR-A activity can also cause tumor regression, e.g., a decrease in the size of a tumor, or in the extent of cancer in the body.

In addition to chemotherapeutic agents, the methods and compositions of the subject invention can incorporate treatments and agents utilizing, for example, angiogenesis inhibitors (Thalidomide, Bevacizumab), Bcl-2 antisense oligonucleotides (G3139), a PSA based vaccine, a PDGF receptor inhibitor (Gleevec), microtubule stabilizers (Epothilones), and a pro-apoptotic agent (Perifosine). Thus, an NP or NP-encoding polynucleotide can be administered to a patient in combination (simultaneously or consecutively) with other agents for useful for treating inflammatory disorders and/or cell proliferation disorders. Likewise, the pharmaceutical compositions of the subject invention can include such agents.

The term “gene therapy”, as used herein, refers to the transfer of genetic material (a polynucleotide, e.g., DNA or RNA) of interest into a host to treat or prevent a genetic or acquired disease or condition phenotype. The genetic material of interest encodes a product (e.g., a protein, polypeptide, peptide, or functional RNA) whose production in vivo is desired, such as NP. In addition to one or more NP, the genetic material of interest can encode a hormone, receptor, enzyme, polypeptide or peptide of therapeutic and/or diagnostic value. For a review see, in general, the text “Gene Therapy (Advances in Pharmacology 40, Academic Press, 1997).

Two basic approaches to gene therapy have evolved: (1) ex vivo and (2) in vivo gene therapy. In ex vivo gene therapy, cells are removed from a patient and, while being cultured, are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to produce the transfected gene product in situ.

In in vivo gene therapy, target cells are not removed from the subject, rather the gene to be transferred is introduced into the cells of the recipient organism in situ, that is within the recipient. Alternatively, if the host gene is defective, the gene is repaired in situ. Thus, these genetically altered cells produce the transfected gene product (e.g., NP) in situ.

The gene expression vector is capable of delivery/transfer of heterologous nucleic acid sequences (e.g., NP-encoding nucleic acid sequences) into a host cell. The expression vector may include elements to control targeting, expression and transcription of the nucleic acid sequence in a cell selective manner as is known in the art. It should be noted that often the 5′UTR and/or 3′UTR of the gene may be replaced by the 5′UTR and/or 3′UTR of the expression vehicle.

The expression vector can include a promoter for controlling transcription of the heterologous material and can be either a constitutive or inducible promoter to allow selective transcription. The expression vector can also include a selection gene.

Vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and include, for example, stable or transient transfection, lipofection, electroporation, and infection with recombinant viral vectors.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of a DNA viral vector for introducing and expressing recombinant sequences is the adenovirus derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur. Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Another aspect of the invention concerns an isolated peptide comprising the amino acid sequence NP₇₃₋₁₀₂ (SEQ ID NO:5), or a biologically active fragment or homolog thereof NP₇₃₋₁₀₂ is amino acids 73-102 of the 151-amino acid long human atrial natriuretic factor (ANF). In another aspect, the present invention concerns an isolated peptide comprising the amino acid sequence of SEQ ID NO:6, or a biologically active fragment or homolog thereof SEQ ID NO:6 is a biologically active fragment of the human ANF. In another aspect, the present invention concerns an isolated nucleic acid molecule encoding the amino acid sequence of NP₇₃₋₁₀₂ (SEQ ID NO:5), or a biologically active fragment or homolog thereof. In another aspect, the present invention concerns an isolated nucleic acid molecule (SEQ ID NO:13) encoding the amino acid sequence of SEQ ID NO:6, or a biologically active fragment or homolog thereof.

As used herein, the terms “peptide”, “polypeptide”, and “protein” refer to amino acid sequences of any length unless otherwise specified.

Assays for Identifying Agents that Reduce Natriuretic Peptide Receptor-A Activity

Methods for identifying agents that reduce the activity of natriuretic peptide receptor-A (also known in the art as NPRA, NPR-A, and guanylate cyclase A) in vitro or in vivo (also referred to herein as the diagnostic method or screening assay). Such agents are potentially useful for treating inflammatory or cell proliferation disorders in a patient. In the therapeutic methods and assays in one example, agents that reduce NPR-A activity include those that, for example, reduce ANP-NPR-A induced c-GMP production, reduce expression of NPR-A, reduce cellular internalization of NPR-A, reduce recycling of NPR-A to the cell membrane, or otherwise interfere with the activity of the receptor.

Production of ANP-NPR-A induced cGMP production can be assayed and used as a high-throughput method for screening agents for anti-proliferative (e.g., anti-cancer) and anti-inflammatory activity. This assay can be carried out using a cell line that transiently or stably expresses the receptor for ANP, NPR-A (Pandey et al., J Biol. Chem. 2002, 277:4618-4627) and libraries of agents, such as peptide and compound libraries, which can be novel or obtained commercially. An assay for cGMP can be performed to select agents that are inhibitors of cGMP. Alternatively, ANP peptide can be linked with a moiety that can antagonize cGMP following internalization, which can be checked using a transiently or stably transfected cell line expressing NPR-A.

In the context of the screening assay, the terms “recombinant host cells”, “host cells”, “genetically modified host cells” “cells”, “cell lines”, “cell cultures”, and other such terms denoting microorganisms or higher eukaryotic cell lines cultured as unicellular entities refer to cells which can be, or have been, used as recipients for recombinant vectors or other transfer DNA, immaterial of the method by which the DNA is introduced into the cell or the subsequent disposition of the cell. The terms include the progeny of the original cell that has been transfected. Cells in primary culture can also be used as recipients. Host cells can range in plasticity and proliferation potential. Host cells can be differentiated cells, progenitor cells, or stem cells, for example.

Host cells can be genetically modified with vectors to express (e.g., overexpress) the NPR-A receptor, or a mutant, isoform, or other variant thereof, which may be a cloning vector or an expression vector, for example. The vector may be in the form of a plasmid, a virus, (e.g., a retrovirus or other virus), a viral particle, a phage, etc. The genetically modified host cells can be cultured in conventional nutrient media modified as appropriate for activating promoters, selecting transformants/transfectants or amplifying the receptor-encoding polynucleotide.

In one embodiment, the host cell is a human cell. In another embodiment, the host cell is a non-human mammalian cell. Both prokaryotic and eukaryotic host cells may be used for expression of desired coding sequences when appropriate control sequences (e.g., promoter sequences) that are compatible with the designated host are used. For example, among prokaryotic hosts, Escherichia coli may be used. Also, for example, expression control sequences for prokaryotes include but are not limited to promoters, optionally containing operator portions, and ribosome binding sites. Eukaryotic hosts include yeast and mammalian cells in culture systems. Pichia pastoris, Saccharomyces cerevisiae and S. carlsbergensis are commonly used yeast hosts. Yeast-compatible vectors carry markers that permit selection of successful transformants by conferring protrophy to auxotrophic mutants or resistance to heavy metals on wild-type strains. Yeast compatible vectors may employ the 2-μ, origin of replication (Broach et al. Meth. Enzymol. 101:307, 1983), the combination of CEN3 and ARS1 or other means for assuring replication, such as sequences that will result in incorporation of an appropriate fragment into the host cell genome. Control sequences for yeast vectors are known in the art and include but are not limited to promoters for the synthesis of glycolytic enzymes, including the promoter for 3-phosphoglycerate kinase. (See, for example, Hess et al. J. Adv. Enzyme Reg. 7:149, 1968; Holland et al. Biochemistry 17:4900, 1978; and Hitzeman J. Biol. Chem. 255:2073, 1980). For example, some useful control systems are those that comprise the glyceraldehyde-3-phosphate dehydrogenase (GAPDH) promoter or alcohol dehydrogenase (ADH) regulatable promoter, terminators also derived from GAPDH, and, if secretion is desired, leader sequences from yeast alpha factor. In addition, the transcriptional regulatory region and the transcriptional initiation region which are operably linked may be such that they are not naturally associated in the wild-type organism.

Host cells useful for expression of polynucleotides encoding the NPR-A receptor may be primary cells or cells of cell lines. The host cells may be tumor cells (transformed cells) or non-tumor cells. Mammalian cell lines available as hosts for expression are known in the art and are available from depositories such as the American Type Culture Collection. These include but are not limited to HeLa cells, human embryonic kidney (HEK) cells, Chinese hamster ovary (CHO) cells, baby hamster kidney (BHK) cells, and others.

The number of host cells used in a particular assay will vary with the objectives of the assay, the solid support used to support or contain the cell(s), if one is utilized etc. Thus, in some protocols, the host cell may be a single cell. In other protocols, a plurality of host cells will be used.

In accordance with the screening assay in one example, the polynucleotide encoding the NPR-A is operably linked to a promoter sequence. Suitable promoters' sequences for mammalian cells also are known in the art and include viral promoters such as that from Simian Virus 40 (SV40), Rous sarcoma virus (RSV), adenovirus (ADV), bovine papilloma virus (BPV) and cytomegalovirus (CMV). Mammalian cells also may require terminator sequences and poly A addition sequences; enhancer sequences which increase expression also may be included, and sequences which cause amplification of the gene also may be desirable. These sequences are known in the art. Vectors suitable for replication in mammalian cells may include viral replicons, or sequences which ensure integration of the appropriate sequences including the NPR-A receptor into the host genome. An example of such a mammalian expression system is described in Gopalakrishnan et al. Eur. J. Pharmacol.—Mol. Pharmacol. 290: 237-246, 1995).

Candidate agents (and treatments) that may be tested by the screening assays of the present invention include polypeptides, non-peptide small molecules, biological agents, and any other source of candidate agents potentially having the ability to modulate (e.g., reduce) NPR-A activity. Candidate agents and treatments may be useful for the treatment of inflammatory and/or cell proliferation disorders, such as cancer. Candidate agents can be virtually any substance and can encompass numerous chemical classes, including organic compounds or inorganic compounds. A candidate agent may be a substance such as genetic material, protein, lipid, carbohydrate, small molecules, a combination of any of two or more of foregoing, or other compositions. Candidate agents may be naturally occurring or synthetic, and may be a single substance or a mixture. Candidate agents can be obtained from a wide variety of sources including libraries of compounds. A candidate agent can be or include, for example, a polypeptide, peptidomimetic, amino acid(s), amino acid analog(s), polynucleotide(s), polynucleotide analog(s), nucleotide(s), nucleotide analog(s), or other small molecule(s). A polynucleotide may encode a polypeptide that potentially reduces NPR-A activity within the cell, or the polynucleotide may be a short interfering RNA (siRNA), a hairpin RNA (shRNA), antisense oligonucleotide, ribozyme, or other polynucleotide that targets an endogenous or exogenous gene for silencing of gene expression and potentially NPR-A activity within the cell. Candidate treatments may include exposure of the host cells to any conditions that potentially reduce NPR-A activity within the host cells. The treatment may involve exposing the cells to an energy source, for example.

According to one example of the screening assay, the method for identifying agents (which is intended to be inclusive of treatments) that reduce NPR-A activity is used to identify an agent that is therapeutic for treating an inflammation disorder and/or cell proliferation disorder, such as cancer. In aspect, the screening assay comprising contacting a host cell with a candidate agent, wherein the host cell expresses NPR-A, or an active fragment or variant thereof, and determining whether activity of the receptor is reduced, wherein a decrease in receptor activity is indicative of a potentially therapeutic agent. The method can optionally include an additional step of comparing NPR-A activity in the presence of the candidate agent, with NPR-A activity in the absence of the candidate agent (e.g., or other positive or negative control). The determination of NPR-A activity may be quantitative, semi-quantitative, or qualitative.

Known methods for over expressing NPR-A in host cells and determining intracellular cGMP may be utilized to determine whether NPR-A activity is reduced (Kumar et al., Hypertension, 1997, 29(part 2):414-421; Khurana M. L. and Pandey K. N., Endocrinology, 1993, 133:2141-2149; Delport C. et al., Eur. J. Pharmacol., 1992, 224(2-3):183-188; Ohyama Y. et al., Biochem. Biophys. Res. Commun., 1992, 189(1):336-342; Sharma G. D. et al., Expression of Atrial Natriuretic Peptide Receptor-A Antagonizes the Mitogen-Activated Protein Kinases (erk2 and P38^{MAPK}) in cultured human vascular Smooth Muscle Cells”, in Molecular and Cellular Biochemistry, Springer Science+Business Media B.V., ISSN:0300-8177, Vol. 233, Number. 1-2, April 2002, pages 165-173; Pandey K. N. et al., Biochem. Biophys. Res. Commun., 2000, 271(2):374-379; Fujiseki Y. et al., Jpn. J. Pharmacol., 1999, 79(3):359-368; Pandey K. N., Can. J. Physiol. Pharmacol., 2001, 79(8):631-639; Pandey K. N., Mol. Cell. Biochem., 2002, 230(1-2):61-72; Sekiguchi T. et al., Gene, 2001, 273:251-257; Chen S. et al., J. Am. Soc. Nephrol., 2005, 16:329-339; Pandey K. N. et al., J. Biol. Chem., 2002, 277(7):4618-4627; Pandey K. N. et al., Biochem. J., 2004, Dec. 1, Epub ahead of print; Roueau N. et al., Poster #P 10144, “Development of a Non-radioactive Homogenous HTS Platform to Measure the Activity of Guanylate Cyclase”, Presented at 10^{th} Annual SBS Conference and Exhibition, Orlando, Fla., September 11-15, 1004, PERKINELMER BIOSIGNAL Inc., Canada) each of which is incorporated herein by reference in its entirety). Functional truncations of NPR-A may also be used in the method in one example (Pandey K. N. et al., Molecular Pharmacology, 2000, 57:259-267, which is incorporated herein by reference in its entirety). For example, using the AlphaScreen, a very sensitive assay platform capable of detecting fmol levels of non-acetylated cGMP has been developed (Rouleau et al., 2004). A biotinylated derivative of cGMP can be used as a tracer in a competitive immunoassay format involving rabbit anti cGMP antibodies. The AlphaScreen signal is generated when streptavidin coated Donor beads and protein A coated Acceptor beads are brought into proximity by the formation of the biotin-cGMP/anti-cGMP IgG complex. Production of cGMP by either particulate or soluble forms of guanylate cyclase leads to a decrease of the AlphaScreen signal by inhibiting the formation of the biotin-cGMP/anti-cGMP IgG complex. Using this assay, the activity of the atrial natriuretic peptide receptor (NPR-A, particulate guanylate cyclase) over expressed in CHO cells has been characterized as well as that of soluble guanylate cyclase. Pharmacological parameters and Z′ values obtained indicate that the assay platform is amenable to HTS.

In addition to determining whether an agent reduces NPR-A activity in vitro (e.g., in a cellular or acellular assay) and/or in vivo (in a human or non-human patient, or an animal model), the method may further comprise determining whether the agent reduces the physiological effects or symptoms associated with an inflammatory disorder and/or cell proliferation disorder, such as cancer, in vitro and/or in vivo (e.g., in an animal model). For example, the method may further comprise determining whether the agent has an apoptotic effect on cancer cells in vitro. These steps may be carried out before, during, or after NPR-A activity is assayed.

Contacting steps in the assays (methods) may involve combining or mixing the candidate agent and the cell in a suitable receptacle, such as a reaction vessel, micro vessel, tube, micro tube, well, or other solid support. Host cells and/or candidate agents may be arrayed on a solid support, such as a multi-well plate. “Arraying” refers to the act of organizing or arranging members of a library, or other collection, into a logical or physical array. Thus, an “array” refers to a physical or logical arrangement of, e.g., library members (candidate agent libraries). A physical array can be any “spatial format” or physically gridded format” in which physical manifestations of corresponding library members are arranged in an ordered manner, lending itself to combinatorial screening. For example, samples corresponding to individual or pooled members of a candidate agent library can be arranged in a series of numbered rows and columns, e.g., on a multiwell plate. Similarly, host cells can be plated or otherwise deposited in microtiter, e.g., 96-well, 384-well, or -1536 well, plates (or trays). Optionally, host cells may be immobilized on the solid support.

A “solid support” (also referred to herein as a “solid substrate”) has a fixed organizational support matrix that preferably functions as an organization matrix, such as a microtiter tray. Solid support materials include, but are not limited to, glass, polacryloylmorpholide, silica, controlled pore glass (CPG), polystyrene, polystyrene/latex, polyethylene, polyamide, carboxyl modified Teflon, nylon and nitrocellulose and metals and alloys such as gold, platinum and palladium. The solid support can be biological, non-biological, organic, inorganic, or a combination of any of these, existing as particles, strands, precipitates, gels, sheets, tubing, spheres, containers, capillaries, pads, slices, films, plates, slides, etc., depending upon the particular application. Other suitable solid substrate materials will be readily apparent to those of skill in the art. The surface of the solid substrate may contain reactive groups, such as carboxyl, amino, hydroxyl, thiol, or the like for the attachment of nucleic acids, proteins, etc. Surfaces on the solid substrate will sometimes, though not always, be composed of the same material as the substrate. Thus, the surface can be composed of any of a wide variety of materials, for example, polymers, plastics, resins, polysaccharides, silica or silica-based materials, carbon, metals, inorganic glasses, membranes, or any of the above-listed substrate materials.

Measurement of NPR-A gene expression can be carried out using RT-PCR, for example. Screening of candidate agents or treatments (e.g., determination of NPR-A receptor activity) can be performed in a high-throughput format using combinatorial libraries, expression libraries, and the like. Other assays can be carried out on the host cells before, during, and/or after detection of NPR-A activity, and any or all assays may be carried out in an automated fashion, in a high-throughput format.

Alternatively, the aforementioned methods can be modified through the use of a cell-free assay. For example, instead of determining whether NPR-A activity in host cells is reduced by a candidate agent, extracts from host cells may be utilized and a fluorochrome or other detectable moiety can be associated with a nanoparticle or bead.

Once an agent has been determined to be one which reduces NPR-A activity, the agent can be combined with a pharmaceutically acceptable carrier. The method may further include a step of manufacturing the agent. The method may further include the step of packaging the agent.

Various methods may include a step that involves comparing a value, level, feature, characteristic, property, etc. to a “suitable control”, referred to interchangeably herein as an “appropriate control”. A “suitable control’ or “appropriate control” is any control or standard familiar to one of ordinary skill in the art useful for comparison purposes. In one embodiment, a “suitable control” or “appropriate control” is a value, level, feature, characteristic, property, etc. determined before, during, or after contacting an NPR-A receptor with a candidate agent, as described herein. For example, a transcription rate, mRNA level, translation rate, protein level, biological activity, cellular characteristic or property, genotype, phenotype, etc. can be determined prior to introducing a candidate into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Measuring expression includes determining or detecting the amount of the polypeptide present in a cell or shed by it, as well as measuring the underlying mRNA, where the quantity of mRNA present is considered to reflect the quantity of polypeptide manufactured by the cell. Furthermore, the gene for the NPR-A can be analyzed to determine whether there is a gene defect responsible for aberrant expression or polypeptide activity.

Polypeptide detection can be carried out by any available method, e.g., by Western blots, ELISA, dot blot, immunoprecipitation, RIA, immunohistochemistry, etc. For instance, a tissue section can be prepared and labeled with a specific antibody (indirect or direct and visualized with a microscope. Amount of a polypeptide can be quantitated without visualization, e.g., by preparing a lysate of a sample of interest, and then determining by ELISA or Western the amount of polypeptide per quantity of tissue. Antibodies and other specific binding agents can be used. There is no limitation on how detection of NPR-A activity is performed.

Assays can be utilized which permit quantification and/or presence/absence detection of a target nucleic acid (e.g., NPR-A) in a sample. Assays can be performed at the single-cell level, or in a sample comprising many cells, where the assay is “averaging” expression over the entire collection of cells and tissue present in the sample. Any suitable assay format can be used, including, but not limited to, e.g., Southern blot analysis, Northern blot analysis, polymerase chain reaction (“PCR”) (e.g., Saiki et al., Science 1988, 241, 53; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene Cloning and Analysis: Current Innovations, Pages 99-115, 1997), ligase chain reaction (“LCR”) (EP 320 308), one-sided PCR (Ohara et al., Proc. Natl. Acad. Sci. 1989, 86, 5673-5677), indexing methods (e.g., U.S. Pat. No. 5,508,169), in situ hybridization, differential display (e.g., Liang et al., Nucl. Acid. Res. 1993, 21, 3269 3275; U.S. Pat. Nos. 5,262,311, 5,599,672 and 5,965,409; WO97/18454; Prashar and Weissman, Proc. Natl. Acad. Sci., 93:659-663, and U.S. Pat. Nos. 6,010,850 and 5,712,126; Welsh et al., Nucleic Acid Res., 20:4965-4970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), Qbeta Replicase (PCT/US87/00880), Strand Displacement Amplification (“SDA”), Repair Chain Reaction (“RCR”), nuclease protection assays, subtraction-based methods, Rapid-Scan, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci. 1991, 88, 7276-7280; U.S. Pat. Nos. 5,210,015 and 5,994,063), real-time fluorescence-based monitoring (e.g., U.S. Pat. No. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). Any method suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol. 1990, 2, 17-25; Eberwine et al., Proc. Natl. Acad. Sci. 1992, 89, 3010-3014; U.S. Pat. No. 5,723,290). These and other methods can be carried out conventionally, e.g., as described in the mentioned publications.

The terms “transfection”, “transformation”, and “introduction”, and grammatical variations thereof, are used interchangeably herein to refer to the insertion of an exogenous polynucleotide (e.g., a nucleic acid sequence encoding an NP, or fragment, homolog, or variant thereof, or a nucleic acid sequence encoding an NPR-A, or fragment, homolog, or variant thereof, into a host cell, irrespective of the method used for the insertion, the molecular form of the polynucleotide that is inserted, or the nature of the cell (e.g., prokaryotic or eukaryotic). The insertion of a polynucleotide per se and the insertion of a plasmid or vector comprised of the exogenous polynucleotide are included. The exogenous polynucleotide may be directly transcribed and translated by the cell, maintained as a nonintegrated vector, for example, a plasmid, or alternatively, may be stably integrated into the host genome. Thus, host cells in one example, include those that have been transfected with polynucleotides encoding an NP, or fragment, variant, or homolog thereof, and those that have been transfected with polynucleotides encoding an NPR-A, or fragment, variant, or homolog thereof.

The phrases “isolated” or “biologically pure” refer to material that is substantially or essentially free from components which normally accompany the material as it is found in its native state.

An “isolated polynucleotide” that encodes a particular polypeptide refers to a polynucleotide that is substantially free of other nucleic acid molecules that do not encode the subject polypeptide; however, the molecule may include functionally and/or structurally conservative mutations as defined herein.

The terms “cell” and “cells” are used interchangeably herein to refer to a single cell or plurality of cells (i.e., at least one cell). In one example, host cells areused in the methods disclosed. However, tissues, and genetically modified or transgenic animals may also be utilized.

The terms “comprising”, “consisting of” and “consisting essentially of” are defined according to their standard meaning. The terms may be substituted for one another throughout the instant application in order to attach the specific meaning associated with each term.

As used in this specification, the singular forms “a”, “an”, and “the” include plural reference unless the context clearly dictates otherwise. Thus, for example, a reference to “a cell” includes more than one such cell. Reference to “a receptor” includes more than one such receptor. Reference to “a polynucleotide” includes more than one such polynucleotide. Reference to “a polypeptide” or “agent” includes more than one such polypeptide or agent, and the like.

The practice of the methods and compositions described herein may employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover ed. 1985); Perbal, B., A Practical Guide to Molecular Cloning (1984); the series, Methods In Enzymology (S. Colowick and N. Kaplan eds., Academic Press, Inc.); Transcription and Translation (Hames et al. eds. 1984); Gene Transfer Vectors For Mammalian Cells (J. H. Miller et al. eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. eds. (1991) IRL Press)).

### Example 1

### PNP 73-102 Inhibits NPRA Expression

The structures of ANP and ANP like molecules with their ring-structure and receptors associated with it are well characterized. However, the N-terminal peptides do not have this structure. Neither KP nor NP73-102 was shown to bind ANP receptor NPRA (Mohapatra et al., J Allergy Clin Immunol, 2004, 114:520-526). The receptors for NP-73-102 are not known.

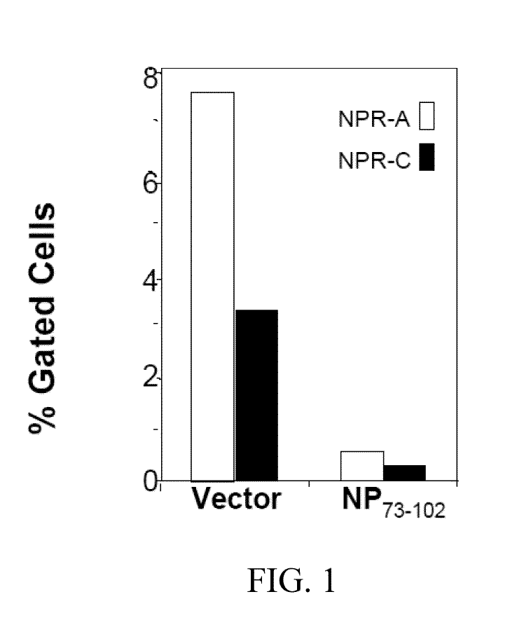

The highest expression of the ANP and ANP receptors is found in neonatal thymus. To test whether the peptide NP73-102 inhibits in vivo the ANP cascade, pregnant (12 days) mice were injected i.p. with pVAX (vector), or pNP73-102. After 1 day, mice were sacrificed and thymi removed from embryo, were homogenized. Cells were centrifuged and erythrocytes lysed by treating the suspension with ACK buffer. Cells were incubated with anti-NPRA or anti-NPRC antibodies for 1 hour, washed and incubated with PE-conjugated 20 Ab. Levels of NPR's were determined by flow cytometry. The results are shown in FIG. 1. The results demonstrate that pNP73-102 inhibited expression of NPRA in thymocytes. Although the mechanism is not clear, this may be due to feedback inhibition at the level intracellular signaling occurring via NPRA.

### Example 2

### NPRA Deficiency Decreases Pulmonary Inflammation

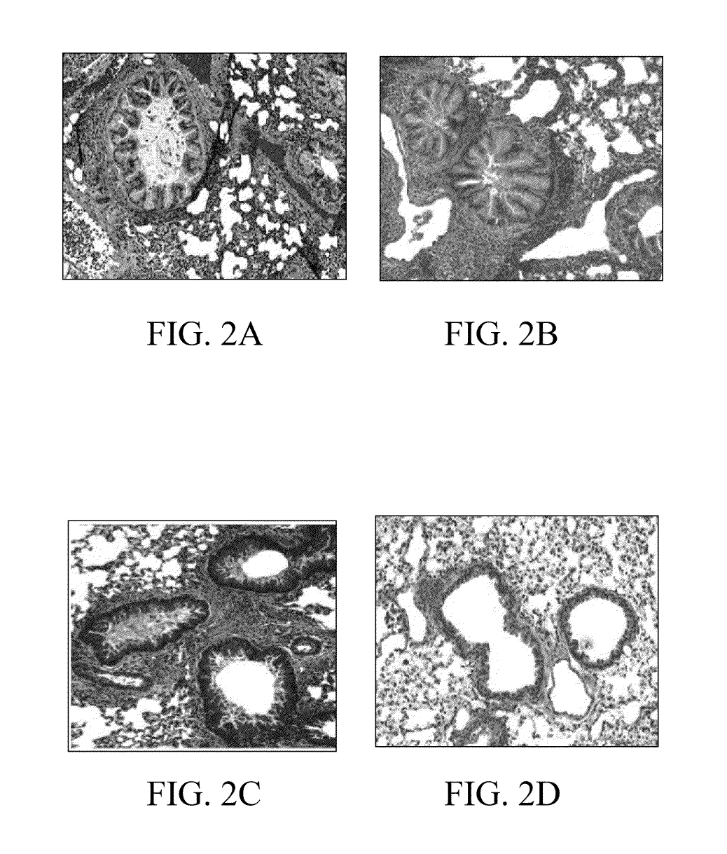

Development and chronicity of cancers has been attributed to the chronic inflammation in the affected organs. ANP was reported to have anti-inflammatory activity, although signaling through NPRA is known to cause a number of different biological activity including cell proliferation, immune activation, inflammation and apoptosis. To determine the role of NPRA signaling in the lung inflammation, groups (n=3) of wild type DBA/2 (wt) and NPR-C (ko) deficient mice and wild type C57/BL6 (wt) and NPR-A (ko) were sensitized with ovalbumin (20 mg/mouse) and after 2 weeks challenged i.n. with ovalbumin (20 mg/mouse). One day later, mice were sacrificed and lung sections were stained with H & E to examine inflammation. As shown in FIGS. 2A-2D, there was no significant difference in pulmonary inflammation between the wild-type and NPRC deficient mice. In sharp contrast, a comparison between wild-type C57BL6 and NPRA deficient mice showed that NPRA deficient mice showed substantially reduced inflammation compared to wild type. These results indicate that ANP-NPRA signaling is involved in increasing inflammation in the lung.

### Example 3

### A549 Cells Transfected with pNP₇₃₋₁₀₂ Show a Significantly Higher Level of Apoptosis Compared Control and pANP or pVAX

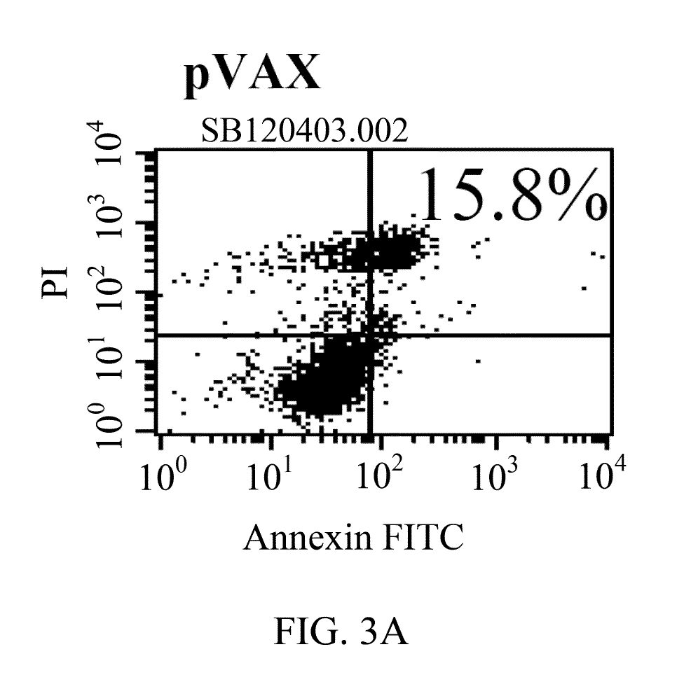

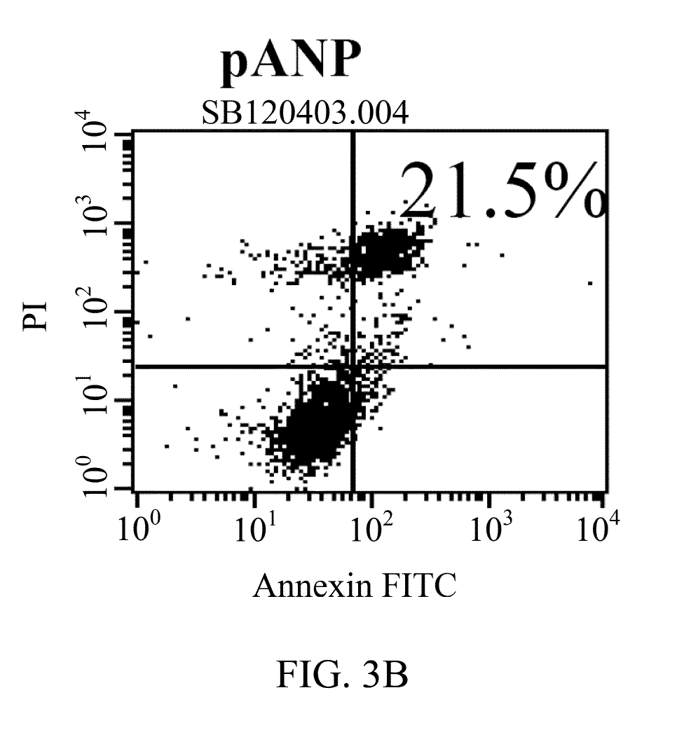

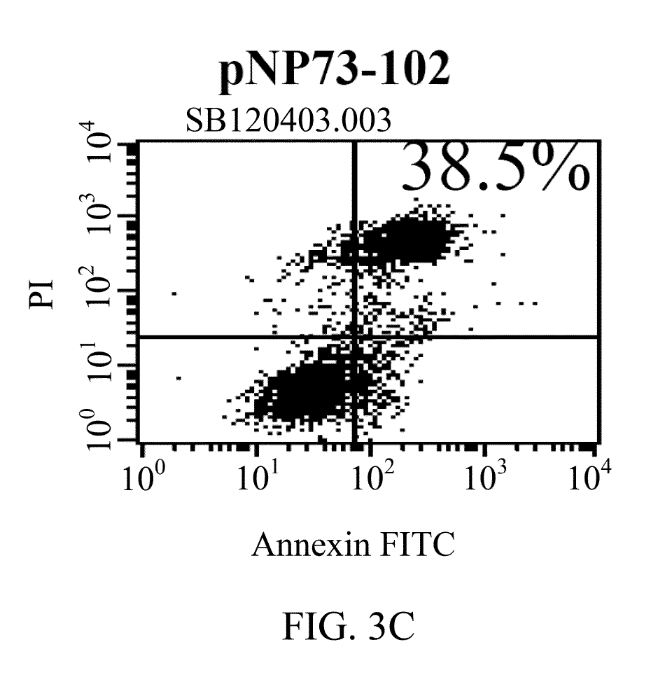

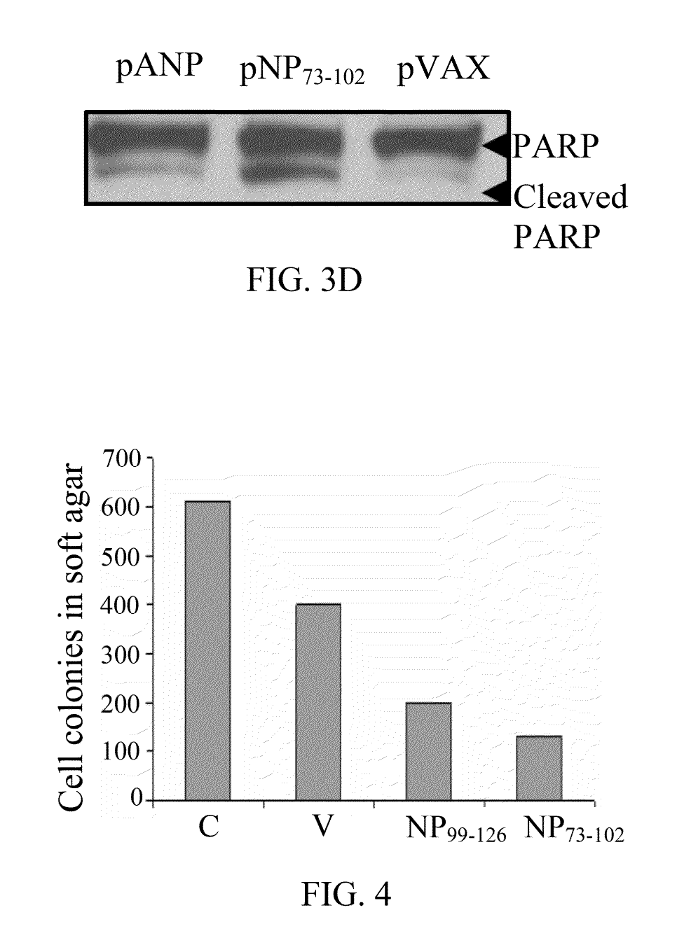

To determine the effect of over expression of NP73-102 on proliferation of A549 lung epithelial cells, cells were transfected with either pNP73-102 or vector, pVAX. Cell cycle analysis was performed using propidium iodide (PI) staining and flow cytometry 48 h after transfection. No significant difference was observed between control and pNP73-102-transfected cells in S1, Go-G1 and G2-M stages of cell cycle (data not shown). However, an analysis of apoptosis using flow-cytometry with PI and annexin V, showed that cells transfected with pNP73-102 exhibited significantly higher apoptosis compared to cells transfected with either the control plasmid or a plasmid encoding ANP (FIGS. 3A-3C). This result was confirmed by (i) staining by TUNEL of A549 cells cultured in 8-chamber slide following a 48-hour transfection with either pANP or pNP73-102 (not shown), (ii) by analysis of PARP cleavage in these cells 48 hours after transfection, which was significantly more prominent in pNP73-102 transfected cells (FIG. 3D). The results show that pNP73-102 shows a higher accumulation of apoptotic cells compared to cells transfected with pANP and pVAX controls. Thus, pNP73-102 induces apoptosis of lung adenocarcinoma cells.

In an effort to identify and characterize molecules participating in early signaling pathways, differential gene expression was analyzed using a microarray (AFFYMETRIX). Altered expression of a large number of genes was found, including genes related to cell growth, cell cycle, and apoptosis. These genes included, among others more than, 6- to 8-fold up-regulation of genes such as Caspase (Casp)-8 and FADD like apoptosis regulator, cyclin E binding protein, CDK inhibitor 1A, CDK7, casp4, casp-10, casp-1, apoptosis facilitator BCL2-like 13 and annexin 43 (data not shown). Together, these studies indicate that pNP73-102 is an inducer of apoptosis in A549 lung adenocarcinoma cells.

### Example 4

### pNP73-102 Decreases Tumorigenesis in a Colony Formation Assay by A549

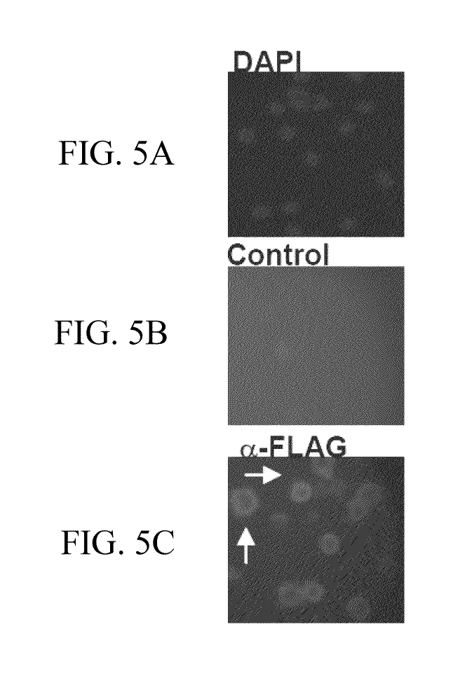

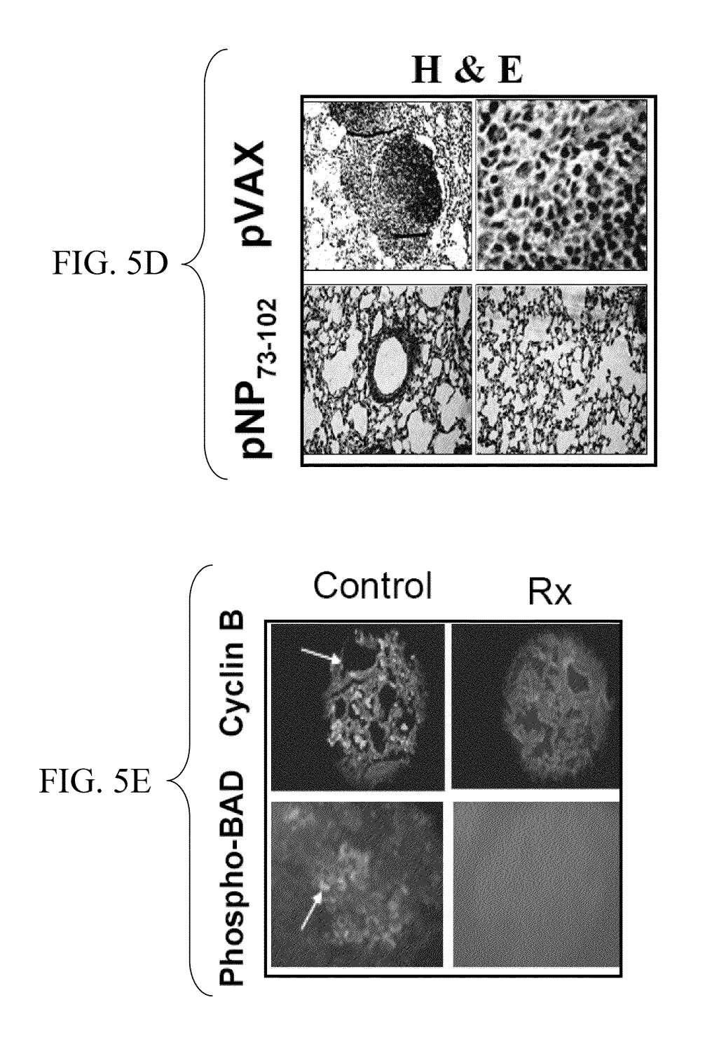

To test the anti-cancer activity of the pNP73-102 construct, a colony forming assay was undertaken. Thus, six cm tissue culture plates were covered with 4 ml of 0.5% soft agar. A549 cells were transfected with pANP, pNP₇₃₋₁₀₂ and pVAX plasmid DNA. After 40 hours of transfection, equal number of cells were suspended in 2 ml of 0.3% soft agar and added to each plate. Cells were plated in duplicate at a density of 2×10⁴ cells/dish and incubated for two weeks. Plates were observed and photographed under a microscope. Cell colonies were counted and plotted. The results of one representative experiment of two experiments performed is shown in FIGS. 5A-5D. The results show that plasmid vector alone caused some reduction in colony formation compared to untransfected control. However, both ANP and pNP₇₃₋₁₀₂ showed substantial reductions in the number of colonies produced compared to vehicle control.

### Example 5

### Chitosan Nanoparticle Containing pNP₇₃₋₁₀₂ Substantially Decrease Tumor Development in the Lung

To test the effect of de novo expression of pNP₇₃₋₁₀₂, the plasmid was coacervated with chitosan nanoparticles, referred to as CPNP73-102. To examine expression of NP73-102 from CPNP73-102, a construct was developed that carried a C-terminal fusion of marker peptide of FLAG. BALB/c mice were given intranasally the NP73-102-FLAG and the expression of NP73-102-FLAG in the BAL cells after i.n. administration of CPNP73-102-FLAG peptide. A bronchial lavage was performed after 24 hours and lavage cells were stained with either the second antibody control or anti-FLAG antibody (Sigma) and then with DAPI. The results show that intranasal administration induces significant expression of the peptide in the lung cells.

To test whether CPNP73-102 is capable of decreasing tumor formation in the lung, BALB/c nude mice were injected i.v. with 5×10⁶ A549 cells, then treated one day afterwards and at weekly intervals with CPNP73-102 or control plasmid. After 4 weeks, mice were examined for lung histology. The control animals showed tumors, whereas no tumors were observed in the CpNP73-102-treated group. Sections were also stained with antibodies to cyclinB and to phospho-Bad. The results show that mice treated with CPNP73-102 had no tumors in the lung and did not show any staining for pro-mitotic Cyclin-B and anti-apoptotic marker phospho-Bad. These results indicate that CPNP73-102 has the potential to decrease tumor formation in the lung.

### Example 6

### Treatment with CPNP73-102 Decreases the Tumor Burden in a Spontaneous Tumorigenesis Model of Immunocompetent Balb/C Mice

The nude mouse model is deemed to be of less predictive value in terms of translating to human cancer, as mice used are immunodeficient. Therefore, to confirm the results obtained on the potential role of pNP73-102, a syngeneic immunocompetent mouse model of human lung carcinoma was used. For this purpose, Line-1 cell line derived from a bronchioalveolar cell carcinoma (a subtype of lung adenocarcinoma that spontaneously arose in BALB/C mouse (Yuhas et al., Cancer Research, 1975, 35:242-244). The cell line forms subcutaneous tumors within 2 to 3 weeks of injection and spontaneously metastasizes to the lung.

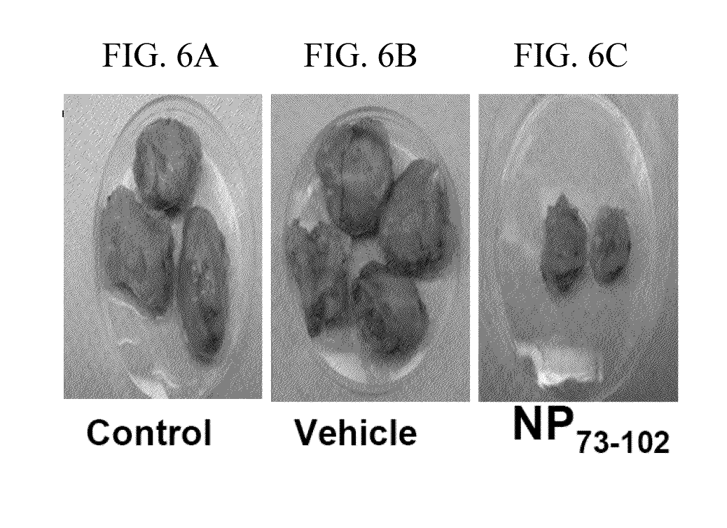

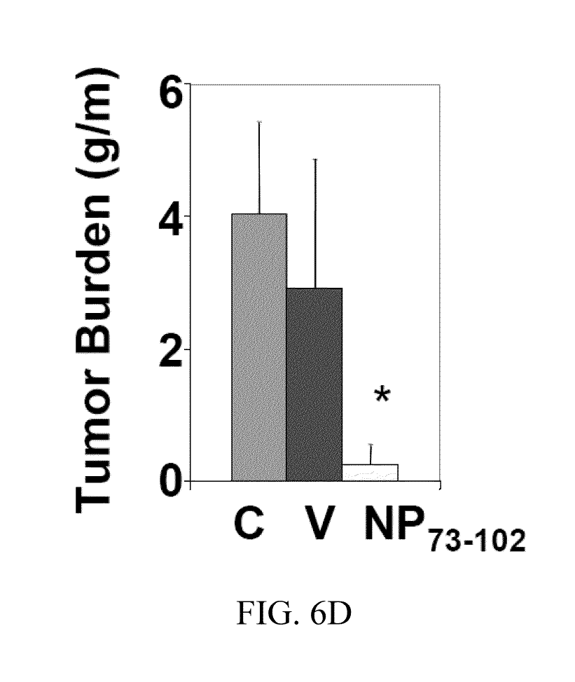

To examine whether de novo synthesis of NP73-102 affects tumor development, two groups of BALB/c mice (n=4) were administered with the Line-1 tumor cells (100,000 cells/mouse) at the flanks One group was administered intranasally with CPNP73-102 the same day, whereas another group was administered with vehicle alone (nanoparticle carrying a plasmid without NP73-102), and the third group was given the saline. Treatment was continued with NP73-102 or controls at weekly intervals for 5 weeks. The tumors were dissected out from each group of mice and photographed (FIGS. 6A-6C) and the tumor burden was calculated by weighing them on a balance (FIG. 6D). The results show that mice administered with CPNP73-102 had significantly decreased tumor burden (P<0.05).

### Example 7

### ppNP73-102 Induces Apoptosis in Chemoresistant Ovarian Cancer Cells

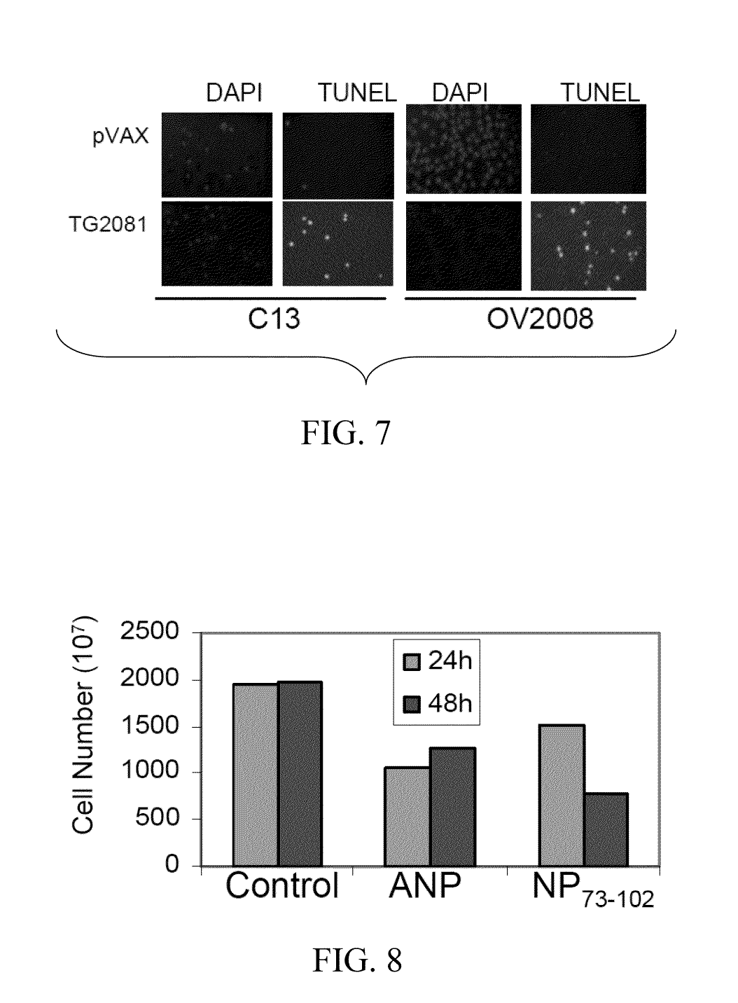

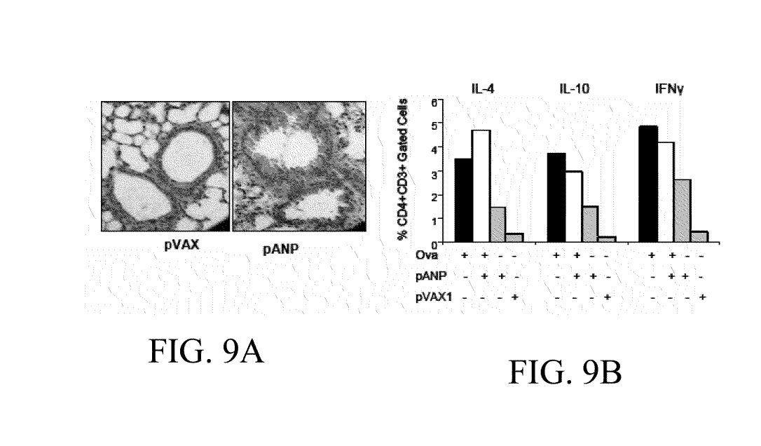

The adenocarcinomas of various tissues such as lung, ovary, and breasts have many characteristics that are similar. Chemoresistance is a major therapeutic problem in many of the cancers and the current knowledge on cellular mechanisms involved is incomplete. Since A549 cells showed differential sensitivity to apoptosis with pVAX and pNP₇₃₋₁₀₂, the effects of pnP73-102 was tested using chemosensitive (OV2008) and chemoresistant (C13) ovarian cancer cells. C-13 and OV2008 ovarian cancer cells were transfected with pNP73-102 or with pVAX as control. Forty-eight hours later, cells were processed to examine apoptosis by TUNEL assay (FIG. 7). The results showed that either of the cells when transfected with pVAX did not exhibit any apoptosis. In contrast, both cell lines exhibited apoptosis as evident from TUNEL positive cells. These results indicate that pNP73-102 may induce apoptosis of epithelial adenocarcinomas irrespective of their degree of chemo-sensitivity.

### Example 8

### MCF-7 Breast Cancer Cells are Also Affected by NP73-102

The effects of de novo synthesis of NP₇₃₋₁₀₂ was examined on the proliferation of the MCF-7 breast cancer cells. Cells were transfected with pVAX, pANP, or pANP₇₃₋₁₀₂. The cells were counted 24 and 48 hours after transfection and their viability was examined by trypan blue staining. The results shown in FIG. 8 indicate that there was a substantial reduction of viable cell numbers in cells transfected with pNP₇₃₋₁₀₂ compared to cells transfected with pANP or control empty vector. To further verify whether this is due to a defect in cell cycle or induction of apoptosis, a cell cycle analysis was undertaken. MCF-7 cells were transfected with pVAX or pANP₇₃₋₁₀₂ and DNA analysis was undertaken by PI staining 48 hours after transfection. Cells transfected with empty vector plasmid as control showed 37.99% cells in G0-G1, 11.28% in G2-M and 50.73% cells in G2-G1 phase. In contrast, cells transfected with pANP₇₃₋₁₀₂ showed 66.01% cells in G0-G1, 7.07% in G2-M, and 26.91% cells in G2-G1 phase. Transfection with pANP showed results similar to the pNP₇₃₋₁₀₂ These results indicate that both pANP and pNP₇₃₋₁₀₂ expression arrests cells in G0-G1 and blocks progression to S phase, suggesting that treatment with pANP and pNP₇₃₋₁₀₂ or the corresponding peptides may be useful in breast cancer patients.

In one example, a method for reducing atrial natriuretic peptide receptor A (NPRA) gene expression and/or function within a subject comprises administering an effective amount of an NPRA inhibitor to the subject. In one embodiment, the NPRA inhibitor is a polynucleotide that is specific for one or more target NPRA genes such that the polynucleotide decreases NPRA gene expression within the subject. In another embodiment, the NPRA inhibitor is a chemical compound; such as an oxindol (e. isatin). The methods may be useful for treating inflammatory diseases in human subjects and non-human subjects suffering from, or at risk for developing, inflammatory reactions. The methods and compositions include, but are not limited to, the following embodiments:

Embodiment 1: an isolated polynucleotide targeted to a target nucleic acid sequence within a natriuretic peptide receptor A (NPRA) gene or NPRA transcript wherein said polynucleotide inhibits expression of said NPRA gene or transcript.

Embodiment 2: the polynucleotide of embodiment 1, wherein the NPRA is human NPRA (e.g., encoded by SEQ ID NO:4).

Embodiment 3: the polynucleotide of embodiment 1, wherein the target nucleic acid sequence is at least a portion of the human NPRA gene or transcript.

Embodiment 4: the polynucleotide of any of embodiments 1 to 3, wherein the target nucleic acid sequence is located in a region selected from the group consisting of the 5′ untranslated region (UTR), transcription start site, translation start site, and 3′ UTR.

Embodiment 5: the polynucleotide of any of embodiments 1 to 4, wherein the polynucleotide is a small interfering RNA (siRNA).

Embodiment 6: the polynucleotide of any of embodiments 1 to 4, wherein the polynucleotide is an antisense molecule.

Embodiment 7: the polynucleotide of any of embodiments 1 to 4, wherein the polynucleotide is a ribozyme.

Embodiment 8: the polynucleotide of embodiment 1, wherein the polynucleotide comprises SEQ ID NO:1, or SEQ ID NO:2, or SEQ ID NO:3.

Embodiment 9: the polynucleotide of embodiment 1, wherein the NPRA gene or NPRA transcript is at least a portion of the mammal gene or transcript.

Embodiment 10: a method for reducing NPRA function in a subject, comprising administering an NPRA inhibitor to the subject, such as the polynucleotide of any of embodiments 1 to 9, wherein the polynucleotide is administered in an effective amount to reduce expression of the NPRA gene or transcript.

Embodiment 11: the method of embodiment 10, wherein the subject is suffering from an inflammatory disease, respiratory allergy, viral infection (such as respiratory virus infection), or cancer (such as melanoma, lung cancer, or ovarian cancer).

Embodiment 12: the method of embodiment 10, wherein the subject is not suffering from an inflammatory disease, respiratory allergy, viral infection, or cancer.

Embodiment 13: the method of anyone of embodiments 10 to 12, wherein the subject is human.

Embodiment 14: the method of any one of embodiments 10 to 12, wherein the subject is a non-human mammal.

Embodiment 15: the method of anyone of embodiments 10 to 14, wherein the NPRA inhibitor is delivered to cells within the subject selected from the group consisting of respiratory epithelial cells, dendritic cells, and monocyte

Embodiment 16: the method of anyone of embodiments 10 to 15, wherein the NPRA inhibitor is administered to the subject intranasally.

Embodiment 17: the method of anyone of embodiments 10 to 16, wherein the NPRA inhibitor is administered intranasally as drops or as an aerosol, or orally or transdermally.

Embodiment 18: the method of anyone of embodiments 10 to 17, wherein step of administering comprises administering a′ combination of NPRA inhibitors that reduce the function of NPRA within the subject (such as a combination of polynucleotide siRNA pool).

Embodiment 19: the method of any one of embodiments 10 to 18, wherein the NPRA inhibitor is a siRNA and wherein the siRNA reduces expression of NPRA within the subject.

Embodiment 20: the method of anyone of embodiments 10 to 18, wherein the NPRA inhibitor is an oxindol such as 5-hydroxyoxindole or isatin or a pharmaceutically acceptable salt thereof (Cane, A. et al. Biochem. Biophy. Res Comm 2000, 276:379-384; Vine, K. L. et al. Bioorg Med Chem 2007, 15(2):931-938; Abadi H. et al. Eur J Med Chem 2006, 41(3):296-305; Igosheva, N. et al. Neurochem Int 2005, 47(3):216-224; Liu, Y. et al. Chem Biol 2003, 10(9):837-846; Levy, L A. et al. Virology, 1976, 74(2):426-431; Popp, F. D. J Med Chem 1969, 12(1):182-184). Isatin also known as 1H-indole-dione) is an indole derivative (Sumpter, W. C. Chem Rev 34(3):393-434; Ogata, A. et al. J Neurol Sci 2003, 206(1):79-83; Glover, V. et al. J 20 Neurochem 1988 51(2):656-659; Filomeni, G. et al. J Biol Chem 2007, 282(16):1201012021).

As used herein, the term “polypeptide” refers to any polymer comprising any number of amino acids, and is interchangeable with “protein gene product”, and “peptide”.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

As used herein, the term “RNA” or “RNA molecule” or “ribonucleic acid molecule” refers generally to a polymer of ribonucleotides. The term “DNA” or “DNA molecule” or deoxyribonucleic acid molecule refers generally to a polymer of deoxyribonucleotides. DNA and RNA molecules can be synthesized naturally (e.g., DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA respectively) or multi-stranded (e.g., double stranded dsRNA and dsDNA respectively). The term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotide molecule, for example, at least one deoxyribonucleotide and/or at least one nucleotide analog.

As used herein, the term “nucleotide analog”, also referred to herein as an “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. As used herein, the term “RNA analog” refers to a polynucleotide (e.g., chemically synthesized polynucleotide) having at least one altered or modified nucleotide as compared to a corresponding unaltered or unmodified RNA but retaining the same or similar nature or function as the corresponding unaltered or unmodified RNA discussed above, the oligonucleotides may be linked with linkages which result in a lower rate of hydrolysis of the RNA analog as compared to an RNA molecule with phosphodiester linkages. Exemplary RNA analogues include sugar and/or backbone modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference or otherwise reduce target gene expression.

As used herein, the term “operably-linked” or “operatively-linked” refers to an arrangement of flaning sequences wherein the flanking sequences so described are configured or assembled so as to perform their usual function. Thus, a flanking sequence operably-linked to a coding sequence may be capable of effecting the replication transcription and/or translation of the coding sequence. For example, a coding sequence is operably-linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence, and the promoter sequence can still be considered “operably-linked” to the coding sequence. Each nucleotide sequence coding for a siRNA will typically have its own operably-linked promoter sequence.

The term “vector” or “vehicle” is used to refer to any molecule (e.g., nucleic acid, plasmid, or virus) used to transfer coding information (e.g., a polynucleotide, in one example) to a host cell. The term “expression vector” refers to a vector that is suitable for use in a host cell (e.g., a subject's cell) and contains nucleic acid sequences which direct and/or control the expression of exogenous nucleic acid sequences. Expression includes, but is not limited to, processes such as transcription, translation, and RNA splicing, if introns are present. The vectors may be conjugated with chitosan or chitosan derivatives. Such chitosan conjugates can be administered to hosts according to the methods. For example, polynucleotide chitosan nanospheres can be generated, as described by Roy, K. et al. (Nat Med, 1999 5:387). Chitosan allows increased bioavailability of the nucleic acid sequences because of protection from degradation by serum nucleases in. the matrix and thus has great potential as a mucosal gene delivery system. Chitosan also has many beneficial effects including anticoagulant activity, wound-healing properties, and immunostimulatory activity, and is capable of modulating immunity of the mucosa and bronchus-associated lymphoid tissue. In one embodiment, the vectors are conjugated with chitosan-derived nanoparticles.

As used herein, the term “RNA interference” or “RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences.

Alternatively, RNAi can be initiated by the hand of man, for example, to silence the expression of target genes. As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference. As used herein, a siRNA having a “sequence sufficiently complementary to a target mRNA sequence to direct target-specific RNA interference (RNAi)” means that the siRNA has a sequence sufficient to trigger the destruction of the target mRNA by the RNAi machinery or process. RSV “mRNA”, “messenger RNA”, and “transcript” each refer to single-stranded RNA that specifies the amino acid sequence of one or more RSV polypeptides. This information is translated during protein synthesis when ribosomes bind to the mRNA.

As used herein, the term “cleavage site” refers to the residues nucleotides, at which RISC* cleaves the target RNA near the center of the complementary portion of the target RNA about 8-12 nucleotides from the 5′ end of the complementary portion of the target RNA. As used herein, the term “mismatch” refers to a base pair consisting of non-complementary bases not normal complementary G:C, A:T or A:U base pairs.

As used herein, the term “isolated” molecule (e. isolated nucleic acid molecule) refers to molecules which are substantially free of other cellular material, or culture medium when produced by recombinant techniques, or substantially free of chemical precursors or other chemicals when chemically synthesized. Preferably, the NPRA inhibitors are administered in an isolated form.

As used herein, the term in vitro involving has its art recognized meaning, purified reagents or extracts cell extracts. The term in vivo also has its art recognized meaning, involving living cells in an organism immortalized cells primary cells, and/or cell lines in an organism.

A gene “involved in” or “associated with” a disorder includes a gene, the normal or aberrant expression or function of which affects or causes a disease or disorder or at least one symptom of the disease or disorder. For example, NPRA protein has been found to have a significant role in pulmonary inflammation and immune modulation. Without being bound by theory, it has been found that signaling through the NPRA protein results in increased cGMP production and activation of protein kinase G, leading to regulation of transcription of many genes involved in the cell cycle, apoptosis, and inflammation. The polynucleotides, genetic constructs, pharmaceutical compositions, and methods are useful in decreasing expression of NPR-A gene in vitro or vivo consequently causing decreased production of the NPRA protein and decreased inflammation. Thus the polynucleotides genetic constructs pharmaceutical compositions, and methods are useful in the treatment of human or nonhuman animal subjects suffering from, or at risk of developing, disorders associated with inflammation including, but not limited to, airway diseases, viral infections, and cancers.

The methods disclosed may include further steps. In some embodiments, a subject with the relevant condition or disease involving aberrant inflammation (e.g., asthma, RSV infection, cancers) is identified, or a subject at risk for the condition or disease is identified. A subject may be someone who has not been diagnosed with the disease or condition (diagnosis, prognosis, and/or staging) or someone diagnosed with the disease or condition (diagnosis, prognosis, monitoring, and/or staging), including someone treated for the disease or condition (prognosis, staging, and/or monitoring).

Alternatively, the subject may not have been diagnosed with the disease or condition but suspected of having the disease or condition based either on patient history or family history, or the exhibition or observation of characteristic symptoms.

As used herein, an “effective amount” of a NPRA inhibitor (e.g., isatin or another

oxindol, an siRNA, an antisense nucleotide sequence or strand a ribozyme), and/or which selectively interferes with expression of the NPRA gene and/or function of the receptor, is that amount effective to bring about the physiological changes desired in the cells to which the polynucleotide is administered in vitro (e.g., ex vivo) or in vivo. The term “therapeutically effective amount” as used herein means that amount of NPRA inhibitor (e.g., isatin or other oxindol, an siRNA, an antisense oligonucleotide and/or ribozyme), which selectively reduces expression of the NPRA gene(s) and/or function of the receptor, alone or in combination with another agent according to the particular aspect that elicits the biological or medicinal response in cells (e.g., tissue(s)) hat is being sought by a researcher, veterinarian, medical doctor or other clinician, which includes alleviation and/or prevention of the symptoms of the disease or disorder being treated.

For example, a NPRA inhibitor can be administered to a subject in combination with other agents effective for alleviating or preventing the symptoms of inflammation such as the gene expression vaccines (Mohapatra et al. 2004). Various methods can include a step that involves comparing a value, level, feature, characteristic, property, to a “suitable control”, etc. referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate mRNA level, translation rate, protein level, biological activity, cellular characteristic or property, genotype, phenotype can be determined prior to introducing a siRNA into a cell or organism. In another embodiment, a “suitable control” or “appropriate control” is a value, level, feature, characteristic, property, determined in a cell or organism a control or normal cell or organism, exhibiting, for example normal traits. In yet another embodiment, a “suitable control” or “appropriate control” is a predefined value, level, feature, characteristic, property, etc.

### RNA Interference

RNAi is an efficient process whereby double-stranded RNA (dsRNA, also referred to herein as siRNAs or ds siRNAs, for double-stranded small interfering RNAs) induces the sequence-specific degradation of targeted mRNA in animal and plant cells (Hutvagner and Zamore Curro Opin. Genet. Dev. 12:225-232 (2002); Sharp, Genes Dev. 15:485-490 (2001). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001), or by micro-RNAs (miRNA), functional small-hairpin RNA (shRNA), or other dsRNAs which can be expressed using in vivo DNA templates with RNA polymerase III promoters (Zeng et al., Mol. Cell. 9:1327-1333 (2002); Paddison et al., Genes Dev. 16:948-958 (2002); Lee et al., Nature Biotechnol. 20:500-505 (2002); Paul et al., Nature Biotechnol. 20:505-508 (2002); Tuschl, T. Nature Biotechnol. 20:440-448 (2002); Yu et al., Proc. Natl. Acad. Sci. USA 99(9):6047-6052 (2002); McManus et al. RNA 8:842-850 (2002); Sui et al., Proc. Natl. Acad. Sci. USA 99(6):5515-5520 (2002).

Accordingly, such molecules that are targeted to NPRA mRNAs encoding at least a portion of one or more of NPRA-like receptors.

siRNA Molecules

The nucleic acid molecules or constructs in the methods and compositions include dsRNA molecules comprising 16-30 nucleotides 17, 18, 19-29, or 30 nucleotides, in each strand, wherein one of the strands is substantially identical at least 80% (or more 85%, 90%, 95%, or 100%) identical having 3, 2, 1, or 0 mismatched nucleotide(s), to a target region in the mRNA of the RSV mRNA, and the other strand is identical or substantially identical to the first strand. The dsRNA molecules may be chemically synthesized, or can be transcribed in vitro in vivo from a DNA template, or from shRA. The dsRNA molecules can be designed using any method known in the art, for instance, by using the following protocol:

1. Beginning with the AUG start codon, look for AA dinucleotide sequences; each AA and the 3′ adjacent 16 or more nucleotides are potential siRNA targets. Further siRNAs with lower content (35-55%) may be more active than those with G/C G/C 20 content higher than 55%. Thus in one embodiment, polynucleotides having 35-55% content are included. In addition, the strands of the siRNA may be G/C be paired in such a way as to have a 3′ overhang of 1 to 4, nucleotides. Thus, in another embodiment, the polynucleotides can have a 3′ overhang of 2 nucleotides. The overhanging nucleotides can be either RNA or DNA.

2. Using any method known in the art, compare the potential targets to the appropriate genome database (human, mouse, rat, etc. and eliminate from consideration any target sequences with significant homology to other coding sequences for which reduced expression is not desired. One such method for such sequence homology searches is known as BLAST, which is available at the National Center for Biotechnology Information web site of the National Institutes of Health.

3. Select one or more sequences that meet your criteria for evaluation. Further general information regarding the design and use of siRNA can be found in “The siRNA User Guide” available at the web site of the laboratory of Dr. Thomas Tuschl at Rockefeller University.

4. Negative control siRNAs preferably have the same nucleotide composition as the selected siRNA, but without significant sequence complimentarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The polynucleotides may include both unmodified siRNAs and modified siRNAs as known in the art. Thus, siRNA derivatives that include siRNA having two complementary strands of nucleic acid, such that the two strands are crosslinked. For example, a 3′ OH terminus of one of the strands can be modified, or the two strands can be crosslinked and modified at the 3′ OH terminus. The siRNA derivative can contain a single crosslink (e. a psoralen crosslink). In some embodiments, the siRNA derivative has at its 3′ terminus a biotin molecule (e.g., photocleavable biotin), a peptide (e.g., a Tat peptide), a nanoparticle, a peptidomimetic organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions may be unconjugated or can be conjugated to another moiety, such as a nanoparticle, to enhance a property of the compositions pharmacokinetic parameter such as absorption efficacy, bioavailability, and/or half-life. The conjugation can be accomplished by methods known in the art using the methods of Lambert et al., Drug Deliv. Rev. 47(1): 99-112 (2001) (describes nucleic acids loaded to olyalkylcyanoacrylate (PACA) nanoparticles); Fattal et al., J Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur J. Biochem. 232(2):404-10 (1995) (describes nucleic acids linked to nanoparticles).

The nucleic acid molecules may also be labeled using any method known in the art; for instance, the nucleic acid compositions can be labeled with a fluorophore Cy3, fluorescein, or rhodamine. The labeling can be carried out using a kit such as the SILENCER siRNA labeling kit (AMBION). Additionally, the siRNA can be radiolabeled using ³H, ³²P, or other appropriate isotope.

The dsRNA molecules may comprise of the following sequences as one of their strands, and the corresponding sequences of allelic variants thereof: SEQ ID NO: 23 or SEQ ID NO:24 or SEQ ID NO: 25.

Moreover, because RNAi is believed to progress via at least one single-stranded RNA intermediate, the skilled artisan will appreciate that ss-siRNAs (e.g., the antisense strand of a ds-siRNA) can also be designed as described herein and utilized according to the claimed methodologies.

siRNA Delivery for Longer-Term Expression

Synthetic siRNAs can be delivered into cells by methods known in the art including cationic liposome transfection and electroporation. However, these exogenous siRNA generally show short-term persistence of the silencing effect (4 to 5 days in 20 cultured cells), which may be beneficial in certain embodiments. To obtain longer term suppression of RSV gene expression and to facilitate delivery under certain circumstances, one or more siRNA duplexes RSV ds siRNA, can be expressed within cells from recombinant DNA constructs. Such systems for expressing siRNA duplexes within cells from recombinant DNA constructs to allow longer-term target gene suppression in cells are known in the art, including mammalian Pol III promoter systems (e.g., HI or U6/snRNA promoter systems (Tuschl (2002), supra) capable of expressing functional double-stranded siRNAs; (Bagella et al., J Cell. Physiol. 177:206-213 (1998); Lee et al. (2002), supra; Miyagishi et al. (2002), supra; Paul et al. (2002), supra; Yu et al. (2002), supra; Sui et al. (2002), supra). Transcriptional termination by RNA Pol III occurs at runs of four consecutive T residues in the DNA template, providing a mechanism to end the siRNA transcript at a specific sequence. The siRNA complementary to the sequence of the target gene in 5′-3′ and 3′-5′ orientations, and the two strands of the siRNA can be expressed in the same construct or in separate constructs. Hairpin siRNAs, driven by an HI or U6 snRNA promoter can be expressed in cells, and can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence(s) under the control of a promoter also make functional siRNAs when co-transfected into the cells with a vector expressing T7. RNA polymerase (Jacque (2002), supra). A single construct may contain multiple sequences coding for siRNAs, such as multiple regions of the RSV NS 1 mRNA and/or other RSV genes, and can be driven, for example, by separate PolIII promoter sites.

Animal cells express a range of non-coding RNAs of approximately 22 nucleotides termed micro RNA (miRNAs) that can regulate gene expression at the post transcriptional or translational level during animal development. One common feature of miRNAs is that they are all excised from an approximately 70 nucleotide precursor RNA 15 stem-loop, probably by Dicer, an RNase Ill-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng (2002), supra) When expressed by DNA vectors containing polymerase III promoters, micro-RNA designed hairpins can silence gene expression (McManus (2002), supra. Viral-mediated delivery mechanisms can also be used to induce specific silencing of targeted genes through expression of siRNA, for example, by generating recombinant adenoviruses harboring siRNA under RNA Pol Il promoter transcription control (Xia et al. (2002), supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. In an animal model, whole-embryo electroporation can efficiently deliver synthetic siRNA into post-implantation mouse embryos (Calegari et al., Proc. Natl. Acad. Sci. USA 99(22): 14236-40 (2002)). In adult mice, efficient delivery of siRNA can be accomplished by the “high-pressure” delivery technique, a rapid injection (within 5 seconds) of a large volume of siRNA-containing solution into animal via the tail vein (Liu (1999); supra; McCaffrey (2002), supra; Lewis Nature Genetics 32:107-108 (2002)). Nanoparticles and liposomes can also be used to deliver siRNA into animals.

### Use of Engineered RNA Precursors to Induce RNAi

Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of the RSV protein (such as RSV NS 1 protein) encoded by that mRNA in the cell or organism. The RNA precursors are typically nucleic acid molecules that individually encode either one strand of a dsRNA or encode the entire nucleotide sequence of an RNA hairpin loop structure.

### Antisense

An “antisense” nucleic acid sequence (antisense oligonucleotide) can include a nucleotide sequence that is complementary to a “sense” nucleic acid sequence encoding a protein, complementary to the coding strand of a double-stranded cDNA molecule or complementary to at least a portion of an RSV gene. The antisense nucleic acid sequence can be complementary to an entire coding strand of a target sequence, or to only a portion thereof (for example, the RSV NS 1 gene, RSV NS 2 gene, or a portion of either or and/or both). In another embodiment, the antisense nucleic acid molecule is antisense to a noncoding region” of the coding strand of a nucleotide sequence within the RSV gene. An antisense oligonucleotide can be, for example, about 7, 8, 9, 10, 15, 20, 25, 30, 35, 40, 45, 25 50, 55, 60, 65, 70, 75, 80, or more nucleotides in length.

An antisense nucleic acid sequence can be designed such that it is complementary to an entire RSV gene, but can also be an oligonucleotide that is antisense to only a portion of an RSV gene. For example, the antisense oligonucleotide can complementary to the region surrounding the translation start site of the target mRNA between the −10 and +10 regions of the target gene nucleotide sequence of interest. An antisense oligonucleotide sequence can be, for example, about 7, 8, 9, 10, 15, 20, 25, 30, 40, 45, 50, 55, 60, 65, 70, 75, 80, or more nucleotides in length.

An antisense nucleic acid sequence, in one example, may be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids; phosphorothioate derivatives and acridine substituted nucleotides can be used. The antisense nucleic acid sequence also can be produced biologically using an expression vector into which a nucleic acid sequence has been subcloned in an antisense orientation (i.e., RNA transcribed from the inserted nucleic acid sequence will be of an antisense orientation to a target nucleic acid sequence of interest, described further in the following subsection).

The antisense nucleic acid molecules are typically administered to a subject systemically or locally by direct injection at a tissue site), or generated in situ such that they hybridize with or bind to mRNA (e.g., RSV mRNA) to thereby inhibit expression of the protein (e.g., a viral protein). Antisense nucleic acid molecules can also be modified to target selected cells (such as respiratory epithelial cells, dendritic cells, and/or monocytes and then administered systemically. For systemic administration, antisense molecules can be modified such that they specifically bind to receptors or antigens expressed on a selected cell surface by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, the antisense oligonucleotide is an alpha-anomeric nucleic acid molecule. An alpha-anomeric nucleic acid molecule forms specific double-stranded hybrids with complementary RNA in which, contrary to the usual beta-units, the strands run parallel to each other (Gaultier et al., Nucleic Acids. Res. 15:6625-6641 (1987)). The antisense nucleic acid molecule can also comprise a 2′-o-methylribonucleotide (Inoue et al. Nucleic Acids Res. 15:6131-6148 (1987)) or a chimeric RNA-DNA analogue (Inoue et al. FEBS Lett. 215:327-330 (1987)).

Gene expression can be inhibited by targeting nucleotide sequences complementary to the regulatory region of the gene to form triple helical structures that prevent expression of the gene in target cells. See generally, Helene, C. Anticancer Drug Des. 6:569-84 (1991); Helene, C. Ann. NY Acad. Sci. 660:27-36 (1992) and Maher Bioassays 14:807-15 (1992). The potential sequences that can be targeted for triple helix formation can be increased by creating a so-called “switchback” nucleic acid molecule. Switchback molecules are synthesized in an alternating 5′-, 3′-5′ manner, such that they base pair with first one strand of a duplex and then the other, eliminating the necessity for a sizeable stretch of either purines or pyrimidines to be present on one strand of a duplex.

### Ribozymes

Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme encoding nucleotide sequences can be introduced into cells through gene-delivery mechanisms known in the art. A ribozyme having specificity for RSV RNA can include one or more sequences complementary to the nucleotide sequence of at least a portion of one or more RSV mRNA (e.g., RSV NSI mRNA), and a sequence having a known catalytic sequence responsible for mRNA cleavage (see U.S. Pat. No. 5,093,246 or Haselhoff and Gerlach Nature 334:585-591 (1988)). For example, a derivative of a Tetrahymena L-19 IVS RNA can be constructed in which the nucleotide sequence of the active site is complementary to the nucleotide sequence to be cleaved in the RSV mRNA, such as RSV NSI mRNA (see Cech et al., U.S. Pat. No. 4,987,071; and Cech et al. U.S. Pat. No. 5,116,742). Alternatively, RSV mRNA encoding an RSV protein can be used to select a catalytic RNA having a specific ribonuclease activity from a pool of RNA molecules (see, e.g., Bartel, D. and Szostak, J. W. Science 261:1411-1418 (1993)).

### Nucleic Acid Targets

The nucleic acid targets of the polynucleotides (e.g., antisense RNAi, and ribozymes) may be ANP receptor gene, or a portion thereof, such as NPRA NPRB or NPRC or portion of any of the foregoing. In some embodiments, the nucleic acid target is the NPRA gene, or a portion thereof. The nucleic acid target may be any location within the NPRA or transcript. Preferably, the nucleic acid target is located at a site selected from the group consisting of the 5′ untranslated region (UTR), transcription start site, translation start site, and the 3′ UTR.

The nucleic acid target may be located within a NPRA gene of any human or mammal. Preferably, the nucleic acid target is at least a portion of a non-structural NPRA gene. More preferably, the nucleic acid target is at least a portion of an NPRA gene encoding a protein. In a particularly preferred embodiment, the nucleic acid target is located within an NPRA that normally down-regulates host inflammation. In another preferred embodiment, the nucleic acid target is located within the human NPRA or mammalian NPRA, selected from the group consisting of the 5′ untranslated region (UTR), transcription start site, translation star site, and the 3′ UTR.

The nucleic acid target may be located within a human NPRA gene (e.g., NCBI accession no. AF190631, which is incorporated herein by reference in its entirety) or an ortholog thereof, such as a non-human, mammalian NPRA gene. For treating and/or preventing inflammation within a particular subject, the polynucleotide selected for administration to the subject is preferably one targeted to a NPRA gene. For example, for treating and/or preventing inflammation within a human subject, the nucleic acid target is preferably located within a human NPRA gene, or the nucleic acid target has sufficient homology with the human NPRA gene, so as to reduce expression of the human NPRA gene. The term “substantially identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%, 91%, 92%, 93%, 94%, 95%, 96%, 97% 98% or 99% identity are defined herein as substantially identical.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes).

In one embodiment, the length of a reference sequence aligned for comparison purposes is at least 50%, at least 60%, at least 70%, at least 80%, at least 90%, at least 95%, or at least 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid identity” is equivalent to amino acid or nucleic acid “homology. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch (J. Mol. Biol. 48:444-453 (1970)) algorithm, which has been incorporated into the GAP program in the GCG software package (available at the official Accelrys web site), using either a Blossum 62 matrix or a PAM250 matrix, and a gap weight of 16, 12, 10, 8, 6, or 4 and a length weight of 1, 2, 3, 4, 5, or 6. In yet another embodiment, the percent identity between two nucleotide sequences is determined using the GAP program in the GCG software package (available at the official Accelrys web site), using a NWSgapdna.CMP matrix and a gap weight of 40, 50, 60, 70, or 80 and a length weight of 1, 2, 3, 4, 5, or 6. One set of parameters (and the one that can be used if the practitioner is uncertain about what parameters should be applied to determine if a molecule is within a sequence identity or homology limitation in one example, are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (CABIOS, 4:11-17 (1989)) which has been incorporated into the ALIGN program (version 2.0), using a PAM 120 weight residue table, a gap length penalty of 12 and a gap penalty of 4.

The nucleic acid and protein sequences described herein can be used as a “query sequence” to perform a search against public databases to, for example, identify other orthologs family members or related sequences. Such searches can be performed using the NBLAST and XBLAST programs (version 2.0) of Altschul et al. J Mol. Biol. 215:403-10 (1990). BLAST nucleotide searches can be performed with the NBLAST program, score=100 word length=12, to obtain nucleotide sequences homologous to known RSV DNA and RNA sequences. BLAST protein searches can be performed with the XBLAST program, score=50, word length=3, to obtain amino acid sequences homologous to known RSV polypeptide products. To obtain gapped alignments for comparison purposes Gapped BLAST can be utilized as described in Altschul et al. Nucleic Acids Res. 25:3389-3402 (1997). When utilizing BLAST and Gapped BLAST programs, the default parameters of the respective programs (e.g., XBLAST and NBLAST) can be used (see the National Center for Biotechnology Information web site of the National Institutes of Health).

Orthologs can also be identified using any other routine method known in the. such as screening a cDNA library, using a probe designed to identify sequences that are substantially identical to a reference sequence.

### Pharmaceutical Compositions and Methods of Administration

The NPRA inhibitors (e.g., isatin or other oxindols, siRNA molecules, antisense molecules, and ribozymes) can be incorporated into pharmaceutical compositions. Such compositions may include the polynucleotide and a pharmaceutically acceptable carrier. As used herein, the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. Formulations (compositions) are described in a number of sources that are well known and readily available to those skilled in the art. (For example, Remington's Pharmaceutical Sciences (Marin E., Easton Pa. Mack Publishing Company, ed., 1995) describes formulations may be used.

A pharmaceutical composition is formulated to be compatible with its intended route of administration. Examples of routes of administration include parenteral intravenous, intradermal, subcutaneous, oral (e.g., inhalation), nasal, topical, transdermal, transmucosal, and rectal administration. Solutions or suspensions used for parenteral intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline bacteriostatic water CREMOPHOR EL (BASF, Parsippany, N.1.) or phosphate buffered saline (PBS). In all cases, the composition should be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, water ethanol, polyol (for example, glycerol, propylene glycol, and liquid polyethylene glycol and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Isotonic agents, for example, sugars, polyalcohols such as manitol, sorbitol, and sodium chloride can also be included in the composition. Prolonged absorption of the injectable compositions can be brought about by including in the composition an agent that delays absorption, such as aluminum monostearate or gelatin.

Sterile injectable solutions can be prepared by incorporating the active compound (e.g., a polynucleotide, in one example) in the required amount in an appropriate solvent with one or a combination of ingredients enumerated above, as required, followed by filtered sterilization. Generally, dispersions are prepared by incorporating the polynucleotide into a sterile vehicle, which contains a basic dispersion medium and the required other ingredients from those enumerated above. In the case of sterile powders for the preparation of sterile injectable solutions, suitable methods of preparation include vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of similar nature: a binder such as microcrystalline cellulose gum tragacanth or gelatin; an excipient such as starch or lactose, a disintegrating agent such as alginic acid, PRIMOGEL, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the NPRA inhibitors can be delivered in the form of drops or an aerosol spray from a pressured container or dispenser that contains a suitable propellant a gas such as carbon dioxide, or a nebulizer. Such methods include those described in U.S. Pat. No. 6,468,798.

Systemic administration can also be by transmucosal or transdermal means. For transmucosal or trans dermal administration, penetrants appropriate to the barrier to be permeated are used in the formulation. Such penetrants are generally known in the art and include, for example, for transmucosal administration, detergents, bile salts, and fusidic acid derivatives. Transmucosal administration can be accomplished through the use of nasal sprays, drops, or suppositories. For trans dermal administration, the active compound (e.g., polynucleotides, for example) are formulated into ointments, salves, gels, or creams, as generally known in the art.

The pharmaceutical compositions can also be prepared in the form of suppositories (e.g. with conventional suppository bases such as cocoa butter and other glycerides) or retention enemas for rectal delivery.

In embodiments in which the NPRA inhibitor is a polynucleotide, the polynucleotides may be administered by transfection or infection using methods known in the art, including but not limited to, the methods described in McCaffrey et al., Nature 418(6893):38-39 (2002) (hydrodynamic transfection); Xia et al., Nature Biotechnol. 20(10):1006-10 (2002) (viral-mediated delivery); or Putnam Am. J Health Syst. Pharm. 15 53(2):151-160 (1996), erratum at Am. J. Health Syst. Pharm. 53(3):325 (1996).

The polynucleotides can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in Hamajima et al., Clin. Immunol. Immunopathol. 88(2):205-10 (1998). Liposomes (e.g., as described in U.S. Pat. No. 6,472,375) and micro encapsulation can also be used. Biodegradable targetable microparticle delivery systems can also be used (e.g., described in U.S. Pat. No. 6,471,996). Preferably, the polynucleotides are administered to the subject such that an effective amount are delivered to the respiratory epithelial cells, DC, and/or monocytes within the subject's airway, resulting in an effective amount of reduction in NPRA gene expression.

In one embodiment, the polynucleotides are prepared with carriers that will protect the polynucleotide against rapid elimination from the body, such as a controlled release formulation including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions (including liposomes targeted to antigen-presenting cells with monoclonal antibodies) can also be used as pharmaceutically. acceptable carriers. These can be prepared according to methods known to those skilled in the art, for example, as described in U.S. Pat. No. 4,522,811.

In one example, the NPRA inhibitors (e.g., compositions containing them) are administered locally or systemically such that they are delivered to target cells, such as cells of the airway, airway epithelial cells, which line the nose as well as the large and small airways. For some disorder, it is preferred that the NPRA inhibitors may be delivered to dendritic cells and/or monocytes.

Toxicity and therapeutic efficacy of compositions can be determined by standard pharmaceutical procedures in cell cultures or experimental animals for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions which exhibit high therapeutic indices can be used. While compositions that exhibit toxic side effects can be used, care should be taken to design a delivery system that targets such compounds to the site of affected tissue in order to minimize potential damage to uninfected cells and, thereby, reduce side effects.

Data obtained from cell culture assays and animal studies can be used in formulating a range of dosage for use in humans. The dosage of such compositions generally lies within a range of circulating concentrations that include the ED50 with little or no toxicity. The dosage can vary within this range depending upon the dosage form employed and the route of administration utilized. For composition used in one example of the method, the therapeutically effective dose may be estimated initially from cell culture assays. A dose can be formulated in animal models to achieve a circulating plasma concentration range that includes the IC₅₀ (i.e., the concentration of the test composition which achieves a half-maximal inhibition of symptoms) as determined in cell culture. Such information can be used to more accurately determine useful doses in humans. Levels in plasma can be measured, for example, by high performance liquid chromatography.

The compositions may be administered on any appropriate schedule from one or more times per day to one or more times per week; including once every other day, for any number of days or weeks 1 day, 2 days, 3 days, 4 days, 5 days, 6 days, 1 week, 10 days, 2 weeks, 3 weeks, 4 weeks, 5 weeks, 6 weeks, 7 weeks, 8 weeks, 2 months, 3 months, 6 months, or more, or any variation thereon. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a NPRA inhibitor can include a single treatment or can include a series of treatments.

Mammalian species that benefit from the disclosed methods include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo bison, horses, donkey, swine, sheep, and goats; exotic animals typically found in zoos such as bear, lions, tigers, panthers, elephants, hippopotamus, rhinoceros, giraffes antelopes, sloth, gazelles, zebras, wildebeests, prairie dogs, koala bears, kangaroo opossums, raccoons, pandas, hyena, seals, sea lions, elephant seals, otters, porpoises dolphins, and whales. As used herein, the terms “subject” “host”, and “patient” are used interchangeably and intended to include such human and non-human mammalian species. Likewise methods of the present invention can be carried out on cells of such in vitro mammalian species. Host cells comprising exogenous polynucleotides, in one example of the methods and the compositions, may be administered to the subject, and may, for example, be autogenic (use of one's own cells), allogenic (from one person to another), or transgenic or xenogeneic (from one species to another), relative to the subject.

The polynucleotides may be inserted into genetic constructs viral vectors, retroviral vectors, expression cassettes, or plasmid viral vectors using methods known in the art, including but not limited to those described in Xia et al. (2002), supra. Genetic constructs can be delivered to a subject by, for example inhalation, orally, intravenous injection, local administration (see U.S. Pat. No. 5,328,470) or by stereotactic injection (see e.g., Chen et al., Proc. Natl. Acad. Sci. USA Chen 91:3054-3057 (1994)). The pharmaceutical preparation of the delivery vector can include the vector in an acceptable diluent, or can comprise a slow release matrix in which the delivery vehicle is imbedded. Alternatively, where the complete delivery vector can be produced intact from recombinant cells retroviral vectors, the pharmaceutical preparation can include one or more cells which produce the polynucleotide delivery system. The polynucleotides, for example, can also include small hairpin RNAs (shRAs), and expression constructs engineered to express shRNAs. Transcription of shRAs is initiated at a polymerase III (pol III) promoter, and is thought to be terminated 10 at position 2 of a 4-thymine transcription termination site. Upon expression, shRNAs are thought to fold into a stem-loop structure with 3′ UU-overhangs; subsequently, the ends of these shRAs are processed, converting the shRAs into siRNA-like molecules of about 21 nucleotides (Brummelkamp et al., Science 296:550-553 (2002); Lee et al. (2002), supra; Miyagishi and Taira Nature Biotechnol. 20:497-500 (2002); Paddison et al. (2002), supra; Paul (2002), supra; Sui (2002) supra; Yu et al. (2002), supra. SiRNAs may be fused to other nucleotide, molecules, or to polypeptides, in order to direct their delivery or to accomplish other functions. Thus, for example, fusion proteins comprising a siRNA oligonucleotide that is capable specifically interfering with expression of NPRA gene may comprise affinity tag polypeptide sequences, which refers to polypeptides or peptides that facilitate detection and isolation of the polypeptide via a specific affinity interaction with a ligand. The ligand may be any molecule, receptor, counter-receptor, antibody or the like with which the affinity tag may interact through a specific binding interaction as provided herein. Such peptides include, for example, poly-His or “FLAG” or the like the antigenic 25 identification peptides described in U.S. Pat. No. 5,011,912 and in Hopp et al. (Bio/Technology 6:1204, 1988), or the XPRESS epitope tag (INVITROGEN, Carlsbad, Calif.) The affinity sequence may be a hexa-histidine tag as supplied, for example, by a pBAD/H is (INVITROGEN) or a pQE-9 vector to provide for purification of the mature polypeptide fused to the marker in the case of a bacterial host, or, for example, the affinity sequence may be a hemagglutinin (HA) tag when a mammalian host COS cells, is used. The HA tag corresponds to an antibody defined epitope derived from the influenza hemagglutinin protein (Wilson et al. 1984 Cell 37:767).

The methods and compositions also relate to vectors and to constructs that include or encode polynucleotides (e.g., siRNA), and in particular to recombinant nucleic acid constructs that include any nucleic acid such as a DNA polynucleotide segment that may be transcribed to yield NPRA mRNA-specific siRNA polynucleotides as provided above; to host cells which are genetically engineered with vectors and/or constructs and to the production of siRNA polynucleotides, polypeptides, and/or fusion proteins of the or fragments or variants thereof, by recombinant techniques. siRNA sequences disclosed herein as RNA polynucleotides may be engineered to produce corresponding DNA sequences using well-established methodologies such as those described herein. Thus, for example, a DNA polynucleotide may be generated from any siRNA sequence described herein, such that the present siRNA sequences will be recognized as also providing corresponding DNA polynucleotides (and their complements). These DNA polynucleotides are therefore encompassed, and can for example, be incorporated into the recombinant nucleic acid constructs from which siRNA may be transcribed.

According to one example, a vector may comprise a recombinant nucleic acid construct containing one or more promoters for transcription of an RNA molecule for example, the human U6 snRA promoter (see et al, Nat. Biotechnol. Miyagishi 20 20:497-500 (2002); Lee et al., Nat. Biotechnol. 20:500-505 (2002); Paul et al., Nat. Biotechnol. 20:505-508 (2002); Grabarek et al., BioTechniques 34:73544 (2003); see also Sui et al, Proc. Natl. Acad. Sci. USA 99:5515-20 (2002)). Each strand of a siRNA polynucleotide may be transcribed separately each under the direction of a separate promoter and then may hybridize within the cell to form the siRNA polynucleotide duplex. Each strand may also be transcribed from separate vectors (see Lee et al., supra).

Alternatively, the sense and antisense sequences specific for an RSV sequence may be transcribed under the control of a single promoter such that the siRNA polynucleotide forms a hairpin molecule (Paul et al., supra). In such an instance, the complementary strands of the siRNA specific sequences are separated by a spacer that comprises at least four nucleotides, but may comprise at least 5, 6, 7, 8, 9, 10, 11, 12, 14, 16, 94 nucleotides or more nucleotides as described herein. In addition, siRNAs transcribed under the control of a U6 promoter that form a hairpin may have a stretch of about four uridines at the 3′ end that act as the transcription termination signal (Miyagishi et al., supra; Paul et al., supra). By way of illustration, if the target sequence is 19 nucleotides the siRNA hairpin polynucleotide (beginning at the 5′ end) has a 19-nucleotide sense sequence followed by a spacer (which has two uridine nucleotides adjacent to the 3′ end of the 19-nucleotide sense sequence), and the spacer is linked to a 19 nucleotide antisense sequence followed by a 4-uridine terminator sequence, which results in an overhang. siRNA polynucleotides with such overhangs effectively interfere with expression of the target polypeptide. A recombinant construct may also be prepared using another RNA polymerase III promoter, the HI RNA promoter, that may be operatively linked to siRNA polynucleotide specific sequences, which may be used for transcription of hairpin structures comprising the siRNA specific sequences or separate transcription of each strand of a siRNA duplex polynucleotide (see Brummelkamp et al., Science 296:550-53 (2002); Paddison et al., supra). DNA vectors useful for insertion of sequences for transcription of an siRNA polynucleotide include pSUPER vector (see Brummelkamp et al, supra); pAV vectors derived from pCWRSVN (see Paul e al., supra); and pIND (see Lee et al., supra), or the like.

Polynucleotides may be expressed in mammalian cells, yeast bacteria, or other cells under the control of appropriate promoters, providing ready systems for evaluation of NPRA polynucleotides that are capable of interfering with expression of NPRA gene, as provided herein. Appropriate cloning and expression vectors for use with prokaryotic and eukaryotic hosts are described, for example, by Sambrook et al. Molecular Cloning: A Laboratory Manual, Third Edition, Cold Spring Harbor, N., (2001).

The appropriate DNA sequence(s) may be inserted into the vector by a variety procedures. In general, the DNA sequence is inserted into an appropriate restriction endonuclease site(s) by procedures known in. the art. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described, for example, in Ausubel et al. (1993 Current Protocols in Molecular Biology, Greene Publ. Assoc. Inc. & John Wiley & Sons Inc., Boston, Mass.); Sambrook et al. (2001 Molecular Cloning, Third Ed., Cold Spring Laboratory, Plainview, N.Y.); Maniatis et al., Harbor Laboratory, Plainview, N.Y. (1982 Molecular Cloning, Cold Spring Harbor Laboratory, Plainview, N.Y.); and elsewhere.

The DNA sequence in the expression vector is operatively linked to at least one appropriate expression control sequences (e.g., a promoter or a regulated promoter) to direct mRNA synthesis. Representative examples of such expression control sequences include LTR or SV40 promoter, the E. coli lac or trp, the phage lambda P promoter and other promoters known to control expression of genes in prokaryotic or eukaryotic cells or their viruses. Promoter regions can be selected from any desired gene using CAT (chloramphenicol transferase) vectors or other vectors with selectable markers. Eukaryotic promoters include CMV immediate early, HSV thymidine kinase, early and late SV40 LTRs from retrovirus, and mouse metallothionein-I. Selection of the appropriate vector and promoter is well within the level of ordinary skill in the art, and preparation of certain particularly preferred recombinant expression constructs composing at least one promoter, or regulated promoter, operably linked to a polynucleotide is described herein.

As noted above, in certain embodiments the vector may be a viral vector such as a mammalian viral vector (e.g., retrovirus, adenovirus, adeno-associated virus, lentivirus). For example, retroviruses from which the retroviral plasmid vectors may be derived include, but are not limited to, Moloney Murine Leukemia Virus, spleen necrosis virus, retroviruses such as Rous Sarcoma Virus, Harvey Sarcoma virus, avian leukosis virus, gibbon ape leukemia virus human immunodeficiency Virus adenovirus Myeloproliferative Sarcoma Virus, and mammary tumor virus.

The viral vector includes one or more promoters. Suitable promoters that may be employed include, but are not limited to, the retroviral L TR; the SV 40 promoter; and the human cytomegalovirus (CMV) promoter described in Miller et al., Biotechniques 7:980-990 (1989), or any other promoter (e.g., cellular promoters such as eukaryotic cellular promoters including, but not limited to, the histone, pol III, and beta-actin promoters). Other viral promoters that may be employed include, but are not limited to adenovirus promoters' adeno-associated virus promoters, thymidine kinase (TK) promoters, and B 19 parvovirus promoters. The selection of a suitable promoter will be apparent to those skilled in the art from the teachings contained herein, and may be from among either regulated promoters (e.g., tissue-specific or inducible promoters) or promoters as described above. A tissue-specific promoter allows preferential expression of the polynucleotide in a given target tissue (such as tissue of the respiratory tract), thereby avoiding expression in other tissues. For example, to express genes specifically in the heart, a number of cardiac-specific regulatory elements can be used. An example, of a cardiac-specific promoter is the ventricular form of MLC-2v promoter (Zhu al., Mol. Cell. Biol. 13:4432-4444, 1993; Navankasattusas et al, Mol. Cell Biol. 12: 1469, 1479, 1992) or a variant thereof such as a 281 bp fragment of the native MLC-promoter (nucleotides −264 to +17 Genebank Accession No. U26708). Examples of other cardiac-specific promoters include alpha myosin heavy chain (Minamino et al., Circ. Res. 88:587-592, 2001) and myosin light chain-2 (Franz et al., Circ. Res. 73:629638, 1993). Endothelial cell gene promoters include endoglin and ICAM-2. See Velasco et al., Gene Ther. 8:897-904 2001. Liver-specific promoters include the human phenylalanine hydroxylase (PAH) gene promoters (Bristeau et al. Gene 274:283-291 2001), hBIF (Zhang et al., Gene 273:239-249, 2001), and the human C-reactive protein (CRP) gene promoter (Ruther et al. Oncogene 8:87, 1993). Promoters that are kidney-specific include CLCN5 (Tanaka et al., Genomics 58:281-292, 1999), renin (Sinn et al., Physical Genomics 3:25-, 2000), androgen-regulated protein, sodium-phosphate cotransporter, renal cytochrome P-450, parathyroid hormone receptor and kidney-specific cadherin. See Am. J Physiol. RenalPhysiol. 279:F383-392, 2000. An example of a pancreas-specific promoter is the pancreas duodenum homeo box 1 (PD X-I) promoter (Samara et al., Mol. Cell. Biol. 22:4702-4713, 2002). A number of brain-specific promoters may be used, for example, and include the thy-1 antigen and gamma-enolase promoters (Vibert et al., Eur. J. Biochem. 181:33-, 1989), the glial-specific glial fibrillary acidic protein (GFAP) gene promoter (Cortez et al., J. Neurosci. Res. 25 59:39-, 2000), and the human FGFI gene promoter (Chiu et al., Oncogene 19:62296239, 2000). The GATA family of transcription factors have promoters directing neuronal and thymocyte-specific expression (see Asnagli et al., J Immunol. 168:42684271, 2002).

In another aspect, host cells containing the above described recombinant constructs. Host cells are genetically engineered/modified (transduced, transformed or transfected) with the vectors and/or expression constructs of that may be, for example, a cloning vector, a shuttle vector, or an expression construct. The vector or construct may be, for example, in the form of plasmid, a viral particle, a phage etc. The engineered host cells can be cultured in conventional nutrient media modified as appropriate for activating promoters, selecting trans formants or amplifying particular genes such as genes encoding siRNA polynucleotides or fusion proteins thereof. The culture conditions for particular host cells selected for expression, such as temperature, pH and the like, will be readily apparent to the ordinarily skilled artisan.

The host cell can be a higher eukaryotic cell, such as a mammalian cell, or a lower eukaryotic cell, such as a yeast cell, or the host cell can be a prokaryotic cell, such as a 10 bacterial cell. Representative examples of appropriate host cells according to the present invention include, but need not be limited to, bacterial cells, such as E. coli, Salmonella typhimurium; Streptomyces fungal cells such as yeast; insect cells, such as Drosophila S2 and Spodoptera Sf9; animal cells, such as CHO COS or 293 cells; adenoviruses; plant cells, or any suitable cell already adapted to in vitro propagation or so 15 established de novo.

Various mammalian cell culture systems can also be employed to produce polynucleotides, for example, from recombinant nucleic acid constructs. A method of producing a polynucleotide, such as a siRNA, by culturing a host cell comprising a recombinant nucleic acid construct that comprises at least one promoter operably linked to a polynucleotide that is specific for NPRA gene, in one example. In certain embodiments, the promoter may be a regulated promoter as provided herein, for example a tetracycline-repressible promoter. In certain embodiments, the recombinant expression construct is a recombinant viral expression construct as provided herein. Examples of mammalian expression systems include the COS-7 lines of monkey kidney fibroblasts, described by Gluzman 23:175 (1981), and other cell lines capable of expressing a compatible Cell vector, for example, the C127, 3T3, CHO, HeLa, HEK, and BHK cell lines. Mammalian expression vectors will comprise an origin of replication, a suitable promoter and enhancer, and also any necessary ribosome binding sites, polyadenylation site, splice donor and acceptor sites transcriptional termination sequences, and 5′ flaning nontranscribed sequences, for example as described herein regarding the preparation of recombinant polynucleotide constructs. DNA sequences derived from the SV 40 splice, and polyadenylation sites may be used to provide the required nontranscribed genetic elements. Introduction of the construct into the host cell can be effected by a variety of methods with which those skilled in the art will be familiar, including but not limited to for example liposomes including cationic liposomes, calcium phosphate transfection DEAE-Dextran mediated transfection, or electroporation (Davis et al. 1986 Basic Methods in Molecular Biology), or other suitable technique.

The expressed polynucleotides may be useful in intact host cells; in intact organelles such as cell membranes, intracellular vesicles or other cellular organelles; or in disrupted cell preparations including but not limited to cell homogenates or lysates microsomes uni- and multilamellar membrane vesicles or other preparations. Alternatively, expressed polynucleotides can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Finally, high performance liquid chromatography (HPLC) can be employed for final purification steps.

As used herein, the terms “administer apply treat”, “transplant”, “implant”, “deliver”, and grammatical variations thereof, are used interchangeably to provide NPRA inhibitors of the subject invention (e.g., vectors containing or encoding polynucleotides of the subject invention) to target cells in vitro or in vivo, or provide genetically modified (engineered) cells of the subject invention to a subject ex vivo.

As used herein, the term “co-administration” and variations thereof refers to the administration of two or more agents simultaneously (in one or more preparations), or consecutively. For example, one or more types of NPRA inhibitors, in one example, (e.g., vectors containing or encoding polynucleotides) can be co-administered with other agents. As used in this specification, including the appended claims, the singular, and the include plural reference unless the contact dictates otherwise. Thus, for example, a reference to “a polynucleotide” includes more than one such polynucleotide reference to “a nucleic acid sequence” includes more than one such sequence. A reference to “a cell” includes more than one such cell.

The terms “comprising”, “consisting of”, and “consisting essentially of” are defined according to their standard meaning. The terms may be substituted for one another throughout the instant application in order to attach the specific meaning associated with each term.

### Example 9

### ANP Over Expression in the Lung Augments Inflammation and Cytokine Production in Splenocytes

ANP has been suspected to play a role in decreasing inflammation, as it was shown to play a role in decreasing TNF-a production from macrophages and slightly decreased NFkB activation (Mohapatra et al. JACI, 2004). Also, NPRA deficient mice did not exhibit. inflammation. Since excess ANP expression activates the clearance receptor, it was hypothesized that ANP actually increases inflammation. To test this naive mice were administered intranasally a plasmid p AX expressing the ANP peptide. The results show that ANP over expression actually increases inflammation.

### Materials and Methods

Six-week old female BALB/c mice from Jackson laboratory (Bar Harbor, Me.) were maintained in pathogen free conditions in accordance with animal research committee regulations. Total RNA was isolated from murine

Construction of ANP expression vector.

Total RNA was isolated from murine heart using Trizol reagent (LIFE TECHNOLOGY, Gaithersburg, Md.) following the manufacturer s protocol. The cDNA sequence for the ANP, residues 99-126 of pro ANP was amplified by R T-PCRA translation initiation codon was inserted in the forward primers, so that the recombinant peptides had an additional amino acid, methionine, as the first amino acid apart from its known content. The product was cloned in p VAX 25 vector (INVITROGEN, Carlsbad, Calif.) at HindIII and XhoI sites. The cloned ANP sequence was verified by DNA sequencing and its expression was checked in A549 human epithelial cells.

### Analysis of Intracellular Cytokine Production in T Cells.

Mouse spleen T cells purified using mouse T-cell enrichment column kit (R & D Systems, Minneapolis, Minn.) were cultured in 6-well plates for 4 days. Finally, cells were stimulated with PMA (50 ng/ml) and ionomycin (500 ng/ml) (SIGMA, Saint Louis, Mo.) for 6 hours in the presence of GOLGISTOP (PHARMINGEN, San Diego, Calif.) and then fixed and stained using CD8 or CD4 mAb (BD BIOSCIENCES, San Diego, Calif.) for flow cytometry analysis.

### Histological Analysis.

Mouse lungs were removed after 24 hours of intranasal pANP administration, fixed, and sections stained with H&E.

### Results.

Normal BALB/c mice were given intranasally by nose drops chitosan nanoparticles carrying pANP or pVAX and their lungs were examined 3 days after by staining the sections (H&E), showing goblet cell hyperplasia. These results directly demonstrate that in normal mice over expression of ANP results in bronchial inflammation. To demonstrate that ANP over expression also stimulates immune system, BALB/c mice were given i.p. OVA (with 10 alum) and then challenged i.n. OV A. Mice were sacrificed, the spleens aseptically removed and the cells were cultured for 48 hours in the presence of OVA (Sigma) and recombinant IL-2. Cells were removed from culture and stained for surface markers CD4 and CD3 and intracellular cytokines IL-, IL-10 and IFN-g (BD Pharmingen). The results show that in normal mice in absence of any antigen sensitization, ANP over expression increases expression of both ANP in general augments inflammation by activating both innate and adaptive immunity.

### Example 10

### Inhibitory Effect of Transfected siRNA Plasmids on NPRA Expression

To determine whether siRNAs can be produced that will effectively decrease NPRA expression, 11 different siRNA oligos were designed and cloned in a pU6 vector. Cells transfected with each of the construct was examined for NPRA protein expression by western blotting.

### Materials and Methods

### Plasmid Constructs.

The nucleotide sequence for each is described previously (SEQ ID NOs: 23-123). Each pair of oligos was inserted into pU6 plasmid at appropriate sites respectively, to generate the corresponding siRNA for siNPRA.

DNA Transfection.

Cells were transfected with siNPRA or controls (siU6) using LIPOFECTAMINE 2000 reagent (INVITROGEN, Carlsbad, Calif.). pEGFP plasmid (STRATAGENE, La Jolla, Calif.) was used for measurement of transfection efficiency.

Protein Expression Analysis by Western Blotting.

Transfected cells were used to prepare whole cell lysates, which were electrophoresed on 120/0 polyacrylamide gels and the proteins were transferred to PVDF membranes (BIO-RAD, Hercules, Calif.). The blot was incubated separately with NPRA polyclonal antibody (SANTA CRUZ BIOTECH Santa Cruz, CA), immunoblot signals were developed by SUPER SIGNAL ULTRA chemiluminescent reagent (PIERCE, Rockford, Ill.).

Results.

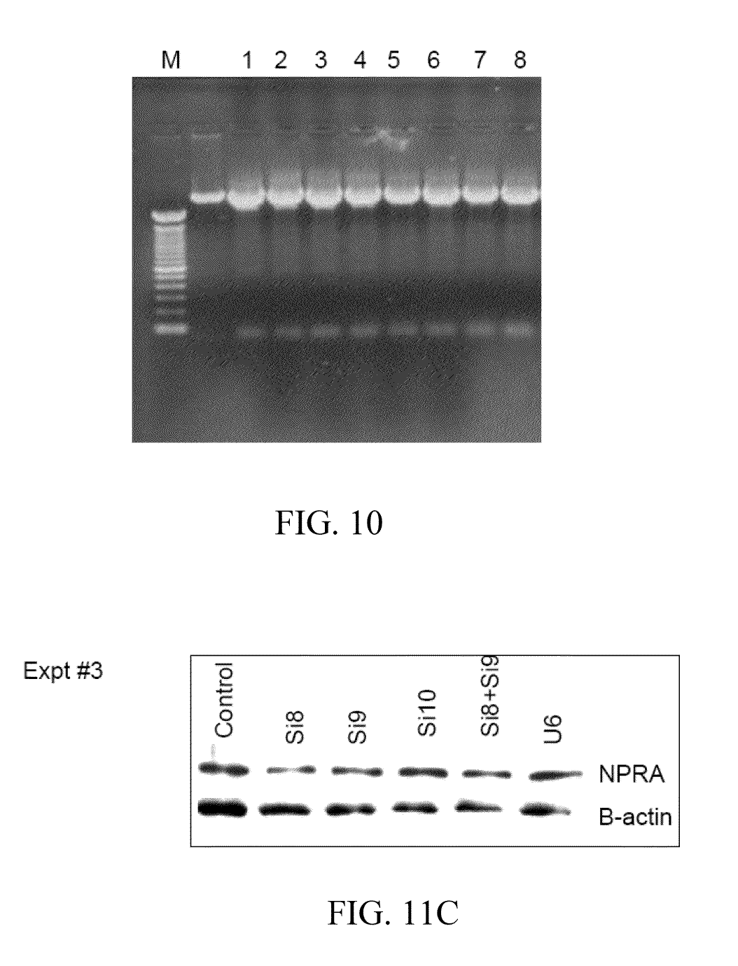

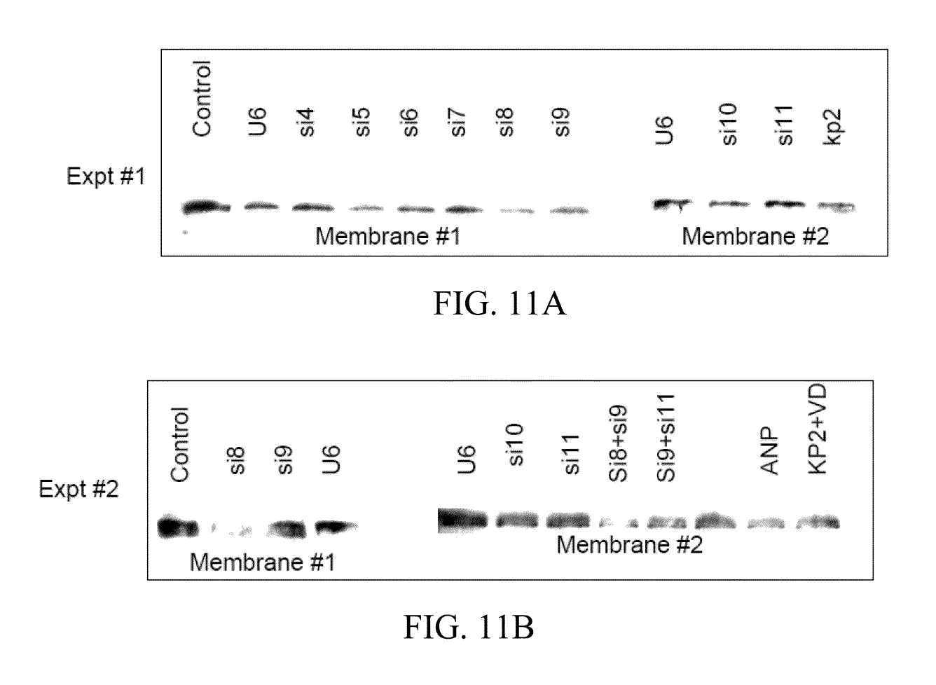

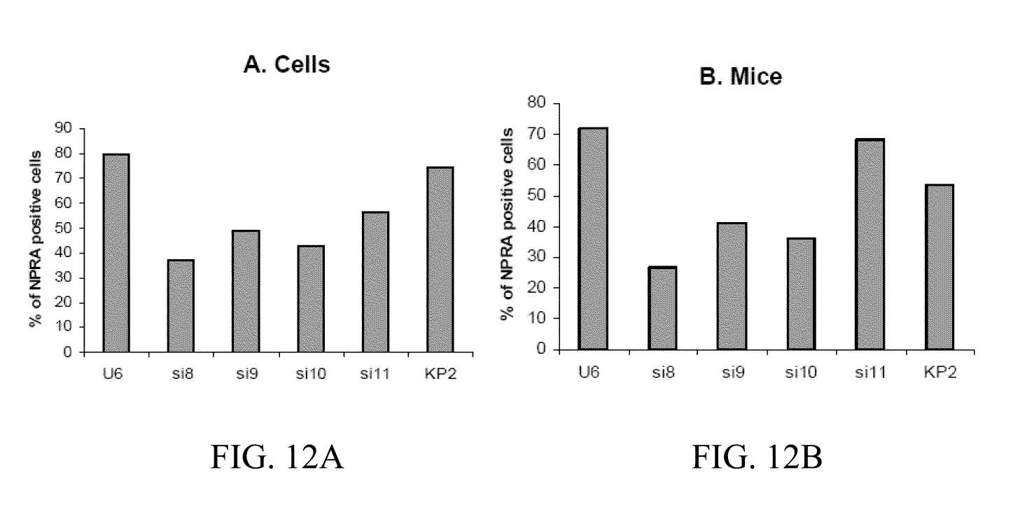

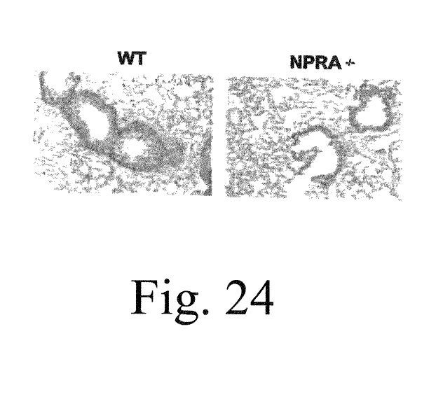

Eleven different siRNA oligos were designed specifically targeting NPRA gene. The siRNA oligos were cloned in pU6 vector. FIG. 10 shows results the inserts being present in the plasmids. The inserts were sequenced to confirm the presence of siRNA inserts in them. Cells in 6-well plates were transfected with psiNPRA (2 ug). Forty eight hours later, total protein were extracted western blotted using an antibody to NPRA. Results from two different experiments are shown in FIGS. 11A-11C. Plasmids encoding ANP, NP₇₃₋₁₀₂ and VD were used as control, since they have been shown to down regulate NPRA expression. In the third experiment, HEKGCA cells grown in 6-well plates were transfected with psiNPRA (2 ug), as indicated and forty eight hours later total protein were extracted western blotted using an antibody to NPRA (FIG. 11C). Untransfected cells and cells transfected with U 6 vector plasmid without any siNPRA were used as control. Also, filters were stripped and reprobed with antibody to beta-actin. The experiments were repeated. The results showed that 3 of 11 siNPRA constructs consistently decreased NPRA protein expression in the HEKGCA cells. To confirm these results, inhibitory effect of siRNA in vitro was examined using HEKGCA cells. Cells grown in 6-well plates were transfected with psiNPRA (2 ug). Forty eight hours later, cells were subjected to flow cytometry to detect NPRA positive cells using an antibody to NPRA (FIG. 12A). U6 plasmid without any siRNA and Plasmid encoding Kp73-02 was used as controls, since the latter has been shown to down regulate NPRA expression. Mice (n=4) were intranasally administered with 25 ug siRNA plasmids complexed with 125 ul of chitosan nanoparticles. BAL was done 72 hours later. Cells were stained by NPRA Ab. NPRA expression cells were counted (FIG. 24). Together the results show that siNPRA8, siNPRA9 and siNPRA10 were the most effective siRNAs that significantly reduced NPRA expression.

### Example 11

### Demonstration that Oral siNPRA Treatment Decreases Inflammation, Eosinophilia and the Cytokines in Balb/c Mice

To determine whether decreased expression of NPRA by siNPRA treatment will reduce inflammation in asthma, the effect of intranasal siNPRA9 was tested in ovalbumin-induced mouse model of asthma.

Materials and Methods.

Six to eight week-old BALB/c mice (n=6) were sensitized by i.p. injection of ovalbumin (50 ug in 2 mg of alum/mouse) and challenged intranasally with OVA (50 μg). Mice were given two siNPRA9 treatments by lavage and challenged 24 hours later. After a further 24 hours of challenge, mice were sacrificed and their lungs removed for histology in a subgroup (n=3) of mice. The remainder of the group were lavaged and a cell differential was performed as described, especially to enumerate the eosinophil numbers in the BAL fluid. Thoracic lymph node cells (A) and spleen cells (B) were removed and cells cultured for 48 hours in the presence of OVA (Sigma Grade V) and recombinant mouse IL-2. Naive mice received no treatment. Cells were treated with GolgiStop (BD Pharmingen) and stained for surface and intracellular cytokines (Antibodies obtained from BD Pharmingen). Percent cytokine secreting cells were quantified by intracellular cytokine staining using flow cytometry, as described.

Results.

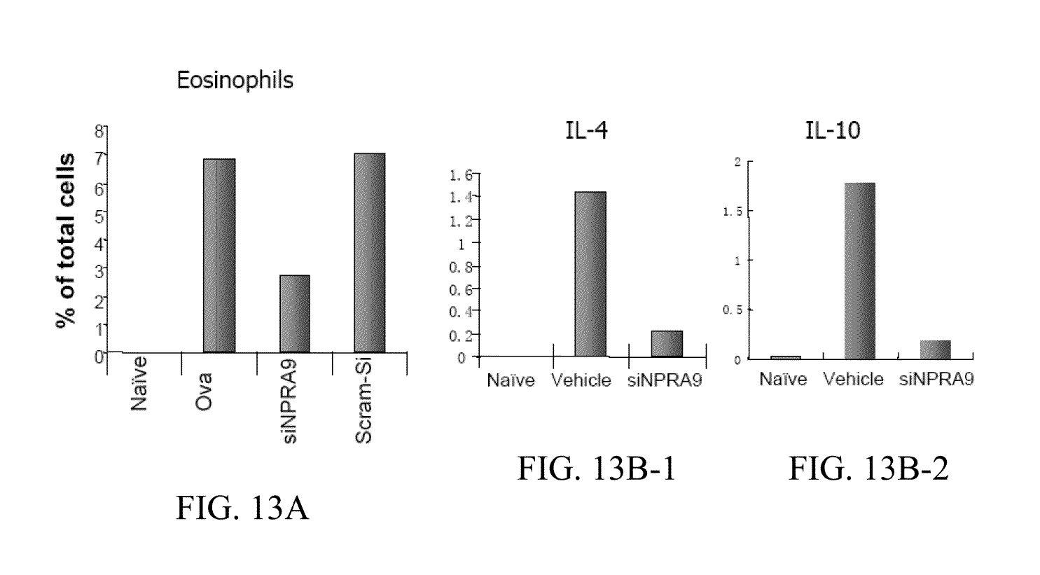

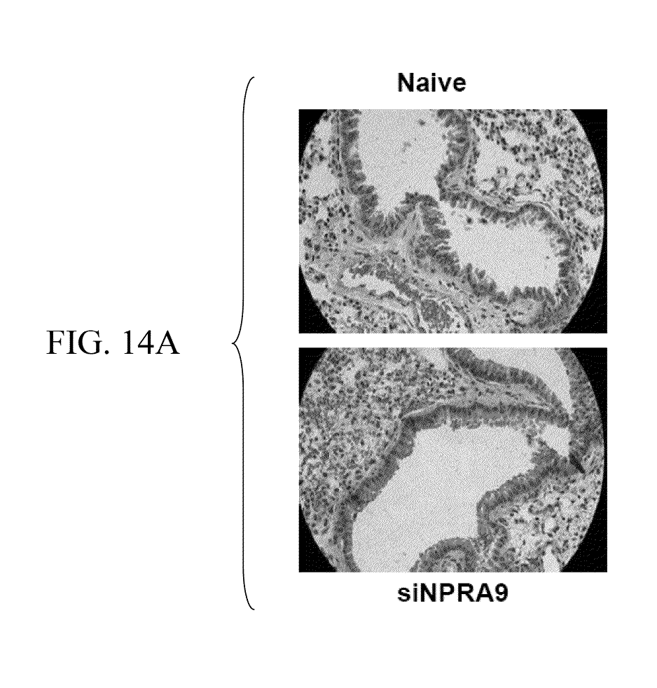

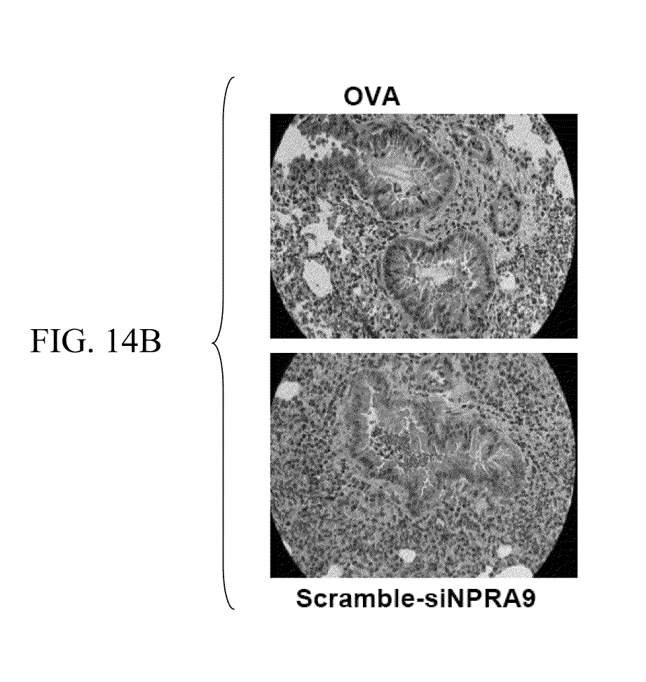

The results of lung histology, lung sections stained by H & E revealed that compared to untreated Ovalbumin-sensitized and mice treated with 20 scrambled si-NPRA group, treated mice showed a significant reduction in lung inflammation. The lung histology was very similar to the naive mice. There was significant reduction in epithelial goblet cell hyperplasia and a significant reduction in peribronchial, perivascular and interstitial infiltration of the inflammatory cells to the lung (FIGS. 14A-14C). There was also a significant reduction in the number of eosinophils in BAL fluid (FIG. 13A) and reduction in Th2 cytokines in thoracic lymph nodes as determined by intracellular cytokine staining (FIGS. 125-1 and 125-2).

### Example 12

### Demonstration that Transdermal siNPRA Treatment Decreases Inflammation, Eosinophilia and Th2 Cytokines in Balb/C Mice

Patients are more compliant when the drug is delivered by transdermal route. Therefore, siNPRA8 delivered by transdermal route was attempted to determine whether such siRNA therapy would decrease pulmonary inflammation in this ovalbumin-induced mouse model of asthma.

Materials and Methods.

BALB/c mice (n=5 each group) were sensitized (i.p.) as in example #11 and challenged (i.n.) with 50 ug of OVA. Mice were given siNPRA (oligonucleotide) treatments by transdermal route (siNPRA8) and challenged 4 hours later. Following 24 hours of challenge two mice were sacrificed to obtain lungs and which were fixed sectioned and immunostained for NPRA expression (A). Mice (n=3) were sacrificed and lavaged and the percentage of eosinophils (B) and IL-4 concentration (C) in the lavage fluid was determined.

Results.

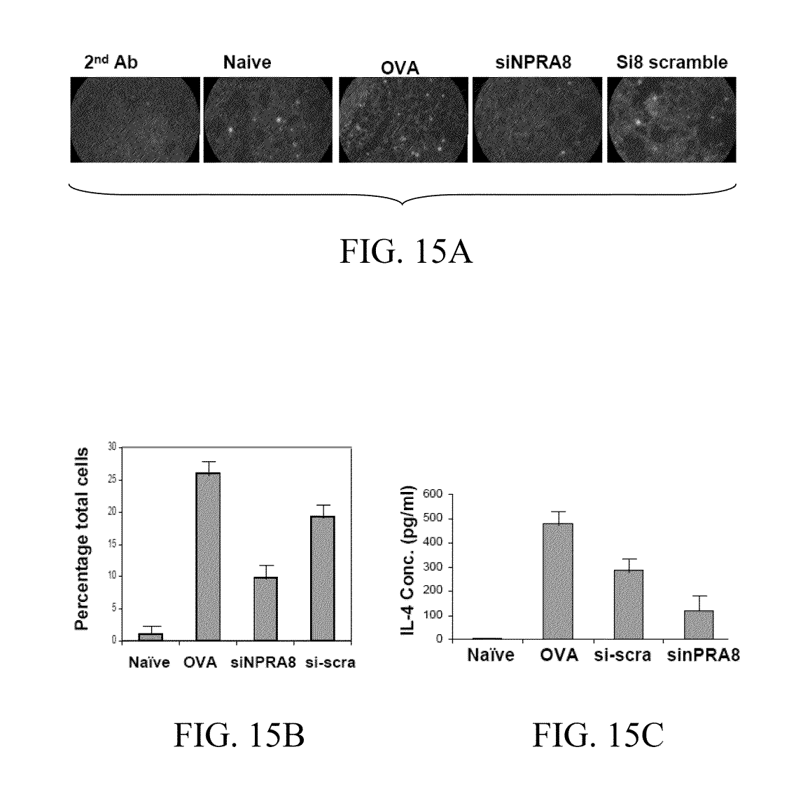

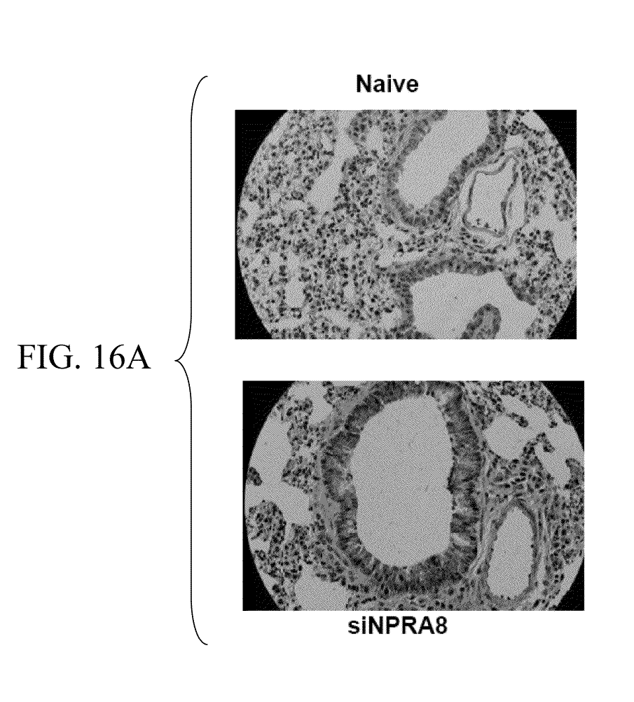

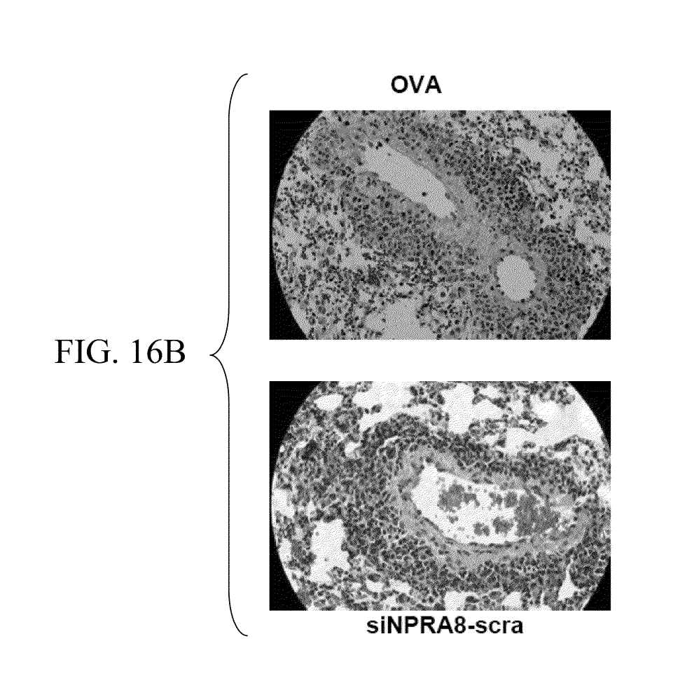

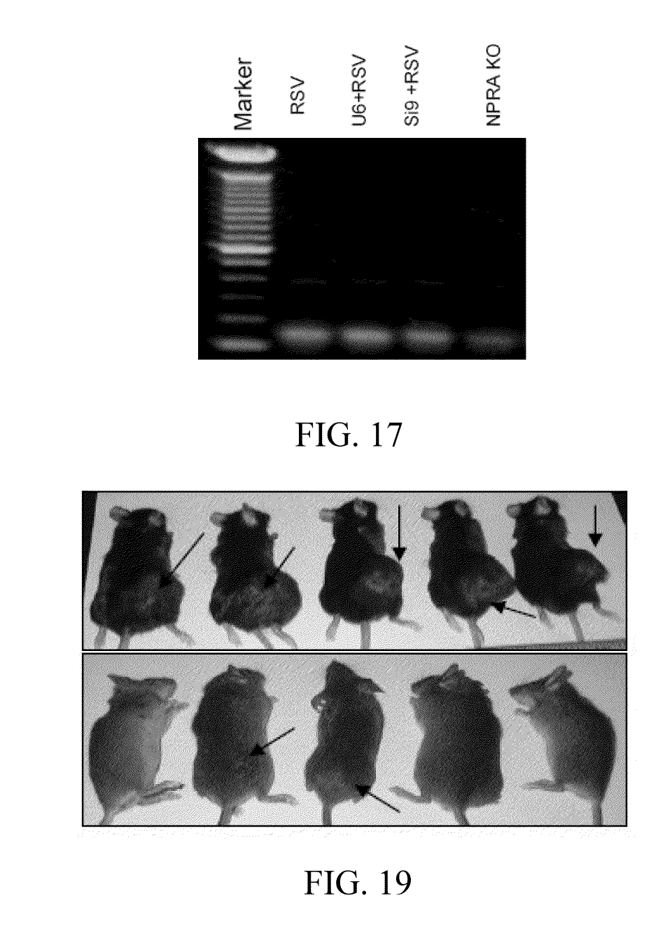

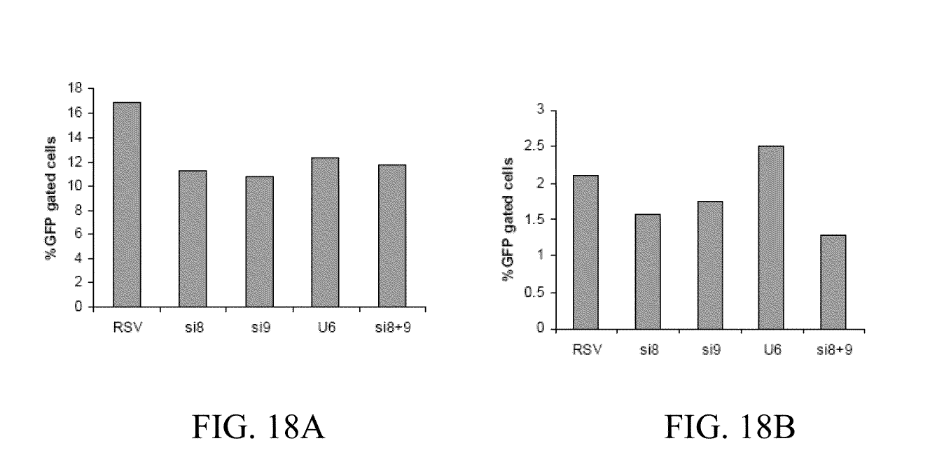

Since intradermal delivery of siRNA has not been shown previously, the lung sections were first checked for the expression of NPRA and whether siRNA delivered by transdermal route decreases NPRA expression. The results are shown in FIG. 15A and indicate that lungs of ova-sensitized mice and mice treated with scrambled si-NPRA8 show higher number of cells expressing NPRA. siNPRA treatment decreased the expression level significantly. Typically, epithelial cells did not express NPRA and although not verified it is the dendritic cells appear to be involved in NPRA expression. The siNPRA8 treated mice also showed a significant reduction in eosinophil numbers (FIG. 15B) and levels of IL-4 (FIG. 15C) in the BAL. The results of H & E staining of lung sections showed that compared to untreated Ovalbumin-sensitized and mice treated with scrambled si-NPRA8 group, treated mice showed a significant reduction in lung inflammation (FIGS. 16A and 16B). There was a significant reduction in epithelial goblet cell hyperplasia and a significant reduction in peribronchial, perivascular and interstitial infiltration of the inflammatory cells to the lung. Together these results show that transdermal delivery of siNPRA8 decreases NPRA expression and inflammation of the lung and reduction of IL-4 and eosinophils in the lung.

### Example 13

### Demonstration that Transfection of A549 Cells with psiNPRA9 Decreases the Number of Respiratory Syncytial Virus (RSV) Infection Infected Cells

Respiratory syncytial virus infection also causes bronchiolitis in newborns and in elderly causing pneumonitis which is characterized severe acute lung inflammation. RSV infection typically requires certain host cell proteins and transcription factors for its replication and subsequent infection of others cells. Since siNPRA treatment decreases pulmonary inflammation, the effect of siNPRA9 transfection on RSV infection was examined in pulmonary type-II epithelial cells was examined.

Materials and Methods.

RT-PCR analysis of NPRA expression in the lung of mice treated with siRNA psiNPRA9 was encapsulated with chitosan nanoparticles and intranasally delivered to mice. Twenty-four hours later, mice were infected with RSV (5×10⁶ pfu/mouse). Four days later, mice were sacrificed and lung cells were collected for RNA extraction. NPRA fragment were amplified by RT-PCR using NPRA specific primers (F:5′ GCA AAG GCC GAG TTA TCT ACA Te- (SEQ ID NO: 27), R:5′ AAC GTA GTC eTC CeC ACA CAA-3) (SEQ ID NO: 28) and analyzed in 1% agarose gel.

To determine the effect of siNPRA9 on RSBV infection of epithelial cells A549 cells were grown in 6 well plate, transfected by siNPRA8 siNPRA9 or control U6 plasmid (2.0 ug) and 2 hours after infected by rgRSV (MOI=0.2). Cells were checked for infection 48 hours later, FACS was done. Also, A549 cells were grown in 6 well plate infected by rgRSV (MOI=0.2) and 24 hours after infection they were transfected by siNPRA8, siNPRA9 or control U6 plasmid (2.0 ug) and further 24 hr later, flow cytometry was performed to estimate percentage of infected cells.

Results.

The RT-PCR analysis showed that both RSV infected mice and mice infected with RSV and intranasally treated with pU6 control plasmid given with chitosan nanoparticles showed NPRA expression in the lung cells. However, mice infected with RSV and intranasally given psiNPRA9 showed an amplification product that was reduced in band intensity compared to cells from mice given pU6 plasmid. The lung cells from NPRA knock-out mice showed the band as well but it was reduced in intensity.

To determine the effect of siNPRA9 on rgRSV infection of A549 cells, either cells were grown in 6 well plate, transfected by siNPRA8, siNPRA9 or control U6 plasmid (2.0 ug) and 2 hours after infected by rgRSV (MOI=0.2) (prophylactic approach), or A549 cells were grown in 6 cell plate infected by rgRSV (MOI=0.2) and 24 hours after infection they were transfected by siNPRA8, siNPRA9 or control U6 plasmid (2.0 ug) (therapeutic approach) and further 24 hr later, flow cytometry was performed to estimate percentage of infected cells. The results showed whether prophylactic approach or therapeutic approach the results showed a 20% reduction in rgRSV infected cells in cells treated with siNPRA8 and/or siNPRA9 compared to siU6 control plasmid. Thus these results show that siNPRA treatment can decrease RSV infection in addition to inflammation as seen in other studies.

### Example 14

### Demonstration that siNPRA Treatment Decreases Melanoma Tumor Formation in B16 Mouse Model

Because siNPRA molecules are deliverable by transdermal route and treatment with siNPRA decreases local and systemic inflammation, which has been recently attributed toward the origin of certain cancers, the effect of siNPRA on melanoma was tested. The neoplastic transformation of the melanocyte involves differential ability of the melanoma cell versus the melanocyte to cope with oxidative stress. Melanocytes produce reactive radicals and have a low level of anti-oxidant enzymes, responding to UV with a large but transient increase in superoxide anion whereas keratinocytes and fibroblasts do not. Also, the comparative resting levels of the subunits forming the transcription factor NFkB are altered between melanocytes and melanoma cells both under resting and UVB stimulated conditions (Chin, L et al. Genes Dev 1998, 12(22):3467-348126). Thus, the effect of the role of NPRA in melanoma was investigated.

Materials and Methods.

B16 melanoma cells (1.3×10⁵) were injected subcutaneously into twelve-week old female C57BL/6 mice or NPRA-deficient mice produced in *B6 background. These mice were then treated with 33 μg of siNPRA-oligos siNPRA9 plasmid, or scrambled oligos. All of these were mixed with Chitosan at ratio of 1:2.5. Mixed chitosan and plasmid or oligos were mixed again with cream before application to the injection area. The control group was given cream only. These treatments were given twice a week. Mice were sacrificed on day twenty second, tumors were removed and weighed.

Results.

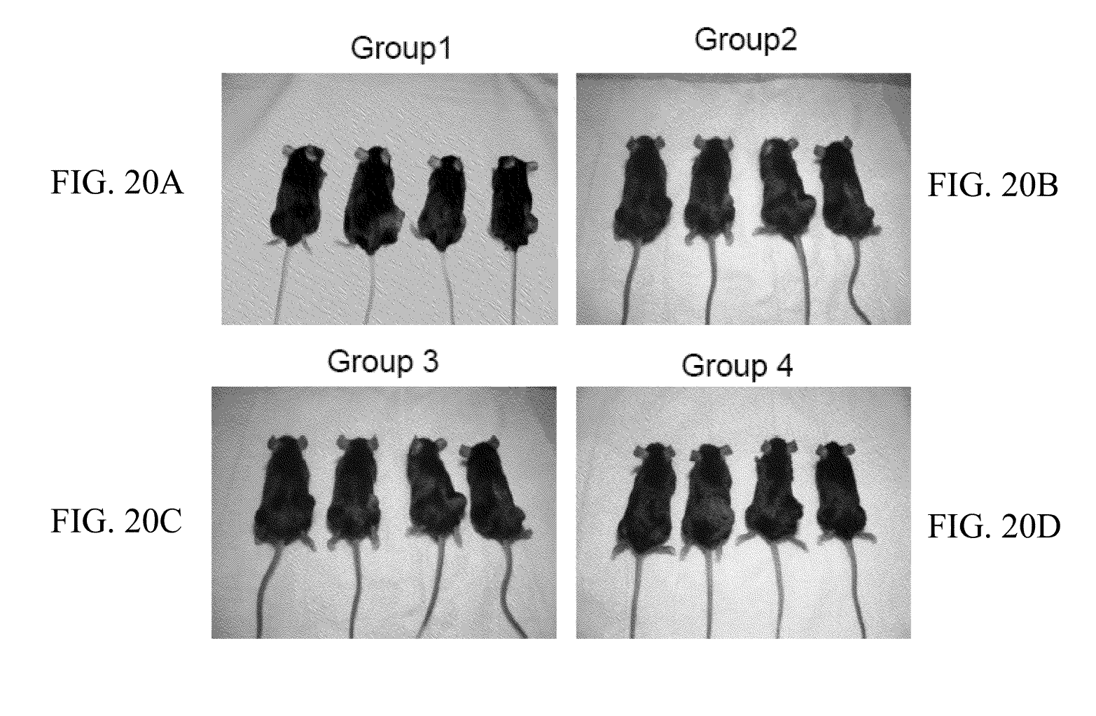

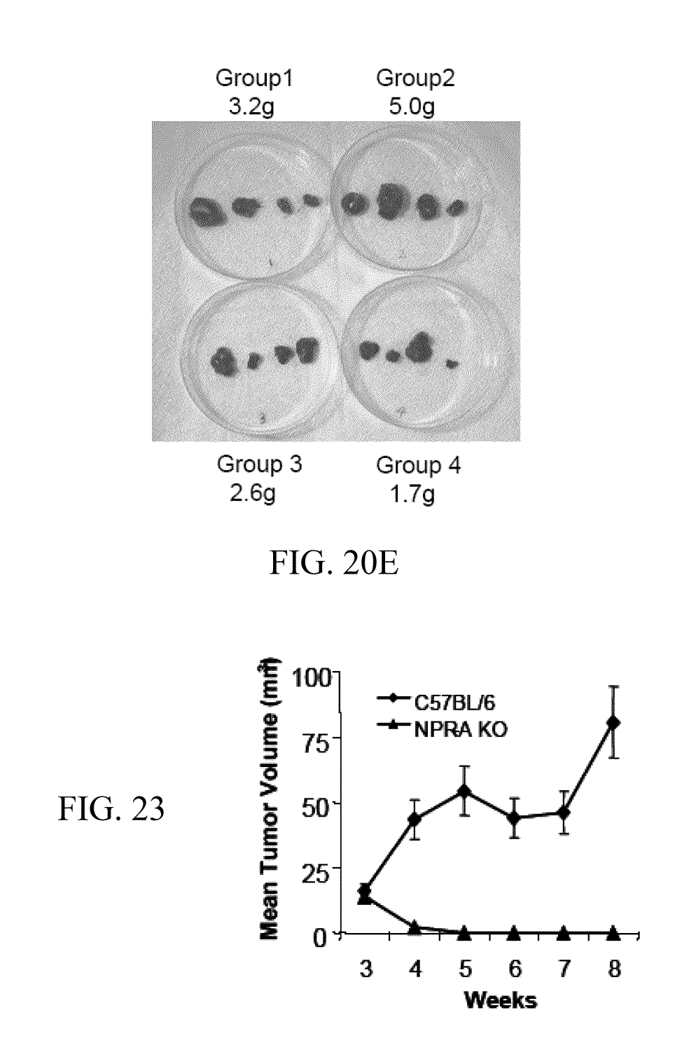

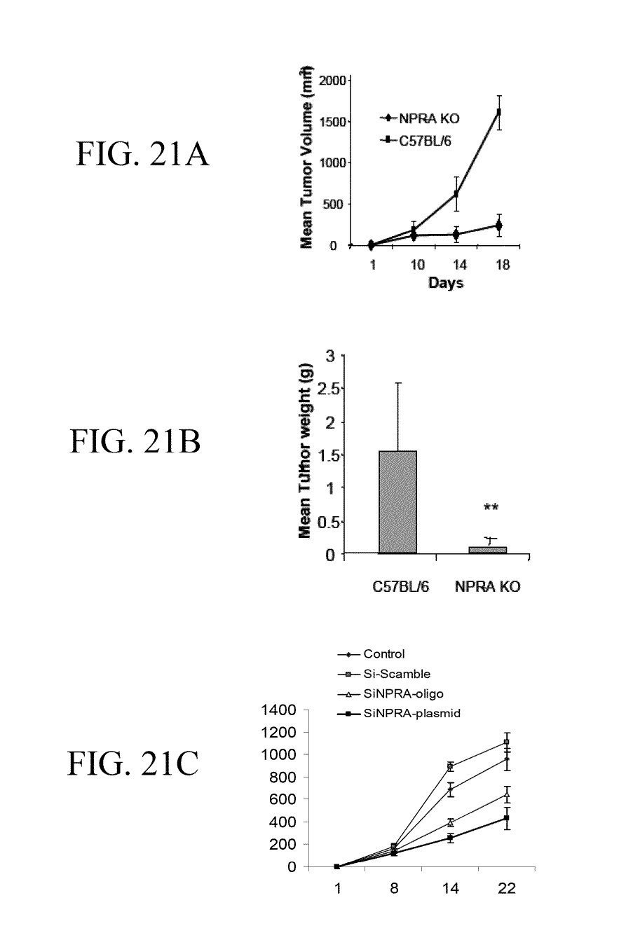

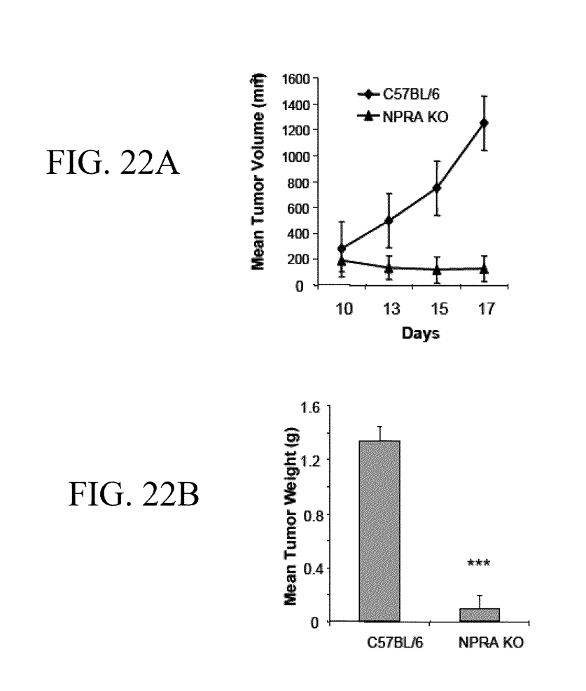

To determine the role of NPRA in melanoma, groups of wild-type (WT) and NPRA^{-/-} mice (n=8) were given subcutaneously 3×10⁵ B16F10.9 cells and the tumor progression was observed after 14 days. The WT mice produced tumors whereas NPRA−/− mice did not have any tumors ANP pathway is a major pathway promoting melanoma tumors in C57BL/6-B16F10. 9 model (FIGS. 20A-20E). To quantify the results, the tumor size and burden were measured in WT and NPRA-mice injected s.c. with B16 melanoma cells. A significant reduction (P<0.01) in mean tumor volume measured over results 18 days after B 16 cell injection and a significant decrease in tumor weight at day 18 was found in NPRA^{-/-} mice (n=12) compared to WT (FIGS. 21A and 21B).

Since, NPRA-deficient mice may have other abnormalities which might make it resistant, the WT mice were injected with 3×10⁵ B16F10.9 cells and were then treated with a cream containing siNPRA 9 given twice a week at the location of tumor cell injection. Three weeks later, both treated and control mice treated with cream alone without siNPRA were compared for their tumor burden. FIG. 21C shows a comparison of both groups of mice. Excision of these tumors revealed that but not siNPRA scrambled, showed significant reductions in tumor burden. These results together show that siNPRA can be used to treat melanomas.

### Example 15

### NPRA Deficiency Decreases Lung Tumor Formation in a Mouse Model

Methods:

For challenge with Lewis lung cancer cells, LLC1 cells grown in DMEM were washed with phosphate buffered saline (PBS) and resuspended in PBS at 2×10⁷ cells per ml. Two groups of mice (n=8 per group) were tested: WT C57BL/6 mice and CS7BL/6 NPRA-deficient mice. Animals were injected subcutaneously with 2×10⁶ LLC1 cells (100 Ill) in the right flank. Tumor sizes were measured at days 10, 13, 15 and 17 after injection. All animals were sacrificed on day 17 and the tumors were removed and weighed.

Results:

Using the Lewis lung carcinoma model, C57BL/6 WT mice and NPRA gene knockout (NPRA^{(-/-)} mice (n=8 for each group) were injected s.c. with 2×10⁶ cells LLC1 cells in the right flank. Tumors appeared within one week after injection and tumor size was measured with a digital caliper beginning on day 10. The tumors in WT mice grew rapidly after day 10, but tumors in NPRA-mice gradually shrank. On day 17, all mice were sacrificed, and tumor sizes and weights were measured. In one of the NPRA-mice there were no visible tumors at all. Significant differences (P<0.001) in tumor size and weight were observed between the two groups

### Example 16

### NPRA Deficiency Decreases Ovarian Tumor Formation in a Mouse Model

Methods:

For challenge with ovarian cancer cells, ID-8 ovarian cancer cells grown in DMEM were washed with PBS and resuspended in PBS at 2×10⁷ cells per ml. Two groups of mice (n=8 per group) were tested: WT C57BL/6 mice and C57BL/6 NPRA-deficient mice. Animals were injected subcutaneously with 2×10⁶ ID8 cells (100 μA) and tumor sizes were measured at days 10, 13, 15 and 17 after injection. All animals were sacrificed on day 17 and the tumors were removed and weighed.

Results:

Groups (n=8) of WT mice and NPRA-deficient C57BL/6 mice were injected 10 with 2×10⁶ ID8 mouse ovarian cancer cells at day 1 and mice were monitored at weekly intervals for tumor growth. By week 8 after cancer cell inoculation, all mice from the WT group developed solid tumors but no tumors were found in NPRA-deficient mice. The results indicate that NPRA deficiency significantly protects mice from ovarian cancer.

### Example 17

### NPRA Deficiency Decreases Lung Inflammation

Materials and Methods.

Cell lines. The mouse Lewis lung carcinoma LLC1 cell line, B16F10.9 melanoma cells, the type II alveolar epithelial adenocarcinoma cell line A549, and the normal human lung fibroblast cell line IMR 90 were purchased from ATCC (Rockville, Md.). Human Prostate cancer cells PC3 and DU145 and mouse ovarian cancer cell line, ID8, were also used. (kindly provided by Dr. Wenlong Bai in the University of South Florida; mouse ovarian cancer cell line, ID8, kindly provided by Dr. Janat-Amsbury at the Baylor College of Medicine.) Both A549 and IMR 90 were grown in Earle's modified Eagle's medium (EMEM) supplemented with 10% fetal bovine serum at 37° C. in a 5% CO₂ incubator. LLC1, ID8 and B16F10.9 cells were grown in Dulbecco's modified Eagle's medium (DMEM) supplemented with 10% fetal bovine serum.

Animals.

Female 8-10 week old BALB/c mice were purchased from Jackson Laboratory (Bar Harbor, Me.). Female nude mice and C57BL/6 mice were from NCI (National Cancer Institute). C57BL/6 NPRA^{-/-} (deficient in natriuretic peptide receptor A) mice were kindly provided by Dr. William Gower (VA Hospital Medical Center, Tampa, Fla.). All mice were maintained in a pathogen-free environment and all procedures were reviewed and approved by the University of South Florida Institutional Animal Care and Use Committee.

Plasmid Constructs and Transfection.

All plasmids used in this study were constructed using the pVAX expression vector (Invitrogen, CA). The pNP73-102 plasmid encodes the natriuretic peptide sequence, amino acids 73 to 102, of the atrial natriuretic prohormone N-terminal fragment. In some experiments the NP73-102 sequence was fused to the FLAG sequence to allow antibody detection of NP73-120 expression in lung sections. An anti-NPRA small interfering RNA plasmid (siNPRA) was constructed as previously described. A549 cells were transfected with plasmids using Lipofectamine 2000 (Invitrogen, CA) according to manufacturer's instructions.

Preparation of Plasmid Nanoparticles and Administration to Mice.

Plasmids pNP73-102 and pVAX1 were encapsulated in chitosan nanoparticles (25 μg of plasmid plus 125 μg of chitosan). Plasmids dissolved in 25 mM Na₂SO₄ and chitosan (Vanson, Redmond, Wash.) dissolved in 25 mM Na acetate (pH 5.4, final concentration 0.02%) were heated separately for 10 min at 55° C. After heating, the chitosan and DNA were mixed, vortexed vigorously for 20-30 sec. and stored at room temperature until use. Plasmid nanoparticles were given to lightly anesthetized mice in the form of nose drops in a volume of 50 μl using a pipetter with the tip inserted into the nostril.

Injection of Mice with Tumor Cells.

For subcutaneous challenge with LLC1, ID8 and B16F10.9 cells, cells were grown in DMEM and washed with PBS and then resuspended in PBS at 2×10⁷ cells per ml for both LLC1 and ID8 or at 3×10⁶ cells per ml for B16F10.9. Two groups of mice (n=8 or 12 per group) were tested: wild type C57BL/6 and C57BL/6 NPRA-deficient mice. Animals were injected subcutaneously with 100 μl of suspended cancer cells in the right flank. Tumor sizes were measured regularly and the tumors were removed and weighed at the end of experiment. For the A549/nude mouse model, two groups of nude mice (n=4 per group) were given 5×10⁶ A549 cells by intravenous injection and treated intranasally with 25 μg of pNP73-102 or pVAX1 control nanoparticles once a week. Three weeks later, mice were sacrificed and lung sections were stained with hematoxylin and eosin and examined for tumor nodules. Lung sections were also stained with antibodies to cyclin B and phospho-Bad.

For the Line-1/BALB/c mouse model, 25 μg of pNP73-102 or pVAX1 control nanoparticles was injected intraperitoneally into two groups of BALB/c mice (n=4 per group) on days 1 and 3. A week later, these mice were injected subcutaneously with 10⁵ Line-1 lung adenocarcinoma cells in the right flanks Additional treatment with pNP73-102 or pVAX1 nanoparticles was continued at weekly intervals from week 2. A third group of four mice received only Line-1 cells as control. In each set of experiments, the mice were sacrificed on day 40 and their tumor burden was determined based on tumor size (measured by digital caliper) and weight.

Western Blots.

A549 cells were harvested and resuspended in lysis buffer containing 50 mM HEPES, 150 mM NaCl, 1 mM EDTA, 1 mM EGTA, 10% glycerol, 0.5% NP-40, 0.1 mM phenylmethylsulfonyl fluoride, 2.5 μg/ml leupeptin, 0.5 mM NaF, and 0.1 mM sodium vanadate to extract whole cell protein. Fifty μg of protein was separated by sodium dodecylsulfate polyacrylamide gel electrophoresis (SDS-PAGE) on a 10% polyacrylamide gel and transferred onto nitrocellulose membranes. Western immunoblots were performed according to the manufacturer's instructions (Cell Signaling Technology). Antibodies against NFκB p65, phosphorylated NFκB p65 (Ser536) and phosphorylated pRb were purchased from Cell Signaling, MA; antibodies against VEGF or NPRA were ordered from Santa Cruz, CA.

Knockdown of NPRA Expression with siNPRA.

Small interfering RNA constructs that targeted the NPRA transcript were prepared and tested for effectiveness by immunoblot for NPRA levels in cells transfected with the psiNPRA plasmid. The siNPRA9 construct was selected for tumorigenesis experiments. B16 melanoma cells (1.5×10⁵) were injected s.c. into twelve-week old female C57BL/6 mice. The mice were then given intranasal suspensions of 33 μg of siNPRA oligos, siNPRA plasmid, or scrambled oligos encapsulated in chitosan nanoparticles at a ratio of 1:2.5. In experiments to determine the efficacy of topical siNPRA, chitosan nanoparticles containing siNPRA plasmid or oligos were mixed with cream and applied to the injection area. Cream containing siNPRA nanoparticles was applied twice a week and the control group received cream only. Mice were sacrificed on day 22 and tumors were removed and weighed.

Apoptosis assays.

A549 or normal IMR90 cells were grown in 6-well plates and transfected with pVAX1 or pNP73-102. Forty-eight hours after transfection, cells were examined for apoptosis by Terminal transferase dUTP nick end labeling (TUNEL) assay, and poly-ADP ribose polymerase (PARP)-cleavage by Western blotting. In the TUNEL assay, cell nuclei were stained with DAPI (diaminopimelimidate) to enable counting of total cell numbers and determination of the percentage of TUNEL-positive cells. For the PARP cleavage, whole-cell protein was isolated and equal amounts were western-blotted using an antibody to PARP. Experiments were done in duplicate.

Statistics.

The number of mice used in each test group was a minimum of 4 and usually 8 or 12. Experiments were repeated at least once and measurements were expressed as means plus or minus standard error of the mean or standard deviation. Comparisons of groups were done using a two-tailed Student's t test.

Results: NPRA Deficiency Decreases Lung Inflammation.

To determine whether the ANP-NPRA pathway contributes to pulmonary inflammation, we compared the lungs of mice deficient in NPRA (NPRA^{-/}) with those of wild type mice following immunization with OVA i.p. and subsequent challenge with OVA intranasally. C57BL/6 wild type mice (n=8) showed substantially higher inflammation, blocked airways and goblet cell metaplasia than did NPRA^{-/-} mice (FIG. 24). Bronchoalveolar lavage (BAL) fluid from NPRA^{-/-} mice had significant reduced levels of the inflammatory cytokines IL-4, IL-5 and IL-6 relative to those in wild type mice (data not shown).

### Example 18

### NPRA Deficiency Protects Mice Against Lung, Skin and Ovarian Cancers

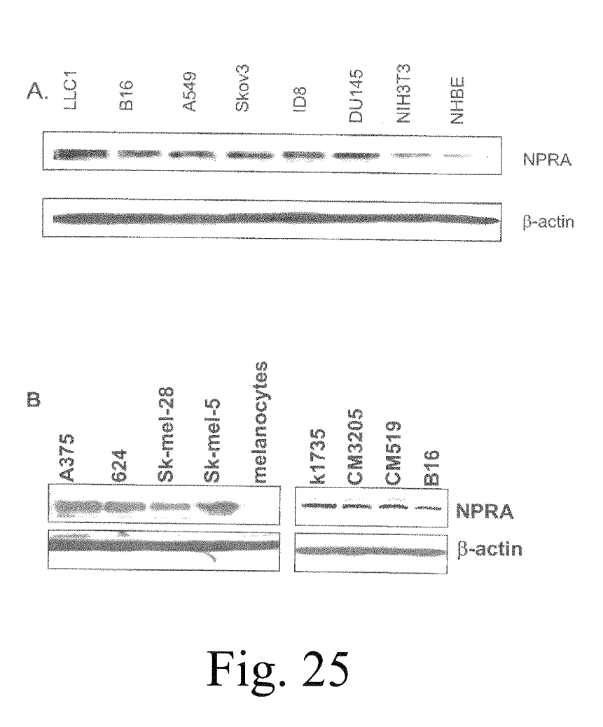

Recent research suggests that alterations in the lung microenvironment caused by inflammation are related to carcinogenesis. (Schwartz A G, Prysak G M, Bock C H, Cote M L. The molecular epidemiology of lung cancer. Carcinogenesis 2007; 28:507-18.) Pro-inflammatory conditions, especially those related to chronic pulmonary irritation, may contribute to the development of lung cancer. (Martey C A, Pollock S J, Turner C K, et al. Cigarette smoke induces cyclooxygenase-2 and microsomal prostaglandin E2 synthase in human lung fibroblasts: implications for lung inflammation and cancer. Am J Physiol Lung Cell Mol Physiol 2004; 287:L981-91.). A direct link between inflammation and lung tumors can be seen in the particle-induced lung cancer murine model (Knaapen A M, Borm P J, Albrecht C, Schins R P. Inhaled particles and lung cancer. Part A: Mechanisms. Int J Cancer 2004; 109:799-809.) Integral to the involvement of inflammation in the development of lung cancer is the profile of cytokines produced. (Arenberg D. Chemokines in the biology of lung cancer. J Thorac Oncol 2006; 1:287-8.). Since ANP-NPRA signaling is involved in lung inflammation, the data presented investigate the role of the ANP-NPRA signaling pathway in the development of cancers of the lung and other organs. To illustrate the role of the ANP-NPRA signaling pathway in cancer development, NPRA expression in various tumor cells and normal cells were compared. NPRA is expressed at a higher level in all tumor cells, including cells of lung carcinoma (A549, LLC1), melanoma (B16), ovarian cancer (SKOV3, ID8) and prostate cancer cells (DU145), compared to that in normal human bronchial epithelial (NHBE) cells (FIG. 25A)

FIGS. 25A-B shows that NPRA is over-expressed in various cancer cells compared to normal cells. All cancer cells used showed increased expression of NPRA and the normal cells showed detectable or barely detectable expression of NPRA. Whole proteins were extracted from different cell lines and subjected to Western blot using primary antibodies against NPRA. Beta ctin is used as a loading control. Cell lines used are as follows. (FIG. 25A) Normal cells: Mouse cell (NIH3T3), Normal human bronchial epithelial cells (NHBE). Cancer cells: LLC-1, Mouse lewis lung carcinoma; A549, human lung adenocarcinoma; B16, mouse melanoma; Skov3, human ovarian cancer, ID8, mouse ovarian cancer cells; DU145, mouse prostate cancer cells and (FIG. 3B) Normal cells, melanocytes; human melanoma cells: A375, 624, Sk-mel-28, Sk-mel-5; and mouse melanoma cells: K1735, CM3205, CM519. NPRA is expressed at a higher level in all tumor cells, including cells of lung carcinoma (A549, LLC1), melanoma (B16, A375, 624, sk-mel-28, sk-mel-5, K1735, CM3205, CM519), ovarian cancer (SKOV3, ID8) and prostate cancer cells (DU145), compared to that in normal human bronchial epithelial (NHBE) cells, NIH3T3 cells and melanocytes.

### Example 19

### Blockade of ANP Signalling has a Protective Effect Against Development of Cancer

To determine whether blockade of ANP signaling could have a protective effect against development of cancer, various C57/BL6 murine models of tumorigenesis were evaluated. Using the Lewis lung carcinoma model, C57BL/6 wild type and NPRA^{-/-} mice (n=8 for each group) were injected s.c. with 2×10⁶ LLC1 cells in the right flank. Tumors appeared within one week after injection, and tumor size was measured with a digital caliper beginning on day 10. The tumors in wild type mice grew rapidly after day 10, but tumors in NPRA^{-/-} mice gradually shrank. On day 17, all mice were sacrificed, and tumor sizes and weights were measured. In one of the NPRA^{-/-} mice, no visible tumors were observed. Significant differences in tumor size and weight were observed between the two groups (FIGS. 25B and 25C). As a further test of the anti-tumor effects of NPRA deficiency, mice were injected s.c. with B16 melanoma cells. A significant reduction in mean tumor volume, measured over 18 d after B16 cell injection, and a significant decrease in tumor weight at day 18 were observed in NPRA^{-/-} mice (n=12) but not in wild type mice (FIGS. 25D and 25E). The potential of NPRA deficiency to inhibit the growth of ovarian cancer cells was also tested. Groups of wild type (n=8) and NPRA-deficient (n=8) C57BL/6 mice were injected with 2×10⁶ ID8 mouse ovarian cancer cells at day 1 and were monitored at weekly intervals for tumor growth. By week 8 after cancer cell inoculation, all mice from the wild type group developed solid tumors, but no tumors were observed in NPRA-deficient mice (FIG. 25F). Again NPRA^{-/-} mice exhibited a significant reduction in ovarian cancer development compared to that in wild type mice. These results indicate that NPRA deficiency significantly protects mice from tumorigenesis and tumor progression.

### Example 20

### Inhibition of Melanoma by siNPRA Nanoparticles

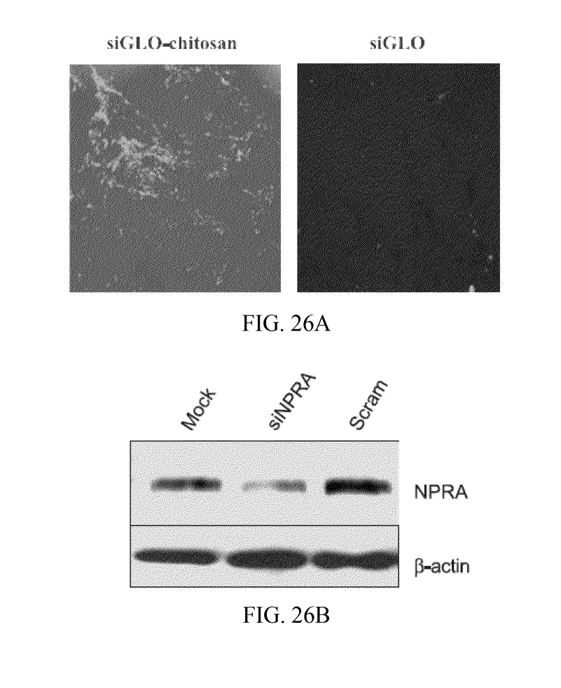

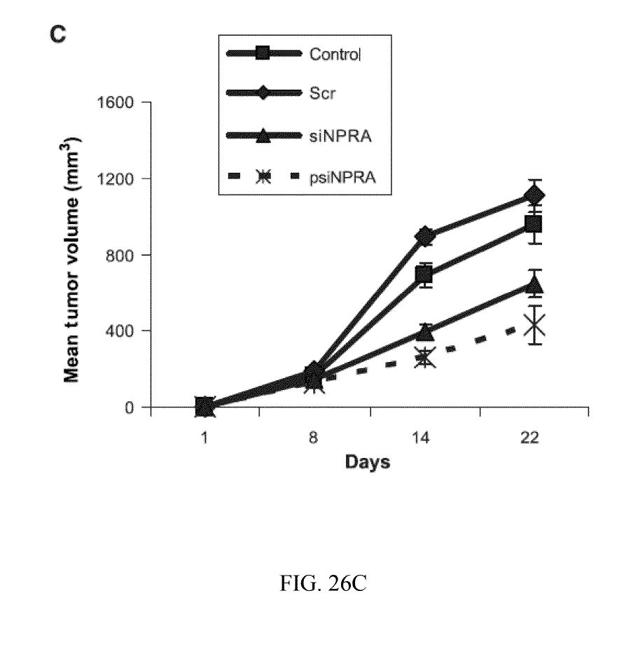

siRNA was used to knock down NPRA expression C57BL/6 mice and tested their ability to inoculate B16 melanoma cells. To test whether nanoparticle-mediated siRNA transfer could be utilized for this purpose, chitosan-siGLO nanocomplexes was intratumorally injected into the PC3-induced prostate tumors in BALB/c nude mice and siGLO was examined 48 h after injection. Fluorescence microscopy revealed that siGLO was only present in tumors when delivered in nanocomplexes but not when delivered in naked form (FIG. 26A). To identify the most effective siRNA, several candidates were screened and identified three that inhibited NPRA expression. (siNPRA 8, 9, and 10 as previously described) HEK293-GCA cells that overexpress NPRA were transfected with one of these siNPRAs or with scrambled siNPRA, and cell lysates were examined at 48 h for NPRA expression by western blotting. As shown in FIG. 26B, siNPRA9 decreased NPRA expression by about 60%. Since NPRA-deficient C57BL/6 mice may have abnormalities that make them resistant to tumor development, wild type mice were injected with 3×10⁵ B 16F10.9 melanoma cells and were then treated twice a week with a cream containing either synthetic siNPRA, Vector driven siNPRA (psiNPRA) or scrambled siNPRA (Scr), respectively, for four consecutive weeks at the site of tumor cell injection. Four weeks later, tumor burden from each group was compared. A significant reductions in tumor growth was observed in mice treated with siNPRA 9 (either with synthetic or vector-driven siNPRA), but not those given scrambled siNPRA (FIG. 26C), indicating that siNPRA can be used to treat melanomas.

Because siNPRA molecules are deliverable by transdermal route and treatment with siNPRA decreases local and systemic inflammation, which has been recently attributed toward the origin of certain cancers, the effect of siNPRA on melanoma was tested. The neoplastic transformation of the melanocyte involves differential ability of the melanoma cell versus the melanocyte to cope with oxidative stress. Melanocytes produce reactive radicals and have a low level of anti-oxidant enzymes, responding to UV with a large but transient increase in superoxide anion whereas keratinocytes and fibroblasts do not. Also, the comparative resting levels of the subunits forming the transcription factor NFkB are altered between melanocytes and melanoma cells both under resting and UVB stimulated conditions (Chin, L et al. Genes Dev 1998, 12(22):3467-348126). Thus, the effect of the role of NPRA in melanoma was investigated.

Materials and Methods.

B 16F10 melanoma cells (1.3×10⁵) were injected subcutaneously into twelve-week old female C57BL/6 mice. These mice were then treated with 33 μg of siNPRA9-oligo, siNPRA9 plasmid, or scrambled oligos. All of these were mixed with Chitosan at ratio of 1:2.5. Mixed chitosan and plasmid or oligos were mixed again with cream, before application to the injection area. The control group was given cream only. These treatments were given twice a week. Mice were sacrificed on day twenty second, tumors were removed and weighed.

Results.

To determine the function of siNPRA9, HEK293GCA cells were transfected with siNPRA9 or scrambled siRNA and 24 h after transfection the cell lysate was examined for NPRA expression. The results showed that siNPRA inhibited the NPRA expression as detected by western blot. Beta-actin ws used as control (FIG. 26B).

To determine the in vivo effects of siNPRA9, groups C57B1/6 mice of (n=16) were injected with 3×10⁵ B16F10.9 cells and then treated with a cream containing siNPRA9 given twice a week at the location of tumor cell injection. Three weeks later, both treated and control mice treated with cream alone without siNPRA9 were compared for their tumor burden. FIG. 26C shows a comparison of both groups of mice. Comparison of tumor burden from different groups revealed that siNPRA9, but not siNPRA scrambled, showed significant reductions (p<0.01) in tumor burden compared to control. These results show that siNPRA can be used to treat melanomas.

### Example 21

### Suppression of Lung Cancer Tumorigenesis by NP73-102 Nanoparticles

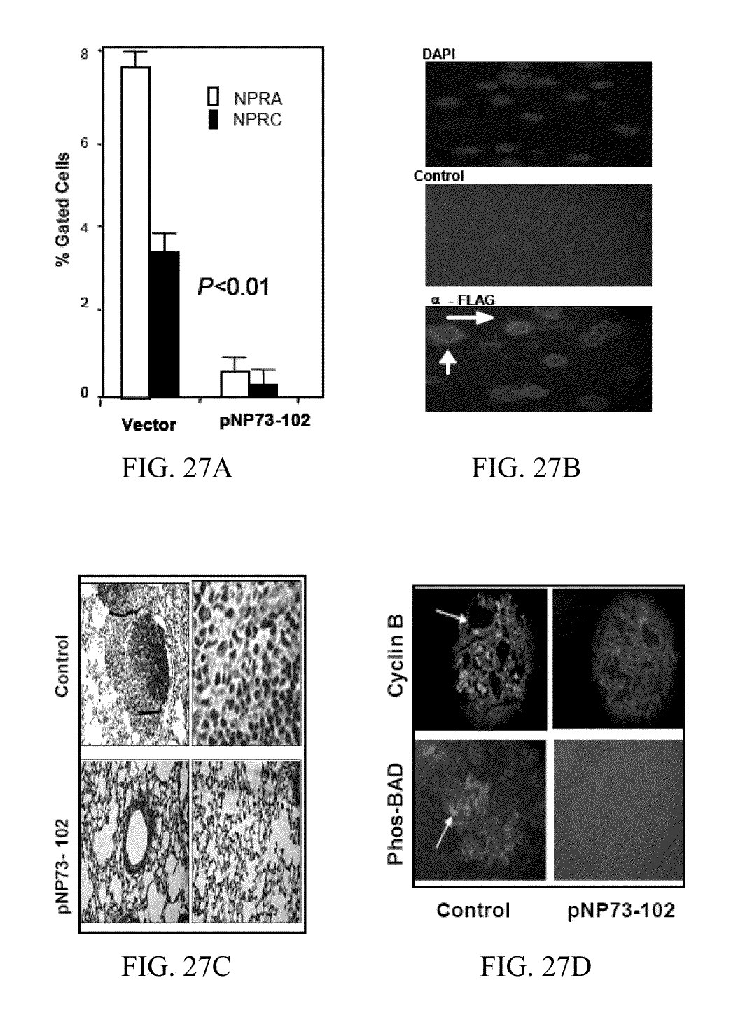

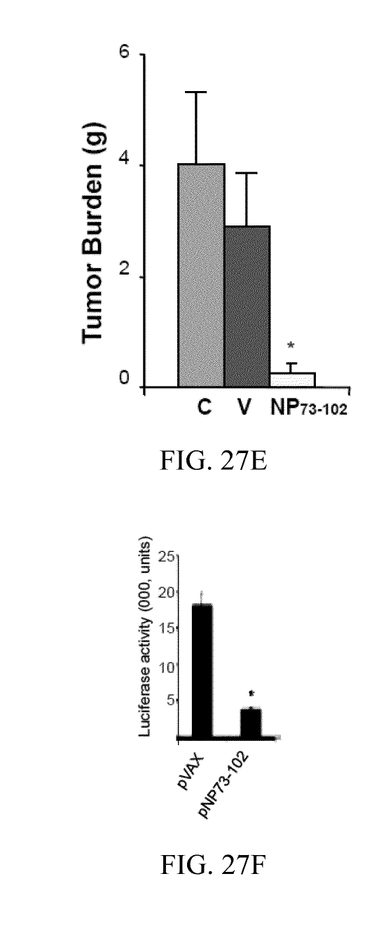

NP73-102 decreases activation of several transcription factors, including NFκB which promote tumorigenesis. To test whether over expression of NP73-102 affects NPRA expression in vivo, pregnant mice were injected i.p. with pNP73-102 or pVAX1. After 3-5 days, mice were sacrificed, and thymocytes were isolated from embryos. NPRA or NPRC levels were quantitated by flow cytometry with gating on CD4+ cells. Expression of both NPRA and NPRC in embryonic thymi was significantly reduced by pNP73-102 when compared to that in control mice injected with pVAX1 (FIG. 27A). Because NPRA-deficient mice had reduced tumorigenicity, it was reasoned that NP73-102 might have anti-tumor activity, and this was evaluated in vitro in A549 cells using a soft agar assay. A549 cells were transfected with pVAX1, pANP or pNP73-102. The results from the soft agar assay (data not shown) indicated that cells transfected with pNP73-102 exhibited significantly decreased colony formation compared to that of nontransfected cells or cells transfected with pVAX1. To test whether over-expression of a plasmid DNA encoding NP73-102 could express the peptide in vivo in the lung, a pNP73-102-FLAG was constructed, in which NP73-102 was fused to a FLAG epitope to verify expression of NP73-102 in lung cells. The pNP73-102-FLAG, encapsulated in chitosan nanoparticles, was administered to mice intranasally, and 24 hr later, a bronchoalveolar lavage (BAL) was performed. BAL cells were stained with anti-FLAG antibody and substantial numbers of cells expressing NP73-102-FLAG were observed (FIG. 27B).

To determine whether intranasal NP73-102 nanoparticle administration abrogates metastasis in mice, 12 nude mice were separated into three groups (n=4 per group). Mice were given 5×10⁶ A549 cells intravenously and weekly instillations of PBS (control) or nanoparticles carrying pNP73-102 or pVAX1. Three weeks later, mice were sacrificed and lung sections were stained with hematoxylin and eosin and examined for lung nodules. Control animals receiving only PBS showed nodules and tumors, while the NP73-102-treated group had no tumors (FIG. 27C). Additionally, the lung sections were stained with antibodies to pro-mitotic cyclin B and to anti-apoptotic phospho-Bad (biomarkers of lung tumors), and mice treated with NP73-102 did not show any staining for cyclin-B or phospho-Bad (FIG. 27D). To test whether NP73-102 nanoparticles could attenuate tumor burden in an immunocompetent mouse lung cancer model, BALB/c mice (4-6 week old, female, n=3 to 4 per group) were given pNP73-102 (25 μg/mouse, i.p.) on days 1 and 3 and then s.c. injected with 10⁵ Line-1 cells in the right flank on day 7. Thereafter, mice were i.p. injected with pNP73-102 nanoparticles at weekly intervals. The mice were sacrificed on day 40 and the size and weight of tumors was measured. The results show that the tumor burden in pNP73-102-treated mice was significantly reduced compared to the tumor burden in those treated with PBS or pVAX1 control vector (FIG. 27E).

The highest expression of the ANP and ANP receptors is found in neonatal thymus. To test whether the peptide NP73-102 inhibits in vivo the ANP cascade, pregnant (12 days) mice were injected i.p. with pVAX (vector), or pNP73-102. After 1 day, mice were sacrificed and thymi removed from embryo, were homogenized. Cells were centrifuged and erythrocytes lysed by treating the suspension with ACK buffer. Cells were incubated with anti-NPRA or anti-NPRC antibodies for 1 hour, washed and incubated with PE-conjugated 20 Ab. Levels of NPR's were determined by flow cytometry. The results are shown in FIG. 27A. The results demonstrate that pNP73-102 inhibited expression of NPRA in thymocytes. Although the mechanism is not clear, this may be due to feedback inhibition at the level intracellular signaling occurring via NPRA.

To further show whether pNP73-102 downregulates the expression of NPRA gene, a reporter plasmid with NPRA promoter linked to Luciferase was used. HEK293 cells were cotransfected with pNPRA-Luc and pNP73-102 or pVAX1. Forty-eight hrs later, cells were harvested and lyzed with luciferase reporter lysis buffer. The supernatants were subjected to luciferase assay (*p<0.05, **p<0.01) (FIG. 27B). The results demonstrate that pNP73-102 significantly downregulates NPRA promoter activity.

To determine the effect of over expression of NP73-102 on proliferation of A549 lung epithelial cells, cells were transfected with either pNP73-102 or vector, pVAX. Cell cycle analysis was performed using propidium iodide (PI) staining and flow cytometry 48 h after transfection. No significant difference was observed between control and pNP73-102-transfected cells in S1, Go-G1 and G2-M stages of cell cycle (data not shown). However, an analysis of apoptosis using flow-cytometry with PI and annexin V, showed that cells transfected with pNP73-102 exhibited significantly higher apoptosis compared to cells transfected with either the control plasmid or a plasmid encoding ANP (FIG. 7A). A significantly higher apoptosis is seen in A549 adenocarcinoma cells compared to normal IMR-90 cells, as shown by TUNEL assay of A549 cells cultured in 8-chamber slide following a 48-hour transfection with either pANP or pNP73-102 (FIG. 7B) and by analysis of PARP cleavage in these cells 48 hours after transfection, which was significantly more prominent in pNP73-102 transfected cells compared to pANP or pVAX transfected cells (FIG. 7C). The results show that pNP73-102 shows a higher accumulation of apoptotic cells compared to cells transfected with pANP and pVAX controls. Thus, pNP73-102 induces apoptosis of lung adenocarcinoma cells.

In an effort to identify and characterize molecules participating in early signaling pathways, differential gene expression was analyzed using a microarray (AFFYMETRIX). Altered expression of a large number of genes was found, including genes related to cell growth, cell cycle, and apoptosis. These genes included, among others more than, 6- to 8-fold up-regulation of genes such as Caspase (Casp)-8 and FADD like apoptosis regulator, cyclin E binding protein, CDK inhibitor 1A, CDK7, casp4, casp-10, casp-1, apoptosis facilitator BCL2-like 13 and annexin 43 (data not shown). Together, these studies indicate that pNP73-102 is an inducer of apoptosis in A549 lung adenocarcinoma cells.

### Example 22

### NP73-102 Induces Apoptosis of A549 Adenocarcinoma and B16 Melanoma Cells

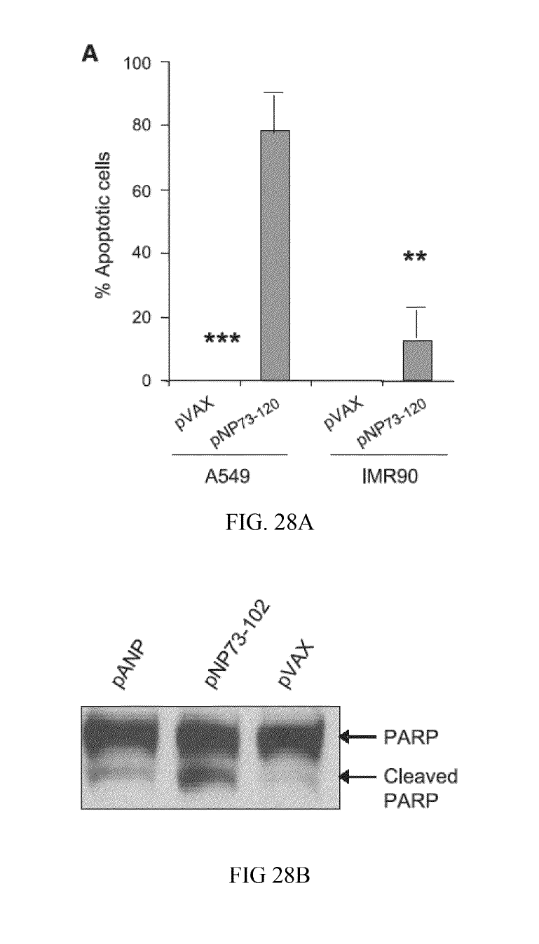

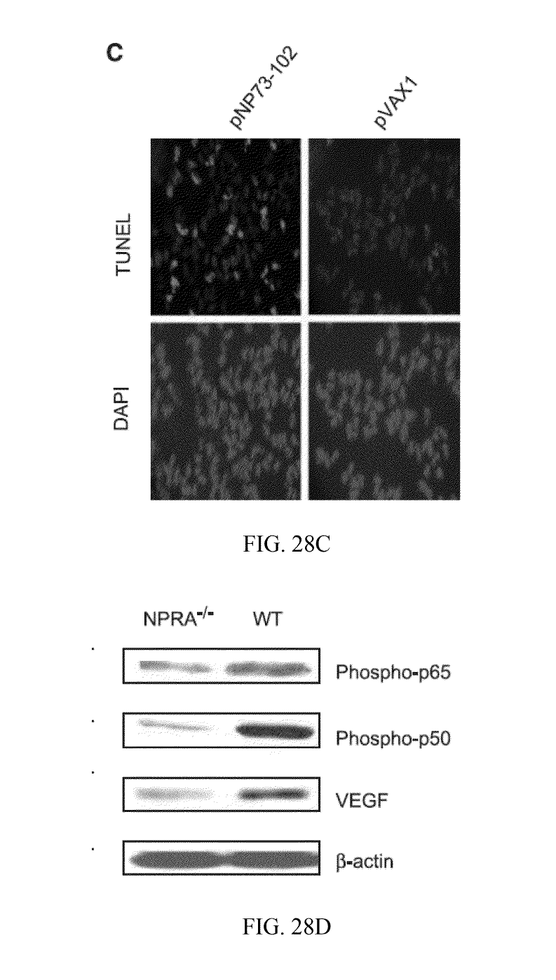

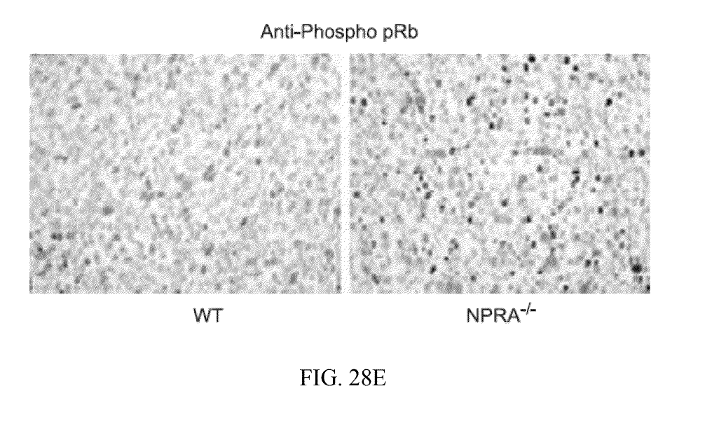

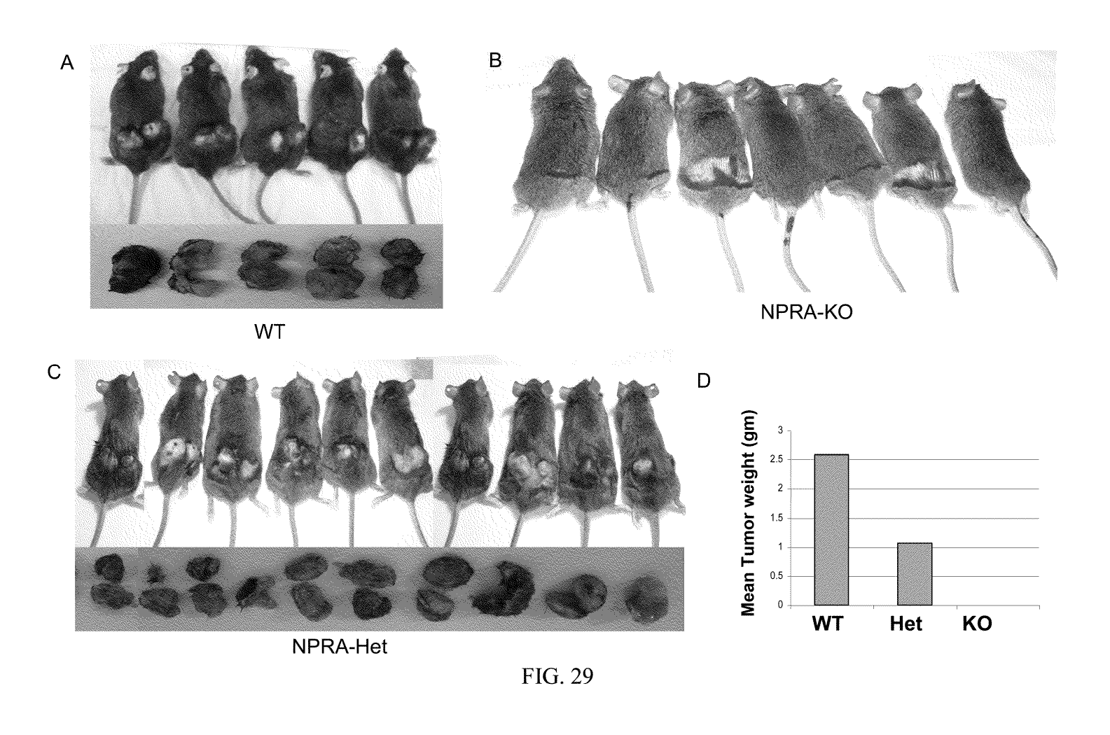

To verify whether anti-tumor effects of pNP73-102 can be attributed to loss of cell viability, A549 and normal WI-138 cells were examined for apoptosis by TUNEL assay following 24 h of transfection. The results indicated that approximately 80% of A549 cells transfected with pNP73-102 underwent apoptosis compared to only 10% of WI-138 cells (FIG. 28A). In addition, more A549 cells were observed to be TUNEL-positive when treated with pNP73-102 than were observed among cells treated with pVAX1 (not shown). Apoptosis was further confirmed by examining for the cleavage of the caspase 3 substrates, PARP, by Western blotting. A549 cells transfected with pNP73-102 showed more cleaved PARP than controls (FIG. 28B). A microarray analysis of gene expression of A549 cells following transfection with either pVAX1 or pNP73-102 was performed. The results showed that pNP73-102 significantly altered, both positively and negatively, the expression of a number of genes (data not shown). The upregulated genes were predominantly from the family of IFN-regulated genes or related signal transduction pathways. Similarly, the down regulated genes included some involved in inflammation, suggesting that NP73-102 has anti-inflammatory, in addition to anti-tumor, properties. To determine whether apoptosis induction was the dominant explanation for the anti-tumor activity of pNP73-102, we tested the effect of over expressing pNP73-102 in B16 melanoma and normal NIH3T3 cells. The results showed significant apoptosis of B16 cells as measured by flow cytometry assay but not of the normal cells (data not shown). Also, significantly more B16 cells were observed to be TUNEL-positive when they were treated with pNP73-102 compared to the number observed among cells treated with pVAX1 (FIG. 28C). These results indicated that a decrease in ANP-NPRA signaling may result in the induction of apoptosis in cancer cells but not in normal cells. Activation of the NFkB pathway enhances tumor development and may act primarily during the late stages of tumorigenesis. To determine whether the lungs of NPRA^{-/-} mice differ in NFκB activation when compared to wild type mice, we examined the lung extracts for signs of NFkB activation by Western blotting. Whole proteins were extracted from the lungs of wild type and NPRA^{-/-} mice and then probed using primary antibodies against p50, p65, and phospho-p50, -p65. No significant difference in NFκB expression in the lungs was observed between wild type and NPRA^{-/-} mice (FIG. 28A). However, the level of the activated form of NFκB, phospho-NFκB (both phospho-p65 and phospho-p50), was decreased in NPRA^{-/-} mice (FIG. 2A). Accordingly, NPRA's role in lung inflammation may involve NFκB activation.

pRb, the protein product of the retinoblastoma cancer suppressor gene, was then tested in order to determine its role in the suppression of tumor growth in NPRA^{-/-} mice. pRb and other retinoblastoma family members, such as pRb2/p130 and p107, are involved in controlling four major cellular processes of growth arrest, apoptosis, differentiation and angiogenesis. Inactivation of pRb has been demonstrated to play an important role in the pathogenesis of human cancers. The expression of pRb in the lungs of wild type C57BL/6 and NPRA^{-/-} mice by immunohistochemistry analysis was then compared. It was revealed that NPRA deficiency induced over expression of pRb (FIG. 28D). In addition, expression of vascular endothelial growth factor (VEGF), which is important in angiogenesis, was decreased in the lungs of NPRA-deficient mice, as observed by Western blotting (FIG. 28E). The differential expression of pRb and VEGF may show why several types of cancer were inhibited in NPRA^{-/-} mice but not in wild type mice. The expression of another major tumor suppressor gene, p53, was also compared in the lungs of wild type and NPRA^{-/-} mice by Western blot analysis and no significant difference was observed (data not shown).

Other mechanistic studies were performed to understand why lung tumor growth was inhibited in NPRA^{-/-} mice by comparing gene expression in the lungs of wild type and NPRA^{-/-} mice. Super array analysis revealed that the expression of several genes, such as hexokinase 2, glycogen synthase 1, and matrix metallopeptidase 10 were down regulated about 4-17-fold in the lungs of NPRA^{-/-} mice. Interestingly, the expression of cellular retinol binding protein 1 (CRBP-I) was upregulated about 5.5-fold in the lungs of NPRA^{-/-} mice. A significant finding of these studies is the demonstration that signaling through NPRA, which is the receptor for ANP and BNP, plays a pivotal role in tumorigenesis.

### Example 23

### NFκB and PRB are Involved in Tumor Suppression in NPRA-Deficient Mice

Activation of the NFkB pathway enhances tumor development and may act primarily during the late stages of tumorigenesis. To determine whether the lungs of NPRA^{-/-} mice differ in NFκB activation when compared to wild type mice, we examined the lung extracts for signs of NFkB activation though Western blot. Whole proteins were extracted from the lungs of wild type and NPRA^{-/-} mice and then probed using primary antibodies against p50, p65, and phospho-p50, -p65. No significant difference in NFκB expression in the lungs was observed between wild type and NPRA^{-/-} mice (FIG. 28D). However, the level of the activated form of NFκB, phospho-NFκB (both phospho-p65 and phospho-p50), was decreased in NPRA^{-/-} mice (FIG. 28D). Accordingly, NPRA's role in lung inflammation may involve NFκB activation.

pRb, the protein product of the retinoblastoma cancer suppressor gene, was then tested in order to determine its role in the suppression of tumor growth in NPRA^{-/-} mice. pRb and other retinoblastoma family members, such as pRb2/p130 and p107, are involved in controlling four major cellular processes of growth arrest, apoptosis, differentiation and angiogenesis. Inactivation of pRb has been demonstrated to play an important role in the pathogenesis of human cancers. The expression of pRb in the lungs of wild type C57BL/6 and NPRA^{-/-} mice by immunohistochemistry analysis was then compared. It was revealed that NPRA deficiency induced over expression of pRb (FIG. 28E). In addition, expression of vascular endothelial growth factor (VEGF), which is important in angiogenesis, was decreased in the lungs of NPRA-deficient mice, as observed by Western blotting (FIG. 28D). The differential expression of pRb and VEGF may show why several types of cancer were inhibited in NPRA^{-/-} mice but not in wild type mice. The expression of another major tumor suppressor gene, p53, was also compared in the lungs of wild type and NPRA^{-/-} mice through Western blot analysis and no significant difference was observed (data not shown).

Other mechanistic studies were performed to understand why lung tumor growth was inhibited in NPRA^{-/-} mice by comparing gene expression in the lungs of wild type and NPRA^{-/-} mice. Super array analysis revealed that the expression of several genes, such as hexokinase 2, glycogen synthase 1, and matrix metallopeptidase 10 were down regulated about 4-17-fold in the lungs of NPRA^{-/-} mice. Interestingly, the expression of cellular retinol binding protein 1 (CRBP-I) was upregulated about 5.5-fold in the lungs of NPRA^{-/-} mice. A significant finding of these studies is the demonstration that signaling through NPRA, which is the receptor for ANP and BNP, plays a pivotal role in tumorigenesis. As a key signaling molecule, NPRA produces the second messenger cGMP and activates cGMP-dependent protein kinase (PKG). PKG activation in turn activates ion transporters and transcription factors, which together affect cell growth and proliferation, apoptosis, and inflammation. The finding that NPRA^{-/-} mice showed reduced lung inflammation indicates that ANP-NPRA signaling is involved in the inflammatory process. These data are supported by an observed decrease in eosinophil numbers and in Th1-like and Th2-like cytokines in BAL fluid from NPRA^{-/-} mice compared to levels in wild type mice (data not shown). These results demonstrate that ANP-NPRA signaling promotes inflammation in rodent models.

To test the hypothesis that the increased inflammation contributes to the genesis of cancer, three different cancer models were investigated in C57BL/6 wild type mice and NPRA^{-/-} mice, as previously described. These include the Lewis-lung carcinoma model, the B16-induced melanoma model and the ID8-induced spontaneous model for ovarian cancer. In all these models, the NPRA^{-/-} mice showed little or no tumor growth compared to wild type mice. ANP was reported to possess anti-cancer properties (See Vesely D L. Atrial natriuretic peptides: anticancer agents. J Investig Med 2005; 53:360-5.) and our data are consistent with this, since ANP over expression is known to decrease NPRA levels in cells (See Pandey K N, Nguyen H T, Sharma G D, Shi S J, Kriegel A M. Ligand-regulated internalization, trafficking, and down-regulation of guanylyl cyclase/atrial natriuretic peptide receptor-A in human embryonic kidney 293 cells. J Biol Chem 2002; 277:4618-27.) by feedback inhibition, in one example. Natriuretic peptides, such as KP and VD (Sun Y, Eichelbaum E J, Wang H, Vesely D L. Atrial natriuretic peptide and long acting natriuretic peptide inhibit ERK 1/2 in prostate cancer cells. Anticancer Res 2006; 26:4143-8.); (Sun Y, Eichelbaum E J, Wang H, Vesely D L. Vessel dilator and kaliuretic peptide inhibit ERK 1/2 activation in human prostate cancer cells. Anticancer Res 2006; 26:3217-22.) have also been reported to inhibit cancer cell proliferation and have shown anticancer activities, although the mechanism of their inhibition is not known. Since these peptides down regulate NPRA expression also, those peptides may also function by regulating NPRA signaling, therefore, NPRA, accordingly, is a target for cancer treatment.

To further validate NPRA as a drug target for cancer therapy, siRNA was used to knock down NPRA expression in immunocompetent C57BL/6 mice. Plasmids were designed that induce degradation of NPRA transcripts and block expression of NPRA. To protect the siNPRA plasmid from degradation and to facilitate its entry into tumor cells, the DNA was complexed with chitosan nanoparticles, and this represents a significant improvement in the delivery of siRNA to tumor cells. In a B 16 melanoma model, mice treated with siNPRA nanoparticles showed a significant reduction in tumors compared to those of mice given scrambled siNPRA as a control. To further test this approach, siNPRA was used to treat mice injected with ovarian cancer cells. Again, the growth of the tumor xenograft was inhibited significantly in these mice (not shown). Treatment with siNPRA however was not as complete as seen in NPRA−/− mice; this could be because siRNA knockdown was not complete or that a large enough dose of siNPRA was not used. Nonetheless, NPRA inhibitors may be used as an anti-cancer agent.

The finding that pNP73-102 inhibits NPRA expression led to examination of its role in treating lung cancer using chitosan nanoparticle-based intranasal gene therapy. A549 cells injected into BALB/c nude mice induced lung micrometastasis in the control mice but not in pNP73-102-treated mice. The location of the lung tumors, as indicated by cyclin-B and phospho-BAD biomarkers, was in agreement with the tissue staining data. In addition, tests of spontaneous lung tumorigenesis induced with Line-1 cells in immunocompetent BALB/c mice showed that treatment with pNP73-102 significantly reduced tumors compared to those observed after treatment with pVAX vector alone. These findings confirm the potential utility of pNP73-102 for the treatment of lung cancers. Though the mechanism of tumor inhibition by NP73-102 is unknown, the evidence that pNP73-102 decreases significantly the expression of NPRA serves as an explanation for its anti-tumor effect.

Localized inflammation involving pro-inflammatory transcription factors such as NFκB has been implicated in the development of cancers. (Karin M. Mitogen activated protein kinases as targets for development of novel anti-inflammatory drugs Ann Rheum Dis 2004; 63 Suppl 2:ii 62-64)

Several groups have reported in mouse models of intestinal (Greten F R, Eckmann L, Greten T F, et al. IKK beta links inflammation and tumorigenesis in a mouse model of colitis-associated cancer. Cell 2004; 118:285-96.); liver (Pikarsky E, Porat R M, Stein I, et al. NF-kappaB functions as a tumour promoter in inflammation-associated cancer. Nature 2004; 431:461-6) and mammary cancer that activation of the NFκB pathway enhances tumor development and may act primarily in the late stages of tumorigenesis. (Massion P P, Carbone D P. The molecular basis of lung cancer: molecular abnormalities and therapeutic implications. Respir Res 2003; 4:12.)

Many tumor cell lines show constitutive activation of NFκB, but there has been conflicting evidence as to whether it promotes or inhibits tumorigenesis. Several groups have reported that activation of the NFκB pathway enhances tumor development and may act primarily in the late stages of tumorigenesis in mouse models of intestinal, liver and mammary cancer. Inhibition of NFκB signaling uniformly suppressed tumor development but, depending on the model studied, this salutary effect was attributed to an increase in tumor cell apoptosis, reduced expression of tumor cell growth factors supplied by surrounding stromal cells, or abrogation of a tumor cell dedifferentiation program that is critical for tumor invasion/metastasis (Ahn K S, Sethi G, Aggarwal B B. Simvastatin potentiates TNF-alpha-induced apoptosis through the down-regulation of NF-kappaB-dependent antiapoptotic gene products: role of IkappaBalpha kinase and TGF-beta-activated kinase-1. J Immunol 2007; 178:2507-16; Ashworth T, Roy A L. Cutting Edge: TFII-I controls B cell proliferation via regulating NF-kappaB. J Immunol 2007; 178:2631-5; Inoue J, Gohda J, Akiyama T, Semba K. NF-kappaB activation in development and progression of cancer. Cancer Sci 2007; 98:268-74; Kim S, Millet I, Kim H S, Kim J Y, et al. NF-kappa B prevents beta cell death and autoimmune diabetes in NOD mice. Proc Natl Acad Sci USA 2007; 104:1913-8; Oka D, Nishimura K, Shiba M, et al. Sesquiterpene lactone parthenolide suppresses tumor growth in a xenograft model of renal cell carcinoma by inhibiting the activation of NF-kappaB. Int J Cancer 2007; 120:2576-81; Saccani A, Schioppa T, Porta C, et al. p50 nuclear factor-kappaB over expression in tumor-associated macrophages inhibits M1 inflammatory responses and antitumor resistance. Cancer Res 2006; 66:11432-40; Vilimas T, Mascarenhas J, Palomero T, et al. Targeting the NF-kappaB signaling pathway in Notch1-induced T-cell leukemia. Nat Med 2007; 13:70-7; Schmidt D, Textor B, Pein O T, et al. Critical role for NF-kappaB-induced JunB in VEGF regulation and tumor angiogenesis. Embo J 2007; 26:710-9.).

The demonstration that pNP73-102 inhibited activation of NFκB and that NFκB activation was reduced in the lungs of NPRA^{-/-} mice may represent another additional mechanism underlying its anti-cancer activity. Moreover, we observed less lung inflammation in NPRA^{-/-} mice than was observed in wild type counterparts when they were challenged by OVA in an asthma model. The results presented here provide evidence of a critical role for natriuretic peptides and NPRA signaling in many different cancers, including lung cancer, ovarian cancer and melanoma. Interestingly, NFκB binding activity was 4-fold greater in the nuclear extracts of NPRA^{-/-} mouse hearts than in those of wild type mouse hearts (See Vellaichamy E, Sommana NK, Pandey K N. Reduced cGMP signaling activates NF-kappaB in hypertrophied hearts of mice lacking natriuretic peptide receptor-A. Biochem Biophys Res Commun 2005; 327:106-11.)

Reduced inflammation was also reported in the hearts of NPRA^{-/-} mice (Oliveira A M, Ross J S, Fletcher J A. Tumor suppressor genes in breast cancer: the gatekeepers and the caretakers. Am J Clin Pathol 2005; 124 Suppl: S16-28.).

In order to identify the mechanism by which NPRA deficiency suppresses the growth of several types of tumors, the expression of tumor suppressor genes, including p53 and pRb were analyzed. Tumor suppressor genes participate in a variety of critical and highly conserved cell functions, including regulation of the cell cycle and apoptosis, differentiation, surveillance of genomic integrity and repair of DNA errors, signal transduction, and cell adhesion.

The p53 gene is the best known, but other tumor suppressor genes of interest include the retinoblastoma gene (pRb), PTEN, p16, nm23, and maspin (Oliveira A M, Ross J S, Fletcher J A. Tumor suppressor genes in breast cancer: the gatekeepers and the caretakers. Am J Clin Pathol 2005; 124 Suppl: S16-28.).

There was no significant difference in the level of p53 in the lungs of NPRA^{-/-} and wild type mice. However, the phosphorylation of pRb was upregulated in the lungs of NPRA^{-/-} mice, as indicated by Western blot assays. pRb plays a critical role in the control of cell proliferation and in DNA damage checkpoints and inhibits cell cycle progression through interactions with the E2F family of transcription factors. In tumorigenesis, loss of Rb function is an important event caused by gene mutation, promoter hypermethylation, deregulation of Rb phosphorylation and viral protein sequestration. Dysfunctional pRb has been reported in many different types of tumors, including those of the eye, bone, lung, breast and genitourinary system. In our investigation, we found that NPRA deficiency did not affect pRb expression but did upregulate pRb phosphorylation.

The Rb gene family is also involved in tumor angiogenesis (See Gabellini C, Del Bufalo D, Zupi G. Involvement of RB gene family in tumor angiogenesis. Oncogene 2006; 25:5326-32.). Angiogenesis represents a fundamental step in tumor progression and metastasis. The induction of vasculature is important for tumor growth because it ensures an adequate supply of oxygen and metabolites to the tumor. pRb regulates the expression of pro- and anti-angiogenic factors, such as the vascular endothelial growth factor (VEGF), through an E2F-dependent mechanism. Some natural and synthetic compounds demonstrate their anti-angiogenic activity through a mechanism of action involving pRb. Consistent with the activation of pRb in the lungs of NPRA^{-/-} mice, the expression of VEGF was down regulated in NPRA^{-/-} mice when compared to that in wild type mice. This indicated that angiogenesis was attenuated in NPRA^{-/-} mice, which may contribute to the suppression of tumor growth in NPRA^{-/-} mice. Although the differential expression of pRb and VEGF may play an important role in the mechanism of tumor suppression in NPRA^{-/-} mice, as shown in our examples, additional studies are underway to determine which of the several signal transduction pathways in which NPRA is involved are important for the anti-tumor effect, (Gabellini C, Del Bufalo D, Zupi G. Involvement of RB gene family in tumor angiogenesis. Oncogene 2006; 25:5326-32.). Clinical studies of the natriuretic peptides have not indicated any incompatibility reactions or toxic effects, (Fluge, T, Forssmann W G, Kunkel G, et al. Bronchodilation using combined urodilatin-albuterol administration in asthma: a randomized, double-blind, placebo-controlled trial. Eur J Med Res 1999; 4:411-5). Accordingly, combining the advantage of chitosan nanoparticles in targeted delivery of anti-cancer drugs with gene therapy based on the novel pNP73-102 nanoparticles or siNPRA nanoparticles pose a safe and effective treatment for a wide range of cancers in the future.

### Example 24

### NPRA-Knockout Mice are Resistant to Propagate Tramp-C1 Prostrate Tumor Cells

The TRAMP-C1 (ATCC-CRL-2730) cell line was derived in 1996 from a heterogeneous 32 week primary tumor in the prostate of a PB-Tag C57BL/6 (TRAMP) mouse. TRAMP is a transgenic line of C57BL/6 mice harboring a construct comprised of the minimal −426/+28 rat probasin promoter (426 base pairs of the rat probasin (PB) gene promoter and 28 base pairs of 5′-untranslated region) to target expression of the SV40 large T antigen to prostatic epithelium. Neither the cells grown in culture, nor the tumors arising from the cells in vivo, express SV40 T antigen (Tag). TRAMP-C1 is tumorigenic when grafted into syngeneic C57BL/6 hosts.

The protocols for the prostate tumor cells, are similar in to those used for Example 27. C57BL/6 mice were injected with TRAMP-C1 cells (5×10⁶) subcutaneously to wt, NPRA knockout (NPRA-KO) and NPRA heterozygous (NPRA-het) and seven weeks later mice were sacrificed and tumors removed. Tumors from each mouse is shown. Tumors from C57BL/6 wild-type mice are shown in FIG. 5A, and NPRA heterozygous (NPRA-het) mice are shown in FIG. 5C. None of the seven NPRA-KO mice show any tumors. Mean tumor weights are shown in FIG. 5D. Results show that NPRA-knockout mice, in which the NPRA gene was deleted showed no tumors, even after injection with TRAMP-C1 prostate tumor cancer cells. In contrast all of the NPRA-het mice show tumors; however the mean tumor weight of heterozygous mice was significantly less than the wild type mice, suggesting a dose dependent role of NPRA in tumorigenesis. Together, the results show that NPRA-knockout mice are resistant to propagate TRAMP-C1 prostate tumor cells.

### Example 25

### NPRA-Knockout Mice are Resistant to Propagate E0771 Breast Carcinoma Cells; and Human MCF-7 Breast Cancer Cells Transfected with pNP₇₃₋₁₀₂ and psiNPRA8 Showed Apoptosis

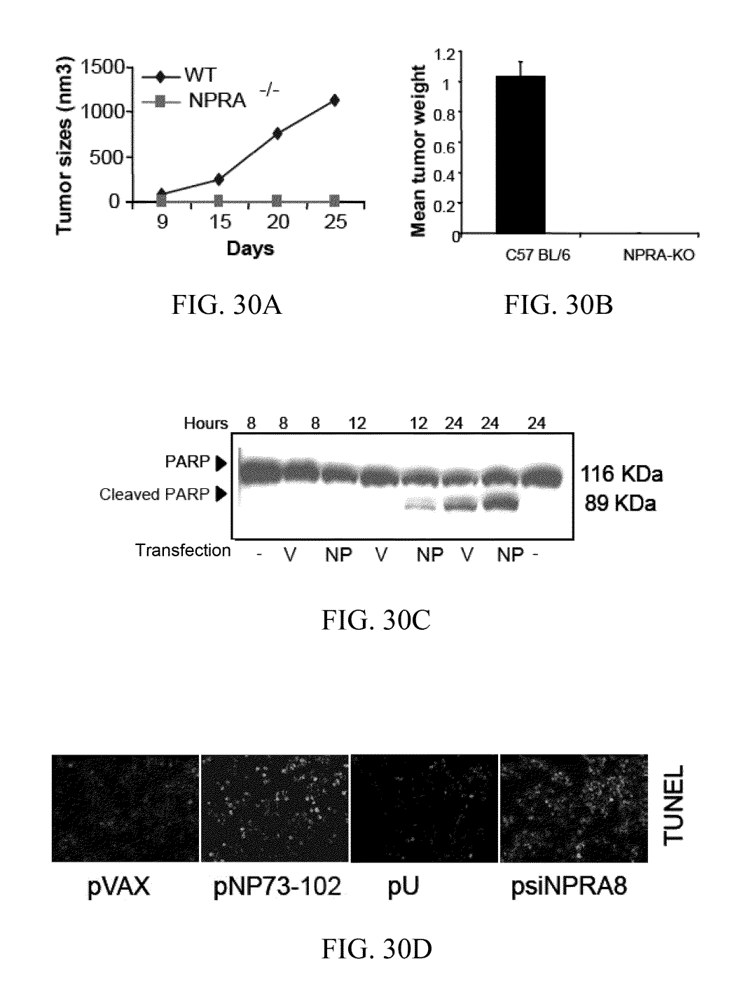

The protocols for the breast tumor cells, are similar in to those used for Examples 15-21. In FIG. 30A, both wild type (WT, n=8) and NPRA knockout (KO, n=8) mice were subcutaneously injected with 1 million of mouse breast carcinoma E0771 cells. Tumor sizes were measured from day 9 until day 25.

In FIG. 30B, mice were sacrificed on day 25 and tumors were removed and weighed. As with the results with prostate tumor cells, NPRA-knockout mice, in which the NPRA gene was silenced, showed no tumors, even after injection with breast carcinoma E0771 cancer cells.

In FIG. 30C, human breast cancer MCF-7 cells grown on 6-well plates were transfected with 1 ug of pNP73-102 (NP), and pVAX1 (V), respectively. Cells were harvested at 8, 12 and 24 hr post transfection and whole cell proteins were extracted. Apoptosis of MCF-7 induced by NP and V were analyzed by Western blot using antibodies against PARP. PARP cleavage, is “commonly used as a marker to prove cell death by apoptosis.” The presence of cleaved PARP in NP treated cells, is indicative of apoptosis, as compared to the control, which showed no PARP cleavage.

In FIG. 30D, human breast cancer MCF-7 cells grown on 6-well plates were transfected with 1 ug of pNP73-102, and pVAX1, and pU (control) vs psiNPRA8. Apoptosis of MCF-7 cells following transfection was evaluated by TUNEL assay. As the results of the TUNE L assay show, human MCF-7 breast cancer cells transfected with pNP₇₃₋₁₀₂ and psiNPRA8 showed apoptosis, unlike the controls.

Accordingly, the results show that either the compositions that reduce the activity of the trial natriuretic peptide receptor-A such as pNP73-102 or siNPRA molecules pose a safe and effective treatment for inflammatory and cell proliferation disorders. Combining them with chitosan pose another safe alternative.

Matsukawa et al. reported that natriuretic peptide receptor C modulates the availability of natriuretic peptides such as ANP, such as removing natriuretic peptides from circulation. (See, Matusaka et al., The natriuretic peptide clearance receptor locally modulates the physiological effects of the natriuretic peptide system, Proc. Natl. Acad. Sci. USA, Vol. 96, pgs. 7403-7408, Genetics, June 1999.) Furthermore, NPR-C interacts with all three natriuretic peptides in the order, ANP>CNP>BNP, and the half-life of [125I ANP in homozygote mice lacking the NPR-C receptor is two-thirds longer, thus suggesting its role in modulating its circulation. (Id.). Accordingly, reducing the activity of NPR-C may allow for more natriuretic peptide circulation, such as ANP, thereby allowing for its effects on cells, such as anti-proliferative effects on cancer cells. In one embodiment, a polynucleotide complementary with a portion of a natriuretic peptide receptor C gene is selected, and a polynucleotide complementary with a portion of a natriuretic peptide receptor A gene is selected, such that the combination may produce a synergistic effect.

### Example 26

### NPRA Expression Affects Pulmonary Inflammation

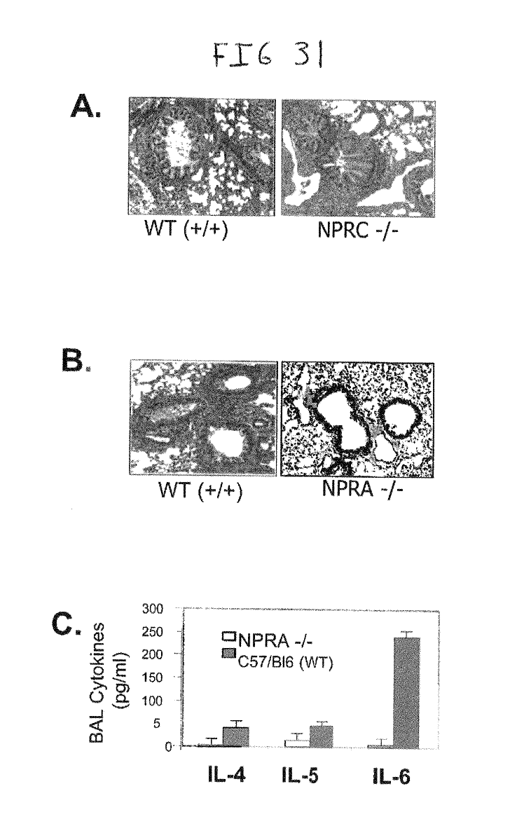

Development and chronicity of cancers has been attributed to the chronic inflammation in the affected organs. ANP was reported to have anti-inflammatory activity, although signaling through NPRA is known to cause a number of different biological activity including cell proliferation, immune activation, inflammation and apoptosis. To determine the role of NPRA signaling in the lung inflammation, groups (n=3) of wild type DBA/2 (wt) and NPR-C (ko) deficient mice and wild type C57/BL6 (wt) and NPR-A (ko) were sensitized with ovalbumin (20 mg/mouse) and after 2 weeks challenged i.n. with ovalbumin (20 mg/mouse). One day later, mice were sacrificed and lung sections were stained with H & E to examine inflammation. As shown in FIG. 31A, there was no significant difference in pulmonary inflammation between the wild-type and NPRC deficient mice. In sharp contrast, a comparison between wild-type C57BL6 and NPRA deficient mice showed that NPRA deficient mice showed substantially reduced inflammation compared to wild type (FIG. 31B). These results indicate that ANP-NPRA signaling is involved in increasing inflammation in the lung. Results shown in FIG. 31C show that the cytokines such as IL-4, IL-5 and IL-6 which contribute to inflammation are also decreased in lungs of NPRA−/− as revealed by analysis of bronchoalveolar lavage fluid for the levels of these specific cytokines. Furthermore, in a reverse experiment increased expression of a plasmid encoded ANP (FIG. 31D), delivered intranasally with chitosan nanoparticles, induced increased inflammation compared to control plasmid (FIG. 31D). To examine the role of the ANP pathway in lung inflammation and antigen-induced asthma, wild type C57/BL6 and NPRA^{-/-} mice were sensitized with ovalbumin (OVA), the allergen used in the mouse model of allergic asthma. Mice were immunized with OVA intraperitoneally (i.p.) and then challenged with OVA intranasally (i.n.). Mice were sacrificed, single-cell splenocyte suspensions were prepared, cultured 48 h in the presence of OVA and rIL-2 and stained for CD4, CD3 (gating markers) and intracellular cytokines IL-4, IL-10 and IFN-γ. Analysis of cytokines released by CD4+ splenocytes showed that a combination of NPRA deficiency and OVA exposure decreased production of IL-4, IL-10 and IFN-γ compared to NPRA^{+/+}.

### Example 27

### NPRA Gene Plays a Critical Role in Promoting Cancer

This example illustrates the role of ANP-NPRA signaling pathway in cancer development by comparing tumorigenesis in wild-type and NPRA−/− mice. Since NPRA is expressed at a higher level in all tumor cells including cells of lung carcinoma (A549, LLC1), melanoma (B16), ovarian cancer (SKOV3, ID8) and prostate cancer cells (DU145) compared to normal cells, tumorigenesis was studied in related models.

Methods: To test for the role of NPRA in different cancers the following methodologies were used. (FIG. 32A,B) Groups of wild type and NPRA^{-/-} mice (n=8 per group) were injected s.c. with 2×10⁶ LLC1 cells. Tumor sizes (A) were measured on day 10, 13, and 17 and tumor weights (B) at day 17 were compared (p<0.01). (C,D) Groups of wild type and NPRA^{-/-} mice (n=12) were injected s.c. with 2×10⁶ B16 melanoma cells and tumor sizes (C) were measured on day 10, 13, 15 and 17 and tumor weight (D) were measured and compared at day 18 (p<0.01). Data from one of the two repeated experiments is presented. (E,F) Groups of wild type and NPRA^{-/-} mice (n=12) were injected s.c. with 2×10⁶ MCF7 breast cancer cells and tumor sizes (E) were measured on day 9, 15, 20, and 25 and tumor weight (F) were measured and compared at day 25 (p<0.01). Data from one of the two repeated experiments is presented. (G) Groups of wild type and NPRA^{-/-} mice (n=8) were injected s.c. with 2×10⁶ mouse ovarian cancer ID8 cells and tumor sizes were measured every week after ID8 injection.

Using the Lewis lung carcinoma model, C57BL/6 wild type and NPRA gene knockout (NPRA^{-/-}) mice (n=8 for each group) were injected s.c. with 2×10⁶ cells LLC1 cells in the right flank. Tumors appeared within one week after injection and tumor size was measured with a digital caliper beginning on day 10. The tumors in wild type mice grew rapidly after day 10, but tumors in NPRA^{-/-} mice gradually shrank. On day 17, all mice were sacrificed, and tumor sizes and weights were measured. In one of the NPRA^{-/-} mice, there were no visible tumors at all. Significant differences in tumor size and weight were observed between the two groups (FIG. 32A-B).

As a further test of the antitumor activity of NPRA^{-/-} mice in relation to melanomas, mice were injected s.c. with B16F10 melanoma cells. Groups of wild type and NPRA^{-/-} mice (n=12) were injected s.c. with 2×10⁶ B16 melanoma cells. Tumors appeared within one week after injection and tumor size was measured with a digital caliper beginning on day 10. The tumors in wild type mice grew rapidly after day 10, but tumors in NPRA^{-/-} mice gradually shrank. On day 18, all mice were sacrificed, and tumor sizes and weights were measured. A significant reduction in mean tumor volume measured over 18 d after B16 cell injection and a significant decrease in tumor weight at day 18 was found in NPRA^{-/-} mice (n=12) compared to wild type (FIGS. 32C, D).

The potential of NPRA deficiency to inhibit growth of E0771 breast carcinoma cells was also tested. Groups of wild type and NPRA^{-/-} mice (n=12) were injected s.c. with 2×10⁶ MCF7 breast cancer cells and tumor sizes (E) were measured on day 9, 15, 20, and 25 and tumor weight (F) were measured and compared at day 25. NPRA^{-/-} mice exhibited a significant reduction in tumor growth compared to wild type (FIGS. 32E,F).

The potential of NPRA deficiency to inhibit growth of ovarian cancer cells was also tested, and again NPRA^{-/-} mice exhibited a significant reduction in tumor growth compared to wild type (FIG. 32G). Groups (n=8) of wild type and NPRA-deficient C57BL/6 mice were injected with 2×10⁶ ID8 mouse ovarian cancer cells at day 1 and mice were monitored at weekly intervals for tumor growth. By week 8 after cancer cell inoculation, all mice from the wild type mice developed solid tumors but no tumors were found in NPRA-deficient mice (FIG. 32G). The results indicate that NPRA deficiency significantly protects mice from tumorigenesis and progression.

### Example 28

### A549 Cells Transfected with pNP₇₃₋₁₀₂ Show a Significantly Higher Level of Apoptosis Compared Control and pANP or pVAX

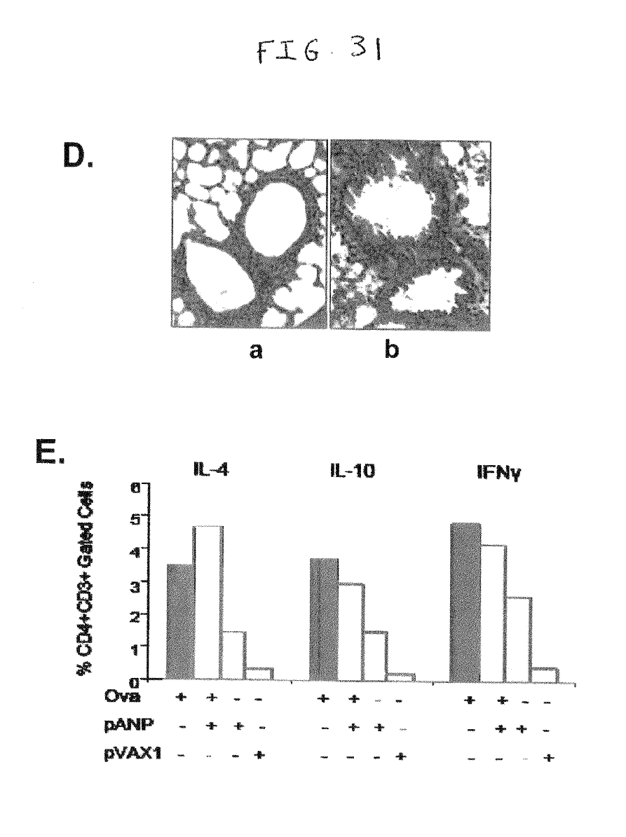

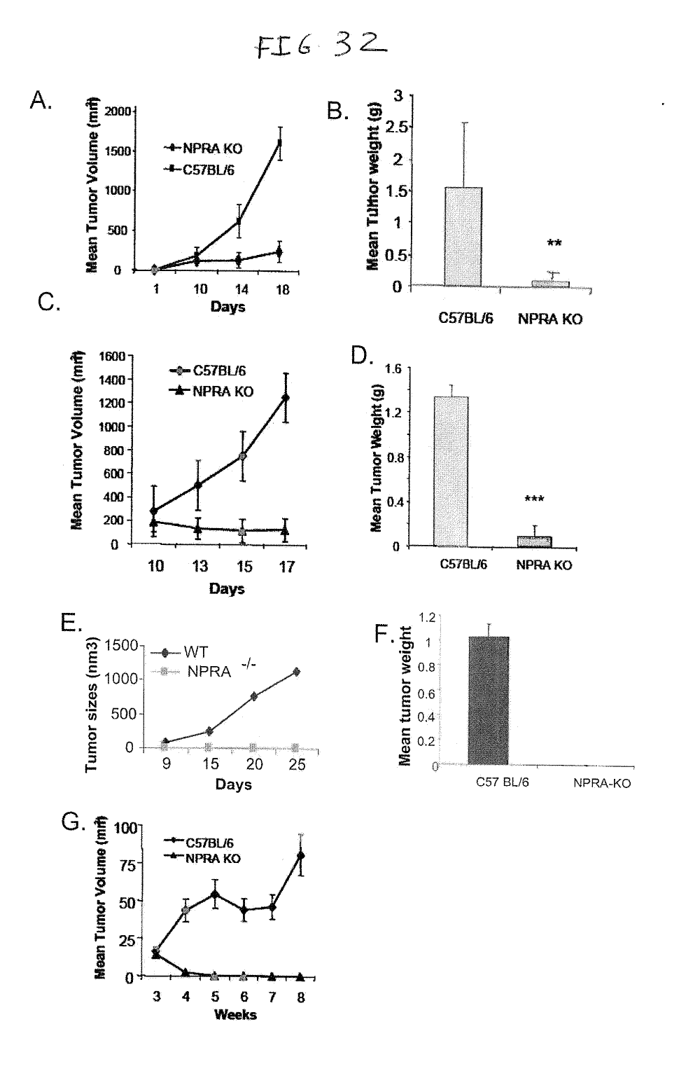

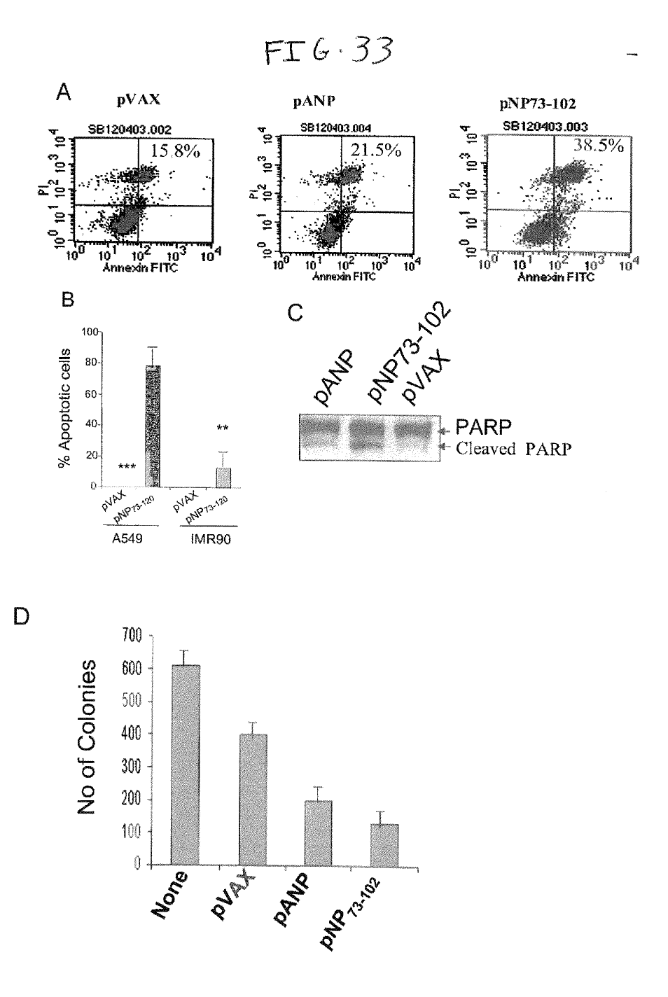

To determine the effect of over expression of NP73-102 on proliferation of A549 lung epithelial cells, cells were transfected with either pNP73-102 or vector, pVAX. Cell cycle analysis was performed using propidium iodide (PI) staining and flow cytometry 48 h after transfection. No significant difference was observed between control and pNP73-102-transfected cells in S5, Go-G1 and G2-M stages of cell cycle (data not shown). However, an analysis of apoptosis using flow-cytometry with PI and annexin V, showed that cells transfected with pNP73-102 exhibited significantly higher apoptosis compared to cells transfected with either the control plasmid or a plasmid encoding ANP (FIG. 33A). A significantly higher apoptosis is seen in A549 adenocarcinoma cells compared to normal IMR-90 cells, as shown by TUNEL assay of A549 cells cultured in 8-chamber slide following a 48-hour transfection with either pANP or pNP73-102 (FIG. 33B) and by analysis of PARP cleavage in these cells 48 hours after transfection, which was significantly more prominent in pNP73-102 transfected cells compared to pANP or pVAX transfected cells (FIG. 33C). The results show that pNP73-102 shows a higher accumulation of apoptotic cells compared to cells transfected with pANP and pVAX controls. Thus, pNP73-102 induces apoptosis of lung adenocarcinoma cells.

In an effort to identify and characterize molecules participating in early signaling pathways, differential gene expression was analyzed using a microarray (AFFYMETRIX). Altered expression of a large number of genes was found, including genes related to cell growth, cell cycle, and apoptosis. These genes included, among others more than, 6- to 8-fold up-regulation of genes such as Caspase (Casp)-8 and FADD like apoptosis regulator, cyclin E binding protein, CDK inhibitor 1A, CDK7, casp4, casp-10, casp-1, apoptosis facilitator BCL2-like 13 and annexin 43 (data not shown). Together, these studies indicate that pNP73-102 is an inducer of apoptosis in A549 lung adenocarcinoma cells.

To test the anti-cancer activity of the pNP73-102 construct, a colony forming assay was undertaken. Thus, six cm tissue culture plates were covered with 4 ml of 0.5% soft agar. A549 cells were transfected with pANP, pNP₇₃₋₁₀₂ and pVAX plasmid DNA. After 40 hours of transfection, equal number of cells were suspended in 2 ml of 0.3% soft agar and added to each plate. Cells were plated in duplicate at a density of 2×10⁴ cells/dish and incubated for two weeks. Plates were observed and photographed under a microscope. Cell colonies were counted and plotted. The results of one representative experiment of two experiments performed is shown in FIG. 33D. The results show that plasmid vector alone caused some reduction in colony formation compared to untransfected control. However, both ANP and pNP₇₃₋₁₀₂ showed substantial reductions in the number of colonies produced compared to vehicle control.

### Example 29

### Transfection with pNP₇₃₋₁₀₂ Induces a Significantly Higher Level of Apoptosis Compared to Control and pANP or pVAX in Several Cancer Cell Types

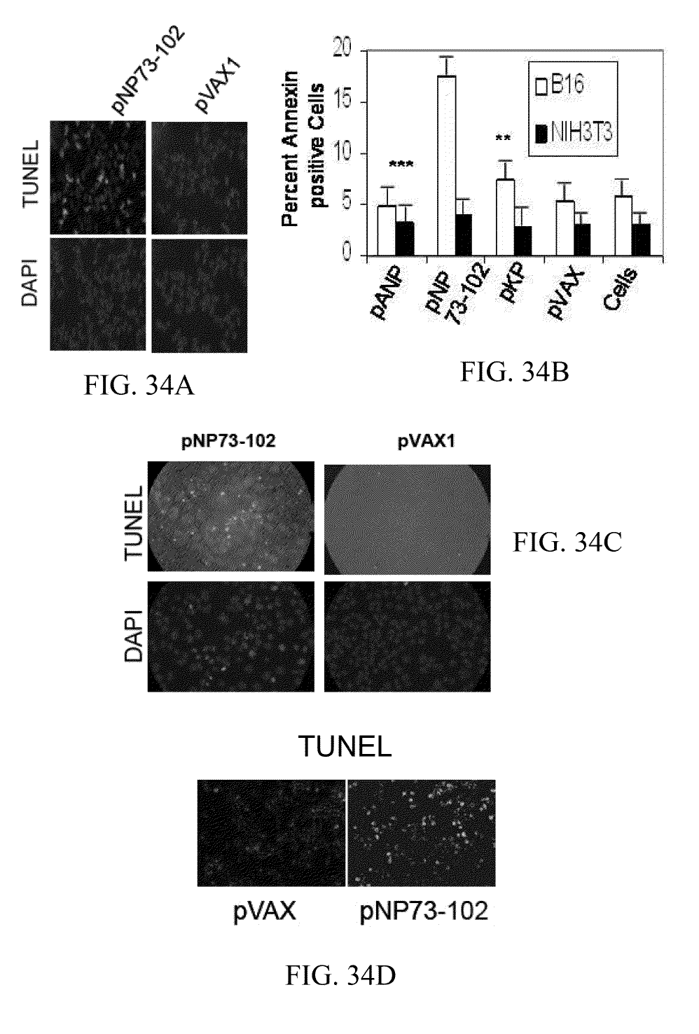

FIGS. 34A-E show that cells transfected with pNP₇₃₋₁₀₂ undergo a significantly higher level of apoptosis compared to pANP or pVAX control in melanoma, ovarian and breast cancer cells. To determine whether apoptosis induction was the dominant explanation for the anti-tumor activity of pNP73-102, we tested the effect of ectopic expression of pNP73-102 in B16 melanoma and normal NIH3T3 cells (FIGS. 34A-B). Plasmids encoding ANP (pANP) and KP (pKP) were used as controls in this experiment. The results showed significant apoptosis of B16 cells as measured by Annexin binding assay but not of the normal NIH 3T3 cells (FIG. 34B). Also, significantly more B16 cells were observed to be TUNEL-positive when they were treated with pNP73-102 compared to the number observed among cells treated with pVAX as control (FIG. 34A). These results indicate that a decrease in NPRA signaling may result in the induction of apoptosis in melanoma cells but not in normal cells.

Chemoresistance is a major therapeutic problem in many of the cancers and the current knowledge on cellular mechanisms involved is incomplete. Since A549 cells showed differential sensitivity to apoptosis with pVAX and pNP₇₃₋₁₀₂, the effects of pNP73-102 was tested using chemosensitive (OV2008) and chemoresistant (C13) ovarian cancer cells. C-13 and OV2008 ovarian cancer cells were transfected with pNP73-102 or with pVAX as control. Forty-eight hours later, cells were processed to examine apoptosis by TUNEL assay (FIG. 34C). The results showed that either of the cells when transfected with pVAX did not exhibit any apoptosis. In contrast, both cell lines exhibited apoptosis as evident from TUNEL positive cells. These results indicate that pNP73-102 may induce apoptosis of ovarian epithelial adenocarcinomas irrespective of their degree of chemo-sensitivity.

Similarly, we examined the potential of pNP73-102 in inducing apoptosis of MCF-7 breast cancer cells. Cells were transfected with pNP₇₃₋₁₀₂ or pVAX and apoptosis was analysed by TUNEL assay and Western blotting for PARP cleavage. FIG. 34D show a significantly higher level of TUNEL-positive MCF7 cells transfected with pNP73-102 compared to pVAX control. Furthermore, PARP cleavage was seen in these cells 12 hours after transfection, which was significantly more prominent in pNP73-102 transfected cells compared to pVAX transfected cells (FIG. 30C). Collectively, these results show that pNP73-102 induces a higher accumulation of apoptotic cells compared to cells transfected with pVAX controls. Thus, pNP73-102 induces apoptosis of breast adenocarcinoma cells.

### Example 30

### pNP73-102 Decreases Lung Inflammation and Asthma in Experimental Models

ANP has been suspected to play a role in decreasing inflammation, as it was shown to play a role in decreasing TNF-a production from macrophages and slightly decreased NFkB activation (Mohapatra et al. JACI, 2004). Also, NPRA deficient mice exhibit reduced inflammation. Since excess ANP expression activates the clearance receptor, it was hypothesized that ANP actually increases inflammation. To test this naive mice were administered intranasally (i.n.) a plasmid pVAX expressing the ANP peptide. The results show that ANP over expression actually increases inflammation (FIG. 1).

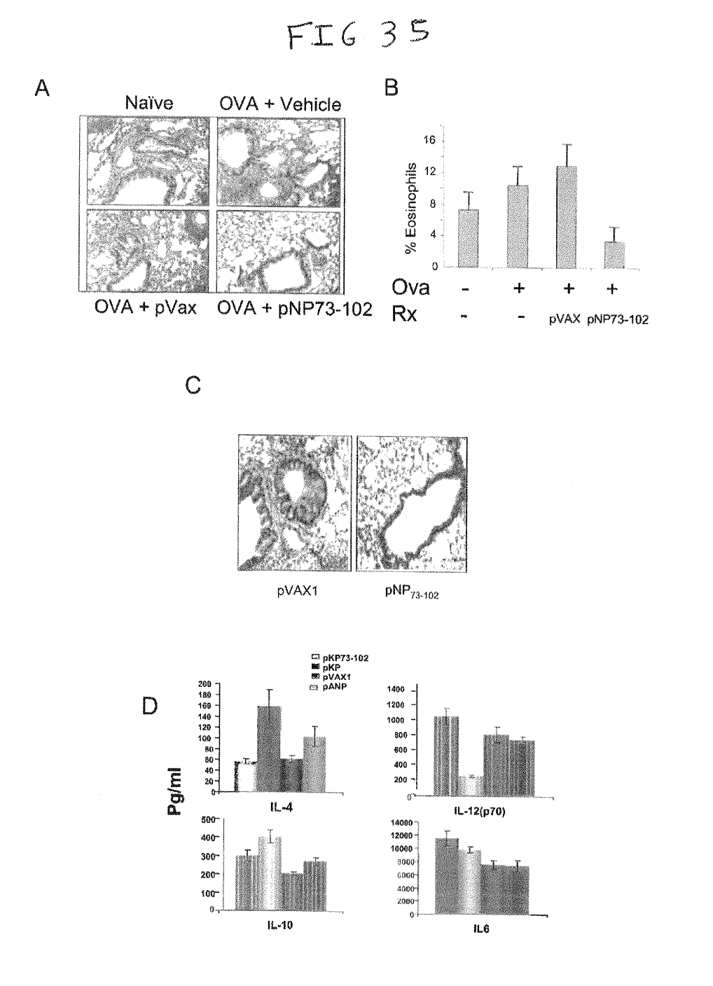

To determine whether decreased expression of NPRA by pN73-102 treatment will reduce inflammation in asthma, the effect of pNP73-102 administered by gavage (FIGS. 35A-B) or intranasal route (FIGS. 35C-E) was tested in ovalbumin-induced mouse model of asthma.

Materials and Methods.

Six to eight week-old BALB/c mice (n=6) were sensitized by i.p. injection of ovalbumin (50 ug in 2 mg of alum/mouse) and challenged intranasally with OVA (50 μg·mouse). Mice were given two treatments of chitosan nanocomplexes of pNP73-102, pVAX or vehicle by gavage or intranasally and challenged 24 hours later. After a further 24 hours of challenge, mice were sacrificed and their lungs removed for histology in a subgroup (n=3) of mice. The remainder of the group were lavaged and a cell differential was performed as described, especially to enumerate the eosinophil numbers in the BAL fluid.

Results.

The results of histology of lung sections stained by H & E revealed Ovalbumin-sensitized and challenged mice treated with pNP73-102 showed a significant reduction in lung inflammation compared to those treated with pVAX (control) (FIG. 35A). The lung histology of pNP73-102 treated group was very similar to the naive mice. There was significant reduction in epithelial goblet cell hyperplasia and a significant reduction in peribronchial, perivascular and interstitial infiltration of the inflammatory cells to the lung (FIG. 35A). There was also a significant reduction in the number of eosinophils in BAL fluid (FIG. 35B).

The effects of intranasal treatment with nanocomplexes of pNP73-102 versus pVAX (control) was tested in groups of mice. Mice treated with pNP73-102 showed a significant reduction in lung inflammation compared to those treated with pVAX (control) (FIG. 10C). The lung histology of pNP73-102 treated group was very similar to the naive mice. There was significant reduction in epithelial goblet cell hyperplasia and a significant reduction in peribronchial, perivascular and interstitial infiltration of the inflammatory cells to the lung (FIG. 35C). There was also a significant reduction in the number of eosinophils in BAL fluid (data not shown).

To verify whether the reduction in inflammation and airway eosinophilia was due to reduction in Th2-like cytokine production, a human dendritic cell model was used. Human monocyte derived dendritic cells were cultured with IL-4 and GM-CSF and four days of cultured they were transfected with plasmids encoding either pANP, pNP73-102 or pVAX (control). The transfected DCs were co-cultured (1 DC: 10 T cells) with naïve cordblood T cells and the cytokine profile in the supernatant was measured after 48 h of co-culture. The levels IL-4, IL-10, IL-12 and IL-6 were measured in the supernatant. The results showed that pANP transfected DCs prompted the overproduction of IL-4 and IL-10 cytokines (markers of Th2) compared to pVAX-transfected DCs, whereas pNP73-102 transfected DCs induced increased IL-12, an inducer of Th1 response (FIG. 35D).

### Example 31

### Inhibitory Effect of Transfected siRNA Plasmids on NPRA Expression

Although NPRA−/− mice show decreased inflammation and decreased TH2 response, it was unclear whether this was specifically due to loss of NPRA gene or other genes or physiologic conditions associated with NPRA loss in this specific strain background. It was reasoned that knockdown of NPRA by short-intererence RNA will confirm that these changes were due to NPRA loss alone and also it might provide therapeutic anti-inflammatory effects.

To determine whether siRNAs can be produced that will effectively decrease NPRA expression, 11 different siRNA oligos were designed and cloned in a pU6 vector. Cells transfected with each of the construct was examined for NPRA protein expression by western blotting.

The nucleotide sequence for each is described previously (SEQ ID NOs: 23-33). Each pair of oligos was inserted into pU6 plasmid at appropriate sites respectively, to generate the corresponding siRNA for siNPRA.

Cells were transfected with siNPRA or controls (siU6) using LIPOFECTAMINE 2000 reagent (INVITROGEN, Carlsbad, Calif.). pEGFP plasmid (STRATAGENE, La Jolla, Calif.) was used for measurement of transfection efficiency.

Protein expression analysis by Western blotting

Transfected cells were used to prepare whole cell lysates, which were electrophoresed on 12% polyacrylamide gels and the proteins were transferred to Pvdf membranes (BIO-RAD, Hercules, Calif.). The blot was incubated separately with NPRA polyclonal antibody (SANTA CRUZ BIOTECH Santa Cruz, CA), immunoblot signals were developed by SUPER SIGNAL ULTRA chemiluminescent reagent (PIERCE, Rockford, Ill.).

### Results

Eleven different siRNA oligos were designed specifically targeting NPRA gene. The siRNA oligos were cloned in pU6 vector. FIG. 11 shows results the inserts being present in the plasmids. FIG. 11A shows the results of an experiment with 8 clones having their inserts analysed by gel electrophoresis. The inserts were sequenced to confirm the presence of siRNA inserts in them.

In additional experiments, HEKGCA cells grown in 6-well plates were transfected with psiNPRA (2 ug), as indicated and forty eight hours later total protein were extracted western blotted using an antibody to NPRA (FIG. 36B). Untransfected cells and cells transfected with U6 vector plasmid without any siNPRA were used as control. Also, filters were stripped and reprobed with antibody to beta-actin. The experiments were repeated. Results showed that si8, si9 and silo are most effective in decreasing NPRA expression in the HEKGCA cells. To confirm these results, inhibitory effect of siRNA in vitro was examined using HEKGCA cells. Cells grown in 6-well plates were transfected with psiNPRA (2 μg). Forty eight hours later, cells were subjected to flow cytometry to detect NPRA positive cells using an antibody to NPRA (FIG. 36C). U6 plasmid without any siRNA was used as control.

Mice (n=4) were intranasally administered as nasal drops with 25 ug siRNA plasmids complexed with 125 μl of chitosan nanoparticles. BAL was done 72 hours later. Cells were stained by NPRA Ab. NPRA expression cells were counted (FIG. 36D). Together the results show that siNPRA8, siNPRA9 and siNPRA10 were the most effective siRNAs that significantly reduced NPRA expression.

### Example 32

### Demonstration that siNPRA Treatment Decreases Melanoma Tumor Formation in B16 Mouse Model

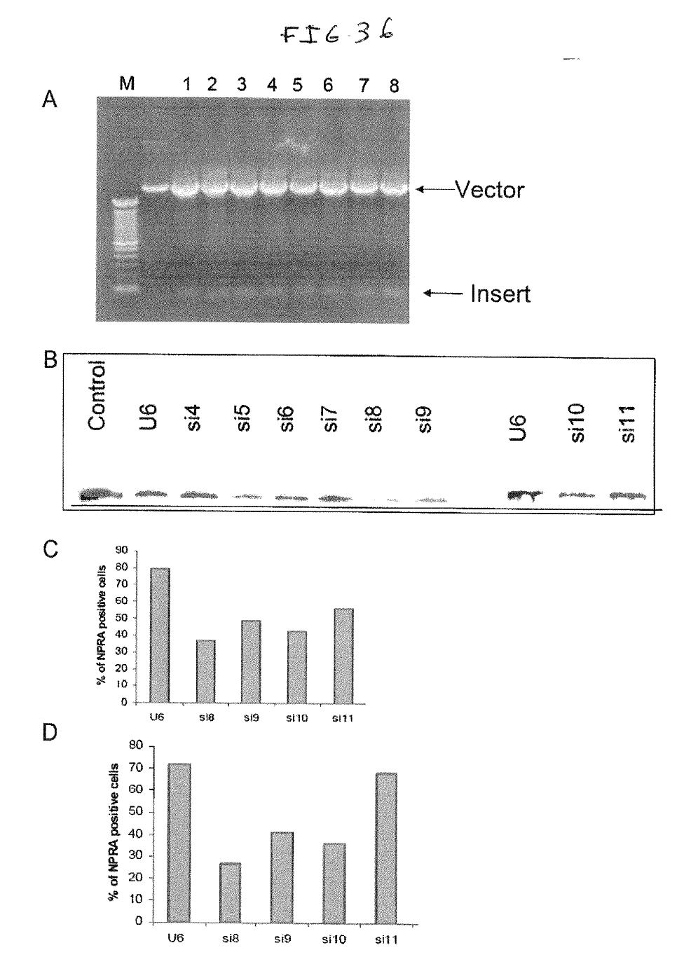

In order to develop a nanoparticle-based topical delivery system, chitosan polymers were tested to verify that it can aid in transfection of cells with siRNA in vitro using siGLO as fluorescent siRNA marker. To prepare siGLO-chitosan nanoparticles, 0.2 nmol of siGLO were complexed with 5 mg of chitosan polymers (33 kD a) before transfection. HEK293 cells were transfected and the incorporation of siGLO into HEK293 cells was monitored by fluorescence microscopy 24-48 hrs after transfection (FIG. 37A). HEK293 cells were also transfected with pEGFP-N2 chitosan nanoparticles as a positive control.

In this experiment to test whether chitosan plays a critical role in siRNA in vivo delivery, chitosan-siGLO nanocomplexes (2 nmol of siGLO mixed with 50 mg of chitosan) were intratumorally injected into the PC3-induced prostate tumors in BALB/c nude mice and siGLO was examined 48 h after injection. Fluorescence microscopy revealed that siGLO was only present in tumors when delivered in chitosan nanocomplexes but not when delivered in naked form (FIG. 37B).

Lung sections were also prepared from siGLO-treated mice and the presence of siGLO in the lung was confirmed by fluorescence microscopy (FIG. 37C). To test whether chitosan nanoparticles could deliver siGLO transdermally or topically in mice, siGLO chitosan nanoparticles (2 nmol siGLO plus 50 mg of chitosan) with 62.5 mg of 5% imiquimod cream was applied to the back of a BALB/c nude mouse. Another application was done on the same location 24 hrs later. Distribution of siGLO in vivo was detected through whole-body fluorescence imaging using a Xenogen IVIS system, siGLO was found to reach the lung 48 hrs after treatment (FIG. 37D). Intranasally-delivered pEGFP-N2 nanoparticles (without cream) were included as a positive control for the presence of fluorescence (FIGS. 37C and 37D). Together the results show that siRNA can be delivered topically with a combination of nanoparticles and cream.

### Example 33

### Demonstration that the Topical (Transdermal) Route Decreases NPRA Expression, Eosinophilia of the Lung and Bal IL-4 Cytokin

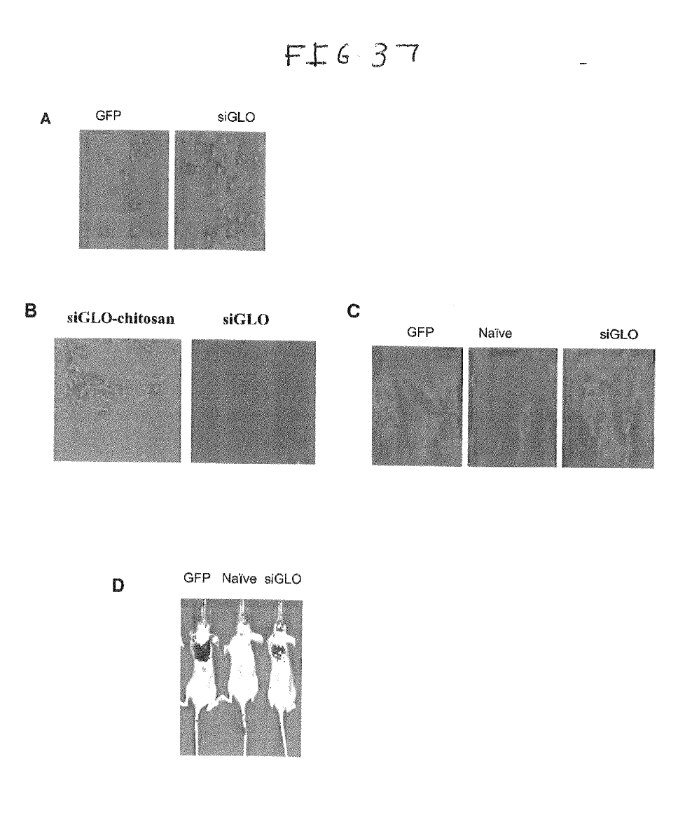

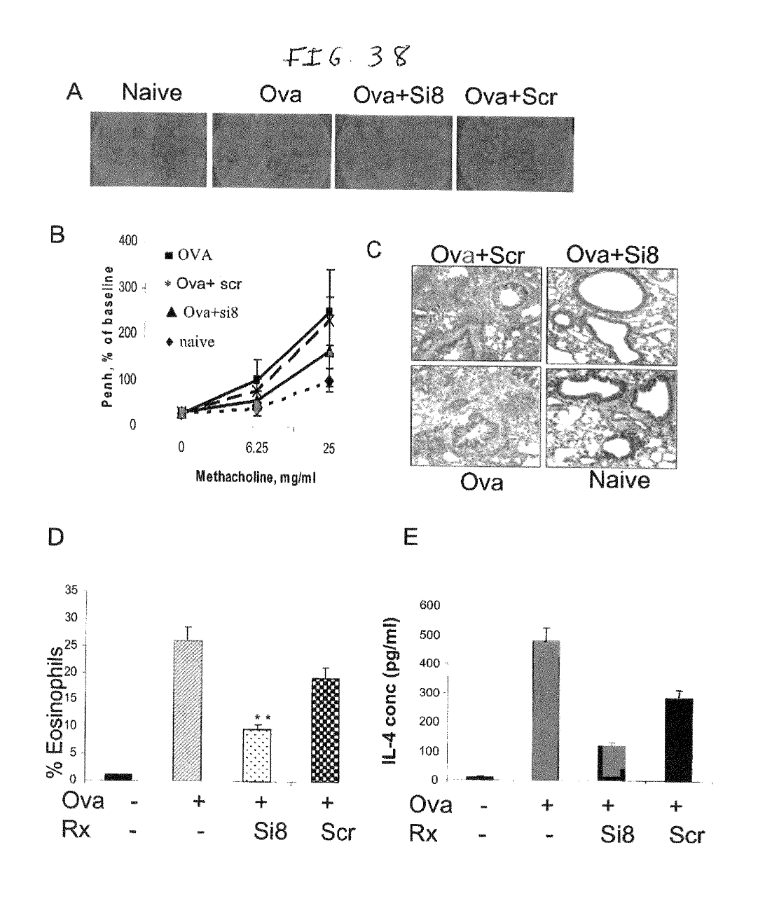

An siNPRA cream decreases NPRA expression in the lung. BALB/c mice (n=5 each group) were sensitized (i.p.) and challenged (i.n.) with 50 μg of OVA. Mice were given siNPRA8 oligonucleotide treatments by transdermal route and challenged 4 hours later. Following 24 hours of challenge two mice were sacrificed to obtain lungs and which were fixed sectioned and immunostained for NPRA expression. The results show that lung sections from siNPRA8 treated mice show significantly decreased expression of NPRA compared to scrambled control (FIG. 38A).

Transdermally-delivered siNPRA reduced airway hyperreactivity. AHR was recorded on day 22 in a whole-body plethysmograph which measures the enhanced pause (PENH). The Penh values were averaged and expressed for each MCh concentration as a percentage of the PBS baseline reading. The results show that siNPRA8 treatment decreased airway hyperreactivity (FIG. 38B).

Lungs were obtained 24 hours after challenge, fixed in formalin, sectioned and stained with hematoxylin and eosin. The results show that lung sections from siNPRA8 treated mice showed a substantial reduction in inflammation compared to untreated mice and scramble siRNA treated mice. The siNPRA8-treated lungs were similar to those of lungs from naïve mice (FIG. 38C).

Reduction of eosinophils by siNPRA-imiquimod treatment. Mice (n=4) were sacrificed and lavaged and the percentage of eosinophils recorded. BAL cells were air dried and stained with a modified Wright's stain. Total cell numbers were approximately the same in each group and the number of eosinophils is given as percentage of the total (**p<0.01) (FIG. 38D).

IL-4 in BAL fluid was measured by IL-4 ELISA. Significant reduction of IL-4 (**p<0.01) was achieved by siNPRA-imiquimod treatment when compared with OVA controls (FIG. 38E).

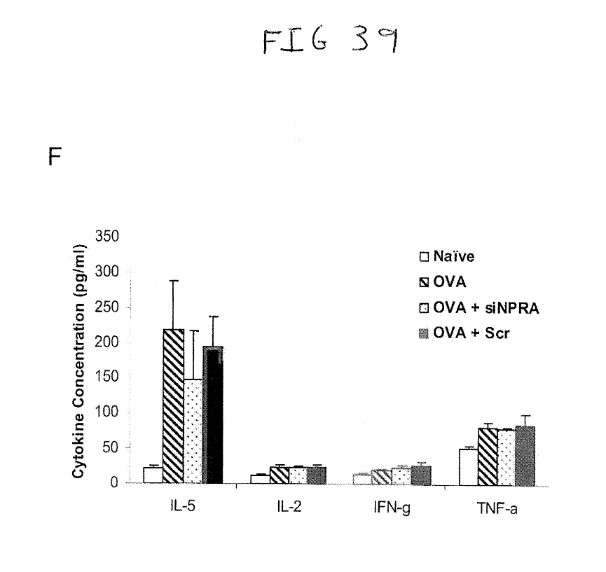

Lungs of all animals from the four groups were removed and homogenized. The levels of IL-2, IL-5, IFN-γ and TNFα in lung homogenate were measured using a mouse Th1/Th2 Cytokine CBA kit following the manufacturer's instruction (BD Bioscience, CA). IL-5 was also significantly downregulated by siNPRA treatment (*p<0.05) (FIG. 39). The results show effectiveness of nanoparticle creams containing siNPRA8 as the active principle.

### Example 34

### Demonstration that Intranasal siNPRA Treatment Decreases Inflammation Eosinophilia and Th2 Cytokines in Balb/C Mice

Asthma is a chronic inflammatory lung disease that involves both upper and lower airways. Current drugs for athma are delivered as intranasal sprays or inhaled formulations. Patients are more compliant when the drug is delivered by these routes. Therefore, it was attempted to determine whether such siRNA therapy would decrease pulmonary inflammation in this ovalbumin-induced mouse model of asthma.

Materials and Methods.

BALB/c mice (n=5 each group) were sensitized (i.p.) as in example #11 and challenged (i.n.) with 50 μg of OVA. Mice were given siNPRA (oligonucleotide) treatments by transdermal route (siNPRA9) and challenged 4 hours later. To determine whether siNPRA can prevent AHR, groups of mice were challenged with 6.25% and 25% methacholine on day 22 and AHR was measured (FIG. 39A). Following 24 hours of challenge two mice were sacrificed to obtain lungs and which were fixed sectioned and immunostained for NPRA expression (FIG. 39B). Mice (n=3) were sacrificed and lavaged and the percentage of eosinophils (FIG. 39C) and IL-4 and IL-10 concentration (FIG. 39D) in the lavage fluid was determined.

Results.

To confirm that decreasing expression of NPRA reduces allergen-induced lung inflammation, we designed siRNAs to knockdown NPRA expression and tested them as nanocomplexes on OVA-allergic BALB/c mice. To determine whether siNPRA can prevent AHR, groups of mice were challenged with 6.25% and 25% methacholine on day 22 and AHR was measured. It was found that the siNPRA-treated mice had significantly lower AHR than the untreated group or the control group receiving scrambled siRNA (FIG. 39A).

Lung sections from siNPRA-treated mice stained with hematoxylin/eosin (H & E) showed a significant reduction in lung inflammation compared to mice treated with a scrambled siNPRA. The lung histology in siNPRA-treated OVA-allergic mice was very similar to that of naïve mice. There was a significant reduction in epithelial goblet cell hyperplasia and in peribronchial, perivascular and interstitial infiltration of inflammatory cells to the lung (FIG. 39B).

The number of eosinophils in BAL fluid from siNPRA-treated mice was also significantly lower than controls (data not shown). (FIG. 39C).

The levels of IL-4 and IL-10 was examined in splenocyte cultures. The results showed that groups of mice treated with siNPRA9 intranasally decreased significantly both IL-4 and IL-10 suggesting a shift away from Th2-response, the latter is a characteristic of asthma (FIG. 39D). Therefore, inhibition of NPRA by siNPRA nanoparticles may provide a new treatment for allergic asthma

### Example 35

### Demonstration that Transfection of A549 Cells with psiNPRA9 Decreases the Number of Respiratory Syncytial Virus (RSV) Infection Infected Cells

Respiratory syncytial virus infection also causes bronchiolitis in newborns and in elderly causing pneumonitis which is characterized severe acute lung inflammation. RSV infection typically requires certain host cell proteins and transcription factors for its replication and subsequent infection of others cells. Since siNPRA treatment decreases pulmonary inflammation, the effect of siNPRA9 transfection on RSV infection was examined in pulmonary type-II epithelial cells was examined.

RT-PCR analysis of NPRA expression in the lung of mice treated with siRNA psiNPRA9 was encapsulated with chitosan nanoparticles and intranasally delivered to mice. Twenty-four hours later, mice were infected with RSV (5×10⁶ pfu/mouse). Four days later, mice were sacrificed and lung cells were collected for RNA extraction. NPRA fragments were amplified by RT-PCR using NPRA specific primers (F:5′ GCA AAG GCC GAG TTA TCT ACA Te (SEQ ID NO:27), R:5′ AAC GTA GTC eTC CeC ACA CAA-3 (SEQ ID NO:28)) and analyzed in 1% agarose gel.

To determine the effect of siNPRA9 on RSBV infection of epithelial cells A549 cells were grown in 6 well plate, transfected by siNPRA8 siNPRA9 or control U6 plasmid (2.0 ug) and 2 hours after infected by rgRSV (MOI=0.2). Cells were checked for infection 48 hours later, FACS was done. Also, A549 cells were grown in 6 well plate infected by rgRSV (MOI=0.2) and 24 hours after infection they were transfected by siNPRA8, siNPRA9 or control U6 plasmid (2.0 μg) and further 24 hr later, flow cytometry was performed to estimate percentage of infected cells.

### Results

RT-PCR analysis show that both RSV infected mice and mice infected with RSV and intranasally treated with pU6 control plasmid given with chitosan nanoparticles showed NPRA expression in the lung cells. However, mice infected with RSV and intranasally given psiNPRA9 showed an amplification product that was reduced in band intensity compared to cells from mice given pU6 plasmid. The lung cells from NPRA knock-out mice showed the band as well but it was reduced in intensity.

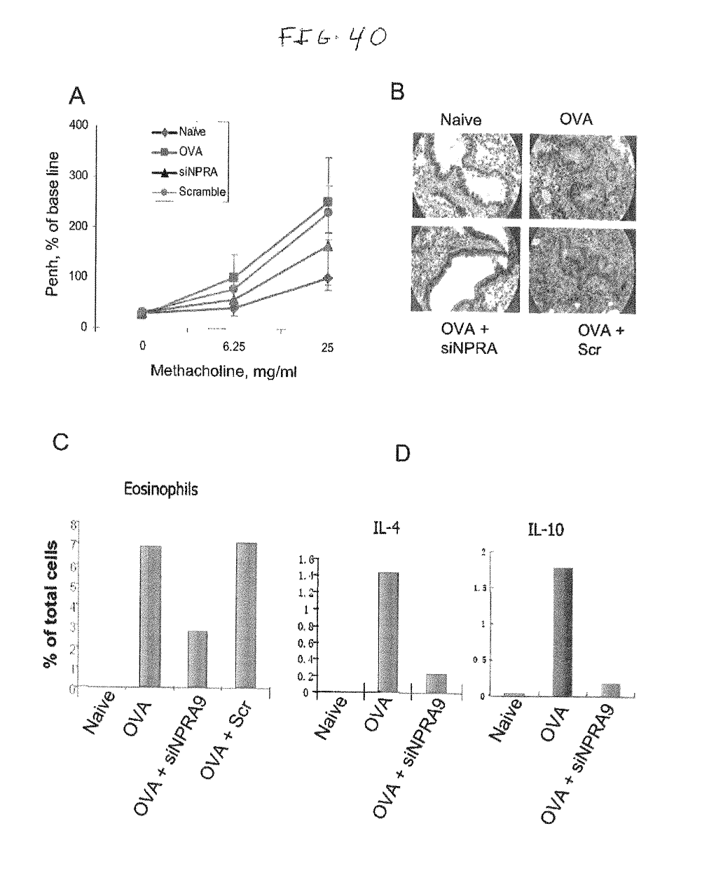

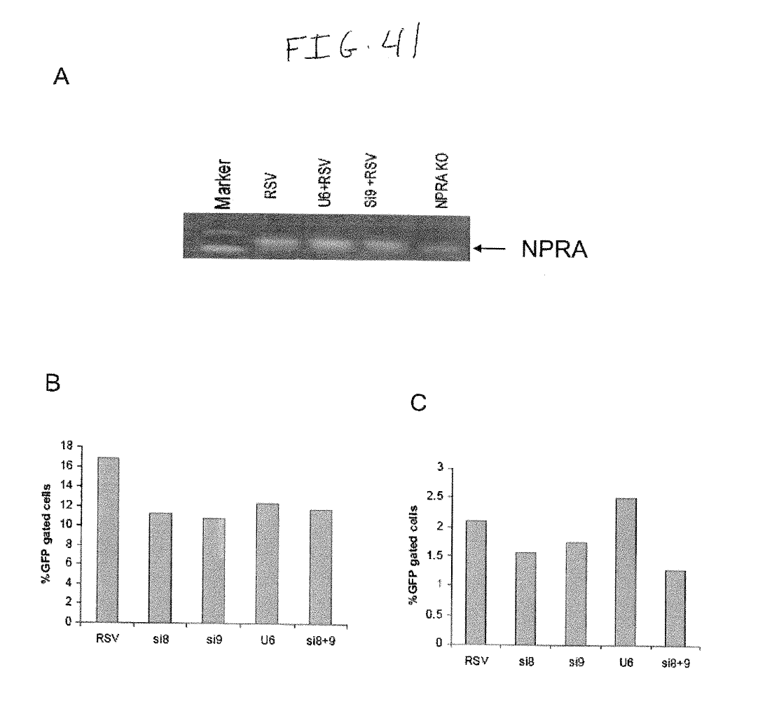

To show the effect of siNPRA on rgRSV infection of A549 cells, A549 cells were grown in 6 well plate, transfected by 2 μg of siNPRA8, siNPRA9 or control U6 plasmid, and 2 hours after infected by rgRSV (MOI=0.2) (prophylactic approach), or A549 cells were grown in 6 cell plate infected by rgRSV (MOI=0.2) and 24 hours after infection they were transfected by siNPRA8, siNPRA9 or control U6 plasmid (2.0 μg) (therapeutic approach). After 24 hours, flow cytometry was performed to estimate percentage of infected cells. The results show a 20% reduction in rgRSV infected cells in cells treated with siNPRA8 and/or siNPRA9 compared to siU6 control plasmid (FIG. 40B). Thus these results show that siNPRA treatment decreases RSV infection. The treatment also reduced inflammation.

### Discussion

Increased inflammation may contribute to the genesis of cancer. Three different cancer models were investigated in C57BL/6 wild type mice and NPRA^{-/-} mice, as previously described: the Lewis-lung carcinoma model, the B16-induced melanoma model and the ID8-induced spontaneous model for ovarian cancer. The NPRA^{-/-} mice (i.e. nutriuretic peptice receptor A suppressed) showed little or no tumor growth compared to wild type mice. It is believed that ANP over expression decreases NPRA levels in cells. See Pandey K N, Nguyen H T, Sharma G D, Shi S J, Kriegel A M. Ligand-regulated internalization, trafficking, and down-regulation of guanylyl cyclase/atrial natriuretic peptide receptor-A in human embryonic kidney 293 cells is shown to be correlated with a biological feedback inhibition response, perhaps. See J Biol Chem 2002; 277:4618-27. Natriuretic peptides, such as KP, VD, atrial natriuretic peptide and long acting natriuretic peptide, may inhibit ERK 1/2 in prostate cancer cells. See Anticancer Res 2006; 26:4143-8. Vessel dilator and kaliuretic peptide inhibit ERK 1/2 activation in human prostate cancer cells. See Anticancer Res 2006; 26:3217-22. The cause of inhibition of cancer cell proliferation and a method of treatment or prevention of cancer have not been known. However, it is now believed, without being limiting in any way that these peptides regulate NPRA expression, in some cases down regulating receptor expression in cells. Alternatively, one or more peptides may function by regulating NPRA signaling. Regardless, it is shown by the examples and results presented here that NPRA expression is a target for cancer treatment and prevention. Examples are provided that effectively regulate the expression of NPRA to therapeutically treat a variety of cancer types, such as breast, lung, pancreatic, melanoma and ovarian, using a variety of pathways, such as subcutaneous injection, transdermal cream, oral gavage, intravaginal, and intranasal.

In one method of therapeutic treatment of cell proliferation disorders, siRNA is delivered to reduce NPRA expression in immunocompetent C57BL/6 mice. Plasmids including siRNA sequences are disclosed that induce degradation of NPRA transcripts and block expression of NPRA in cells. It is believed, without being limiting, that the siRNA sequences in the plasmids are protected from degradation, and the plasmids facilitate entry of the siRNA into tumor cells. Thus, the siRNA may be targeted to tumor cells. Examples are provided where the treatment is targeted to specific tissues, such as ovaries, melanocytes, lung tissues, and the like. This tissue specific targeting may be useful in avoiding unintended side effects of a therapy that may regulate NPRA and/or NPRC expression, as presented in the examples.

DNA, RNA, or plasmid sequences may be complexed with chitosan particles or derivatives of chitosan particles for effectively delivering siRNA or other plasmid sequences to effectively inhibit expression of NPRA or NPRC or to stimulate expression of ANP, for example. This permits many effective pathways for delivery of these plasmids including transdermal, intranasal, and intravaginal, for example. This represents a significant improvement in the delivery of plasmids and siRNA to tumor cells.

The examples presented, including a B16 melanoma model and an ovarian cancer model, show that siNPRA nanoparticles are delivered to cancer cells, cause a significant reduction in tumors (i.e. compared to mice given scrambled siNPRA as a control), and provide an effective therapeutic treatment, which has not been achieved by administering ANP as an intravenous drug (probably due to its short half life in the body). An effective amount of siNPRA was delivered to mice injected with ovarian cancer cells. Growth of the tumor xenograft is inhibited significantly in these mice. Treatment with this effective amount of siNPRA was not as complete as seen in NPRA−/− mice, which are NPRA deficient. It is believed, without being limiting in any way, that a larger dose of siNPRA will provide even better results than those reported in the examples. Nonetheless, a person of ordinary skill in the art will be able to design effective therapies for NPRA inhibition and ANP expression using the examples provided. It is believed, without being limiting, that these therapies are applicable to a large number of cell proliferation disorders using the variety of pathways provided as examples herein.

For example, pNP73-102 inhibits NPRA expression in targeted cells. Lung cancer is effectively treated using a chitosan nanoparticle-based intranasal gene therapy. A549 cells injected into BALB/c nude mice induced lung micrometastasis in the control mice but not in pNP73-102-treated mice. Location of lung tumors is indicated by cyclin-B and phospho-BAD biomarkers and agrees with tissue staining data. An example of spontaneous lung tumorigenesis was induced with Line-1 cells in immunocompetent BALB/c mice. This example shows that a therapeutic treatment with pNP73-102, using pVAX as a plasmid carrier, significantly reduces tumors compared to those observed after treatment with a pVAX vector, alone. Delivery of pNP73-102 is an effective therapy for the treatment of lung cancers. It is shown that pNP73-102 decreases significantly the expression of NPRA, and it is thought that this mechanism explains its anti-tumor effect. By combining the examples and delivery methods presented, pNP73-102 may be used to therapeutically treat a wide variety of cell proliferation disorders treatable by inducing apoptosis in target cancer cells, for example.

Localized inflammation involving pro-inflammatory transcription factors such as NFκB has been implicated in the development of cancers. See Karin M. Mitogen activated protein kinases as targets for development of novel anti-inflammatory drugs, and Ann Rheum Dis 2004; 63 Suppl 2:ii 62-64. However, effective therapies have never been presented. NFκB is linked for colon and intenstinal cancers. See Greten F R, Eckmann L, Greten T F, et al. IKK beta and Cell 2004; 118:285-96. NFκB is reported to be a tumor promoter in the liver and in inflammation-associated cancers. See Pikarsky E, Porat R M, Stein I, et al. and Nature 2004; 431:461-6. NFκB is linked to enhancing tumor development in mammary cancers, primarily in the late stages of tumorigenesis. See Massion P P, Carbone D P, “the molecular basis of lung cancer: molecular abnormalities and therapeutic implications,” Respir Res 2003; 4:12. While many tumor cell lines show constitutive activation of NFκB, there has been conflicting evidence as to whether it promotes or inhibits tumorigenesis, and no effective therapy has been based on NFκB regulation. It is believed, without being limiting in any way, that activation of the NFκB pathway enhances tumor development in the late stages of tumorigenesis, and this belief is supported by mouse models of intestinal, liver and mammary cancer. It is thought that inhibition of NFκB signaling uniformly suppresses tumor development. Depending on the model studied, this salutary effect may be attributable to an increase in tumor cell apoptosis, reduced expression of tumor cell growth factors supplied by surrounding stromal cells, or abrogation of a tumor cell dedifferentiation program that is critical for tumor invasion/metastasis. For example, see Ahn K S, Sethi G, Aggarwal B B, “Simvastatin potentiates TNF-alpha-induced apoptosis through the down-regulation of NF-kappaB-dependent antiapoptotic gene products: role of IkappaBalpha kinase and TGF-beta-activated kinase-1,” J Immunol 2007; 178:2507-16; Ashworth T, Roy A L, “Cutting Edge: TFII-I controls B cell proliferation via regulating NF-kappaB,” J Immunol 2007; 178:2631-5; and Inoue J, Gohda J, Akiyama T, Semba K, “NF-kappaB activation in development and progression of cancer,” Cancer Sci 2007; 98:268-74; Kim S, Millet I, Kim H S, Kim J Y, et al., “NF-kappa B prevents beta cell death and autoimmune diabetes in NOD mice,” Proc Natl Acad Sci USA 2007; 104:1913-8; Oka D, Nishimura K, Shiba M, et al., “Sesquiterpene lactone parthenolide suppresses tumor growth in a xenograft model of renal cell carcinoma by inhibiting the activation of NF-kappaB,” Int. J Cancer 2007; 120:2576-81; Saccani A, Schioppa T, Porta C, et al., “p50 nuclear factor-kappaB over expression in tumor-associated macrophages inhibits Ml inflammatory responses and antitumor resistance,” Cancer Res 2006; 66:11432-40; Vilimas T, Mascarenhas J, Palomero T, et al., “Targeting the NF-kappaB signaling pathway in Notch1-induced T-cell leukemia.” Nat Med 2007; 13:70-7; and Schmidt D, Textor B, Pein O T, et al., “Critical role for NF-kappaB-induced JunB in VEGF regulation and tumor angiogenesis,” Embo J 2007; 26:710-9. This is an active area of cancer research, but therapies based on this discovery are not living up to promosing laboratory results.

It has now been demonstrated that effective delivery of pNP73-102 using the methods disclosed herein inhibits activation of NFκB. Furthermore, NFκB activation is reduced in the lungs of NPRA^{-/-} mice (i.e. NPRA deficient). Therefore, reducing NFκB activation using the methods provided herein is a method of therapeutically treating cancer, for example. Moreover, observations are presented that show less lung inflammation in NPRA^{-/-} mice than was observed in wild type counterparts when they were challenged by OVA in an asthma model. Thus, these methods are effective therapies for asthma, as well.

Interestingly, NFκB binding activity was 4-fold greater in the nuclear extracts of NPRA^{-/-} mouse hearts than in those of wild type mouse hearts. See Vellaichamy E, Sommana N K, Pandey K N, “Reduced cGMP signaling activates NF-kappaB in hypertrophied hearts of mice lacking natriuretic peptide receptor-A,” Biochem Biophys Res Commun 2005; 327:106-11. This reported observation contraindicates the down regulation of NPRA for inactivating NFκB as a method of therapeutically treating cancer. Reduced inflammation was reported in the hearts of NPRA^{-/-} mice by Oliveira A M, Ross J S, Fletcher J A, “Tumor suppressor genes in breast cancer: the gatekeepers and the caretakers,” Am J Clin Pathol 2005; 124 Suppl: S16-28. However, no effective therapy has been disclosed and no link was made to inhibiting NFκB, previously.

Examples showing expression of tumor suppressor genes, including p53 and pRb, provide evidence of broad effectiveness of the examples in therapeutic treatment of a wide variety of cancers. It is thought, without being limiting in any way, that tumor suppressor genes participate in a variety of critical and highly conserved cell functions, including regulation of the cell cycle and apoptosis, differentiation, surveillance of genomic integrity and repair of DNA errors, signal transduction, and cell adhesion. The p53 gene is the best known, but other tumor suppressor genes of interest include the retinoblastoma gene (pRb), PTEN, p16, nm23, and maspin. See Oliveira A M, Ross J S, Fletcher J A, “Tumor suppressor genes in breast cancer: the gatekeepers and the caretakers,” Am J Clin Pathol 2005; 124 Suppl: S16-28. There was no significant difference in the level of p53 in the lungs of NPRA^{-/-} and wild type mice; however, the phosphorylation of pRb was upregulated in the lungs of NPRA^{-/-} mice, as indicated by Western blot assays. It is thought that pRb plays a critical role in the control of cell proliferation and in DNA damage checkpoints and inhibits cell cycle progression through interactions with the E2F family of transcription factors. In tumorigenesis, loss of Rb function is an important event caused by gene mutation, promoter hypermethylation, deregulation of Rb phosphorylation and viral protein sequestration. Dysfunctional pRb has been reported in many different types of tumors, including those of the eye, bone, lung, breast and genitourinary system. In our investigation, NPRA deficiency did not affect pRb expression but did upregulate pRb phosphorylation.

It is thought that the Rb gene family is also involved in tumor angiogenesis. See Gabellini C, Del Bufalo D, Zupi G, “Involvement of RB gene family in tumor angiogenesis,” Oncogene 2006; 25:5326-32. Angiogenesis represents a fundamental step in tumor progression and metastasis. The induction of vasculature is important for tumor growth because it ensures an adequate supply of oxygen and metabolites to the tumor. It is thought that pRb regulates the expression of pro- and anti-angiogenic factors, such as the vascular endothelial growth factor (VEGF), through an E2F-dependent mechanism. Some natural and synthetic compounds demonstrate their anti-angiogenic activity through a mechanism of action involving pRb. Consistent with the activation of pRb in the lungs of NPRA^{-/-} mice, the expression of VEGF was down regulated in NPRA^{-/-} mice when compared to that in wild type mice. This indicates that angiogenesis is attenuated in NPRA^{-/-} mice, which surely contributes to observed suppression of tumor growth in NPRA^{-/-} mice. Additional studies are underway to determine which of the several signal transduction pathways in which NPRA is involved are important for the anti-tumor effect. See Gabellini C, Del Bufalo D, Zupi G, “Involvement of RB gene family in tumor angiogenesis,” Oncogene 2006; 25:5326-32.

Clinical studies of the natriuretic peptides have not indicated any incompatibility reactions or toxic effects. See Fluge, T, Forssmann W G, Kunkel G, et al., “Bronchodilation using combined urodilatin-albuterol administration in asthma: a randomized, double-blind, placebo-controlled trial,” Eur J Med Res 1999; 4:411-5. Accordingly, combining the advantage of chitosan nanoparticles in targeted delivery of anti-cancer drugs with gene therapy, such as delivery of pNP73-102 or siNPRA, poses a safe and effective treatment for a wide range of cancers.

### Materials and Methods

Cell lines. The mouse Lewis lung carcinoma LLC1 cell line, B16F10.9 melanoma cells, the type II alveolar epithelial adenocarcinoma cell line A549, and the normal human lung fibroblast cell line IMR 90 were purchased from ATCC (Rockville, Md.). Human Prostate cancer cells PC3 and DU145 and mouse ovarian cancer cell line, ID8, were also used. (kindly provided by Dr. Wenlong Bai in the University of South Florida; mouse ovarian cancer cell line, ID8, kindly provided by Dr. Janat-Amsbury at the Baylor College of Medicine.) Both A549 and IMR 90 were grown in Earle's modified Eagle's medium (EMEM) supplemented with 10% fetal bovine serum at 37° C. in a 5% CO₂ incubator. LLC1, ID8 and B16F10.9 cells were grown in Dulbecco's modified Eagle's medium (DMEM) supplemented with 10% fetal bovine serum.

### Animals

Female 8-10 week old BALB/c mice were purchased from Jackson Laboratory (Bar Harbor, Me.). Female nude mice and C57BL/6 mice were from NCI (National Cancer Institute). C57BL/6 NPRA^{-/-} (deficient in natriuretic peptide receptor A) mice were kindly provided by Dr. William Gower (VA Hospital Medical Center, Tampa, Fla.). All mice were maintained in a pathogen-free environment and all procedures were reviewed and approved by the University of South Florida Institutional Animal Care and Use Committee.

### Plasmid Constructs and Transfection

All plasmids used in this study were constructed using the pVAX expression vector (Invitrogen, CA). The pNP73-102 plasmid encodes the natriuretic peptide sequence, amino acids 73 to 102, of the atrial natriuretic prohormone N-terminal fragment. In some experiments the NP73-102 sequence was fused to the FLAG sequence to allow antibody detection of NP73-120 expression in lung sections. An anti-NPRA small interfering RNA plasmid (siNPRA) was constructed as previously described. A549 cells were transfected with plasmids using Lipofectamine 2000 (Invitrogen, CA) according to manufacturer's instructions.

### Preparation of Plasmid Nanoparticles and Administration to Mice

Plasmids pNP73-102 and pVAX1 were encapsulated in chitosan nanoparticles (25 μg of plasmid plus 125 μg of chitosan). Plasmids dissolved in 25 mM Na₂SO₄ and chitosan (Vanson, Redmond, Wash.) dissolved in 25 mM Na acetate (pH 5.4, final concentration 0.02%) were heated separately for 10 min at 55° C. After heating, the chitosan and DNA were mixed, vortexed vigorously for 20-30 sec. and stored at room temperature until use. Plasmid nanoparticles were given to lightly anesthetized mice in the form of nose drops in a volume of 50 μl using a pipetter with the tip inserted into the nostril.

Injection of Mice with Tumor Cells

For subcutaneous challenge with LLC1, ID8 and B16F10.9 cells, cells were grown in DMEM and washed with PBS and then resuspended in PBS at 2×10⁷ cells per ml for both LLC1 and ID8 or at 3×10⁶ cells per ml for B16F10.9. Two groups of mice (n=8 or 12 per group) were tested: wild type C57BL/6 and C57BL/6 NPRA-deficient mice. Animals were injected subcutaneously with 100 μl of suspended cancer cells in the right flank. Tumor sizes were measured regularly and the tumors were removed and weighed at the end of experiment. For the A549/nude mouse model, two groups of nude mice (n=4 per group) were given 5×10⁶ A549 cells by intravenous injection and treated intranasally with 25 μg of pNP73-102 or pVAX1 control nanoparticles once a week. Three weeks later, mice were sacrificed and lung sections were stained with hematoxylin and eosin and examined for tumor nodules. Lung sections were also stained with antibodies to cyclin B and phospho-Bad.

For the Line-1/BALB/c mouse model, 25 μg of pNP73-102 or pVAX1 control nanoparticles was injected intraperitoneally into two groups of BALB/c mice (n=4 per group) on days 1 and 3. A week later, these mice were injected subcutaneously with 10⁵ Line-1 lung adenocarcinoma cells in the right flanks Additional treatment with pNP73-102 or pVAX1 nanoparticles was continued at weekly intervals from week 2. A third group of four mice received only Line-1 cells as control. In each set of experiments, the mice were sacrificed on day 40 and their tumor burden was determined based on tumor size (measured by digital caliper) and weight.

### Western Blots

A549 cells were harvested and resuspended in lysis buffer containing 50 mM HEPES, 150 mM NaCl, 1 mM EDTA, 1 mM EGTA, 10% glycerol, 0.5% NP-40, 0.1 mM phenylmethylsulfonyl fluoride, 2.5 μg/ml leupeptin, 0.5 mM NaF, and 0.1 mM sodium vanadate to extract whole cell protein. Fifty μg of protein was separated by sodium dodecylsulfate polyacrylamide gel electrophoresis (SDS-PAGE) on a 10% polyacrylamide gel and transferred onto nitrocellulose membranes. Western immunoblots were performed according to the manufacturer's instructions (Cell Signaling Technology). Antibodies against NFκB p65, phosphorylated NFκB p65 (Ser536) and phosphorylated pRb were purchased from Cell Signaling, MA; antibodies against VEGF or NPRA were ordered from Santa Cruz, CA.

Knockdown of NPRA Expression with siNPRA

Small interfering RNA (siRNA) constructs that targeted the NPRA transcript (siNPRA) were prepared and tested for effectiveness by immunoblot for NPRA levels in cells transfected with a siNPRA plasmid (psiNPRA). A siNPRA9 construct is selected for anti-tumorigenesis examples, for example. B16 melanoma cells (1.5×10⁵) were injected subcutaneously (s.c.) into twelve-week old female C57BL/6 mice. The mice were then given intranasal suspensions of 33 μg of siNPRA oligos, siNPRA plasmid, or scrambled oligos encapsulated in chitosan nanoparticles at a ratio of 1:2.5. In experiments to determine the efficacy of topical application of siNPRA, chitosan nanoparticles containing siNPRA plasmid or oligos are mixed with transdermal cream and are applied to the injection area. Transdermal cream may be any transdermal cream, such as imiquimod cream sold by 3M Pharmaceuticals, Northridge, CA. Imiquimod cream containing siNPRA nanoparticles was applied twice a week and the control group received only imiquimod cream without nanoparticles and psiNPRA. Mice were sacrificed on day 22 and tumors were removed and weighed for comparison.

### Apoptosis Assays

A549 or normal IMR90 cells were grown in 6-well plates and transfected with pVAX1 or pNP73-102. Forty-eight hours after transfection, cells were examined for apoptosis by Terminal transferase dUTP nick end labeling (TUNEL) assay, and poly-ADP ribose polymerase (PARP)-cleavage by Western blotting. In the TUNEL assay, cell nuclei were stained with DAPI (diaminopimelimidate) to enable counting of total cell numbers and determination of the percentage of TUNEL-positive cells. For the PARP cleavage, whole-cell protein was isolated and equal amounts were western-blotted using an antibody to PARP. Experiments were done in duplicate.

### Construction of ANP Expression Vector

Total RNA was isolated from murine heart using Trizol reagent (LIFE TECHNOLOGY, Gaithersburg, Md.) following the manufacturer s protocol. The cDNA sequence for the ANP, residues 99-126 of pro ANP was amplified by R T-PCRA translation initiation codon was inserted in the forward primers, so that the recombinant peptides had an additional amino acid, methionine, as the first amino acid apart from its known content. The product was cloned in p VAX 25 vector (INVITROGEN, Carlsbad, Calif.) at HindIII and XhoI sites. The cloned ANP sequence was verified by DNA sequencing and its expression was checked in A549 human epithelial cells.

### Analysis of Intracellular Cytokine Production in T Cells

Mouse spleen T cells purified using mouse T-cell enrichment column kit (R & D Systems, Minneapolis, Minn.) were cultured in 6-well plates for 4 days. Finally, cells were stimulated with PMA (50 ng/ml) and ionomycin (500 ng/ml) (SIGMA, Saint Louis, Mo.) for 6 hours in the presence of GOLGISTOP (PHARMINGEN, San Diego, Calif.) and then fixed and stained using CD8 or CD4 mAb (BD BIOSCIENCES, San Diego, Calif.) for flow cytometry analysis.

Natriuretic Peptide Expression Plasmids and siNPRA Construct

The cDNAs encoding ANP, VD and NP73-102 were cloned into the mammalian expression vector pVAX (Clontech, Palo Alto, Calif.), respectively, using standard molecular biology procedures. The pANP plasmid encodes the human atrial natriuretic peptide consisting of the amino acids 98 to 126 of the C-terminal portion of the prohormone. The novel natriuretic peptide, NP73-102, was derived from the N-terminal part of the natriuretic peptide prohormone, amino acids 73 to 102, which encompasses the naturally occurring human kaliuretic peptide (KP, amino acids 79-98). The plasmid encoding the FLAG protein was purchased from BD Bioscience (Palo Alto, Calif.). In order to block expression of NPRA, a plasmid encoding a small interfering RNA against the NPRA mRNA was constructed.

### Nanoparticle Complexation of Plasmids

We have developed a nanoparticle delivery system utilizing the polysaccharide chitosan that allows intranasal administration of peptides, plasmids, and drugs. Protection of the natriuretic peptide expression plasmids from nuclease degradation and delivery to cells was achieved by complex coacervation of the DNA with chitosan (33 kDa) at a chitosan:DNA ratio of 3:1 (weight:weight) and vortexed for 20 min. Coacervates were allowed to stand 30 min at room temperature and were used immediately after preparation.

Treatment of Mice with Plasmid Nanocomplexes

Mice were lightly anesthetized by isoflurane inhalation and freshly prepared chitosan-plasmid coacervates were administered either by intraperitoneal (i.p.) injection or intranasally (i.n.) as nose drops. The volume given per dose i.n. was 50 μl and contained 20 μg of plasmid. The dose for i.p. administration was 25 μg in a volume of 100 μl.

Regulation of Lung Inflammation by Chitosan Nanoparticles Containing Plasmids Expressing Natriuretic Peptides or siNPRA

Sixteen Balb/c mice were divided into four groups (n=4 per group). One group served as naïve control with no OVA sensitization or challenge and no siRNA nanoparticle treatment. The second group received Ova sensitization (50 μg OVA i.p. injected on day 1 and day 7) and OVA challenge (25 μg intranasally on day 18, 19, 20 and 21). Animals in the third group got OVA sensitization, Ova challenge and intranasal treatment with natriuretic peptide nanoparticles or siNPRA (5 nmol of siNPRA or 20 μg of natriuretic peptide plasmids on day 18, 19, 20, and 21). The last group was OVA sensitized and challenged, but treated with control plasmid pVAX or scrambled siRNA (on day 18, 19, 20 and 21). All mice were sacrificed on day 22 to collect BAL fluid, and to remove lungs for lung pathology analysis by staining with hematoxylin and eosin (H & E). Mouse lungs were rinsed with intratracheal injections of PBS then perfused with 10% neutral buffered formalin. Lungs were removed, paraffin-embedded, sectioned at 20 μm and stained.

### Cell Enumeration of Bronchoalveolar Lavage Fluid

Bronchoalveolar lavage (BAL) fluid was collected and differential cell counts were performed as previously described [12]. Briefly, BAL was centrifuged and the cell pellet was suspended in 200 μl of PBS and counted using a hemocytometer. The cell suspensions were then centrifuged onto glass slides using a cytospin centrifuge at 1000 rpm for 5 min at room temperature. Cytocentrifuged cells were air dried and stained with a modified Wright's stain (Leukostat, Fisher Scientific, Atlanta, Ga.) which allows differential counting of monocytes and lymphocytes. At least 300 cells per sample were counted by direct microscopic observation.

### Determination of Airway Hyperreactivity (AHR)

AHR, expressed as enhanced pause (Penh), was measured in unrestrained mice by whole body plethysmography (Buxco, Troy, N.Y.). Groups of mice (n=4) were exposed for 5 min to nebulized PBS to establish a baseline then to increasing concentrations (6-25 mg/ml) of nebulized methacholine (MCh; Sigma, St. Louis, Mo.) in PBS. Challenges were done for 5 min followed by recordings of Penh for 5 min. The Penh values were averaged and expressed for each MCh concentration as a percentage of the PBS baseline reading.

### Detection of NP Receptors, NPRA and NPRC

NPRA was detected using a polyclonal antibody against a synthetic peptide sequence from the mouse NPRA receptor (Santa Cruz Biotech, Inc., Santa Cruz Calif.). Polyclonal antibody to mouse NPRA or NPRC (Santa Cruz Biotech. Inc., Santa Cruz Calif.) and measurement by flow cytometry (BD FACScan).

### Isolation and Culture of Splenocytes and Intracellular Cytokine Staining

Mice were euthanized by isoflurane inhalation, and spleens were removed into DMEM and held at 4° C. Spleens were macerated, passed through a cell strainer (40 micron; BD Bioscience, San Diego Calif.) and cells were collected by 10 min centrifugation at 4° C. and 700×g. Erythrocytes were removed by treating the spleen cell suspensions with ice cold buffer (ACK) containing 0.15 M NH₄C1, 1.0 mM KHCO₃ and 0.1 mM Na₂EDTA. Cells were counted by hemocytometer, and 10⁷ cells were seeded in 100 mm tissue culture dishes precoated with anti-CD3 in DMEM plus 10% FBS and cultured at 37° C. in 5% CO₂/air. The splenocytes were cultured for 24 h then brefeldin A was added (5 μg/ml) to block the secretion of cytokines Thymocytes were labelled with FITC-conjugated anti-CD4 or anti-CD8, then fixed and stained with PE-labeled Ab's to IL-4, IL-10 and IFN-γ and quantitated by flow cytometry using a FACS-Calibur (BD Biosciences). All antibodies were from BD-Biosciences (San Diego Calif.).

### Histological Analysis

Mouse lungs were removed after 24 hours of intranasal pANP administration, fixed, and sections stained with H&E.

### Statistics

The number of mice used in each test group was a minimum of 4 and usually 8 or 12. Experiments were repeated at least once and measurements were expressed as means plus or minus standard error of the mean or standard deviation. Comparisons of groups were done using a two-tailed Student's t test.

All patents, patent applications, provisional applications, and publications referred to or cited herein are incorporated by reference in their entirety, including all figures and tables, to the extent they are not inconsistent with the explicit teachings of this specification. It should be understood that the examples and embodiments described herein are for illustrative purposes only and that various modifications or changes in light thereof will be suggested to persons skilled in the art and are to be included within the spirit and purview of this application.

Alternative combinations and variations of the examples provided will become apparent based on this disclosure. It is not possible to provide specific examples for all of the many possible combinations and variations of the embodiments described, but such combinations and variations may be claims that eventually issue.

1-19. (canceled)

20. A method for treating respiratory syncytial virus (RSV) or asthma, comprising administering an interfering RNA specific for mRNA encoding natriuretic peptide receptor-A (NPR-A) in a mammalian subject having an RSV infection or asthma.

21. The method of claim 20, wherein the subject has the RSV infection.

22. The method of claim 20, wherein the subject has asthma.

23. The method of claim 20, wherein the interfering RNA is a small interfering RNA (siRNA) or a polynucleotide encoding an siRNA.

24. The method of claim 20, wherein the interfering RNA is complexed with a chitosan or chitosan derivative as a nanoparticle.

25. The method of claim 20, wherein the interfering RNA is administered intranasally or by inhalation.

26. The method of claim 20, wherein the interfering is administered transdermally.

27. The method of claim 20, wherein the subject is human.

28. A method for delivery of interfering RNA, comprising transdermally administering a composition to a mammalian subject, wherein the composition comprises an interfering RNA specific for a target mRNA in the subject; and imiquimod.

29. The method of claim 28, wherein the interfering RNA is a small interfering RNA (siRNA) or a polynucleotide encoding an siRNA.

30. The method of claim 28, wherein the composition is an ointment, salve, gel, or cream.

31. The method of claim 28, wherein the target mRNA encodes natriuretic peptide receptor-A (NPR-A) in the subject.

32. The method of claim 28, wherein the composition further comprises chitosan or a chitosan derivative, and the interfering RNA is complexed with the chitosan or chitosan derivative as a nanoparticle.

33. The method of claim 28, wherein the subject has pulmonary inflammation.

34. The method of claim 28, wherein the interfering RNA reduces lung inflammation and eosinophilia in the subject.

35. The method of claim 28, wherein the subject has cancer.

36. The method of claim 35, wherein the cancer is melanoma.

37. The method of claim 28, wherein the subject is human.
